(19) United States
(12) Patent Application Publication
(10) Pub. No.: US 2019/0389843 A1
(43) Pub. Date: Dec. 26, 2019

(54) PYRROLE mTORC INHIBITORS AND USES THEREOF

(71) Applicant: Navitor Pharmaceuticals, Inc., Cambridge, MA (US)

(72) Inventors: David John O'Neill, Arlington, MA (US); Eddine Saiah, Brookline, MA (US); Seong Woo Anthony Kang, Somerville, MA (US); Andrew Brearley, Oxfordshire (GB); Jonathan Bentley, Oxfordshire (GB)

(73) Assignee: Navitor Pharmaceuticals, Inc., Cambridge, MA (US)

(21) Appl. No.: 16/348,198

(22) PCT Filed: Nov. 8, 2017
(86) PCT No.: PCT/US17/60640

§ 371 Date: May 8, 2019

Related U.S. Application Data
(60) Provisional application No. 62/419,046, filed on Nov. 8, 2016.

(51) Int. Cl. C07D 403/14; C07D 401/14; C07D 413/14; C07D 487/04; C07D 487/10; C07D 405/14; C07D 487/08

(52) U.S. Cl. CPC C07D 403/14; C07D 401/14; C07D 413/14; C07D 487/08; C07D 487/10; C07D 405/14; C07D 487/04

(57) ABSTRACT

The present invention provides compounds, compositions thereof, and methods of using the same.

### TECHNICAL FIELD OF THE INVENTION

The present invention relates to compounds and methods useful for modulating mTORC1 activity. The invention also provides pharmaceutically acceptable compositions comprising provided compounds of the present invention and methods of using such compositions in the treatment of various disorders.

### BACKGROUND OF THE INVENTION

The mechanistic target of rapamycin (mTOR) signaling pathway integrates both intracellular and extracellular signals and serves as a central regulator of cell metabolism, growth, proliferation and survival. Discoveries that have been made over the last decade show that the mTOR pathway is activated during various cellular processes (e.g. tumor formation and angiogenesis, insulin resistance, adipogenesis and T-lymphocyte activation) and is deregulated in human diseases such as cancer and type 2 diabetes. These observations have attracted broad scientific and clinical interest in mTOR. This is highlighted by the growing use of mTOR inhibitors [rapamycin and its analogues (rapalogues)] in pathological settings, including the treatment of solid tumors, organ transplantation, coronary restenosis and rheumatoid arthritis.

In particular, mTOR complex 1 (mTORC1) positively regulates cell growth and proliferation by promoting many anabolic processes, including biosynthesis of proteins, lipids and organelles, and by limiting catabolic processes such as autophagy. Much of the knowledge about mTORC1 function comes from the use of the bacterial macrolide rapamycin. Upon entering the cell, rapamycin binds to FK506-binding protein of 12 kDa (FKBP12) and interacts with the FKBP12-rapamycin binding domain (FRB) of mTOR, thus inhibiting mTORC1 functions (reviewed by Guertin and Sabatini, 2007). In contrast to its effect on mTORC1, FKBP12-rapamycin cannot physically interact with or acutely inhibit mTOR complex 2 (mTORC2)(Jacinto et al., 2004; Sarbassov et al., 2004). On the basis of these observations, mTORC1 and mTORC2 have been respectively characterized as the rapamycin-sensitive and rapamycin-insensitive complexes. However, this paradigm might not be entirely accurate, as chronic rapamycin treatment can, in some cases, inhibit mTORC2 activity by blocking its assembly (Sarbassov et al., 2006). In addition, recent reports suggest that important mTORC1 functions are resistant to inhibition by rapamycin (Choo et al., 2008; Feldman et al., 2009; Garcia-Martinez et al., 2009; Thoreen et al., 2009). Therefore, selective inhibition of mTORC1 would enable the treatment of diseases that involve dysregulation of protein synthesis and cellular metabolism. Furthermore, this detailed understanding of regulating mTORC1 activation pathways will permit the discovery of new strategies for regulating abnormal disease processes by modulating mTORC1 activity across its spectrum of function.

Many diseases are associated with abnormal cellular responses triggered by events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases.

The mechanistic target of rapamycin complex 1 (mTORC1) is a master growth regulator that senses diverse environmental cues, such as growth factors, cellular stresses, and nutrient and energy levels. When activated, mTORC1 phosphorylates substrates that potentiate anabolic processes, such as mRNA translation and lipid synthesis, and limits catabolic ones, such as autophagy. mTORC1 dysregulation occurs in a broad spectrum of diseases, including diabetes, epilepsy, neurodegeneration, immune response, suppressed skeletal muscle growth, and cancer among others (Howell et al., (2013) Biochemical Society transactions 41, 906-912; Kim et al., (2013) Molecules and cells 35, 463-473; Laplante and Sabatini, (2012) Cell 149, 274-293). Accordingly, there remains a need to find protein kinase inhibitors useful as therapeutic agents.

Additionally, Glucose transporters (GLUT) are a family of membrane proteins (GLUT1, 2, 3, 4, and 5) that facilitate the transport of glucose and other hexoses across cell membranes. The transport of glucose into cells is one of the most important cellular transport events because of the role in maintaining normal cellular respiration and metabolism (Gould and Holman, (1993) Biochem J., 295, 329-341). Dysfunction or dysregulation of glucose transporters may contribute to, or directly result in, disease states because of the central role the transporters play in cellular homeostasis and metabolism. For example, mutations in the GLUT1 gene are responsible for the rare autosomal disorder De Vivo disease, which is characterized by impaired glucose transport into the brain. Relatedly, elevated levels of GLUT1 in neutrophils has been found to contribute to the inflammatory response in cystic fibrosis (CF) patients (Laval et al., (2013) J. Immunol, 190(12), 6043-50). GLUT inhibition may normalize cellular metabolism and response in affected cells, including immune cells such as neutrophils. Therefore, GLUT inhibition would enable the treatment of cystic fibrosis, as well as autoimmune diseases characterized by abnormal GLUT expression or activity.

### SUMMARY OF THE INVENTION

It has now been found that compounds of this invention, and pharmaceutically acceptable compositions thereof, are effective as mTORC1 inhibitors. Such compounds have the general Formula I:

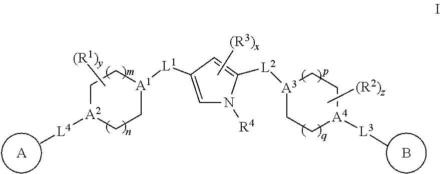

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with mTORC1. Such diseases, disorders, or conditions include those described herein.

### DETAILED DESCRIPTION OF CERTAIN EMBODIMENTS

### 1. General Description of Certain Embodiments of the Invention:

In certain embodiments, the present invention provides a compound of Formula I:

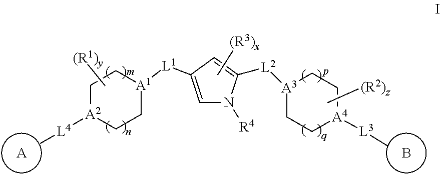

or a pharmaceutically acceptable salt thereof, wherein:
A¹ is N or CH;
A² is N or CR′, provided at least one of A¹ and A² comprises a nitrogen;
A³ is N or CH;
A⁴ is N or CH;
R′ is H or C₁₋₆ aliphatic; or
   R′ and L⁴ are optionally taken together with their intervening atoms to form a 5-8 membered saturated spiro-fused ring having 0-2 heteroatoms independently selected from nitrogen, oxygen or sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each of m, n, p, q, and x is independently 0, 1, or 2;
each of y and z is independently 0, 1, 2, 3, or 4;
each of R¹ and R² is independently R, or:
   two R¹ groups are optionally taken together to form ═O;
   two R² groups are optionally taken together to form ═O;
   two R¹ groups are optionally taken together to form a covalent bond or a bivalent C₁₋₄ alkylene chain;
   two R² groups are optionally taken together to form a covalent bond or a bivalent C₁₋₄ alkylene chain;
   an R¹ group and Ring A are optionally taken together with their intervening atoms to form a 5-8 membered aryl fused ring having 0-2 heteroatoms independently selected from nitrogen, oxygen or sulfur;
   an R² group and Ring B are optionally taken together with their intervening atoms to form a 5-8 membered aryl fused ring having 0-2 heteroatoms independently selected from nitrogen, oxygen or sulfur; or
   an R² group and L³ are optionally taken together with their intervening atoms to form a 5-8 membered aryl fused ring having 0-2 heteroatoms independently selected from nitrogen, oxygen or sulfur;
each of R³ is independently hydrogen, C₁₋₆ aliphatic, or —CN;
R⁴ is hydrogen or an optionally substituted C₁₋₆ aliphatic group;
Ring A is an optionally substituted ring selected from 6-membered aryl containing 0-2 nitrogen atoms, 5-membered heteroaryl with 1-4 heteroatoms independently selected from nitrogen, oxygen or sulfur, or 8-10 membered bicyclic aryl or heteroaryl with 1-4 heteroatoms independently selected from nitrogen, oxygen or sulfur;
Ring B is an optionally substituted ring selected from 6-membered aryl containing 0-2 nitrogen atoms, 5-membered heteroaryl with 1-4 heteroatoms independently selected from nitrogen, oxygen or sulfur, or 8-10 membered bicyclic aryl or heteroaryl with 1-4 heteroatoms independently selected from nitrogen, oxygen or sulfur;
L¹ is a covalent bond or a C₁₋₃ bivalent straight or branched saturated or unsaturated hydrocarbon chain wherein 1-2 methylene units of the chain are independently and optionally replaced with —C(O)—, —N(R)—, or —CH(R)—;
L² is a covalent bond or a C₁₋₃ bivalent straight or branched saturated or unsaturated hydrocarbon chain wherein 1-2 methylene units of the chain are independently and optionally replaced with —C(O)— or —CH(R)—;
L³ is a covalent bond or —N(R)—; and
L⁴ is a covalent bond or —N(R)—.

### 2. Compounds and Definitions:

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, di azepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘}C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘}S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched)alkylene)O—N(R^{∘})₂; or —(C₁₋₄ straight or branched) alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

The terms “measurable affinity” and “measurably inhibit,” as used herein, means a measurable change in mTORC1 activity between a sample comprising a compound of the present invention, or composition thereof, and mTORC1, and an equivalent sample comprising mTORC1 in the absence of said compound, or composition thereof

### 3. Description of Exemplary Embodiments:

As described above, in certain embodiments, the present invention provides a compound of Formula I:

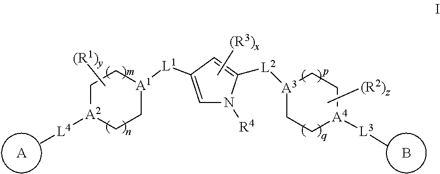

or a pharmaceutically acceptable salt thereof, wherein:
A¹ is N or CH;
A² is N or CR′, provided at least one of A¹ and A² comprises a nitrogen;
A³ is N or CH;
A⁴ is N or CH;
R′ is H or C₁₋₆ aliphatic; or
   R′ and L⁴ are optionally taken together with their intervening atoms to form a 5-8 membered saturated spiro-fused ring having 0-2 heteroatoms independently selected from nitrogen, oxygen or sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each of m, n, p, q, and x is independently 0, 1, or 2;
each of y and z is independently 0, 1, 2, 3, or 4;
each of R¹ and R² is independently R, or:
   two R¹ groups are optionally taken together to form ═O;
   two R² groups are optionally taken together to form ═O;
   two R¹ groups are optionally taken together to form a covalent bond or a bivalent C₁₋₄ alkylene chain;
   two R² groups are optionally taken together to form a covalent bond or a bivalent C₁₋₄ alkylene chain;
   an R¹ group and Ring A are optionally taken together with their intervening atoms to form a 5-8 membered aryl fused ring having 0-2 heteroatoms independently selected from nitrogen, oxygen or sulfur;
   an R² group and Ring B are optionally taken together with their intervening atoms to form a 5-8 membered aryl fused ring having 0-2 heteroatoms independently selected from nitrogen, oxygen or sulfur; or
   an R² group and L³ are optionally taken together with their intervening atoms to form a 5-8 membered aryl fused ring having 0-2 heteroatoms independently selected from nitrogen, oxygen or sulfur;
each of R³ is independently hydrogen, C₁₋₆ aliphatic, or —CN;
R⁴ is hydrogen or an optionally substituted C₁₋₆ aliphatic group;
Ring A is an optionally substituted ring selected from 6-membered aryl containing 0-2 nitrogen atoms, 5-membered heteroaryl with 1-4 heteroatoms independently selected from nitrogen, oxygen or sulfur, or 8-10 membered bicyclic aryl or heteroaryl with 1-4 heteroatoms independently selected from nitrogen, oxygen or sulfur;
Ring B is an optionally substituted ring selected from 6-membered aryl containing 0-2 nitrogen atoms, 5-membered heteroaryl with 1-4 heteroatoms independently selected from nitrogen, oxygen or sulfur, or 8-10 membered bicyclic aryl or heteroaryl with 1-4 heteroatoms independently selected from nitrogen, oxygen or sulfur;
L¹ is a covalent bond or a C₁₋₃ bivalent straight or branched saturated or unsaturated hydrocarbon chain wherein 1-2 methylene units of the chain are independently and optionally replaced with —C(O)—, —N(R)—, or —CH(R)—;
L² is a covalent bond or a C₁₋₃ bivalent straight or branched saturated or unsaturated hydrocarbon chain wherein 1-2 methylene units of the chain are independently and optionally replaced with —C(O)— or —CH(R)—;
L³ is a covalent bond or —N(R)—; and
L⁴ is a covalent bond or —N(R)—.

As defined above and described herein, A¹ is N or CH. In some embodiments, A¹ is N. In some embodiments, A¹ is CH.

In some embodiments, A¹ is selected from those depicted in Table 1, below.

As defined above and described herein, A² is N or CR′, provided at least one of A¹ and A¹ comprises a nitrogen. In some embodiments, A² is N. In some embodiments, A² is CR′. In some embodiments, at least one of A¹ and A² comprises a nitrogen.

In some embodiments, A² is selected from those depicted in Table 1, below.

As defined above and described herein, each of m, and n is independently 0, 1, or 2. In some embodiments, m is 0. In some embodiments, n is 0. In some embodiments, m is 1. In some embodiments, n is 1. In some embodiments, m is 2. In some embodiments, n is 2.

As defined above and described herein, each of p, q, and x is independently 0, 1, or 2. In some embodiments, p is 0. In some embodiments, q is 0. In some embodiments, x is 0. In some embodiments, p is 1. In some embodiments, q is 1. In some embodiments, x is 1. In some embodiments, p is 2. In some embodiments, q is 2. In some embodiments, x is 2.

As defined above and described herein, each of y and z is independently 0, 1, 2, 3, or 4. In some embodiments, y is 0. In some embodiments, z is 0. In some embodiments, y is 1. In some embodiments, z is 1. In some embodiments, y is 2. In some embodiments, z is 2. In some embodiments, y is 3. In some embodiments, z is 3. In some embodiments, y is 4. In some embodiments, z is 4.

As defined above and described herein, A³ is N or CH. In some embodiments, A³ is N. In some embodiments, A³ is CH.

In some embodiments, A³ is selected from those depicted in Table 1, below.

As defined above and described herein, A⁴ is N or CH. In some embodiments, A⁴ is N. In some embodiments, A⁴ is CH.

In some embodiments, A⁴ is selected from those depicted in Table 1, below.

As defined above and described herein, R′ is H or C₁₋₆ aliphatic, or R′ and L⁴ are optionally taken together with their intervening atoms to form a 5-8 membered saturated spiro-fused ring having 0-2 heteroatoms independently selected from nitrogen, oxygen or sulfur. In some embodiments, R′ is H. In some embodiments, R′ is C₁₋₆ aliphatic. In some embodiments, R′ and L⁴ are optionally taken together with their intervening atoms to form a 5-8 membered saturated spiro-fused ring having 0-2 heteroatoms independently selected from nitrogen, oxygen or sulfur. In some embodiments, R′ and L⁴ are optionally taken together with their intervening atoms to form a 5 membered saturated spiro-fused ring having one nitrogen.

In some embodiments, R′ is selected from those depicted in Table 1, below.

As defined above and described herein, each of R¹ and R² is independently R, or: two R¹ groups are optionally taken together to form ═O; two R² groups are optionally taken together to form ═O; two R¹ groups are optionally taken together to form a covalent bond or a bivalent C₁₋₄ alkylene chain; two R² groups are optionally taken together to form a covalent bond or a bivalent C₁₋₄ alkylene chain; an R¹ group and Ring A are optionally taken together with their intervening atoms to form a 5-8 membered fused ring having 0-2 heteroatoms independently selected from nitrogen, oxygen or sulfur; an R² group and Ring B are optionally taken together with their intervening atoms to form a 5-8 membered fused ring having 0-2 heteroatoms independently selected from nitrogen, oxygen or sulfur; or an R² group and L³ are optionally taken together with their intervening atoms to form a 5-8 membered aryl fused ring having 0-2 heteroatoms independently selected from nitrogen, oxygen or sulfur.

In some embodiments, each R¹ is hydrogen. In some embodiments R¹ is methyl. In some embodiments, two R¹ groups are optionally taken together to form ═O. In some embodiments, two R¹ groups are optionally taken together to form a covalent bond. In some embodiments, two R¹ groups are optionally taken together to form a bivalent C₁₋₄ alkylene chain. In some embodiments, an R¹ group and Ring A are optionally taken together with their intervening atoms to form a 5-8 membered fused ring having 0-2 heteroatoms independently selected from nitrogen, oxygen, or sulfur.

In some embodiments, each R² is hydrogen. In some embodiments, R² is methyl. In some embodiments, two R² groups are optionally taken together to form ═O. In some embodiments, two R² groups are optionally taken together to form a covalent bond. In some embodiments, two R² groups are optionally taken together to form a bivalent C₁₋₄ alkylene chain. In some embodiments, an R² group and Ring B are optionally taken together with their intervening atoms to form a 5-8 membered fused ring having 0-2 heteroatoms independently selected from nitrogen, oxygen, or sulfur. In some embodiments, an R² group and L³ are optionally taken together with their intervening atoms to form a 5-8 membered aryl fused ring having 0-2 heteroatoms independently selected from nitrogen, oxygen or sulfur.

In some embodiments, each of R¹ and R² is independently selected from those depicted in Table 1, below.

As defined above and described herein, each of R³ is independently hydrogen, C₁₋₆ aliphatic, or —CN. In some embodiments, R³ is hydrogen. In some embodiments, R³ is C₁₋₆ aliphatic. In some embodiments, R³ is —CN. In some embodiments, R³ is methyl.

In some embodiments, R³ is independently selected from those depicted in Table 1, below.

As defined above and described herein, R⁴ is hydrogen or an optionally substituted C₁₋₆ aliphatic group. In some embodiments, R⁴ is hydrogen. In some embodiments, R⁴ is n optionally substituted C₁₋₆ aliphatic group. In some embodiments, R⁴ is methyl. In some embodiments, R⁴ is ethyl. In some embodiments, R⁴ is butyl. In some embodiments, R⁴ is —CH₂CF₃. In some embodiments, R⁴ is

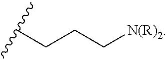

In some embodiments, R⁴ is

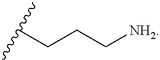 [SMILES: *CCCN]

In some embodiments, R⁴ is

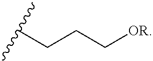

In some embodiments, R⁴ is

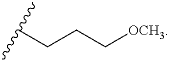 [SMILES: *CCCOC]

In some embodiments, R⁴ is

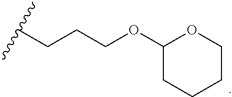 [SMILES: *CCCOC1CCCCO1]

In some embodiments, R⁴ is

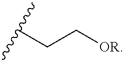

In some embodiments, R⁴ is

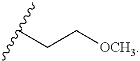 [SMILES: *CCOC]

In some embodiments, R⁴ is

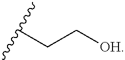 [SMILES: *CCO]

In some embodiments, R⁴ is

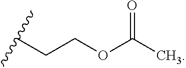 [SMILES: *CCOC(C)=O]

In some embodiments, R⁴ is is selected from those depicted in Table 1, below.

As defined above and described herein, Ring A is an optionally substituted ring selected from 6-membered aryl containing 0-2 nitrogen atoms, 5-membered heteroaryl with 1-4 heteroatoms independently selected from nitrogen, oxygen or sulfur, or 8-10 membered bicyclic aryl or heteroaryl with 1-4 heteroatoms independently selected from nitrogen, oxygen or sulfur.

In some embodiments, Ring A is phenyl. In some embodiments, Ring A is

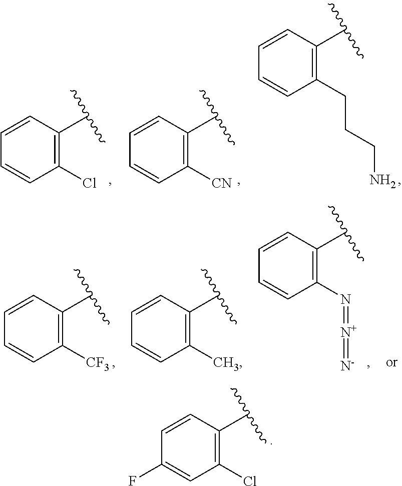

In some embodiments, Ring A is

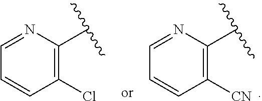 [SMILES: *c1ncccc1Cl ; *c1ncccc1C#N]

As defined above and described herein, Ring B is an optionally substituted ring selected from 6-membered aryl containing 0-2 nitrogen atoms, 5-membered heteroaryl with 1-4 heteroatoms independently selected from nitrogen, oxygen or sulfur, or 8-10 membered bicyclic aryl or heteroaryl with 1-4 heteroatoms independently selected from nitrogen, oxygen or sulfur.

In some embodiments, Ring B is phenyl, pyridyl, pyrimidinyl, pyrazinyl, pyridazinyl, or indolyl. In some embodiments, Ring B is

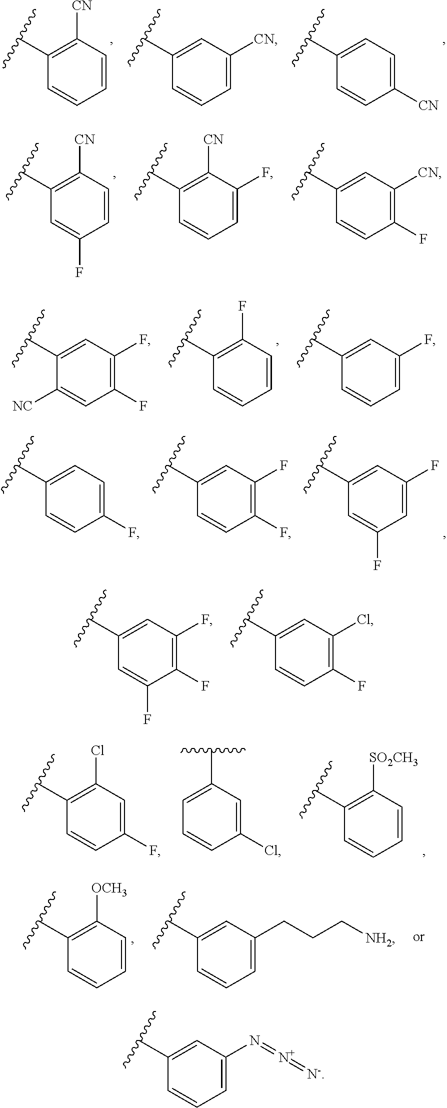

In some embodiments, Ring B is

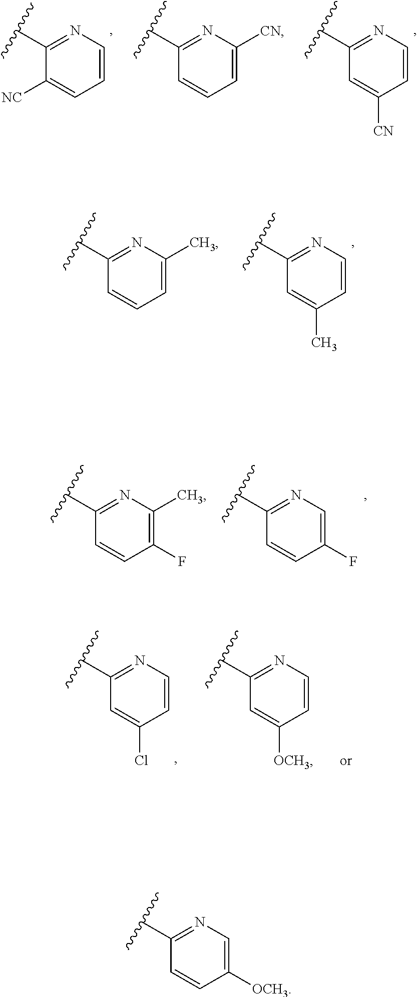

In some embodiments, Ring B is

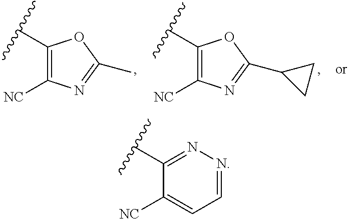

In some embodiments, Ring B is

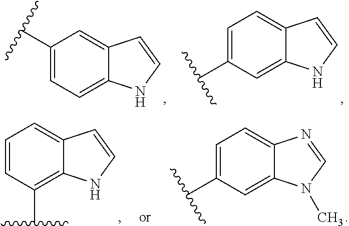

As defined above and described herein, L¹ is a covalent bond or a C₁₋₃ bivalent straight or branched saturated or unsaturated hydrocarbon chain wherein 1-2 methylene units of the chain are independently and optionally replaced with —C(O)—, —N(R)—, or —CH(R)—.

In some embodiments, L¹ is a covalent bond. In some embodiments, L¹ is —CH₂—. In some embodiments, L¹ is

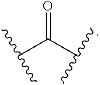 [SMILES: *C(*)=O]

In some embodiments, L¹ is

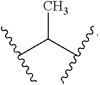 [SMILES: *C(*)C]

In some embodiments, L¹ is

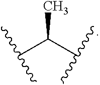

In some embodiments, L¹ is

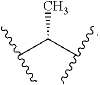

In some embodiments, L¹ is —CH₂NH—.

In some embodiments, L¹ is selected from those depicted in Table 1, below.

As defined above and described herein, L² is a covalent bond or a C₁₋₃ bivalent straight or branched saturated or unsaturated hydrocarbon chain wherein 1-2 methylene units of the chain are independently and optionally replaced with —C(O)— or —CH(R)—.

In some embodiments, L² is a covalent bond. In some embodiments, L² is —CH₂—. In some embodiments, L² is

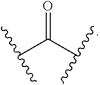 [SMILES: *C(*)=O]

In some embodiments, L² is selected from those depicted in Table 1, below.

As defined above and described herein, L³ is a covalent bond or —N(R)—.

In some embodiments, L³ is a covalent bond. In some embodiments, L³ is —N(R)—. In some embodiments, L³ is —NH—.

In some embodiments, L³ is selected from those depicted in Table 1, below.

As defined above and described herein, L⁴ is a covalent bond or —N(R)—.

In some embodiments, L⁴ is a covalent bond. In some embodiments, L⁴ is —N(R)—. In some embodiments, L⁴ is —NH—.

In some embodiments, L⁴ is selected from those depicted in Table 1, below.

In some embodiments, the present invention provides a compound of Formulae Ia, I-b, or I-c:

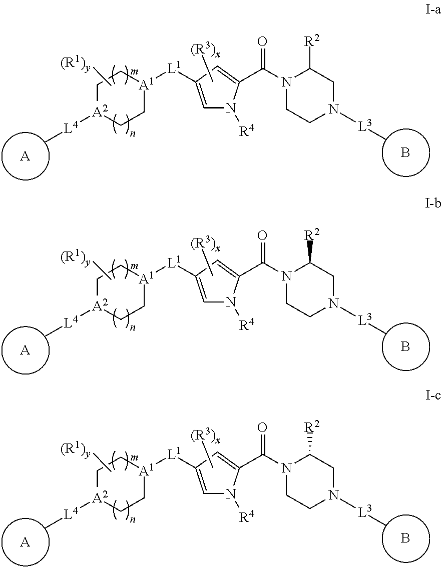

or a pharmaceutically acceptable salt thereof, wherein each of Ring A, R¹, R², R³, R⁴, L¹, L³, L⁴, A¹, A², Ring B, m, n, x, and y is defined above and described in embodiments herein.

In some embodiments, the present invention provides a compound of Formula I-d:

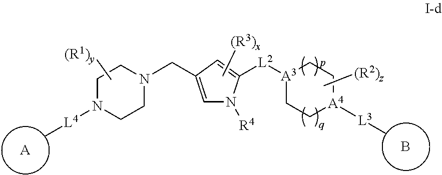

or a pharmaceutically acceptable salt thereof, wherein each of Ring A, A³, A⁴, R¹, R², R³, R⁴, L², L³, L⁴, Ring B, p, q, x, y, and z is defined above and described in embodiments herein.

Exemplary compounds of the invention are set forth in Table 1, below.

TABLE 1
Exemplary Compounds

| | |
|---|---|
| 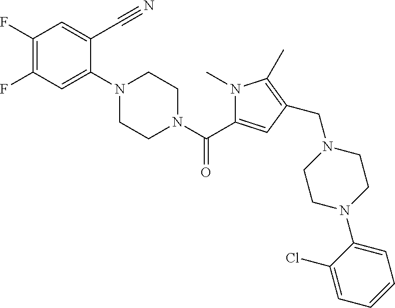 [SMILES: Cc1c(CN2CCN(c3ccccc3Cl)CC2)cc(C(=O)N2CCN(c3cc(F)c(F)cc3C#N)CC2)n1C] | I-1 |
| 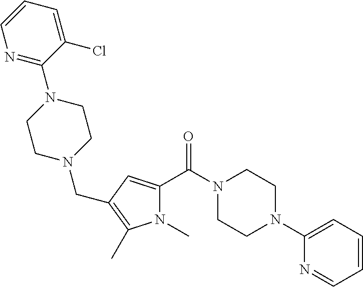 [SMILES: Cc1c(CN2CCN(c3ncccc3Cl)CC2)cc(C(=O)N2CCN(c3ccccn3)CC2)n1C] | I-2 |
| 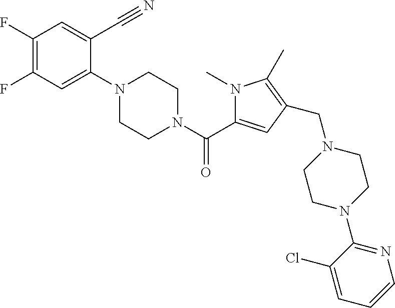 [SMILES: Cc1c(CN2CCN(c3ncccc3Cl)CC2)cc(C(=O)N2CCN(c3cc(F)c(F)cc3C#N)CC2)n1C] | I-3 |
| 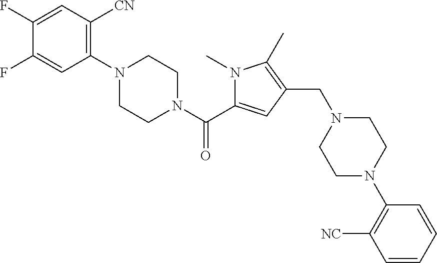 [SMILES: Cc1c(CN2CCN(c3ccccc3C#N)CC2)cc(C(=O)N2CCN(c3cc(F)c(F)cc3C#N)CC2)n1C] | I-4 |
| 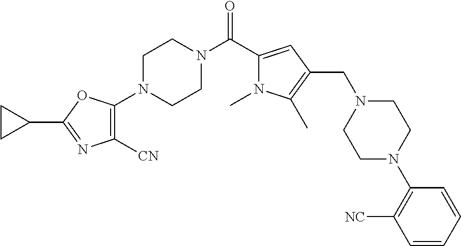 [SMILES: Cc1c(CN2CCN(c3ccccc3C#N)CC2)cc(C(=O)N2CCN(c3oc(C4CC4)nc3C#N)CC2)n1C] | I-14 |
| 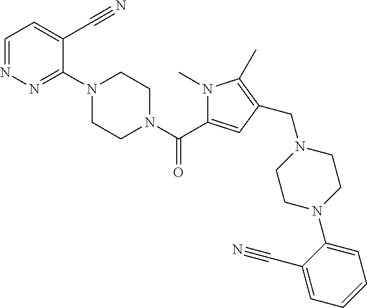 [SMILES: Cc1c(CN2CCN(c3ccccc3C#N)CC2)cc(C(=O)N2CCN(c3nnccc3C#N)CC2)n1C] | I-15 |
| 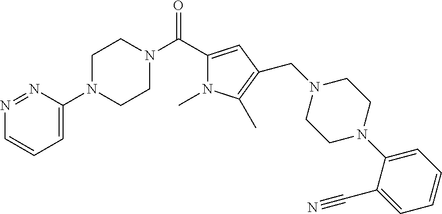 [SMILES: Cc1c(CN2CCN(c3ccccc3C#N)CC2)cc(C(=O)N2CCN(c3cccnn3)CC2)n1C] | I-16 |
| 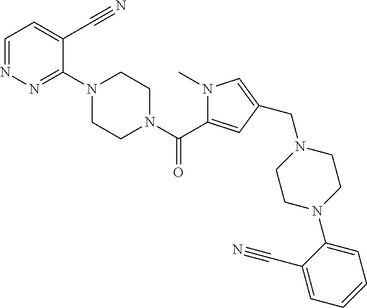 [SMILES: Cn1cc(CN2CCN(c3ccccc3C#N)CC2)cc1C(=O)N1CCN(c2nnccc2C#N)CC1] | I-17 |
| 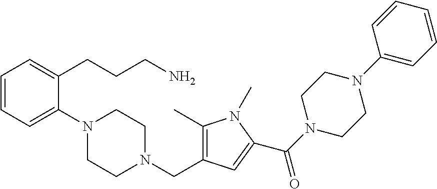 [SMILES: Cc1c(CN2CCN(c3ccccc3CCCN)CC2)cc(C(=O)N2CCN(c3ccccc3)CC2)n1C] | I-18 |
| 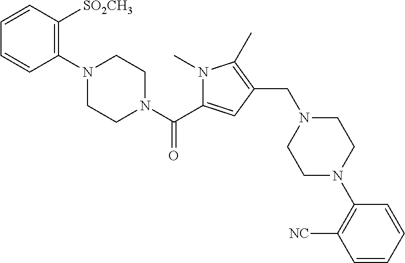 [SMILES: Cc1c(CN2CCN(c3ccccc3C#N)CC2)cc(C(=O)N2CCN(c3ccccc3S(C)(=O)=O)CC2)n1C] | I-21 |
| 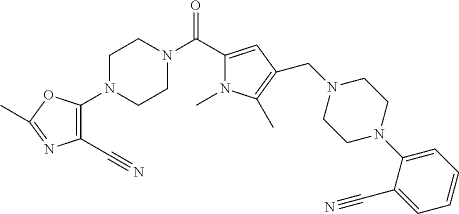 [SMILES: Cc1nc(C#N)c(N2CCN(C(=O)c3cc(CN4CCN(c5ccccc5C#N)CC4)c(C)n3C)CC2)o1] | I-22 |
| 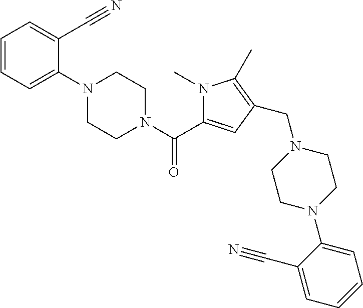 [SMILES: Cc1c(CN2CCN(c3ccccc3C#N)CC2)cc(C(=O)N2CCN(c3ccccc3C#N)CC2)n1C] | I-23 |
| 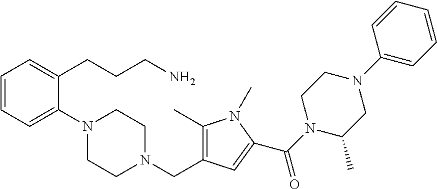 [SMILES: Cc1c(CN2CCN(c3ccccc3CCCN)CC2)cc(C(=O)N2CCN(c3ccccc3)C[C@@H]2C)n1C] | I-24 |
| 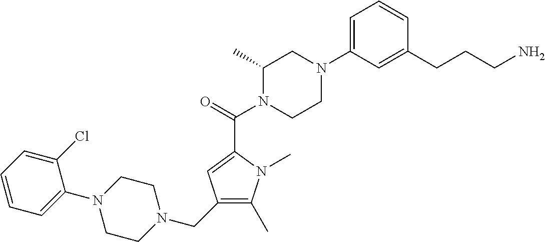 [SMILES: Cc1c(CN2CCN(c3ccccc3Cl)CC2)cc(C(=O)N2CCN(c3cccc(CCCN)c3)C[C@H]2C)n1C] | I-27 |
| 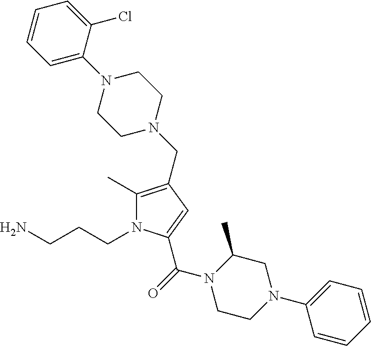 [SMILES: Cc1c(CN2CCN(c3ccccc3Cl)CC2)cc(C(=O)N2CCN(c3ccccc3)C[C@@H]2C)n1CCCN] | I-29 |
| 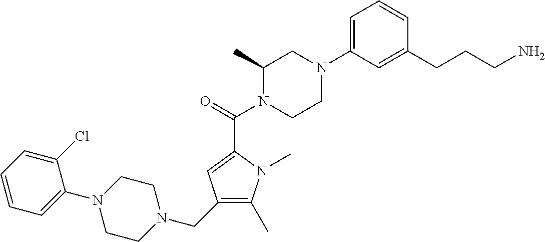 [SMILES: Cc1c(CN2CCN(c3ccccc3Cl)CC2)cc(C(=O)N2CCN(c3cccc(CCCN)c3)C[C@@H]2C)n1C] | I-30 |
| 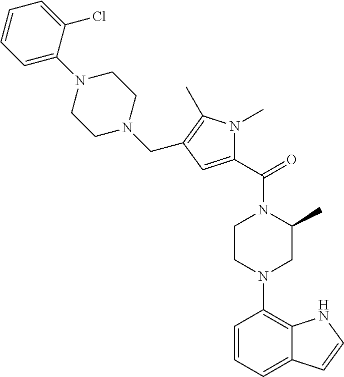 [SMILES: Cc1c(CN2CCN(c3ccccc3Cl)CC2)cc(C(=O)N2CCN(c3cccc4cc[nH]c34)C[C@@H]2C)n1C] | I-31 |
| 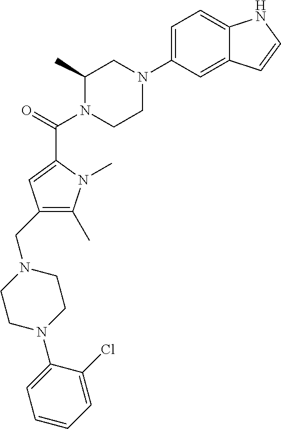 [SMILES: Cc1c(CN2CCN(c3ccccc3Cl)CC2)cc(C(=O)N2CCN(c3ccc4[nH]ccc4c3)C[C@@H]2C)n1C] | I-33 |
| 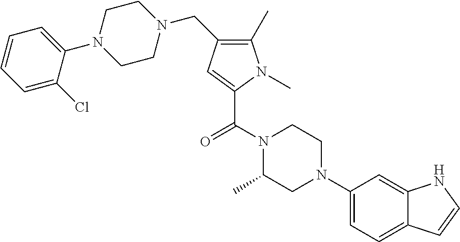 [SMILES: Cc1c(CN2CCN(c3ccccc3Cl)CC2)cc(C(=O)N2CCN(c3ccc4cc[nH]c4c3)C[C@@H]2C)n1C] | I-34 |
| 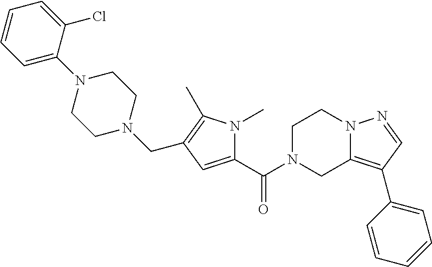 [SMILES: Cc1c(CN2CCN(c3ccccc3Cl)CC2)cc(C(=O)N2CCn3ncc(-c4ccccc4)c3C2)n1C] | I-35 |
| 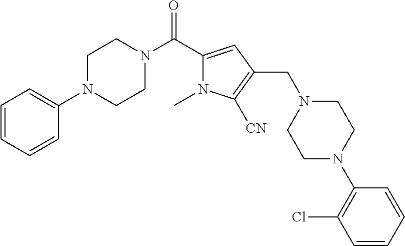 [SMILES: Cn1c(C(=O)N2CCN(c3ccccc3)CC2)cc(CN2CCN(c3ccccc3Cl)CC2)c1C#N] | I-36 |
| 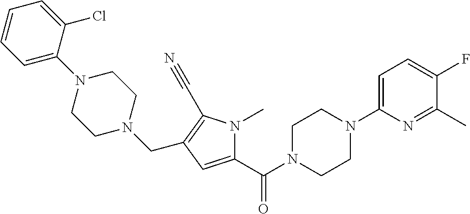 [SMILES: Cc1nc(N2CCN(C(=O)c3cc(CN4CCN(c5ccccc5Cl)CC4)c(C#N)n3C)CC2)ccc1F] | I-37 |
| 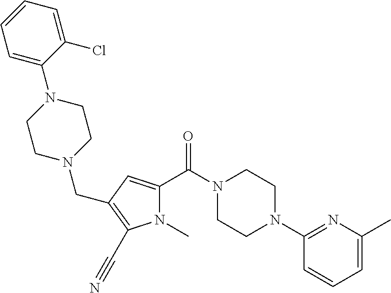 [SMILES: Cc1cccc(N2CCN(C(=O)c3cc(CN4CCN(c5ccccc5Cl)CC4)c(C#N)n3C)CC2)n1] | I-38 |
| 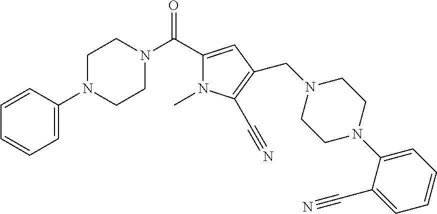 [SMILES: Cn1c(C(=O)N2CCN(c3ccccc3)CC2)cc(CN2CCN(c3ccccc3C#N)CC2)c1C#N] | I-39 |
| 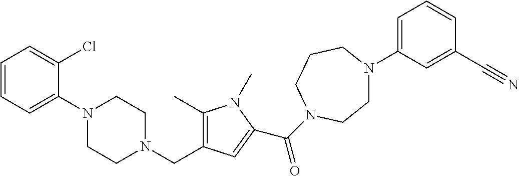 [SMILES: Cc1c(CN2CCN(c3ccccc3Cl)CC2)cc(C(=O)N2CCCN(c3cccc(C#N)c3)CC2)n1C] | I-40 |
| 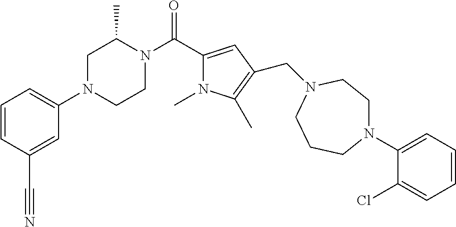 [SMILES: Cc1c(CN2CCCN(c3ccccc3Cl)CC2)cc(C(=O)N2CCN(c3cccc(C#N)c3)C[C@@H]2C)n1C] | I-41 |
| 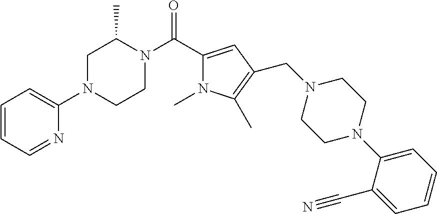 [SMILES: Cc1c(CN2CCN(c3ccccc3C#N)CC2)cc(C(=O)N2CCN(c3ccccn3)C[C@@H]2C)n1C] | I-42 |
| 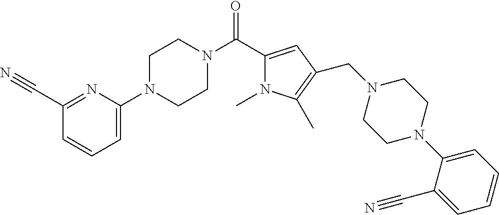 [SMILES: Cc1c(CN2CCN(c3ccccc3C#N)CC2)cc(C(=O)N2CCN(c3cccc(C#N)n3)CC2)n1C] | I-43 |
| 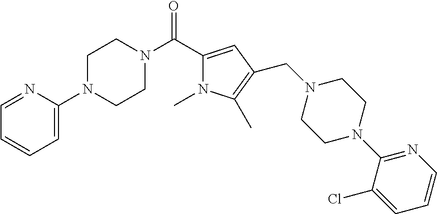 [SMILES: Cc1c(CN2CCN(c3ncccc3Cl)CC2)cc(C(=O)N2CCN(c3ccccn3)CC2)n1C] | I-44 |
| 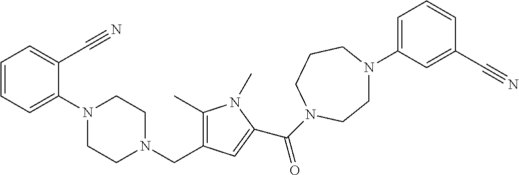 [SMILES: Cc1c(CN2CCN(c3ccccc3C#N)CC2)cc(C(=O)N2CCCN(c3cccc(C#N)c3)CC2)n1C] | I-45 |
| 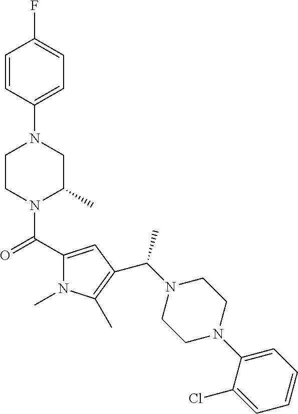 [SMILES: Cc1c([C@H](C)N2CCN(c3ccccc3Cl)CC2)cc(C(=O)N2CCN(c3ccc(F)cc3)C[C@@H]2C)n1C] | I-46 |
| 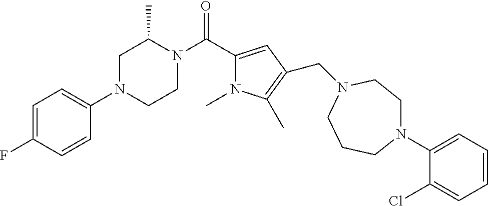 [SMILES: Cc1c(CN2CCCN(c3ccccc3Cl)CC2)cc(C(=O)N2CCN(c3ccc(F)cc3)C[C@@H]2C)n1C] | I-47 |
| 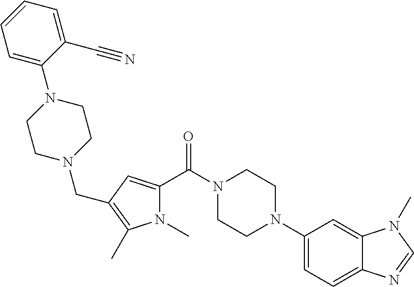 [SMILES: Cc1c(CN2CCN(c3ccccc3C#N)CC2)cc(C(=O)N2CCN(c3ccc4ncn(C)c4c3)CC2)n1C] | I-48 |
| 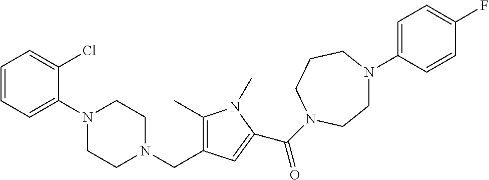 [SMILES: Cc1c(CN2CCN(c3ccccc3Cl)CC2)cc(C(=O)N2CCCN(c3ccc(F)cc3)CC2)n1C] | I-49 |
| 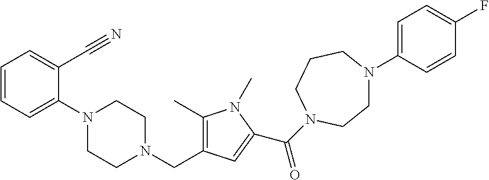 [SMILES: Cc1c(CN2CCN(c3ccccc3C#N)CC2)cc(C(=O)N2CCCN(c3ccc(F)cc3)CC2)n1C] | I-50 |
| 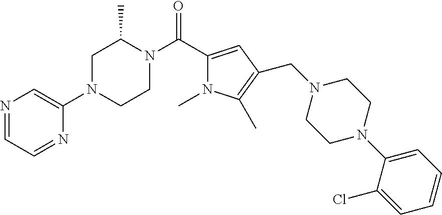 [SMILES: Cc1c(CN2CCN(c3ccccc3Cl)CC2)cc(C(=O)N2CCN(c3cnccn3)C[C@@H]2C)n1C] | I-51 |
| 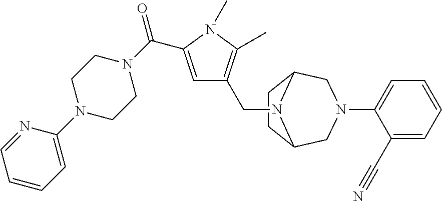 [SMILES: Cc1c(CN2C3CCC2CN(c2ccccc2C#N)C3)cc(C(=O)N2CCN(c3ccccn3)CC2)n1C] | I-52 |
| 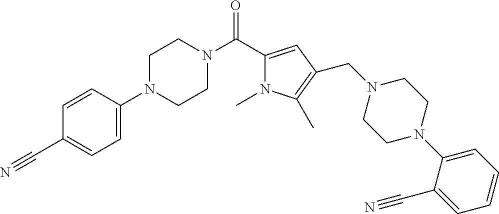 [SMILES: Cc1c(CN2CCN(c3ccccc3C#N)CC2)cc(C(=O)N2CCN(c3ccc(C#N)cc3)CC2)n1C] | I-53 |
| 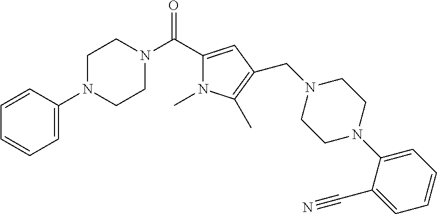 [SMILES: Cc1c(CN2CCN(c3ccccc3C#N)CC2)cc(C(=O)N2CCN(c3ccccc3)CC2)n1C] | I-54 |
| 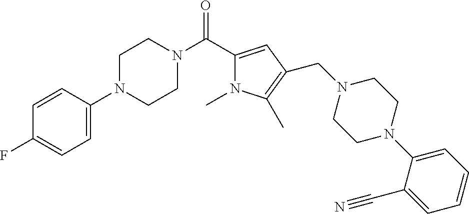 [SMILES: Cc1c(CN2CCN(c3ccccc3C#N)CC2)cc(C(=O)N2CCN(c3ccc(F)cc3)CC2)n1C] | I-55 |
| 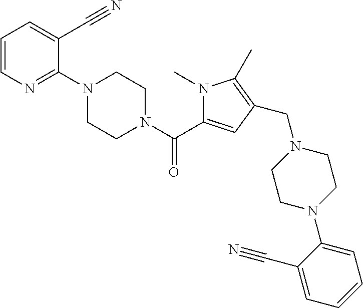 [SMILES: Cc1c(CN2CCN(c3ccccc3C#N)CC2)cc(C(=O)N2CCN(c3ncccc3C#N)CC2)n1C] | I-56 |
| 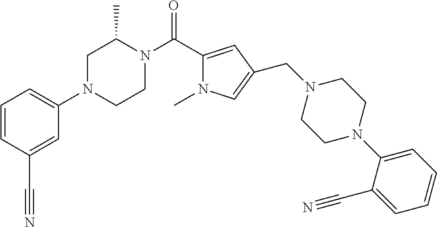 [SMILES: C[C@H]1CN(c2cccc(C#N)c2)CCN1C(=O)c1cc(CN2CCN(c3ccccc3C#N)CC2)cn1C] | I-57 |
| 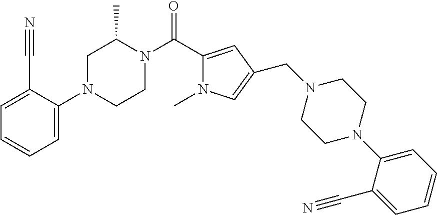 [SMILES: C[C@H]1CN(c2ccccc2C#N)CCN1C(=O)c1cc(CN2CCN(c3ccccc3C#N)CC2)cn1C] | I-58 |
| 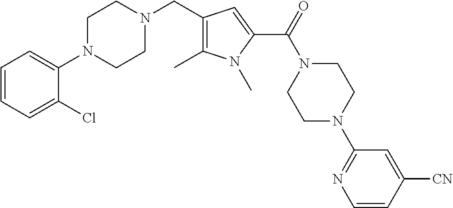 [SMILES: Cc1c(CN2CCN(c3ccccc3Cl)CC2)cc(C(=O)N2CCN(c3cc(C#N)ccn3)CC2)n1C] | I-59 |
| 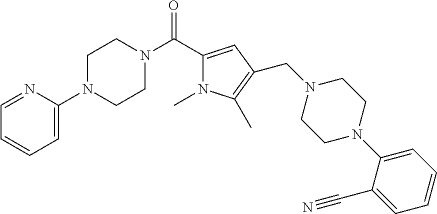 [SMILES: Cc1c(CN2CCN(c3ccccc3C#N)CC2)cc(C(=O)N2CCN(c3ccccn3)CC2)n1C] | I-60 |
| 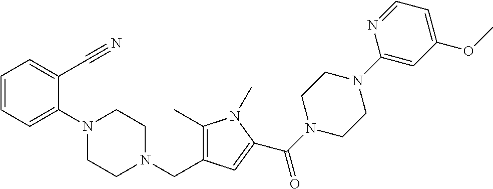 [SMILES: COc1ccnc(N2CCN(C(=O)c3cc(CN4CCN(c5ccccc5C#N)CC4)c(C)n3C)CC2)c1] | I-61 |
| 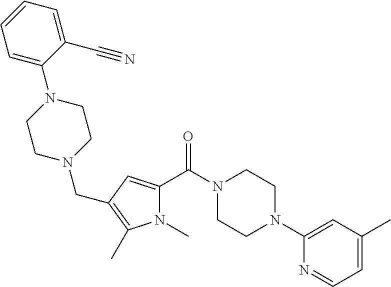 [SMILES: Cc1ccnc(N2CCN(C(=O)c3cc(CN4CCN(c5ccccc5C#N)CC4)c(C)n3C)CC2)c1] | I-62 |
| 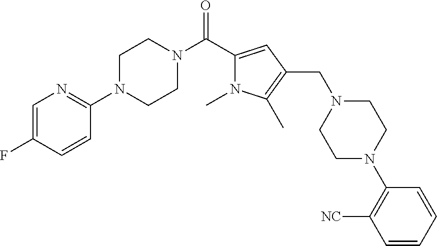 [SMILES: Cc1c(CN2CCN(c3ccccc3C#N)CC2)cc(C(=O)N2CCN(c3ccc(F)cn3)CC2)n1C] | I-63 |
| 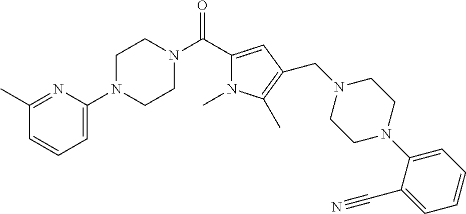 [SMILES: Cc1cccc(N2CCN(C(=O)c3cc(CN4CCN(c5ccccc5C#N)CC4)c(C)n3C)CC2)n1] | I-64 |
| 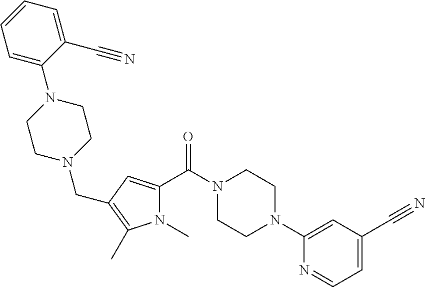 [SMILES: Cc1c(CN2CCN(c3ccccc3C#N)CC2)cc(C(=O)N2CCN(c3cc(C#N)ccn3)CC2)n1C] | I-65 |
| 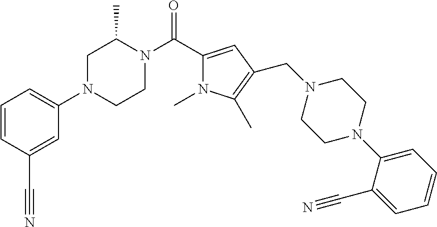 [SMILES: Cc1c(CN2CCN(c3ccccc3C#N)CC2)cc(C(=O)N2CCN(c3cccc(C#N)c3)C[C@@H]2C)n1C] | I-66 |
| 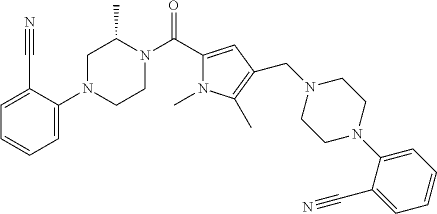 [SMILES: Cc1c(CN2CCN(c3ccccc3C#N)CC2)cc(C(=O)N2CCN(c3ccccc3C#N)C[C@@H]2C)n1C] | I-67 |
| 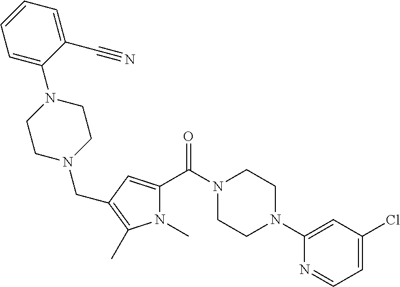 [SMILES: Cc1c(CN2CCN(c3ccccc3C#N)CC2)cc(C(=O)N2CCN(c3cc(Cl)ccn3)CC2)n1C] | I-68 |
| 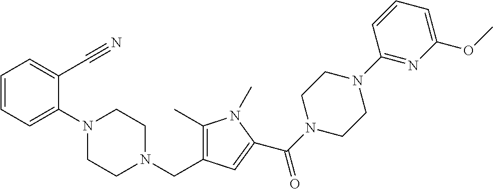 [SMILES: COc1cccc(N2CCN(C(=O)c3cc(CN4CCN(c5ccccc5C#N)CC4)c(C)n3C)CC2)n1] | I-69 |
| 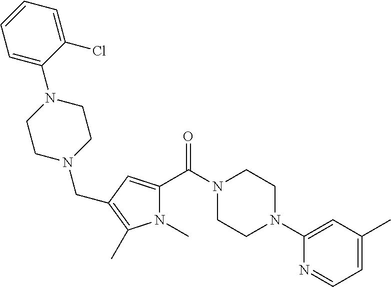 [SMILES: Cc1ccnc(N2CCN(C(=O)c3cc(CN4CCN(c5ccccc5Cl)CC4)c(C)n3C)CC2)c1] | I-70 |
| 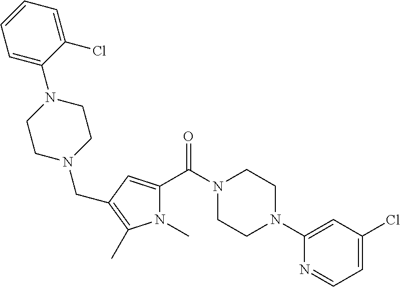 [SMILES: Cc1c(CN2CCN(c3ccccc3Cl)CC2)cc(C(=O)N2CCN(c3cc(Cl)ccn3)CC2)n1C] | I-71 |
| 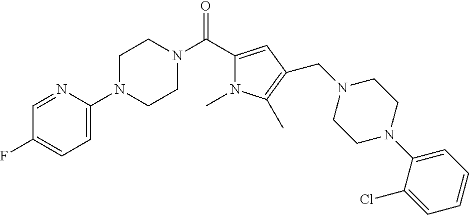 [SMILES: Cc1c(CN2CCN(c3ccccc3Cl)CC2)cc(C(=O)N2CCN(c3ccc(F)cn3)CC2)n1C] | I-72 |
| 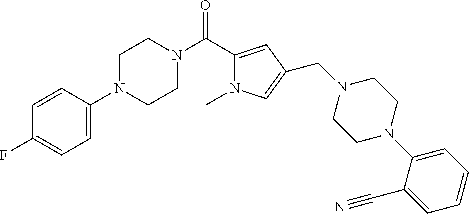 [SMILES: Cn1cc(CN2CCN(c3ccccc3C#N)CC2)cc1C(=O)N1CCN(c2ccc(F)cc2)CC1] | I-73 |
| 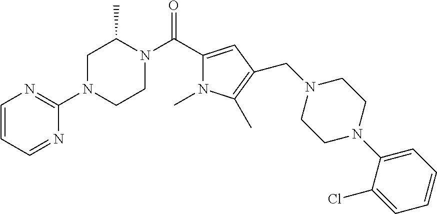 [SMILES: Cc1c(CN2CCN(c3ccccc3Cl)CC2)cc(C(=O)N2CCN(c3ncccn3)C[C@@H]2C)n1C] | I-74 |
| 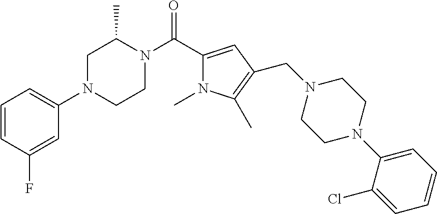 [SMILES: Cc1c(CN2CCN(c3ccccc3Cl)CC2)cc(C(=O)N2CCN(c3cccc(F)c3)C[C@@H]2C)n1C] | I-75 |
| 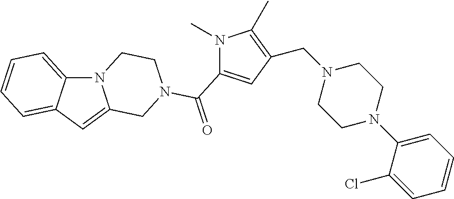 [SMILES: Cc1c(CN2CCN(c3ccccc3Cl)CC2)cc(C(=O)N2CCn3c(cc4ccccc43)C2)n1C] | I-76 |
| 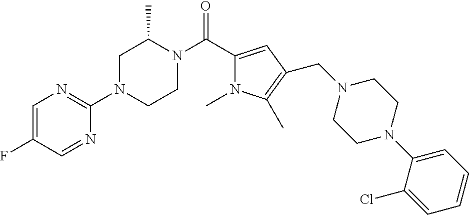 [SMILES: Cc1c(CN2CCN(c3ccccc3Cl)CC2)cc(C(=O)N2CCN(c3ncc(F)cn3)C[C@@H]2C)n1C] | I-77 |
| 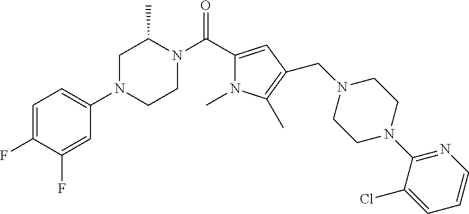 [SMILES: Cc1c(CN2CCN(c3ncccc3Cl)CC2)cc(C(=O)N2CCN(c3ccc(F)c(F)c3)C[C@@H]2C)n1C] | I-78 |
| 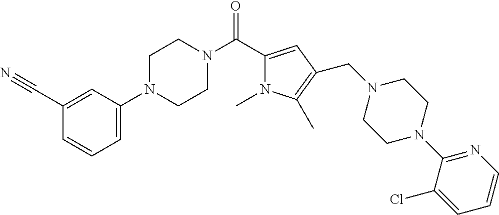 [SMILES: Cc1c(CN2CCN(c3ncccc3Cl)CC2)cc(C(=O)N2CCN(c3cccc(C#N)c3)CC2)n1C] | I-79 |
| 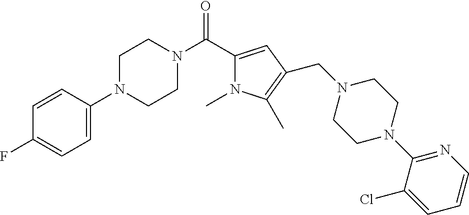 [SMILES: Cc1c(CN2CCN(c3ncccc3Cl)CC2)cc(C(=O)N2CCN(c3ccc(F)cc3)CC2)n1C] | I-80 |
| 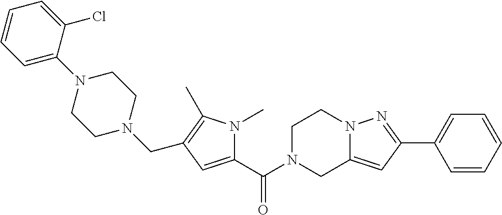 [SMILES: Cc1c(CN2CCN(c3ccccc3Cl)CC2)cc(C(=O)N2CCn3nc(-c4ccccc4)cc3C2)n1C] | I-81 |
| 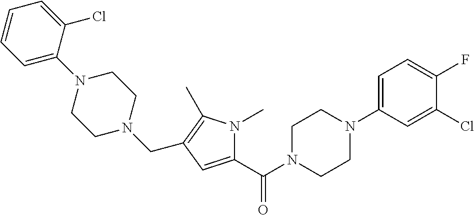 [SMILES: Cc1c(CN2CCN(c3ccccc3Cl)CC2)cc(C(=O)N2CCN(c3ccc(F)c(Cl)c3)CC2)n1C] | I-82 |
| 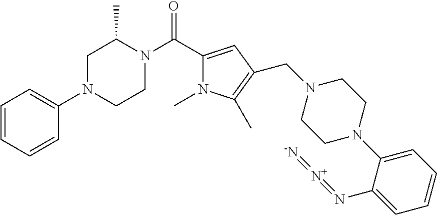 | I-83 |
| 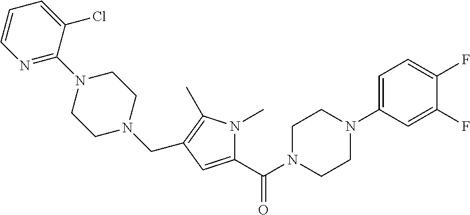 [SMILES: Cc1c(CN2CCN(c3ncccc3Cl)CC2)cc(C(=O)N2CCN(c3ccc(F)c(F)c3)CC2)n1C] | I-84 |
| 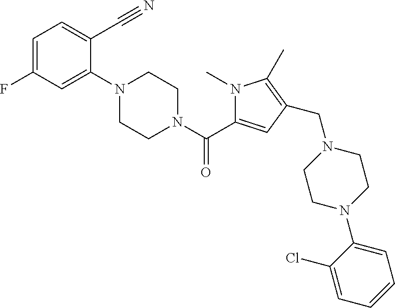 [SMILES: Cc1c(CN2CCN(c3ccccc3Cl)CC2)cc(C(=O)N2CCN(c3cc(F)ccc3C#N)CC2)n1C] | I-85 |
| 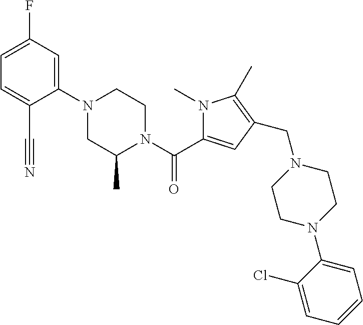 [SMILES: Cc1c(CN2CCN(c3ccccc3Cl)CC2)cc(C(=O)N2CCN(c3cc(F)ccc3C#N)C[C@@H]2C)n1C] | I-86 |
| 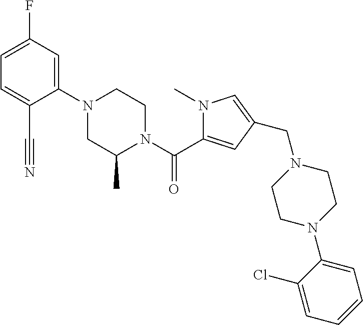 [SMILES: C[C@H]1CN(c2cc(F)ccc2C#N)CCN1C(=O)c1cc(CN2CCN(c3ccccc3Cl)CC2)cn1C] | I-87 |
| 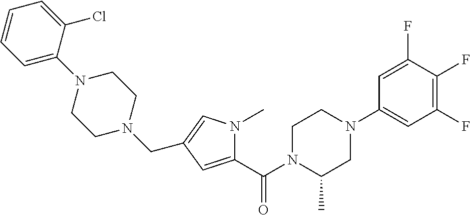 [SMILES: C[C@H]1CN(c2cc(F)c(F)c(F)c2)CCN1C(=O)c1cc(CN2CCN(c3ccccc3Cl)CC2)cn1C] | I-88 |
| 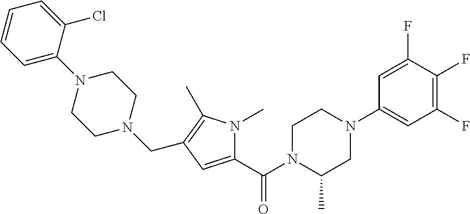 [SMILES: Cc1c(CN2CCN(c3ccccc3Cl)CC2)cc(C(=O)N2CCN(c3cc(F)c(F)c(F)c3)C[C@@H]2C)n1C] | I-89 |
| 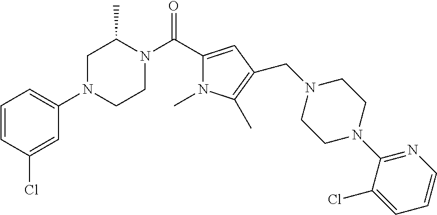 [SMILES: Cc1c(CN2CCN(c3ncccc3Cl)CC2)cc(C(=O)N2CCN(c3cccc(Cl)c3)C[C@@H]2C)n1C] | I-90 |
| 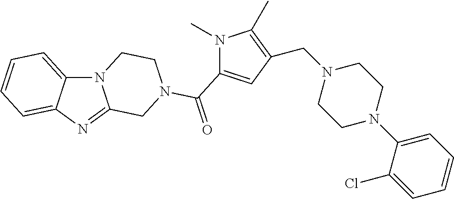 [SMILES: Cc1c(CN2CCN(c3ccccc3Cl)CC2)cc(C(=O)N2CCn3c(nc4ccccc43)C2)n1C] | I-91 |
| 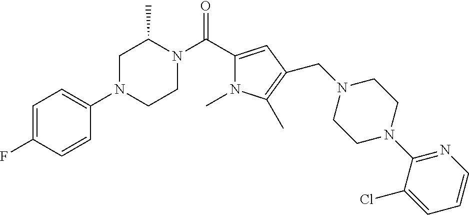 [SMILES: Cc1c(CN2CCN(c3ncccc3Cl)CC2)cc(C(=O)N2CCN(c3ccc(F)cc3)C[C@@H]2C)n1C] | I-92 |
| 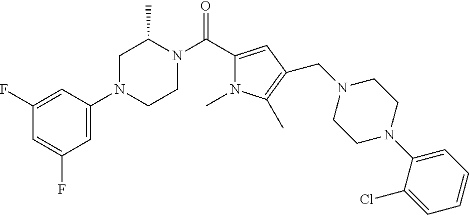 [SMILES: Cc1c(CN2CCN(c3ccccc3Cl)CC2)cc(C(=O)N2CCN(c3cc(F)cc(F)c3)C[C@@H]2C)n1C] | I-93 |
| 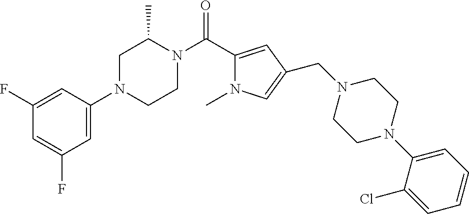 [SMILES: C[C@H]1CN(c2cc(F)cc(F)c2)CCN1C(=O)c1cc(CN2CCN(c3ccccc3Cl)CC2)cn1C] | I-94 |
| 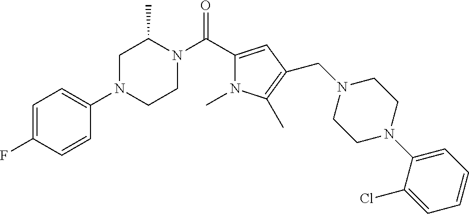 [SMILES: Cc1c(CN2CCN(c3ccccc3Cl)CC2)cc(C(=O)N2CCN(c3ccc(F)cc3)C[C@@H]2C)n1C] | I-95 |
| 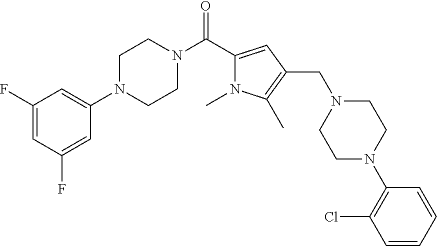 [SMILES: Cc1c(CN2CCN(c3ccccc3Cl)CC2)cc(C(=O)N2CCN(c3cc(F)cc(F)c3)CC2)n1C] | I-96 |
| 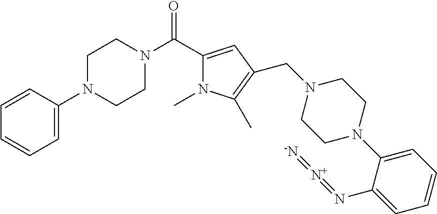 | I-97 |
| 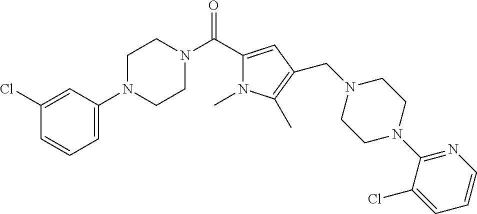 [SMILES: Cc1c(CN2CCN(c3ncccc3Cl)CC2)cc(C(=O)N2CCN(c3cccc(Cl)c3)CC2)n1C] | I-98 |
| 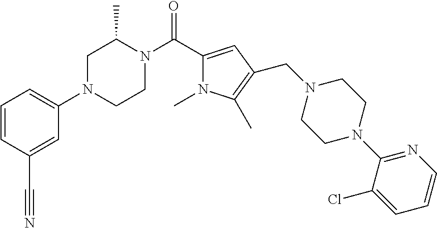 [SMILES: Cc1c(CN2CCN(c3ncccc3Cl)CC2)cc(C(=O)N2CCN(c3cccc(C#N)c3)C[C@@H]2C)n1C] | I-99 |
| 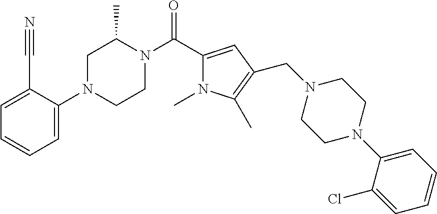 [SMILES: Cc1c(CN2CCN(c3ccccc3Cl)CC2)cc(C(=O)N2CCN(c3ccccc3C#N)C[C@@H]2C)n1C] | I-100 |
| 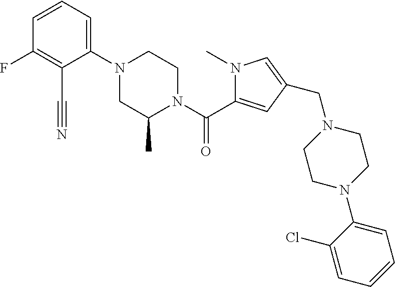 [SMILES: C[C@H]1CN(c2cccc(F)c2C#N)CCN1C(=O)c1cc(CN2CCN(c3ccccc3Cl)CC2)cn1C] | I-101 |
| 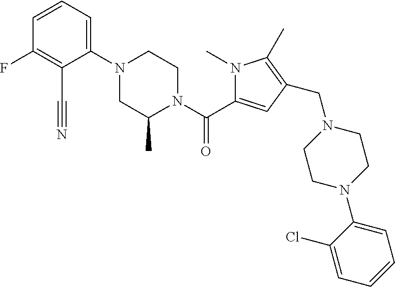 [SMILES: Cc1c(CN2CCN(c3ccccc3Cl)CC2)cc(C(=O)N2CCN(c3cccc(F)c3C#N)C[C@@H]2C)n1C] | I-102 |
| 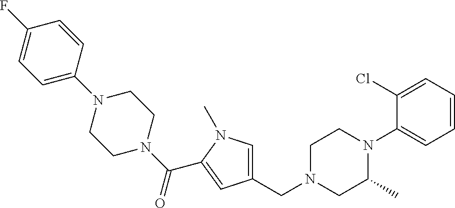 [SMILES: C[C@@H]1CN(Cc2cc(C(=O)N3CCN(c4ccc(F)cc4)CC3)n(C)c2)CCN1c1ccccc1Cl] | I-103 |
| 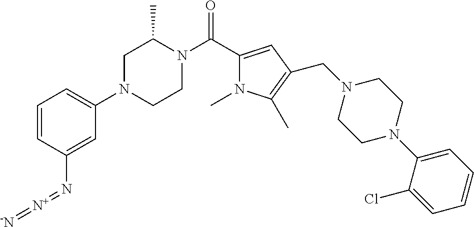 | I-104 |
| 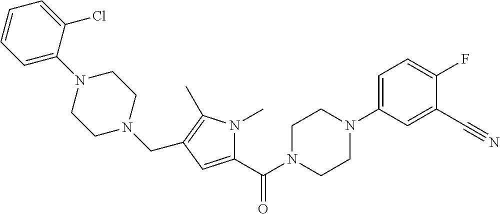 [SMILES: Cc1c(CN2CCN(c3ccccc3Cl)CC2)cc(C(=O)N2CCN(c3ccc(F)c(C#N)c3)CC2)n1C] | I-105 |
| 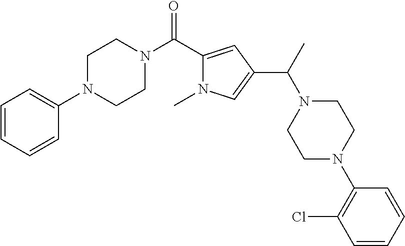 [SMILES: CC(c1cc(C(=O)N2CCN(c3ccccc3)CC2)n(C)c1)N1CCN(c2ccccc2Cl)CC1] | I-106 |
| 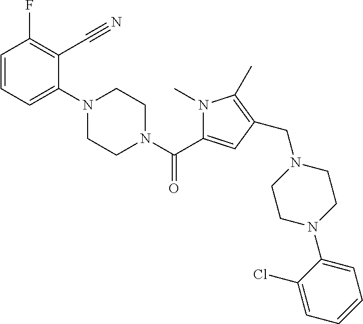 [SMILES: Cc1c(CN2CCN(c3ccccc3Cl)CC2)cc(C(=O)N2CCN(c3cccc(F)c3C#N)CC2)n1C] | I-107 |
| 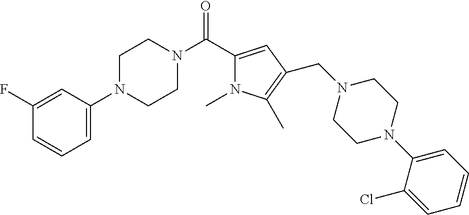 [SMILES: Cc1c(CN2CCN(c3ccccc3Cl)CC2)cc(C(=O)N2CCN(c3cccc(F)c3)CC2)n1C] | I-108 |
| 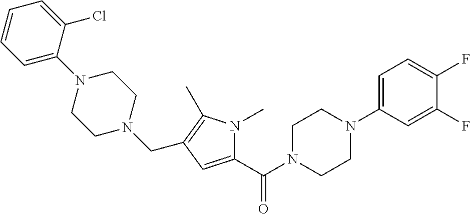 [SMILES: Cc1c(CN2CCN(c3ccccc3Cl)CC2)cc(C(=O)N2CCN(c3ccc(F)c(F)c3)CC2)n1C] | I-109 |
| 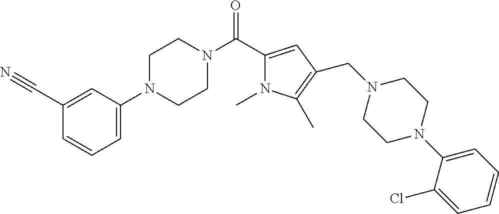 [SMILES: Cc1c(CN2CCN(c3ccccc3Cl)CC2)cc(C(=O)N2CCN(c3cccc(C#N)c3)CC2)n1C] | I-110 |
| 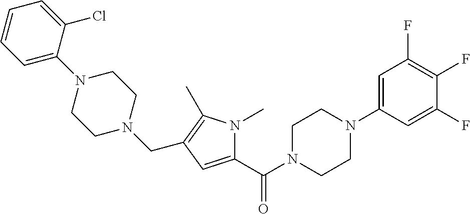 [SMILES: Cc1c(CN2CCN(c3ccccc3Cl)CC2)cc(C(=O)N2CCN(c3cc(F)c(F)c(F)c3)CC2)n1C] | I-111 |
| 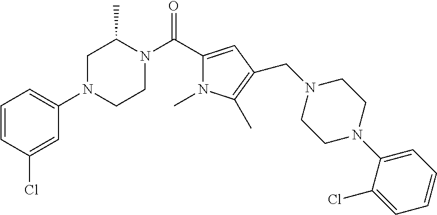 [SMILES: Cc1c(CN2CCN(c3ccccc3Cl)CC2)cc(C(=O)N2CCN(c3cccc(Cl)c3)C[C@@H]2C)n1C] | I-112 |
| 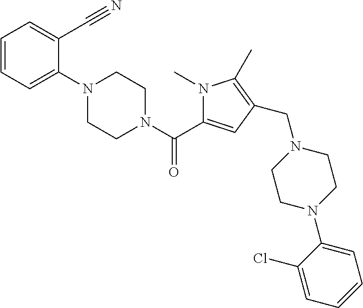 [SMILES: Cc1c(CN2CCN(c3ccccc3Cl)CC2)cc(C(=O)N2CCN(c3ccccc3C#N)CC2)n1C] | I-113 |
| 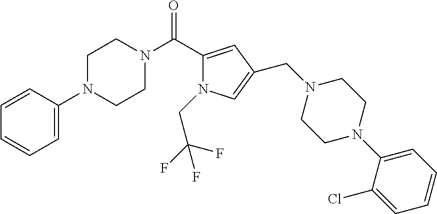 [SMILES: O=C(c1cc(CN2CCN(c3ccccc3Cl)CC2)cn1CC(F)(F)F)N1CCN(c2ccccc2)CC1] | I-114 |
| 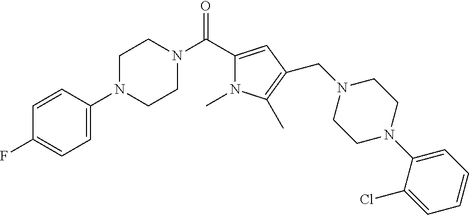 [SMILES: Cc1c(CN2CCN(c3ccccc3Cl)CC2)cc(C(=O)N2CCN(c3ccc(F)cc3)CC2)n1C] | I-115 |
| 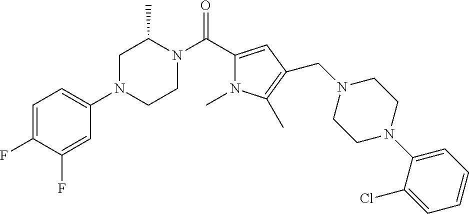 [SMILES: Cc1c(CN2CCN(c3ccccc3Cl)CC2)cc(C(=O)N2CCN(c3ccc(F)c(F)c3)C[C@@H]2C)n1C] | I-116 |
| 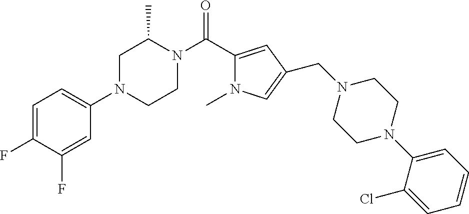 [SMILES: C[C@H]1CN(c2ccc(F)c(F)c2)CCN1C(=O)c1cc(CN2CCN(c3ccccc3Cl)CC2)cn1C] | I-117 |
| 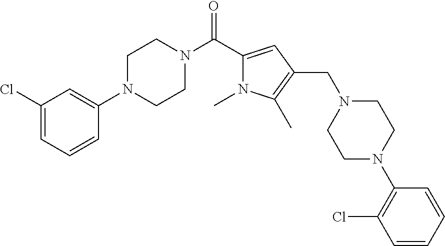 [SMILES: Cc1c(CN2CCN(c3ccccc3Cl)CC2)cc(C(=O)N2CCN(c3cccc(Cl)c3)CC2)n1C] | I-118 |
| 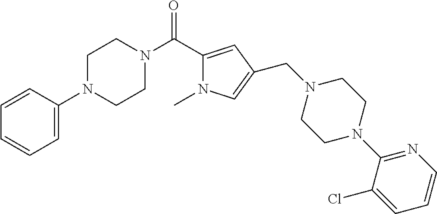 [SMILES: Cn1cc(CN2CCN(c3ncccc3Cl)CC2)cc1C(=O)N1CCN(c2ccccc2)CC1] | I-119 |
| 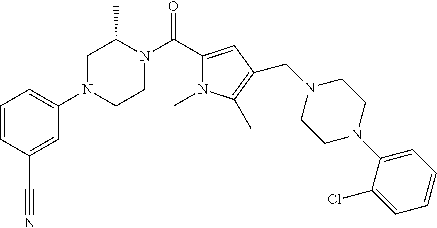 [SMILES: Cc1c(CN2CCN(c3ccccc3Cl)CC2)cc(C(=O)N2CCN(c3cccc(C#N)c3)C[C@@H]2C)n1C] | I-120 |
| 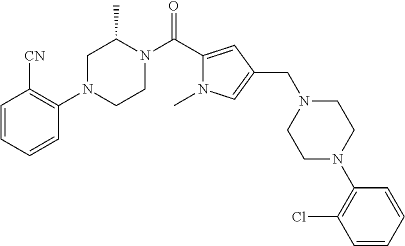 [SMILES: C[C@H]1CN(c2ccccc2C#N)CCN1C(=O)c1cc(CN2CCN(c3ccccc3Cl)CC2)cn1C] | I-121 |
| 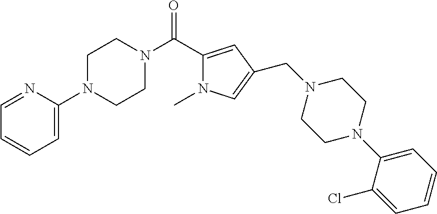 [SMILES: Cn1cc(CN2CCN(c3ccccc3Cl)CC2)cc1C(=O)N1CCN(c2ccccn2)CC1] | I-122 |
| 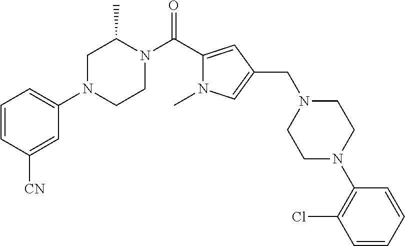 [SMILES: C[C@H]1CN(c2cccc(C#N)c2)CCN1C(=O)c1cc(CN2CCN(c3ccccc3Cl)CC2)cn1C] | I-123 |
| 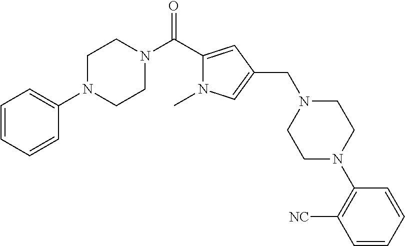 [SMILES: Cn1cc(CN2CCN(c3ccccc3C#N)CC2)cc1C(=O)N1CCN(c2ccccc2)CC1] | I-124 |
| 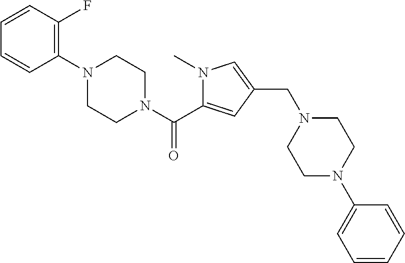 [SMILES: Cn1cc(CN2CCN(c3ccccc3)CC2)cc1C(=O)N1CCN(c2ccccc2F)CC1] | I-125 |
| 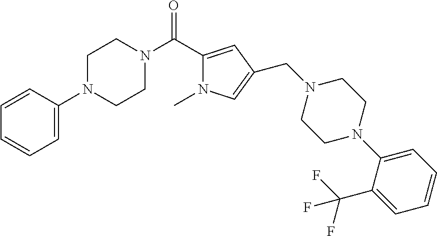 [SMILES: Cn1cc(CN2CCN(c3ccccc3C(F)(F)F)CC2)cc1C(=O)N1CCN(c2ccccc2)CC1] | I-126 |
| 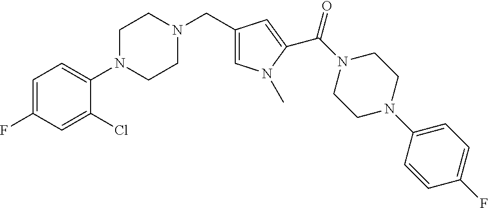 [SMILES: Cn1cc(CN2CCN(c3ccc(F)cc3Cl)CC2)cc1C(=O)N1CCN(c2ccc(F)cc2)CC1] | I-127 |
| 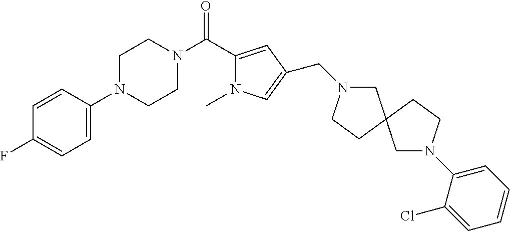 [SMILES: Cn1cc(CN2CCC3(CCN(c4ccccc4Cl)C3)C2)cc1C(=O)N1CCN(c2ccc(F)cc2)CC1] | I-128 |
| 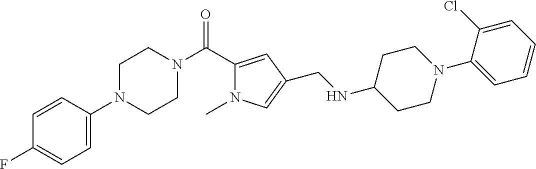 [SMILES: Cn1cc(CNC2CCN(c3ccccc3Cl)CC2)cc1C(=O)N1CCN(c2ccc(F)cc2)CC1] | I-129 |
| 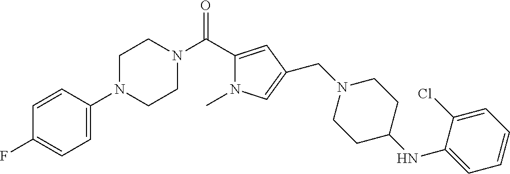 [SMILES: Cn1cc(CN2CCC(Nc3ccccc3Cl)CC2)cc1C(=O)N1CCN(c2ccc(F)cc2)CC1] | I-130 |
| 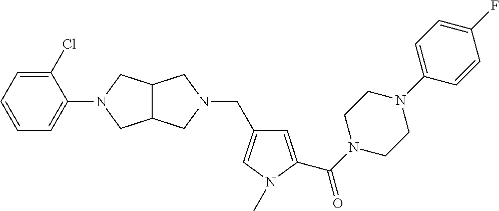 [SMILES: Cn1cc(CN2CC3CN(c4ccccc4Cl)CC3C2)cc1C(=O)N1CCN(c2ccc(F)cc2)CC1] | I-131 |
| 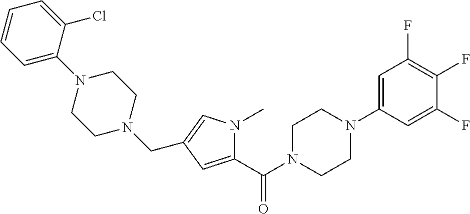 [SMILES: Cn1cc(CN2CCN(c3ccccc3Cl)CC2)cc1C(=O)N1CCN(c2cc(F)c(F)c(F)c2)CC1] | I-132 |
| 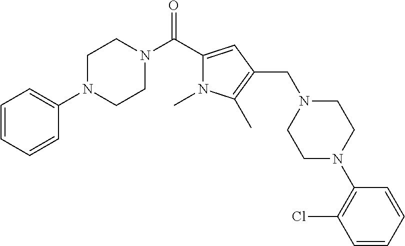 [SMILES: Cc1c(CN2CCN(c3ccccc3Cl)CC2)cc(C(=O)N2CCN(c3ccccc3)CC2)n1C] | I-133 |
| 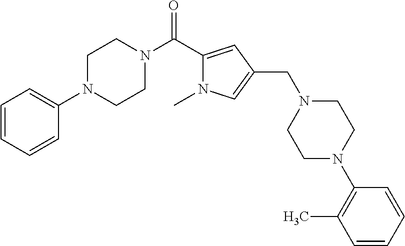 [SMILES: Cc1ccccc1N1CCN(Cc2cc(C(=O)N3CCN(c4ccccc4)CC3)n(C)c2)CC1] | I-134 |
| 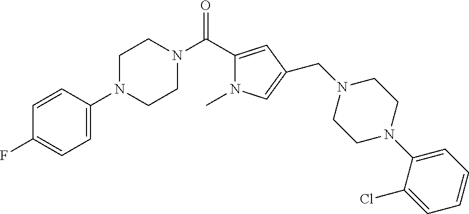 [SMILES: Cn1cc(CN2CCN(c3ccccc3Cl)CC2)cc1C(=O)N1CCN(c2ccc(F)cc2)CC1] | I-135 |
| 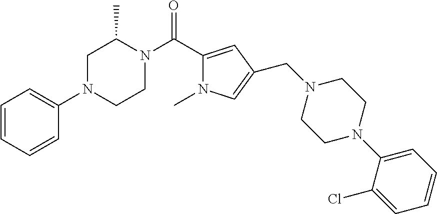 [SMILES: C[C@H]1CN(c2ccccc2)CCN1C(=O)c1cc(CN2CCN(c3ccccc3Cl)CC2)cn1C] | I-136 |
| 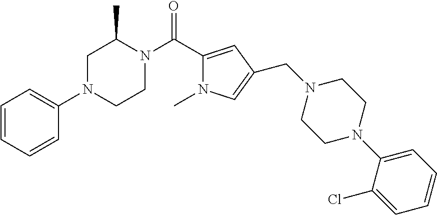 [SMILES: C[C@@H]1CN(c2ccccc2)CCN1C(=O)c1cc(CN2CCN(c3ccccc3Cl)CC2)cn1C] | I-137 |
| 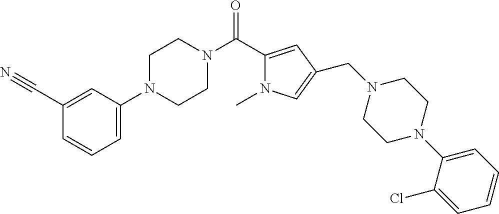 [SMILES: Cn1cc(CN2CCN(c3ccccc3Cl)CC2)cc1C(=O)N1CCN(c2cccc(C#N)c2)CC1] | I-138 |
| 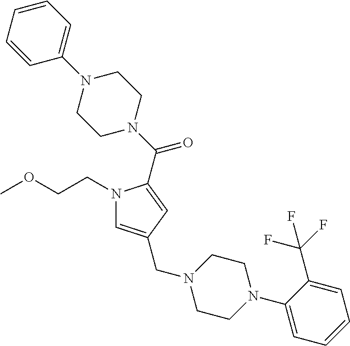 [SMILES: COCCn1cc(CN2CCN(c3ccccc3C(F)(F)F)CC2)cc1C(=O)N1CCN(c2ccccc2)CC1] | I-139 |
| 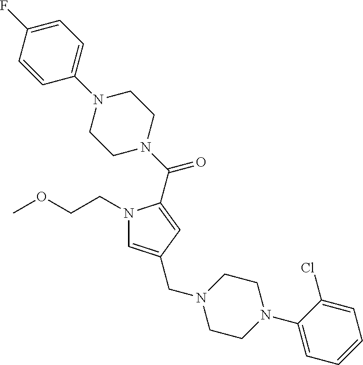 [SMILES: COCCn1cc(CN2CCN(c3ccccc3Cl)CC2)cc1C(=O)N1CCN(c2ccc(F)cc2)CC1] | I-140 |
| 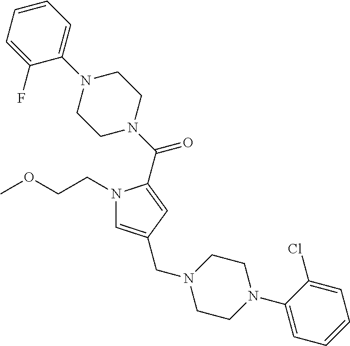 [SMILES: COCCn1cc(CN2CCN(c3ccccc3Cl)CC2)cc1C(=O)N1CCN(c2ccccc2F)CC1] | I-141 |
| 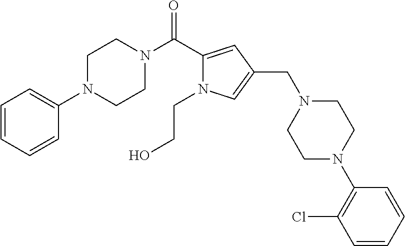 [SMILES: O=C(c1cc(CN2CCN(c3ccccc3Cl)CC2)cn1CCO)N1CCN(c2ccccc2)CC1] | I-142 |
| 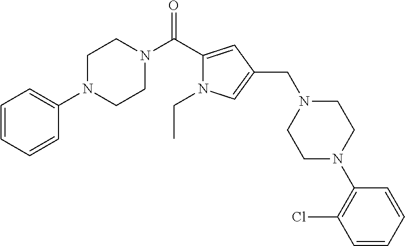 [SMILES: CCn1cc(CN2CCN(c3ccccc3Cl)CC2)cc1C(=O)N1CCN(c2ccccc2)CC1] | I-143 |
| 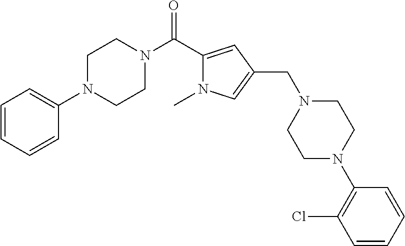 [SMILES: Cn1cc(CN2CCN(c3ccccc3Cl)CC2)cc1C(=O)N1CCN(c2ccccc2)CC1] | I-144 |
| 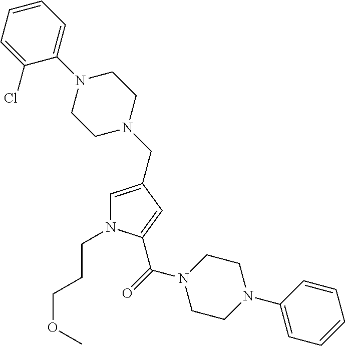 [SMILES: COCCCn1cc(CN2CCN(c3ccccc3Cl)CC2)cc1C(=O)N1CCN(c2ccccc2)CC1] | I-145 |
| 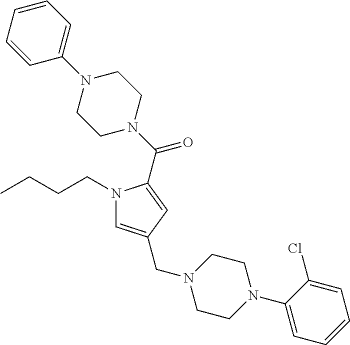 [SMILES: CCCCn1cc(CN2CCN(c3ccccc3Cl)CC2)cc1C(=O)N1CCN(c2ccccc2)CC1] | I-146 |
| 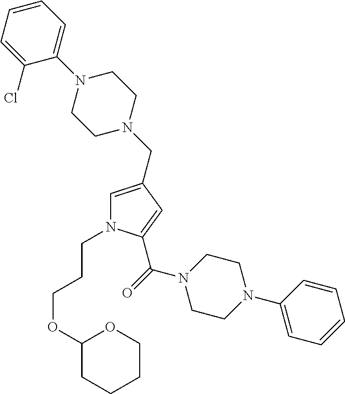 [SMILES: O=C(c1cc(CN2CCN(c3ccccc3Cl)CC2)cn1CCCOC1CCCCO1)N1CCN(c2ccccc2)CC1] | I-147 |
| 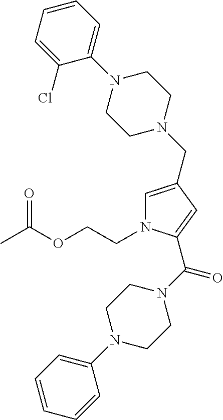 [SMILES: CC(=O)OCCn1cc(CN2CCN(c3ccccc3Cl)CC2)cc1C(=O)N1CCN(c2ccccc2)CC1] | I-148 |
| 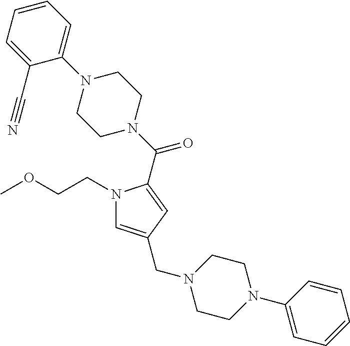 [SMILES: COCCn1cc(CN2CCN(c3ccccc3)CC2)cc1C(=O)N1CCN(c2ccccc2C#N)CC1] | I-149 |
| 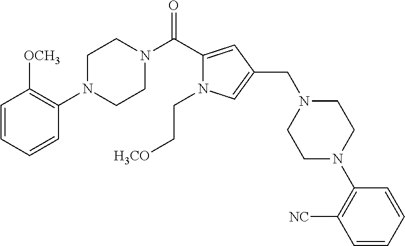 [SMILES: COCCn1cc(CN2CCN(c3ccccc3C#N)CC2)cc1C(=O)N1CCN(c2ccccc2OC)CC1] | I-150 |
| 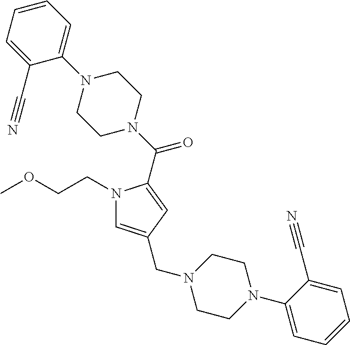 [SMILES: COCCn1cc(CN2CCN(c3ccccc3C#N)CC2)cc1C(=O)N1CCN(c2ccccc2C#N)CC1] | I-151 |
| 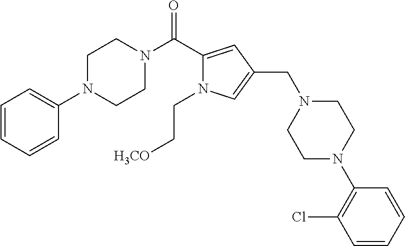 [SMILES: COCCn1cc(CN2CCN(c3ccccc3Cl)CC2)cc1C(=O)N1CCN(c2ccccc2)CC1] | I-152 |

In some embodiments, the present invention provides a compound set forth in Table 1, above, or a pharmaceutically acceptable salt thereof. It will be appreciated that the present invention also provides a compound set forth in Table 1, above, as a racemic mixture at the C7 position, or a pharmaceutically acceptable salt thereof.

### 5. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit mTORC1, in a biological sample or in a patient. In certain embodiments, the amount of compound in compositions of this invention is such that is effective to measurably inhibit mTORC1, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the inhibition of mTORC1.

The activity of a compound utilized in this invention as an inhibitor of mTORC1, may be assayed in vitro, in vivo or in a cell line. In vitro assays include assays that determine the inhibition of mTORC1. Detailed conditions for assaying a compound utilized in this invention as an inhibitor of mTORC1 are well known to one of ordinary skill in the art. Such methods are described in detail by Liu et al., Cancer Research, 73(8), Apr. 15, 2013 and Liu et al., J. Biological Chemistry, vol 287, no. 13, pp 9742-9752 (2012).

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of mTORC1 and are therefore useful for treating one or more disorders associated with activity of mTORC1. Thus, in certain embodiments, the present invention provides a method for treating an mTORC1-mediated disorder comprising the step of administering to a patient in need thereof a compound of the present invention, or pharmaceutically acceptable composition thereof.

As used herein, the terms “mTORC1-mediated” disorders, diseases, and/or conditions as used herein means any disease or other deleterious condition in which mTORC1, is known to play a role. Accordingly, another embodiment of the present invention relates to treating or lessening the severity of one or more diseases in which mTORC1 is known to play a role. In certain embodiments, an mTORC1-mediated disorder, disease, and/or condition is selected from those described by Matt Kaeberlin, Scientifica, vol. 2013, Article ID 849186.

The methods described herein include methods for the treatment of cancer in a subject. As used in this context, to “treat” means to ameliorate or improve at least one symptom or clinical parameter of the cancer. For example, a treatment can result in a reduction in tumor size or growth rate. A treatment need not cure the cancer or cause remission 100% of the time, in all subjects.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancer cells.

Cancers that can be treated or diagnoses using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

In some embodiments, the methods described herein are used for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

In some embodiments, the cancers that are treated by the methods described herein are cancers that have increased levels of mTORC1 or an increased expression or activity of a mTORC1 relative to normal tissues or to other cancers of the same tissues; methods known in the art and described herein can be used to identify those cancers. In some embodiments, the methods include obtaining a sample comprising cells of the cancer, determining the mTORC1 activity in the sample, and administering a treatment as described herein (e.g., a provided inhibitor of mTORC1). In some embodiments, the cancer is one that is shown herein to have increased levels of mTORC1 activity

In some embodiments, the present invention provides a method for treating one or more disorders, diseases, and/or conditions wherein the disorder, disease, or condition includes, but is not limited to, a cellular proliferative disorder.

### Cellular Proliferative Disorders

The present invention features methods and compositions for the diagnosis and prognosis of cellular proliferative disorders (e.g., cancer) and the treatment of these disorders by inhibiting mTORC1 activity. Cellular proliferative disorders described herein include, e.g., cancer, obesity, and proliferation-dependent diseases. Such disorders may be diagnosed using methods known in the art.

### Cancer

Cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). In some embodiments, the cancer is melanoma or breast cancer.

### Fibrotic Diseases

Idiopathic Pulmonary Fibrosis (IPF). The PI3K pathway is activated in fibrotic foci, the cardinal lesions in IPF. mTOR kinase inhibitor GSK2126458 reduces PI3K pathway signaling and functional responses in IPF-derived lung fibroblasts and mTOR inhibition reduces collagen expression in models of IPF patients. In the bleomycin model of pulmonary fibrosis, rapamycin treatment is antifibrotic, and rapamycin also decreases expression of α-smooth muscle actin and fibronectin by fibroblasts in vitro.

In some embodiments, the method of inhibiting mTORC1 activity is used to treat idiopathic pulmonary fibrosis (IPF). (See Thorax. 2016, 71(8), pp. 701-11; PLoS One. 2012, 7(7)). Accordingly, in some embodiments, the present invention provides a method of treating idiopathic pulmonary fibrosis (IPF), in a patient in need thereof, comprising the step of administering to said patient a provided compound or pharmaceutically acceptable salt thereof.

Kidney Fibrosis. mTORC1 is activated in myofibroblasts, a major pathogenic cell type in kidney fibrosis. Inhibition of mTOR with rapamycin in a murine model of kidney fibrosis (UUO), attenuated expression of markers of fibrosis and tubulointerstitial damage.

In some embodiments, the method of inhibiting mTORC1 activity is used to treat kidney fibrosis. (See J Am Soc Nephrol 2013, 24, pp. 1114-1126; Kidney International 2006, 69, pp. 2029-2036; PLoS 2012, 7, Issue 3, e33626; Clin Invest Med 2014, Vol 37, no 3, E142). Accordingly, in some embodiments, the present invention provides a method of treating kidney fibrosis, in a patient in need thereof, comprising the step of administering to said patient a provided compound or pharmaceutically acceptable salt thereof.

In some embodiments, the method of inhibiting mTORC1 activity is used to treat scleroderma. (See J Invest Dermatol. 2015 November; 135(11): 2873-6). Accordingly, in some embodiments, the present invention provides a method of treating scleroderma, in a patient in need thereof, comprising the step of administering to said patient a provided compound or pharmaceutically acceptable salt thereof.

In some embodiments, the method of inhibiting mTORC1 activity is used to treat hypertrophic scarring and keloid disease. (See Am J Pathol. 2012 November; 181(5): 1642-58). Accordingly, in some embodiments, the present invention provides a method of treating hypertrophic scarring and keloid disease, in a patient in need thereof, comprising the step of administering to said patient a provided compound or pharmaceutically acceptable salt thereof.

In some embodiments, the method of inhibiting mTORC1 activity is used to treat cardiac fibrosis. (See J Mol Cell Cardiol. 2016 February; 91: 6-9). Accordingly, in some embodiments, the present invention provides a method of treating cardiac fibrosis, in a patient in need thereof, comprising the step of administering to said patient a provided compound or pharmaceutically acceptable salt thereof.

### Other Proliferative Diseases

Other proliferative diseases include, e.g., obesity, benign prostatic hyperplasia, psoriasis, abnormal keratinization, lymphoproliferative disorders (e.g., a disorder in which there is abnormal proliferation of cells of the lymphatic system), chronic rheumatoid arthritis, arteriosclerosis, restenosis, and diabetic retinopathy. Proliferative diseases that are hereby incorporated by reference include those described in U.S. Pat. Nos. 5,639,600 and 7,087,648.

### Other Disorders

Other disorders include lysosomal storage diseases, including but not limited to Pompe disease, Gaucher disease, mucopolysaccharidosis, multiple sulfatase deficiency; neurodegenerative diseases such as Parkinson's disease, Alzheimer's disease, Huntington's disease, alpha1-anti-trypsin deficiency, and spinal bulbar muscular atrophy. The present invention provides compounds that were shown to cause translocation of TFEB to the nucleus. TFEB translocation to the nucleus promotes exocytosis and/or cellular clearance of accumulating substrates in the above-mentioned diseases.

In some embodiments, the method of inhibiting mTORC1 activity is used to treat asthma. (See Respirology 2015 October; 20(7): 1055-65). Accordingly, in some embodiments, the present invention provides a method of treating asthma, in a patient in need thereof, comprising the step of administering to said patient a provided compound or pharmaceutically acceptable salt thereof.

In some embodiments, the method of inhibiting mTORC1 activity is used to treat a lysosomal storage disease. (See Annals of the New York Academy of Sciences, 2016, Volume 1371, Issue 1, pp. 3-14; Hum Mol Genet. 2015, 24(20), pp. 5775-88; EMBO Mol Med. 2013, 5(5), pp. 691-706; Medina, D. L., et al., Dev Cell. 2011 Sep. 13, 21(3), pp. 421-30). Accordingly, in some embodiments, the present invention provides a method of treating a lysosomal storage disease, in a patient in need thereof, comprising the step of administering to said patient a provided compound or pharmaceutically acceptable salt thereof.

In some embodiments, the method of inhibiting mTORC1 activity is used to treat Parkinson's disease. (See Proc Natl Acad Sci USA. 2013, 110(19):E1817-26). Accordingly, in some embodiments, the present invention provides a method of treating Parkinson's disease, in a patient in need thereof, comprising the step of administering to said patient a provided compound or pharmaceutically acceptable salt thereof.

In some embodiments, the method of inhibiting mTORC1 activity is used to treat Alzheimer's disease. (See EMBO Mol Med. 2014, 6(9), pp. 1142-60). Accordingly, in some embodiments, the present invention provides a method of treating Alzheimer's disease, in a patient in need thereof, comprising the step of administering to said patient a provided compound or pharmaceutically acceptable salt thereof.

In some embodiments, the method of inhibiting mTORC1 activity is used to treat Huntington's disease. (See Sci Transl Med. 2012, 4(142):142ra97). Accordingly, in some embodiments, the present invention provides a method of treating Huntingtons's disease, in a patient in need thereof, comprising the step of administering to said patient a provided compound or pharmaceutically acceptable salt thereof.

In some embodiments, the method of inhibiting mTORC1 activity is used to treat alpha1-anti-trypsin deficiency. (See EMBO Mol Med. 2013, 5(3), pp. 397-412). Accordingly, in some embodiments, the present invention provides a method of treating alpha1-anti-trypsin deficiency, in a patient in need thereof, comprising the step of administering to said patient a provided compound or pharmaceutically acceptable salt thereof.

In some embodiments, the method of inhibiting mTORC1 activity is used to treat spinal bulbar muscular atrophy. (See Nat Neurosci. 2014, 17(9), pp. 1180-9). Accordingly, in some embodiments, the present invention provides a method of treating spinal bulbar muscular atrophy, in a patient in need thereof, comprising the step of administering to said patient a provided compound or pharmaceutically acceptable salt thereof.

The present invention provides compounds that are inhibitors of mTORC1 activity and were shown to selectively inhibit mTORC1 over mTORC2 as measured by pS6K inhibition (a measure of mTORC1 activity) and pAKT activation (a measure of mTORC2 activity). In some embodiments, a provided compound inhibits mTORC1 selectively over mTORC2. In some embodiments, a provided compound does not measurably inhibit mTORC2. In some embodiments, a provided compound has a pAKT activation IC₅₀ of >10 μM. In some embodiments, a provided compound inhibits mTORC1 with >10-fold selectivity over mTORC2. In some embodiments, a provided compound inhibits mTORC1 with >20-fold selectivity over mTORC2. In some embodiments, a provided compound inhibits mTORC1 with >50-fold selectivity over mTORC2. In some embodiments, a provided compound inhibits mTORC1 with >100-fold selectivity over mTORC2. In some embodiments, a provided compound inhibits mTORC1 with >150-fold selectivity over mTORC2. In some embodiments, a provided compound inhibits mTORC1 with >200-fold selectivity over mTORC2. In some embodiments, a provided compound inhibits mTORC1 with >500-fold selectivity over mTORC2. In some embodiments, a provided compound inhibits mTORC1 with >1,000-fold selectivity over mTORC2. Accordingly, in some embodiments, the present invention provides a method of treating a disorder associate with mTORC1 comprising administering to patient a compound that inhibits mTORC1 wherein said compound does not inhibit mTORC2. Such compounds may be employed for indications where rapamycin and rapalogs demonstrated a benefit either in animal models or in a human disease setting. Such indications include:

Treatment of Metabolic Disease (Obesity and Insulin Resistance in Type 2 Diabetes). Inhibition of mTORC1 pathway leads to extension of life span in yeast, fly and mouse, and caloric restriction improves longevity and insulin sensitivity. The underlying mechanism has been proposed to function by regulation of mTORC1 activation. Rapamycin-induced insulin resistance has been shown to be mediated by inhibition of mTORC2 and selective mTORC1 inhibitor is predicted to improve insulin sensitivity and glucose homeostasis.

In some embodiments, the method of inhibiting mTORC1 activity is used to treat metabolic disease (obesity and insulin resistance in type 2 diabetes). (See J Gerontol A Biol Sci Med Sci 2015, 70 (4), pp. 410-20; Aging Cell 2014, 13 (2), pp. 311-9; Diabetologia 2016, 59(3), pp. 592-603; Science 2012, 335, pp. 1638-1643). Accordingly, in some embodiments, the present invention provides a method of treating metabolic disease (obesity and insulin resistance in type 2 diabetes), in a patient in need thereof, comprising the step of administering to said patient a provided compound or pharmaceutically acceptable salt thereof.

Neurofibromatosis. Neurofibromatosis type 1 (NF1) is caused by mutations in the NF1 gene. Its protein product, neurofibromin, functions as a tumor suppressor and ultimately produces constitutive upregulation of mTOR. mTOR inhibitors have been shown to reduce tumor size and induce anti-proliferative effect in NF1-associated plexiform neurofibroma.

In some embodiments, the method of inhibiting mTORC1 activity is used to treat neurofibromatosis. (See Curr Neurol Neurosci Rep. 2012 Jun. 12(3), pp. 294-301; Oncotarget. 2016 Jan. 31). Accordingly, in some embodiments, the present invention provides a method of treating neurofibromatosis, in a patient in need thereof, comprising the step of administering to said patient a provided compound or pharmaceutically acceptable salt thereof.

Cardiomyopathy and skeletal muscle dystrophy, Emery-Dreifuss muscular dystrophy model (LMNA^{-/-}). Mutations in LMNA result in several human diseases including limb-girdle muscular dystrophy (LGMD1B), Emery-Dreifuss muscular dystrophy (EDMD2/3), dilated cardiomyopathy (DCM) and conduction-system disease (CMD1A), lipodystrophy, Charcot-Marie-Tooth disease, and Hutchinson-Gilford progeria syndrome (HGPS). Lmna^{-/-} mice have elevated mTORC1 activity and short-term treatment with rapamycin in Lmna^{-/-} mice results in reduced mTORC1 signaling, improved cardiac and skeletal muscle function and enhanced survival by ˜50%.

In some embodiments, the method of inhibiting mTORC1 activity is used to treat cardiomyopathy and skeletal muscle dystrophy. (See Sci Transl Med. 2012, 4(144):144ra103; Handb Clin Neurol. 2013, 113, pp. 1367-76). Accordingly, in some embodiments, the present invention provides a method of treating cardiomyopathy and skeletal muscle dystrophy, in a patient in need thereof, comprising the step of administering to said patient a provided compound or pharmaceutically acceptable salt thereof.

Leigh syndrome. Ndufs4 knockout (KO) mice are used as a model of Leigh syndrome and exhibit hyperactivation of mTORC1 and metabolic defects. Treatment of Ndufs4 KO mice with rapamycin extended lifespan, improve metabolic and neurological defect associated with this disease.

In some embodiments, the method of inhibiting mTORC1 activity is used to treat Leigh syndrome. (See Science 2013, 342(6165), pp. 1524-8). Accordingly, in some embodiments, the present invention provides a method of treating Leigh syndrome, in a patient in need thereof, comprising the step of administering to said patient a provided compound or pharmaceutically acceptable salt thereof.

Oncology. Inhibition of mTOR with rapalogs has been shown to have antitumor activity in murine cancer models and in cancer patients. Examples of sensitive cancer types include, but are not limited to, hepatocellular carcinoma, breast cancers, mantle cell lymphomas, lung carcinoma, tuberous sclerosis and lymphangioleiomyomatosis.

In some embodiments, the method of inhibiting mTORC1 activity is used to treat cancer and oncologic disorders. (See Trends Cancer 2016; In press). Accordingly, in some embodiments, the present invention provides a method of treating cancer and oncologic disorders, in a patient in need thereof, comprising the step of administering to said patient a provided compound or pharmaceutically acceptable salt thereof.

Non-alcoholic steatohepatitis (NASH). The present invention provides inhibitors that induce autophagy to clear degraded cytoplasmic proteins, and NASH disease is characterized by lipid deposits, inflammation and fibrosis in the liver. The inhibition of mTORC1 pathway induce autophagy and down regulate SREBP-1 to decrease lipid biosynthesis to reduce lipid storage.

In some embodiments, the method of inhibiting mTORC1 activity is used to treat non-alcoholic steatohepatitis (NASH). (See J Clin Exp Hepatol 2014; 4(1), pp. 51-9). Accordingly, in some embodiments, the present invention provides a method of treating non-alcoholic steatohepatitis (NASH), in a patient in need thereof, comprising the step of administering to said patient a provided compound or pharmaceutically acceptable salt thereof.

Tuberous sclerosis (TSC) and lymphangioleiomyomatosis (LAM). Failure in the regulation of mTOR is critical to the pathogenesis of the inherited disorder tuberous sclerosis complex (TSC) and the related lung disease, lymphangioleiomyomatosis (LAM). Both diseases are caused by mutations of TSC1 or TSC2 leading to inappropriate activity of signaling downstream of mTORC1. TSC patients develop nonmalignant tumors in many organs, including the brain, while LAM patients, mostly women, accumulate abnormal, muscle-like cells in certain organs or tissues, especially the lungs, lymph nodes, and kidneys. The rapalogs, Everolimus and Sirolimus, are currently approved for the treatment of both TSC and LAM, respectively, by the US FDA.

In some embodiments, the method of inhibiting mTORC1 activity is used to treat tuberous sclerosis and lymphangioleiomyomatosis. (See J. Clin. Invest. 2011, 121, pp. 1231-1241; J. Clin Epidemiol. 2015, 7, pp. 249-57). Accordingly, in some embodiments, the present invention provides a method of treating tuberous sclerosis and lymphangioleiomyomatosis, in a patient in need thereof, comprising the step of administering to said patient a provided compound or pharmaceutically acceptable salt thereof.

Senescence and diseases of aging. Rapamycin suppresses the mammalian TORC1 complex, which regulates translation, and extends lifespan in diverse species, including mice. Rapamycin was shown to inhibit the pro-inflammatory phenotype of senescent cells. As senescent cells accumulate with age, the senescence-associated secretory phenotype (SASP) can disrupt tissues and contribute to age-related pathologies, including cancer. Inhibition of mTOR suppressed the secretion of inflammatory cytokines by senescent cells. Rapamycin reduced cytokine levels including IL6 and suppressed translation of the membrane-bound cytokine IL1A. Reduced IL1A diminishes NF-κB transcriptional activity, which controls the SASP. Thus, mTORC1 inhibitors might ameliorate age-related pathologies, including late-life cancer, by suppressing senescence-associated inflammation.

In some embodiments, the method of inhibiting mTORC1 activity is used to treat senescence and diseases of aging. (See Nature Cell Biology 17, 2015, pp. 1049-1061; Free Radic Biol Med. 2016 June; 95:133-54). Accordingly, in some embodiments, the present invention provides a method of treating senescence and diseases of aging, in a patient in need thereof, comprising the step of administering to said patient a provided compound or pharmaceutically acceptable salt thereof.

Additional therapeutic indications where mTORC inhibition may be beneficial are: cardiovascular disease (acute coronary syndrome), coronary occlusions with eluting stents, polycystic kidney disease, neurofibromatosis, epilepsy assoc. with TSC1 and/or TSC2 mutations, polycystic liver, pachyonychia congenital, fragile x syndrome, Friedrich ataxia, Peutz-Jeghers syndrome, eye disease including neovascular age-related macular degeneration, uveitis, diabetic macular edema, fibroblast growth including pulmonary fibrosis, renal insufficiency/fibrosis, metabolic syndrome, diseases of the immune system including immune senescence, lupus nephritis, chronic immune thrombocytopenia, multiple sclerosis, cancer including lymphoma, tumors associated with TSC1/2 mutations, angiomyolipoma assoc. with TSC1/2 mutations, breast cancer, hepatocellular cancer, leukemia, glioma, adenoid cystic carcinoma, senescence, autism, and vascular rheumatoid arthritis.

In some embodiments, the method of inhibiting mTORC1 activity is used to treat cardiovascular disease (acute coronary syndrome), coronary occlusions with eluting stents, polycystic kidney disease, neurofibromatosis, epilepsy assoc. with TSC1 and/or TSC2 mutations, polycystic liver, pachyonychia congenital, fragile x syndrome, Friedrich ataxia, Peutz-Jeghers syndrome, eye disease including neovascular age-related macular degeneration, uveitis, diabetic macular edema, fibroblast growth including pulmonary fibrosis, renal insufficiency/fibrosis, metabolic syndrome, diseases of the immune system including immune senescence, lupus nephritis, chronic immune thrombocytopenia, multiple sclerosis, cancer including lymphoma, tumors associated with TSC1/2 mutations, angiomyolipoma assoc. with TSC1/2 mutations, breast cancer, hepatocellular cancer, leukemia, glioma, adenoid cystic carcinoma, senescence, autism, and vascular rheumatoid arthritis. Accordingly, in some embodiments, the present invention provides a method of treating cardiovascular disease (acute coronary syndrome), coronary occlusions with eluting stents, polycystic kidney disease, neurofibromatosis, epilepsy assoc. with TSC1 and/or TSC2 mutations, polycystic liver, pachyonychia congenital, fragile x syndrome, Friedrich ataxia, Peutz-Jeghers syndrome, eye disease including neovascular age-related macular degeneration, uveitis, diabetic macular edema, fibroblast growth including pulmonary fibrosis, renal insufficiency/fibrosis, metabolic syndrome, diseases of the immune system including immune senescence, lupus nephritis, chronic immune thrombocytopenia, multiple sclerosis, cancer including lymphoma, tumors associated with TSC1/2 mutations, angiomyolipoma assoc. with TSC1/2 mutations, breast cancer, hepatocellular cancer, leukemia, glioma, adenoid cystic carcinoma, senescence, autism, and vascular rheumatoid arthritis, in a patient in need thereof, comprising the step of administering to said patient a provided compound or pharmaceutically acceptable salt thereof.

In some embodiments, the present invention is an inhibitor of members of the glucose transporter (GLUT) family. In some embodiments, the present invention is a pan-glucose inhibitor, inhibiting GLUT subtypes 1, 2, 3, 4, and 5. In some embodiments, the present invention is an inhibitor of one or more GLUT subtypes, individually or severally.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The term “biological sample”, as used herein, includes, without limitation, cell cultures or extracts thereof biopsied material obtained from a mammal or extracts thereof; and blood, saliva, urine, feces, semen, tears, or other body fluids or extracts thereof.

In other embodiments, the present invention provides a method for treating a disorder mediated by mTORC1 in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™ Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an anti estrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed.

Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Ab1 family, their gene-fusion products (e.g. BCR-Ab1 kinase) and mutants, such as compounds which target decrease or inhibit the activity of c-Ab1 family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; Ilmofosine; RO 318220 and RO 320432; GO 6976; Isis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB₂, ErbB₃, ErbB₄ as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH—1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

List of common abbreviations used in the experimental section.
4A MS: 4 Å molecular sieves
AcOH: acetic acid
Anhyd: anhydrous
aq: aqueous
BH₃-THF: borane tetrahydrofuran complex
BINAP: (2,2′-bis(diphenylphosphino)-1,1′-binaphthyl)
Bn: benzyl
Boc: tert-butoxycarbonyl
(Boc)₂O: di-tert-butyl dicarbonate
BrettPhos: 2-(Dicyclohexylphosphino)3,6-dimethoxy-2′,4′,6′-triisopropyl-1,1′-biphenyl
CbzCl: benzyl chloroformate
Cbz-OSU: N-(Benzyloxycarbonyloxy)succinimide
CHIRAL-HPLC: chiral high performance liquid chromatography
CMBP: (cyanomethylene)tributylphosphorane
Conc.: concentrated
CuCN: copper cyanide
d: days
DAST: diethylaminosulfur trifluoride
DavePhos: 2-Dicyclohexylphosphino-2′-(N,N-dimethylamino)biphenyl
dba: dibenzylideneacetone
DBU: 1,8-diazobicyclo[5.4.0]undec-7-ene
DCE: 1,2-dichloroethane
DCM: dichloromethane
DEA: diethylamine
DIBAL-H: diisobutylaluminium hydride
DIPEA: N,N-diisopropylethylamine
DMA: N,N-dimethylacetamide
DMAP: 4-dimethylaminopyridine
N,N-dimethylformamide
DMPU: 1,3-dimethyl-3,4,5,6-tetrahydro-2-pyrimidinone
DMSO: dimethyl sulfoxide
DPPA: diphenylphosphoryl azide
dppf: 1,1′-bis(diphenylphosphino)ferrocene
EA: ethyl acetate
EDCI: 1-(3-dimethylaminopropyl)-3-ethylcarbodiimide hydrochloride
EDTA: ethylenediaminetetraacetic acid
ee: enantiomeric excess
ESI: electrospray ionization
Et₃N: triethylamine
Et₂O: diethyl ether
EtOAc: ethyl acetate
EtOH: ethanol
Fmoc: fluorenylmethyloxycarbonyl
Fmoc-OSu: N-(9-fluorenylmethoxycarbonyloxy)succinimide
h: hours
HATU: N,N,N′,N′-tetramethyl-O-(7-azabenzotriazol-1-yl)uranium
hexafluorophosphate
HOBT: Hydroxybenzotriazole
HPLC: high performance liquid chromatography
HCl: hydrochloric acid
IBX: 2-iodoxybenzoic acid
IPA: isopropyl alcohol
JackiePhos: 2-{Bis[3,5-bis(trifluoromethyl)phenyl]phosphino}-3,6-dimethoxy-2′,4′,6′-triisopropyl-1,1′-biphenyl, Bis(3,5-bis(trifluoromethyl)phenyl)(2′,4′,6′-triisopropyl-3,6-dimethoxybiphenyl-2-yl)phosphine
LDA: lithium diisopropylamide
M: molar
mCPBA: meta-chloroperoxybenzoic acid
Me: methyl
MeCN: acetonitrile
MeOH: methanol
MgO: magnesium oxide
min: minutes
mL: milliliters
mM: millimolar
mmol: millimoles
MOM: methoxymethyl
MsCl: Mesyl Chloride
MTBE: methyl tert-butyl ether
NMP: N-methyl-2-pyrrolidone
n-BuLi: n-butyl lithium
NBS: N-bromosuccinimide
NIS: N-iodosuccinimide
NMO: 4-methylmorpholine N-oxide
NMP: N-methylpyrrolidine
NMR: Nuclear Magnetic Resonance
° C.: degrees Celsius
PBS: phosphate buffered saline
Pd/C: palladium on carbon
Pd₂(dba)₃: tris(dibenzylideneacetone)dipalladium(0)
PE: petroleum ether
prep-HPLC: preparative high performance liquid chromatography
P(o-tol)₃: tri(o-tolyl)phosphine
PTFE: polytetrafluoroethylene
Rel: relative
rt: room temperature
RuPhos: 2-dicyclohexylphosphino-2′,6′-diisopropoxybiphenyl
sat: saturated
SFC: supercritical fluid chromatography
SGC: silica gel chromatography
STAB: sodium triacetoxyborohydride
TBAB: Tetra-n-butylammonium bromide
TBAF: Tetra-n-butylammonium fluoride
TBSCl: tert-Butyldimethylsilyl chloride
tBuOK: potassium tert-butoxide
tBuONa: sodium tert-butoxide
TEA: triethylamine
TEBAC: Benzyltriethylammonium chloride
Tf: trifluoromethanesulfonate
TfAA: trifluoromethanesulfonic anhydride
TFA: trifluoracetic acid
TIPS: triisopropylsilyl
TLC: thin layer chromatography
THF: tetrahydrofuran
TMSCN: trimethylsilyl cyanide
pTSA: para-toluenesulfonic acid
TsOH: p-Toluenesulfonic acid
XantPhos: 4,5-Bis(diphenylphosphino)-9,9-dimethylxanthene
XPhos: 2-Dicyclohexylphosphino-2′,4′,6′-triisopropylbiphenyl

Samples were analyzed by LCMS using the following two methods:

Method A: SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous TFA.
Method B: Xbridge C18 3.5 μm 4.6×50 mm column, the elution system used was a gradient of 5%-95% over 1.5 min at 2 ml/min and the solvent was acetonitrile/10 mM ammonium acetate aqueous solution.

Samples were purified by prep-HPLC using the following two methods:

Method C: the crude samples were dissolved in DMF before purification, unless otherwise noted. Boston C18 21*250 mm 10 μm column. The mobile phase was acetonitrile/0.01% aqueous TFA (or 0.01% aqueous HCl).
Method D: the crude samples were dissolved in DMF before purification, unless otherwise noted. Boston C18 21*250 mm 10 μm column. The mobile phase was acetonitrile/10 mM ammonium acetate aqueous solution.

### General Procedures for Intermediate I:

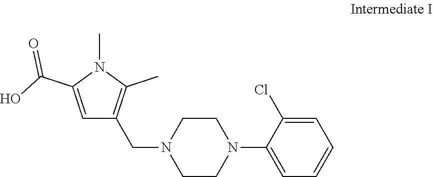 [SMILES: Cc1c(CN2CCN(c3ccccc3Cl)CC2)cc(C(=O)O)n1C]

### Synthetic Scheme:

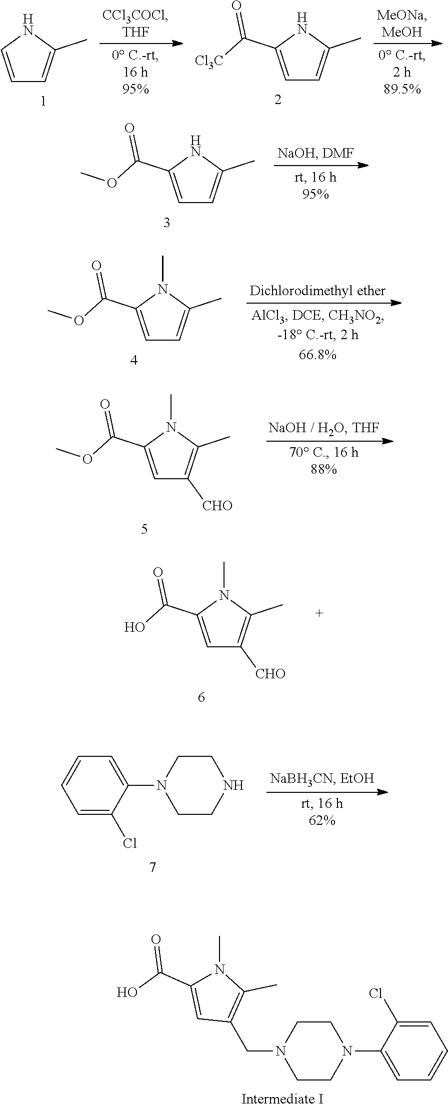

### Procedures and Characterization:

### Step 1: 2,2,2-Trichloro-1-(5-methyl-1H-pyrrol-2-yl)ethanone

To a solution of 2-methyl-1H-pyrrole (10 g, 123.3 mmol) in THF (100 mL) was added 2,2,2-trichloroacetyl chloride (26.9 g, 148 mmol) at 0° C. The mixture was then stirred at rt for 16 hrs. The mixture was poured into H₂O (500 mL), then extracted with EtOAc (500 mL×2), dried, and concentrated to give residue of 2,2,2-trichloro-1-(5-methyl-1H-pyrrol-2-yl) ethanone (26.6 g, 95%) as a yellow solid which was used in the next step without further purification. ESI-MS (EI⁺, m/z): 226.0 [M+H]⁺.

### Step 2: Methyl-5-methyl-1H-pyrrole-2-carboxylate

A mixture of 2,2,2-trichloro-1-(5-methyl-1H-pyrrol-2-yl)ethanone (40 g, 176.6 mmol, crude) in MeONa/MeOH (100 mL) and MeOH (400 mL) was stirred at rt for 2 hrs. The reaction mixture was concentrated, cooled to rt, poured into ice-water, then extracted with EtOAc (500 mL×2). The organic layers were dried and concentrated in vacuo to give residue methyl-5-methyl-1H-pyrrole-2-carboxylate (22 g, 89.5%) as s brown solid which was in the next step without further purification. ESI-MS (EI⁺, m/z): 140.1 [M+H]⁺.

### Step 3: Methyl 1,5-dimethyl-1H-pyrrole-2-carboxylate

A mixture of methyl-5-methyl-1H-pyrrole-2-carboxylate (10 g, 71.9 mmol), iodomethane (11.2 g, 79 mmol) and NaOH (3.45 g, 86 mmol) in DMF (50 mL) was stirred at rt for 2 hrs. Water (500 mL) was added to the mixture, then it was extracted with EtOAc (500 mL×2). The combined organic layers were dried over Na₂SO₄ and concentrated in vacuo. The residue was purified by chromatography (silica, PE/EtOAc=5/1) to afford methyl 1,5-dimethyl-1H-pyrrole-2-carboxylate (10.46 g, 95%). MS (EI⁺, m/z): 154.1 [M+H]⁺.

### Step 4: Methyl 4-formyl-1,5-dimethyl-1H-pyrrole-2-carboxylate

A mixture of methyl-1,5-dimethyl-1H-pyrrole-2-carboxylate (19 g, 124 mmol), dichlorodimethyl ether (18.5 g, 161 mmol) and AlCl₃ (21.5 g, 161 mmol) in DCE (200 mL) and CH₃NO₂ (200 mL) was stirred under N₂ at −18° C. for 2 hrs. The mixture was poured into ice-water (500 mL) then extracted with EtOAc (500 mL×2). The combined organic layers were dried over Na₂SO₄ and concentrated in vacuo. The residue was purified by chromatography (silica, PE/EtOAc=5/1) to afford methyl 4-formyl-1,5-dimethyl-1H-pyrrole-2-carboxylate (15 g, 66.8%). MS (EI⁺, m/z): 182.2 [M+H]⁺.

### Step 5: 4-Formyl-1,5-dimethyl-1H-pyrrole-2-carboxylic acid

A mixture of methyl 4-formyl-1,5-dimethyl-1H-pyrrole-2-carboxylate (14 g, 77.3 mmol) in NaOH/H₂O (4 M, 386 mL) and THF (386 mL) was stirred at 70° C. for 16 hrs. The mixture was filtered, the aqueous layer was adjusted pH=3 with HCl (3 M) at 0° C., then filtered to give 4-formyl-1,5-dimethyl-1H-pyrrole-2-carboxylic acid (11.37 g, 88%) as a yellow solid. MS (EI⁺, m/z): 168.1 [M+H]⁺.

### Step 6: 4-((4-(2-Chlorophenyl)piperazin-1-yl)methyl)-1,5-dimethyl-1H-pyrrole-2-carboxylic acid

To a mixture of 4-formyl-1,5-dimethyl-1H-pyrrole-2-carboxylic acid (7.5 g, 45 mmol) and 1-(2-chlorophenyl)piperazine (10.6 g, 54 mmol) in EtOH (50 mL) was added NaBH₃CN (5.67 g, 90 mmol). The mixture was stirred at rt for 16 hrs. The mixture was concentrated and then poured into H₂O (100 mL). The mixture was adjusted pH=3 with aq. HCl (3 M) at 0° C. and filtered to give 4-((4-(2-chlorophenyl)piperazin-1-yl)methyl)-1,5-dimethyl-1H-pyrrole-2-carboxylic acid (9.7 g, 62%) as a yellow solid. MS (EI⁺, m/z): 348.3 [M+H]⁺.

### General Procedures for Intermediate II

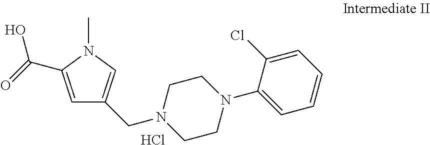 [SMILES: Cn1cc(CN2CCN(c3ccccc3Cl)CC2)cc1C(=O)O ; Cl]

### Synthetic Scheme:

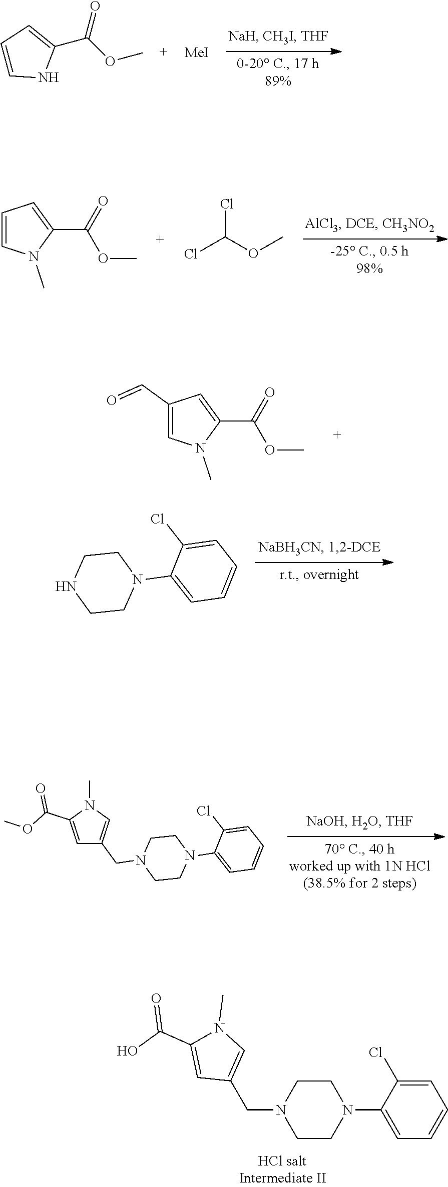

### Procedures and Characterization:

### Step 1: Methyl 1-methyl-1H-pyrrole-2-carboxylate

To a mixture of methyl-1H-pyrrole-2-carboxylate (10 g, 79.9 mmol) in anhydrous THF (200 mL) was added NaH (3.52 g, 60% w.t., 87.9 mmol) at 0° C. The resulting mixture was stirred at 20° C. for 30 min, then CH₃I (13.62 g, 95.9 mmol) was added. The reaction mixture was stirred at 20° C. for 17 hrs. The mixture was then poured into an aqueous solution of NH₄Cl (200 mL). The aqueous layer was extracted with EtOAc (200 mL×2). The combined organic layers were washed with brine (150 mL), dried (Na₂SO₄), filtered, and concentrated in vacuo. The residue was purified by chromatography (silica, EtOAc/PE=1/20) to afford methyl 1-methyl-1H-pyrrole-2-carboxylate (9.88 g, 71 mmol, 89%) as a yellow oil. MS (EI⁺, m/z): 140.1 [M+H]⁺.

### Step 2: Methyl 4-formyl-1-methyl-1H-pyrrole-2-carboxylate

To a mixture of methyl-1-methyl-1H-pyrrole-2-carboxylate (10 g, 71.86 mmol), AlCl₃ (21.08 g, 158.1 mmol) in 1,2-dichloroethane (100 mL), and MeNO₂ (100 mL) was added a solution of dichloro(methoxy)methane (8.26 g, 71.86 mmol) at −25° C. The resulting mixture was stirred at −25° C. for 0.5 hr. The mixture was then poured into ice (100 mL). The mixture was partitioned between CH₂Cl₂ (100 mL) and H₂O (100 mL). The layers were separated. The CH₂Cl₂ layer was dried over Na₂SO₄, filtered, and concentrated in vacuo. The residue was purified by chromatography (silica, EtOAc/PE=1/1) to afford methyl 4-formyl-1-methyl-1H-pyrrole-2-carboxylate (11.77 g, 70.42 mmol, 98%) as a yellow solid. MS (EI⁺, m/z): 168.2 [M+H]⁺. ¹H NMR (400 MHz, DMSO-d₆) δ 9.71 (s, 1H), 7.91 (d, J=1.8 Hz, 1H), 7.22 (d, J=1.8 Hz, 1H), 3.92 (s, 3H), 3.78 (s, 3H). NOESY confirmed this structure.

### Step 3: Methyl 4-((4-(2-chlorophenyl)piperazin-1-yl)methyl)-1-methyl-1H-pyrrole-2-carboxylate

To a mixture of methyl-4-formyl-1-methyl-1H-pyrrole-2-carboxylate (13 g, 77.77 mmol), 1-(2-chlorophenyl)piperazine (22.95 g, 116.66 mmol), and 1,2-dichloroethane (140 mL) was added NaBH₃CN (9.78 g, 155.54 mmol) at 20° C. The resulting mixture was stirred at 20° C. for 17 hrs. The reaction mixture was partitioned between CH₂Cl₂ (300 mL) and H₂O (300 mL). The CH₂Cl₂ layer was dried over Na₂SO₄, filtered, and concentrated in vacuo. The residue was purified by chromatography (silica, EtOAc/PE=1/1) to afford methyl 4-((4-(2-chlorophenyl)piperazin-1-yl)methyl)-1-methyl-1H-pyrrole-2-carboxylate (13.5 g crude) as a yellow oil. MS (EI⁺, m/z): 348.2 [M+H]⁺.

### Step 4: 4-((4-(2-Chlorophenyl)piperazin-1-yl)methyl)-1-methyl-1H-pyrrole-2-carboxylic acid

A mixture of methyl-4-((4-(2-chiorophenyl)piperazin-1-yl)methyl)-1-methyl-1H-pyrrole-2-carboxylate (13.5 g crude), NaOH (30.4 g, 758.97 mmol), H₂O (200 mL), and THF (100 mL) was stirred at 70° C. for 65 hrs. THF was removed by evaporation. The aqueous layer was extracted with 2-methyl-tetrahydrofuran (300 mL). The organic layer was concentrated in vacuo. The residue was partitioned between CH₂Cl₂ (400 mL) and H₂O (200 mL). The organic layer was dried (Na₂SO₄), filtered, acidified with the solution of HCl in dioxane (4 M, 20 mL, 80 mmol) to pH=1-2, and concentrated in vacuo to give 4-((4-(2-chlorophenyl)piperazin-1-yl)methyl)-1-methyl-1H-pyrrole-2-carboxylic acid (10 g, 29.96 mmol, 38.5% for 2 steps) as a yellow solid. MS (EI⁺, m/z): 334.2 [M+H]⁺.

¹H NMR (500 MHz, DMSO-d₆) δ 11.69 (s, 1H), 9.83 (s, 1H), 7.51-7.39 (m, 1H), 7.37-7.25 (m, 2H), 7.20-7.17 (m, 1H), 7.14-7.00 (m, 2H), 4.17-4.16 (m, 2H), 3.86 (s, 3H), 3.40-3.38 (m, 4H), 3.25-3.20 (m, 4H).

### Example 1: (4-((4-(2-Chlorophenyl)piperazin-1-yl)methyl)-1,5-dimethyl-1H-pyrrol-2-yl)(4-phenylpiperazin-1-yl)methanone, I-133

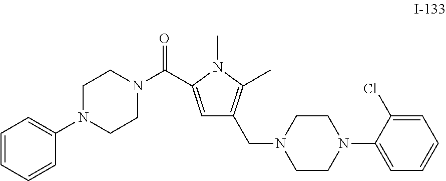 [SMILES: Cc1c(CN2CCN(c3ccccc3Cl)CC2)cc(C(=O)N2CCN(c3ccccc3)CC2)n1C]

### Synthetic Scheme:

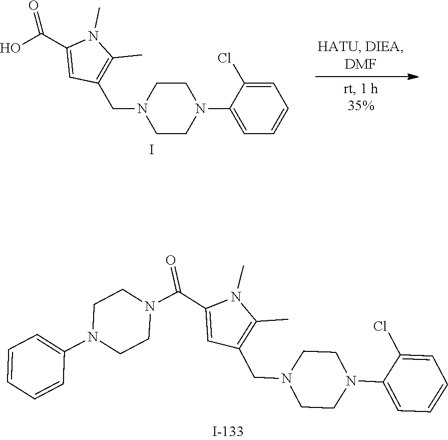

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: (4-((4-(2-Chlorophenyl)piperazin-1-yl)methyl)-1,5-dimethyl-1H-pyrrol-2-yl)(4-phenylpiperazin-1-yl)methanone

A mixture of 4-((4-(2-chlorophenyl)piperazin-1-yl)methyl)-1,5-dimethyl-1H-pyrrole-2-carboxylic acid (200 mg, 0.575 mmol), 1-phenylpiperazine (103 mg, 0.632 mmol), HATU (284 mg, 0.748 mmol) and DIPEA (149 mg, 1.15 mmol) in DMF (5 mL) was stirred at rt for 1 hr. The mixture was purified by prep-HPLC to afford (4-((4-(2-chlorophenyl) piperazin-1-yl) methyl)-1,5-dimethyl-1H-pyrrol-2-yl)(4-phenylpiperazin-1-yl)methanone I-133 (115 mg, 35%). MS (EI⁺, m/z): 492.3 [M+H]⁺.

¹H NMR (500 MHz, CDCl₃) δ 7.34 (dd, J=7.9, 1.1 Hz, 1H), 7.29 (t, J=7.9 Hz, 2H), 7.20 (dd, J=11.2, 4.1 Hz, 1H), 7.04 (d, J=8.0 Hz, 1H), 6.96 (t, J=6.3 Hz, 3H), 6.91 (t, J=7.3 Hz, 1H), 6.33 (s, 1H), 3.92 (t, J=5 Hz, 4H), 3.67 (s, 3H), 3.43 (s, 2H), 3.21 (t, J=5 Hz, 4H), 3.06 (s, 4H), 2.62 (s, 4H), 2.23 (s, 3H).

### Example 2: (4-((4-(2-Chlorophenyl)piperazin-1-yl)methyl)-1,5-dimethyl-1H-pyrrol-2-yl)(4-(3-fluorophenyl)piperazin-1-yl)methanone, I-108

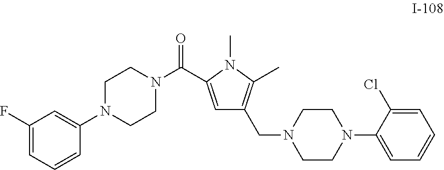 [SMILES: Cc1c(CN2CCN(c3ccccc3Cl)CC2)cc(C(=O)N2CCN(c3cccc(F)c3)CC2)n1C]

### Synthetic Scheme:

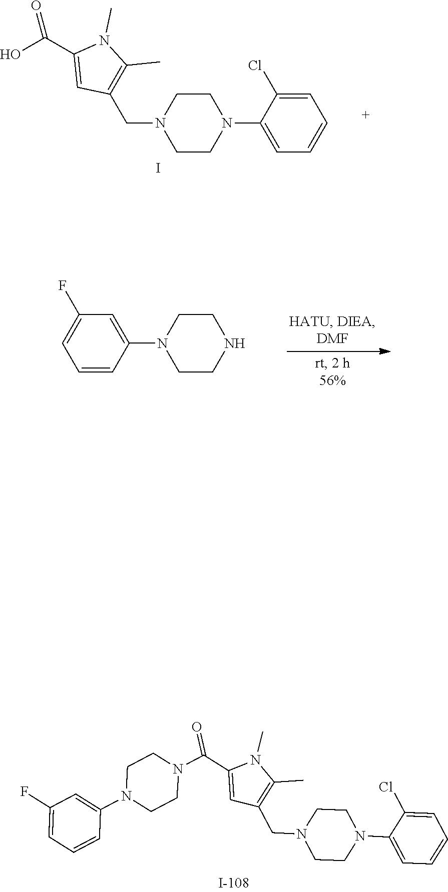

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). The synthesis of (4-((4-(2-Chlorophenyl)piperazin-1-yl)methyl)-1,5-dimethyl-1H-pyrrol-2-yl)(4-(3-fluorophenyl)piperazin-1-yl)methanone I-108 was the same as Example 1.

Analysis was performed following Method B. Separation was performed following Method D. MS (EI⁺, m/z): 510.3 [M+H]⁺. ¹H NMR (500 MHz, CDCl₃) δ 7.34 (dd, J=7.9, 1.3 Hz, 1H), 7.21 (m, 2H), 7.04 (dd, J=8.1, 1.2 Hz, 1H), 6.99-6.92 (m, 1H), 6.69 (dd, J=8.3, 2.0 Hz, 1H), 6.64-6.54 (m, 2H), 6.33 (s, 1H), 3.91 (t, J=5 Hz, 4H), 3.67 (s, 3H), 3.23 (t, J=5 Hz, 4H), 3.06 (s, 4H), 2.62 (s, 4H), 2.23 (s, 3H).

### Example 3: (4-(3-Chlorophenyl)piperazin-1-yl)(4-((4-(2-chlorophenyl) piperazin-1-yl)methyl)-1,5-dimethyl-1H-pyrrol-2-yl)methanone, I-118

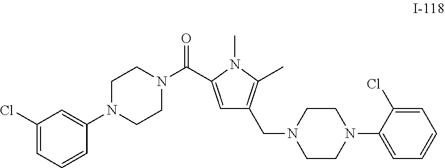 [SMILES: Cc1c(CN2CCN(c3ccccc3Cl)CC2)cc(C(=O)N2CCN(c3cccc(Cl)c3)CC2)n1C]

### Synthetic Scheme:

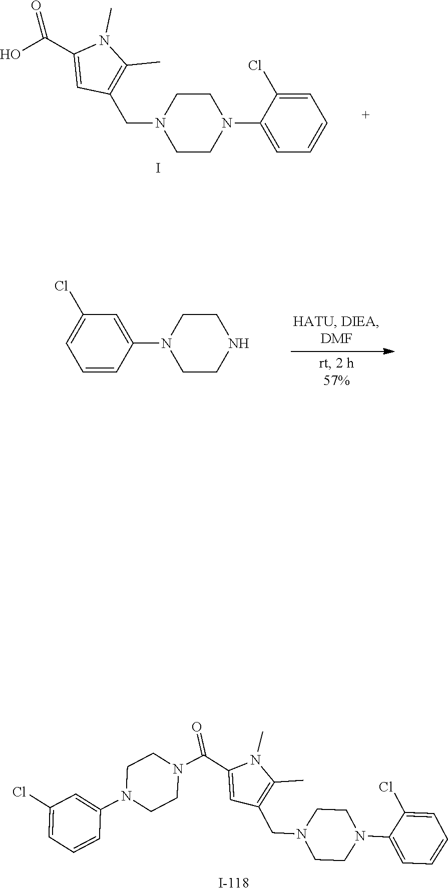

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). The synthesis of (4-(3-Chlorophenyl)piperazin-1-yl)(4-((4-(2-chlorophenyl) piperazin-1-yl)methyl)-1,5-dimethyl-1H-pyrrol-2-yl)methanone I-118 was the same as Example 1. Analysis was performed following Method B. Separation was performed following Method D. MS (EI⁺, m/z): 526.2 [M+H]⁺. ¹H NMR (500 MHz, CDCl₃) δ 7.34 (dd, J=7.9, 1.3 Hz, 1H), 7.20 (dt, J=10.2, 4.8 Hz, 2H), 7.04 (dd, J=8.0, 1.2 Hz, 1H), 6.95 (td, J=7.8, 1.3 Hz, 1H), 6.90 (t, J=2.0 Hz, 1H), 6.85 (dd, J=7.8, 1.2 Hz, 1H), 6.80 (dd, J=8.3, 2.1 Hz, 1H), 6.33 (s, 1H), 3.91 (t, J=5.0 Hz, 4H), 3.67 (s, 3H), 3.42 (s, 2H), 3.22 (t, J=5.0 Hz, 4H), 3.06 (s, 4H), 2.62 (s, 4H), 2.23 (s, 3H).

### Example 4: 3-(4-(4-((4-(2-chlorophenyl)piperazin-1-yl)methyl)-1,5-dimethyl-1H-pyrrole-2-carbonyl)piperazin-1-yl)benzonitrile, I-113

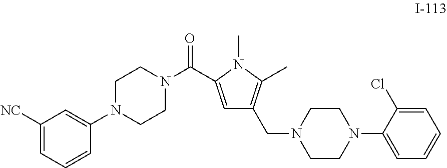 [SMILES: Cc1c(CN2CCN(c3ccccc3Cl)CC2)cc(C(=O)N2CCN(c3cccc(C#N)c3)CC2)n1C]

### Synthetic Scheme:

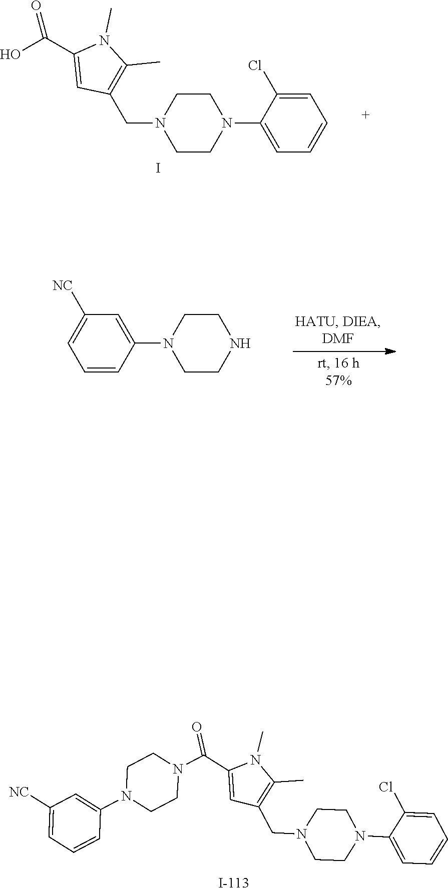

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). The synthesis of 3-(4-(4-((4-(2-Chlorophenyl)piperazin-1-yl)methyl)-1,5-dimethyl-1H-pyrrole-2-carbonyl)piperazin-1-yl)benzonitrile I-113 was the same as Example 1.

Analysis was performed following Method B. Separation was performed following Method D. MS (EI⁺, m/z): 517.3 [M+H]⁺. ¹H NMR (500 MHz, CDCl₃) δ 7.60 (d, J=7.5 Hz, 1H), 7.52 (t, J=7.8 Hz, 1H), 7.34 (d, J=7.8 Hz, 1H), 7.21 (t, J=7.5 Hz, 1H), 7.08-7.02 (m, 3H), 6.95 (t, J=7.5 Hz, 1H), 6.33 (s, 1H), 3.99 (s, 4H), 3.42 (s, 2H), 3.23 (d, J=4.3 Hz, 4H), 3.06 (s, 4H), 2.62 (s, 4H), 2.23 (s, 3H).

### Example 5: (4-((4-(2-Chlorophenyl)piperazin-1-yl)methyl)-1,5-dimethyl-1H-pyrrol-2-yl)(4-(4-fluorophenyl)piperazin-1-yl)methanone, I-115

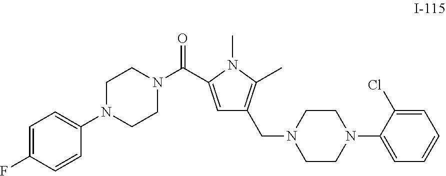 [SMILES: Cc1c(CN2CCN(c3ccccc3Cl)CC2)cc(C(=O)N2CCN(c3ccc(F)cc3)CC2)n1C]

### Procedure and Characterization:

The general procedure for Intermediate I was followed to obtain 4-((4-(2-chlorophenyl)piperazin-1-yl)methyl)-1,5-dimethyl-1H-pyrrole-2-carboxylic acid (I). The synthesis of (4-((4-(2-Chlorophenyl)piperazin-1-yl)methyl)-1,5-dimethyl-1H-pyrrol-2-yl)(4-(4-fluorophenyl)piperazin-1-yl)methanone I-115 was the same as Example 1.

Analysis was performed following Method B. Separation was performed following Method D. ESI-MS (EI⁺, m/z): 510.3 [M+H]⁺. ¹H NMR (500 MHz, DMSO-d₆) δ 7.38 (d, J=8.0 Hz, 1H), 7.28 (s, 1H), 7.14 (d, J=7.5 Hz, 1H), 7.02 (m, 5H), 6.25 (s, 1H), 3.76 (m, 4H), 3.55 (s, 3H), 3.34 (s, 2H), 3.30 (m, 4H), 3.14-3.07 (m, 4H), 2.94 (m, 4H), 2.19 (s, 3H).

### Example 6: (4-((4-(2-chlorophenyl)piperazin-1-yl)methyl)-1,5-dimethyl-1H-pyrrol-2-yl)(4-(3,5-difluorophenyl)piperazin-1-yl)methanone, I-96

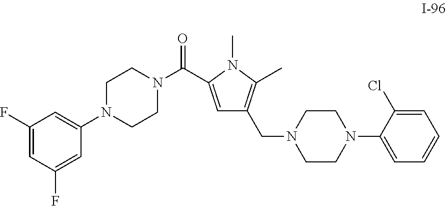 [SMILES: Cc1c(CN2CCN(c3ccccc3Cl)CC2)cc(C(=O)N2CCN(c3cc(F)cc(F)c3)CC2)n1C]

### Synthetic Scheme:

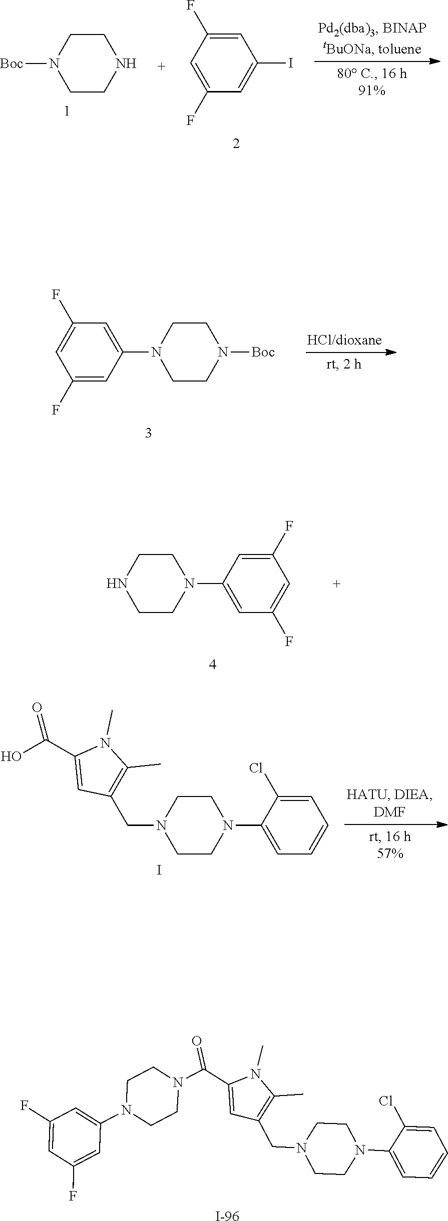

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: tert-Butyl 4-(3,5-difluorophenyl) piperazine-1-carboxylate

A solution of tert-butyl-piperazine-1-carboxylate (1.024 g, 5.5 mmol), 1,3-difluoro-5-iodobenzene (1.58 g, 6.6 mmol), t-BuONa (790 mg, 8.25 mmol), BINAP (137 mg, 0.22 mmol), Pd₂(dba)₃ (100 mg, 0.11 mmol) in dry toluene (40 mL) was stirred under N₂ at 80° C. for 16 hrs. The reaction mixture was concentrated and the residue was purified by chromatography (silica, EtOAc/PE=1/10) to afford 1-(3,5-difluorophenyl)piperazine (1.5 g, 5.03 mmol, 91%) as a yellow oil. ESI-MS (EI⁺, m/z): 243.2 [M−55]⁺.

### Step 2: 1-(3,5-Difluorophenyl) piperazine

The mixture of tert-butyl-4-(3,5-difluorophenyl)piperazine-1-carboxylate (1.4 g, 4.69 mmol) in HCl/dioxane (10 mL) was stirred at rt for 2 h. The reaction mixture was concentrated to give crude product 1-(3,5-difluorophenyl)piperazine (1.1 g) as a yellow solid. ESI-MS (EI⁺, m/z): 199.2 [M+H]⁺.

### Step 3: (4-((4-(2-Chlorophenyl) piperazin-1-yl) methyl)-1,5-dimethyl-1H-pyrrol-2-yl) (4-(3,5-difluorophenyl) piperazin-1-yl) methanone

The procedure for (4-((4-(2-chlorophenyl)piperazin-1-yl)methyl)-1,5-dimethyl-1H-pyrrol-2-yl)(4-(3,5-difluorophenyl)piperazin-1-yl)methanone I-96 was the same as Example 1. ESI-MS (EI⁺, m/z): 528.3 [M+H]⁺. ¹H NMR (500 MHz, CDCl₃) δ 7.34 (dd, J=7.9, 1.0 Hz, 1H), 7.21 (t, J=7.7 Hz, 1H), 7.04 (d, J=7.3 Hz, 1H), 6.96 (t, J=7.6 Hz, 1H), 6.38 (d, J=8.5 Hz, 2H), 6.33 (s, 1H), 6.30 (t, J=8.8 Hz, 1H), 3.90 (4, J=5.0 Hz, 4H), 3.67 (s, 3H), 3.42 (s, 2H), 3.24 (t, J=5.0 Hz, 4H), 3.06 (s, 4H), 2.62 (s, 4H), 2.23 (s, 3H).

### Example 7: (S)-(4-((4-(2-chlorophenyl)piperazin-1-yl)methyl)-1,5-dimethyl-1H-pyrrol-2-yl)(4-(4-fluorophenyl)-2-methylpiperazin-1-yl)methanone, I-95

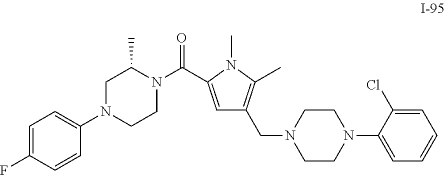 [SMILES: Cc1c(CN2CCN(c3ccccc3Cl)CC2)cc(C(=O)N2CCN(c3ccc(F)cc3)C[C@@H]2C)n1C]

### Synthetic Scheme:

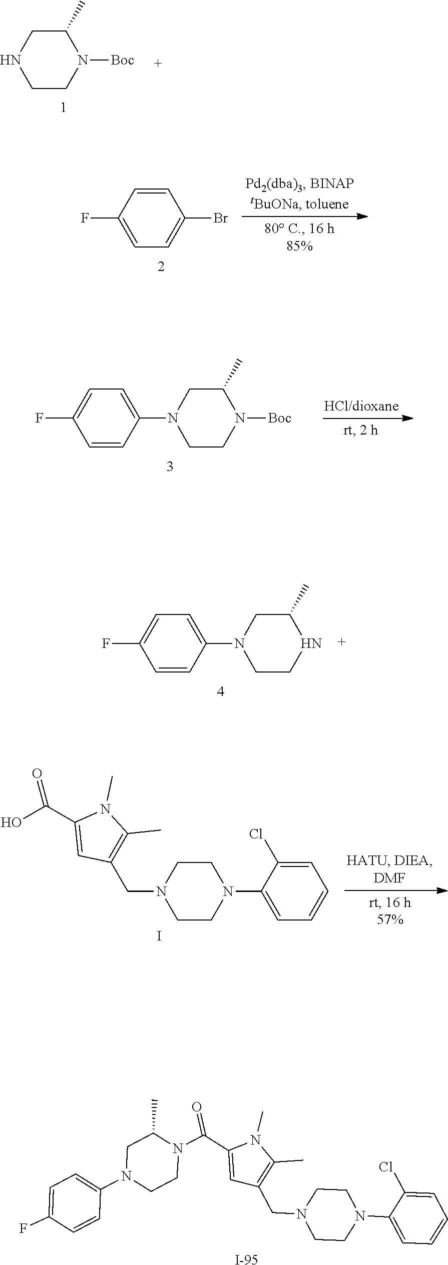

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: (S)-tert-butyl 4-(4-fluorophenyl)-2-methylpiperazine-1-carboxylate

A solution of (S)-tert-butyl 2-methylpiperazine-1-carboxylate (1.2 g, 6 mmol), 1-bromo-4-fluorobenzene (1.26 g, 7.2 mmol), t-BuONa (865 mg, 9 mmol), BINAP (150 mg, 0.24 mmol), Pd₂ (dba)₃ (110 mg, 0.12 mmol) in dry toluene (40 mL) was stirred under N₂ at 80° C. for 16 hrs. The reaction mixture was concentrated and the residue was purified by chromatography (silica, EtOAc/PE=1/10) to afford (S)-tert-butyl-4-(4-fluorophenyl)-2-methylpiperazine-1-carboxylate (1.5 g, 5.1 mmol, 85%) as a yellow oil. ESI-MS (EI⁺, m/z): 295.1 [M+H]⁺.

### Step 2: (S)-1-(4-fluorophenyl)-3-methylpiperazine

A mixture of (S)-tert-butyl-4-(4-fluorophenyl)-2-methylpiperazine-1-carboxylate (1.5 g, 5.1 mmol) in HCl/dioxane (20 mL, 4 M) was stirred at rt for 16 hrs. The reaction mixture was concentrated to give crude product (S)-1-(4-fluorophenyl)-3-methylpiperazine (1.2 g) as a yellow solid. ESI-MS (EI⁺, m/z): 195.2 [M+H]⁺.

### Step 3: (S)-(4-((4-(2-chlorophenyl) piperazin-1-yl) methyl)-1,5-dimethyl-1H-pyrrol-2-yl) (4-(4-fluorophenyl)-2-methylpiperazin-1-yl) methanone

The procedure for (S)-(4-((4-(2-chlorophenyl)piperazin-1-yl)methyl)-1,5-dimethyl-1H-pyrrol-2-yl)(4-(4-fluorophenyl)-2-methylpiperazin-1-yl)methanone I-95 was the same as Example 1.

ESI-MS (EI⁺, m/z): 524.2 [M+H]⁺. ¹H NMR (500 MHz, CDCl₃) δ 7.34 (d, J=7.8 Hz, 1H), 7.21 (t, J=7.6 Hz, 1H), 7.04 (d, J=7.9 Hz, 1H), 6.97 (m, 3H), 6.87 (dd, J=9.0, 4.5 Hz, 2H), 6.30 (s, 1H), 4.86 (s, 1H), 4.41 (d, J=13.4 Hz, 1H), 3.65 (s, 3H), 3.49-3.43 (m, 4H), 3.31 (d, J=11.8 Hz, 1H), 3.06 (s, 4H), 2.91 (dd, J=11.8, 3.5 Hz, 1H), 2.77-2.71 (m, 1H), 2.62 (s, 4H), 2.23 (s, 3H), 1.45 (d, J=6.8 Hz, 3H).

### Example 8: (S)-(4-((4-(2-chlorophenyl)piperazin-1-yl)methyl)-1,5-dimethyl-1H-pyrrol-2-yl)(4-(3,5-difluorophenyl)-2-methylpiperazin-1-yl)methanone, I-93

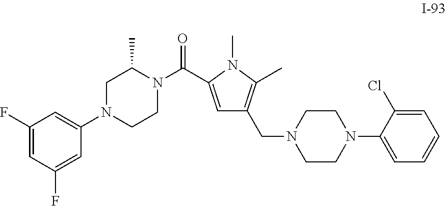 [SMILES: Cc1c(CN2CCN(c3ccccc3Cl)CC2)cc(C(=O)N2CCN(c3cc(F)cc(F)c3)C[C@@H]2C)n1C]

### Synthetic Scheme:

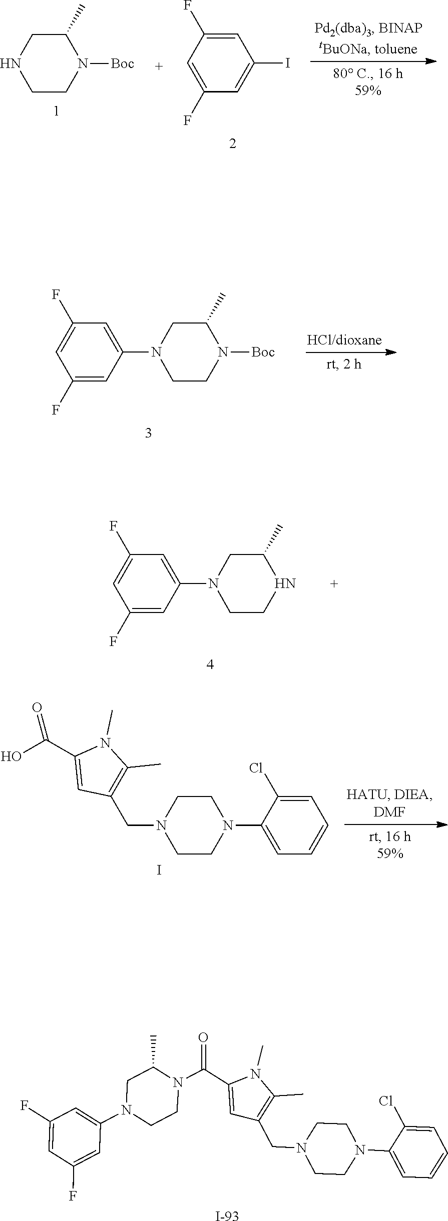

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: (S)-tert-butyl 4-(3,5-difluorophenyl)-2-methylpiperazine-1-carboxylate

A solution of (S)-tert-butyl-2-methylpiperazine-1-carboxylate (1.2 g, 6 mmol), 1,3-difluoro-5-iodobenzene (1.685 g, 7.2 mmol), t-BuONa (865 mg, 9 mmol), BINAP (150 mg, 0.24 mmol), Pd₂(dba)₃ (110 mg, 0.12 mmol) in dry toluene (40 mL) was stirred under N₂ at 80° C. for 16 hrs. The reaction mixture was concentrated and the mixture was purified by chromatography (silica, EtOAc/PE=1/10) to afford (S)-tert-butyl-4-(3,5-difluorophenyl)-2-methylpiperazine-1-carboxylate (1.1 g, 3.52 mmol, 59%) as a yellow oil. ESI-MS (EI⁺, m/z): 257.0 [M−55]⁺.

### Step 2: (S)-1-(3,5-difluorophenyl)-3-methylpiperazine

The mixture of (S)-tert-butyl-4-(3,5-difluorophenyl)-2-methylpiperazine-1-carboxylate (1.1 g, 3.52 mmol) in HCl/dioxane (10 mL) was stirred at rt for 16 hrs. The reaction mixture was concentrated to give crude product (S)-1-(3,5-difluorophenyl)-3-methylpiperazine (1.0 g) as yellow solid. ESI-MS (EI⁺, m/z): 213.1 [M+H]⁺.

### Step 3: (S)-(4-((4-(2-chlorophenyl)piperazin-1-yl)methyl)-1,5-dimethyl-1H-pyrrol-2-yl)(4-(3,5-difluorophenyl)-2-methylpiperazin-1-yl)methanone

The procedure for (S)-(4-((4-(2-chlorophenyl)piperazin-1-yl)methyl)-1,5-dimethyl-1H-pyrrol-2-yl)(4-(3,5-difluorophenyl)-2-methylpiperazin-1-yl)methanone I-93 was the same as Example 1.

ESI-MS (EI⁺, m/z): 542.2 [M+H]⁺. ¹H NMR (500 MHz, CDCl₃) δ 7.34 (d, J=7.9 Hz, 1H), 7.21 (t, J=7.7 Hz, 1H), 7.04 (d, J=8.0 Hz, 1H), 6.96 (t, J=7.3 Hz, 1H), 6.44-6.19 (m, 4H), 4.86 (s, 1H), 4.41 (d, J=13.3 Hz, 1H), 3.65 (s, 3H), 3.55 (d, J=11.7 Hz, 1H), 3.50-3.35 (m, 4H), 3.17-2.96 (m, 5H), 2.86 (td, J=11.7, 3.4 Hz, 1H), 2.62 (s, 4H), 2.23 (s, 3H), 1.39 (d, J=6.7 Hz, 3H).

### Example 9: (4-(3-chloro-4-fluorophenyl)piperazin-1-yl)(4-((4-(2-chlorophenyl)piperazin-1-yl)methyl)-1,5-dimethyl-1H-pyrrol-2-yl)methanone, I-82

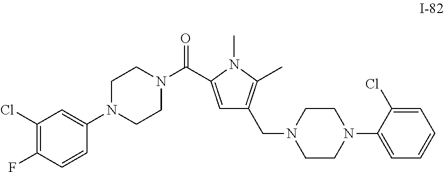 [SMILES: Cc1c(CN2CCN(c3ccccc3Cl)CC2)cc(C(=O)N2CCN(c3ccc(F)c(Cl)c3)CC2)n1C]

### Synthetic Scheme:

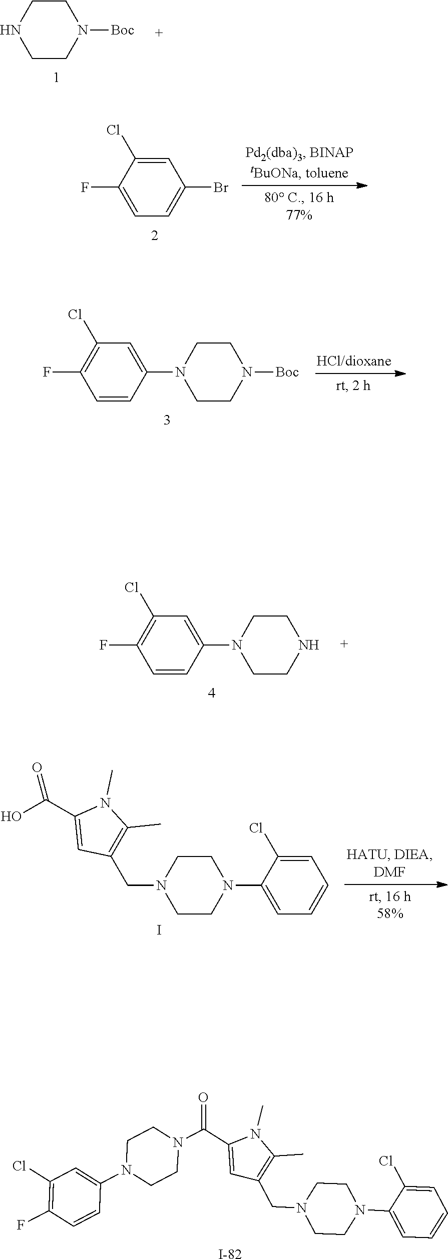

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: tert-Butyl 4-(3-chloro-4-fluorophenyl)piperazine-1-carboxylate

A solution of tert-butyl-piperazine-1-carboxylate (1.0 g, 5.37 mmol), 4-bromo-2-chloro-1-fluorobenzene (1.35 g, 6.44 mmol), t-BuONa (768 mg, 8.06 mmol), BINAP (125 mg, 0.2 mmol), Pd₂(dba)₃ (92 mg, 0.1 mmol) in dry toluene (10 mL) was stirred under N₂ at 80° C. for 16 hrs. The reaction mixture was concentrated and the mixture was purified by chromatography (silica, EtOAc/PE=1/10) to afford tert-butyl-4-(3-chloro-4-fluorophenyl)piperazine-1-carboxylate (1.3 g, 4.13 mmol, 77%) as a yellow oil. ESI-MS (EI⁺, m/z): 259 [M−55]⁺.

### Step 2: 1-(3-Chloro-4-fluorophenyl)piperazine

A mixture of tert-butyl 4-(3-chloro-4-fluorophenyl)piperazine-1-carboxylate (1.3 g, 4.13 mmol) in HCl/dioxane (10 mL) was stirred at rt for 2 hrs. The reaction mixture was concentrated to give crude product 1-(3-chloro-4-fluorophenyl)piperazine (1.1 g) as a yellow solid. ESI-MS (EI⁺, m/z): 215.1 [M+H]⁺.

### Step 3: (4-(3-Chloro-4-fluorophenyl)piperazin-1-yl)(4-((4-(2-chlorophenyl)piperazin-1-yl)methyl)-1,5-dimethyl-1H-pyrrol-2-yl)methanone

The procedure for (4-(3-chloro-4-fluorophenyl)piperazin-1-yl)(4-((4-(2-chlorophenyl)piperazin-1-yl)methyl)-1,5-dimethyl-1H-pyrrol-2-yl)methanone I-82 was the same as Example 1.

ESI-MS (EI⁺, m/z): 544.2 [M+H]⁺. ¹H NMR (500 MHz, CDCl₃) δ 7.34 (d, J=7.7 Hz, 1H), 7.21 (t, J=7.6 Hz, 1H), 7.05 (t, J=8.8 Hz, 2H), 6.99-6.91 (m, 2H), 6.79 (dt, J=8.9, 3.3 Hz, 1H), 6.32 (s, 1H), 3.91 (t, J=5.0 Hz, 4H), 3.67 (s, 3H), 3.42 (s, 2H), 3.13 (t, J=5.0 Hz, 4H), 3.06 (s, 4H), 2.62 (s, 4H), 2.23 (s, 3H).

### Example 10: (4-((4-(2-chlorophenyl)piperazin-1-yl)methyl)-1,5-dimethyl-1H-pyrrol-2-yl)(4-(3,4-difluorophenyl)piperazin-1-yl)methanone, I-109

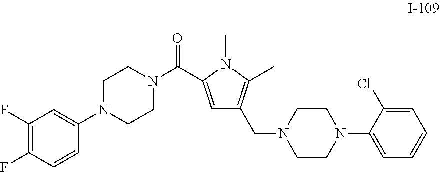 [SMILES: Cc1c(CN2CCN(c3ccccc3Cl)CC2)cc(C(=O)N2CCN(c3ccc(F)c(F)c3)CC2)n1C]

### Synthetic Scheme:

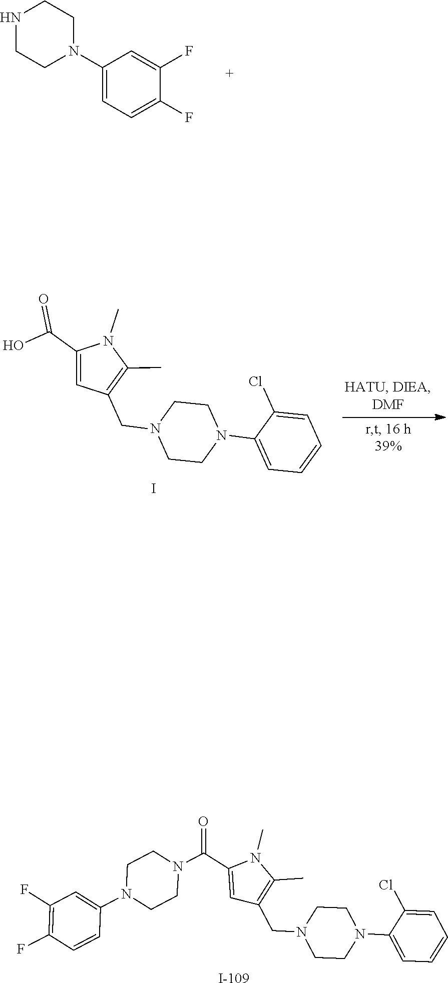

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid. The procedure for (4-((4-(2-chlorophenyl)piperazin-1-yl)methyl)-1,5-dimethyl-1H-pyrrol-2-yl)(4-(3,4-difluorophenyl)piperazin-1-yl)methanone I-109 was the same as Example 1.

Analysis was performed following Method A. Separation was performed following Method D. ESI-MS (EI+, m/z): 528.3 [M+H]⁺. ¹H NMR (400 MHz, CDCl₃) δ 7.35 (d, J=6.9 Hz, 1H), 7.22 (dd, J=17.9, 11.1 Hz, 1H), 7.13-6.90 (m, 3H), 6.81-6.67 (m, 1H), 6.62 (d, J=8.3 Hz, 1H), 6.33 (s, 1H), 3.91 (s, 4H), 3.67 (s, 3H), 3.43 (s, 2H), 3.10 (d, J=29.1 Hz, 8H), 2.63 (s, 4H), 2.20 (d, J=22.7 Hz, 3H).

### Example 11: (4-((4-(2-azidophenyl)piperazin-1-yl)methyl)-1,5-dimethyl-1H-pyrrol-2-yl)(4-phenylpiperazin-1-yl)methanone, I-97

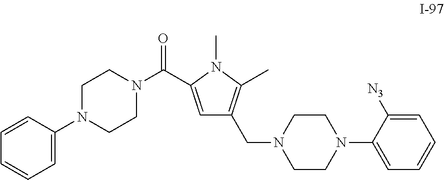 [SMILES: Cc1c(CN2CCN(c3ccccc3N=[N+]=[N-])CC2)cc(C(=O)N2CCN(c3ccccc3)CC2)n1C]

### Synthetic Scheme:

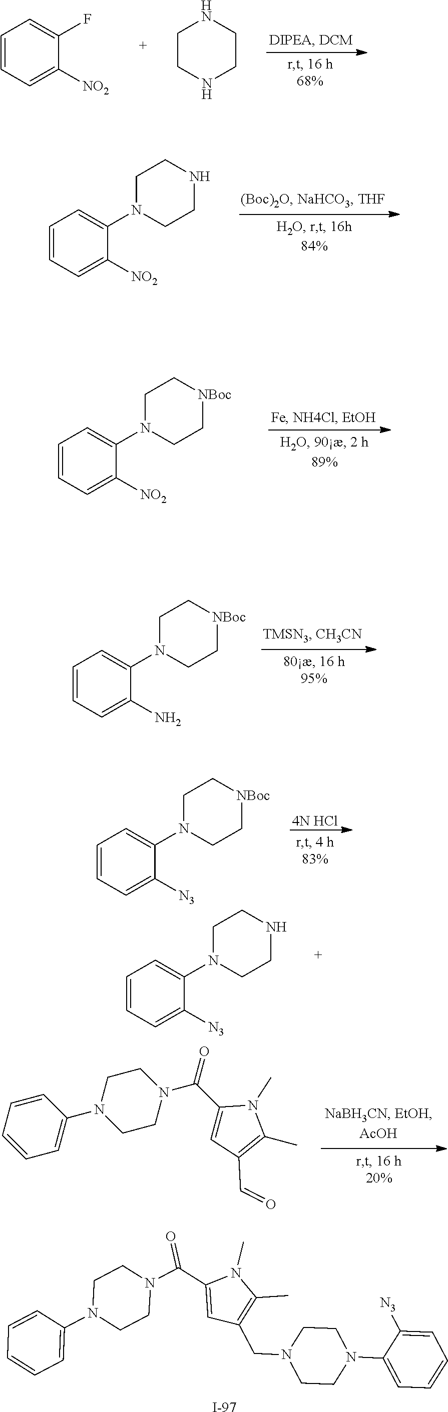

### Procedures and Characterization:

Analysis was performed following Method A. Separation was performed following Method D.

### Step 1: 1-(2-nitrophenyl)piperazine

To a solution of 1-fluoro-2-nitrobenzene (3.0 g, 21.26 mmol) in DCM (60 mL) at rt was added piperazine (2.20 g, 25.51 mmol) and DIPEA (5.50 g, 42.52 mmol). The reaction mixture was stirred at rt for 16 hrs, then the mixture was purified by chromatography (silica, EtOAc/PE=1/2) to afford 1-(2-nitrophenyl)piperazine (3.0 g, 14.48 mmol, 68%) as an oil. ESI-MS (EI+, m/z): 208.2 [M+H]⁺.

### Step 2: tert-butyl 4-(2-nitrophenyl)piperazine-1-carboxylate

To a solution of 1-(2-nitrophenyl)piperazine (800 mg, 3.86 mmol) in H₂O (10 mL) and THF (20 mL) at rt was added (Boc)₂O (1.01 g, 4.63 mmol) and NaHCO₃ (1.62 g, 19.3 mmol). The mixture was stirred at rt for 16 hrs, then diluted with H₂O (200 mL) and extracted with EtOAc (50 mL×3). The combined organic layers were washed with brine, dried over Na₂SO₄, filtered, concentrated, and purified by chromatography (silica, EtOAc/PE=1/10) to afford tert-butyl-4-(2-nitrophenyl)piperazine-1-carboxylate (1.0 g, 3.25 mmol, 84%). ESI-MS (EI+, m/z): 308.4 [M+H]⁺.

### Step 3: tert-butyl 4-(2-aminophenyl)piperazine-1-carboxylate

To a solution of tert-butyl-4-(2-nitrophenyl)piperazine-1-carboxylate (1.0 g, 3.25 mmol) in EtOH (30 mL) and H₂O (15 mL) at rt was added NH₄Cl (870 mg, 16.25 mmol) and Fe (1.09 g, 19.5 mmol). The reaction mixture was stirred at 90° C. for 2 hrs, then cooled to rt and extracted with EtOAc (50 mL×3). The combined organic layers were washed with brine, dried over Na₂SO₄, filtered, concentrated, and purified by chromatography (silica, EtOAc/PE=1/5) to afford tert-butyl-4-(2-aminophenyl)piperazine-1-carboxylate (800 mg, 2.88 mmol, 89%) as an oil. ESI-MS (EI+, m/z): 278.4 [M+H]⁺.

### Step-4: tert-butyl 4-(2-azidophenyl)piperazine-1-carboxylate

To a solution of tert-butyl-4-(2-aminophenyl)piperazine-1-carboxylate (700 mg, 2.52 mmol) in CH₃CN (10 mL) at rt was added isoamyl nitrite (444 mg, 3.79 mmol) and TMSN₃ (436 mg, 3.79 mmol). The mixture was stirred at 80° C. for 16 hrs, then cooled to rt. The mixture was diluted with H₂O (100 mL), extracted with EtOAc (50 mL×3). The combined organic layers were washed with brine, dried over Na₂SO₄, filtered, concentrated, and purified by chromatography (silica, EtOAc/PE=1/10) to afford tert-butyl-4-(2-azidophenyl)piperazine-1-carboxylate (730 mg, 2.41 mmol, 95%) as an oil. ESI-MS (EI+, m/z): 304.2 [M+H]⁺.

### Step 5: 1-(2-azidophenyl)piperazine

A solution of tert-butyl-4-(2-azidophenyl)piperazine-1-carboxylate (2.33 g, 7.68 mmol) in 4N HCl/dioxane (19.2 mL, 76.8 mmol) was stirred at rt for 4 hrs, then concentrated to give a crude residue, which was diluted with H₂O (100 mL) and washed with aq. NaHCO₃ (50 mL). The mixture was extracted with EtOAc (80 mL×3). The combined organic layers were washed with brine, dried over Na₂SO₄, filtered, concentrated, and purified by chromatography (silica, EtOAc/PE=1/1) to afford 1-(2-azidophenyl)piperazine (1.3 g, 6.4 mmol, 83%) as an oil. ESI-MS (EI+, m/z): 204.4 [M+H]⁺.

### Step 6: (4-((4-(2-azidophenyl)piperazin-1-yl)methyl)-1,5-dimethyl-1H-pyrrol-2-yl)(4-phenylpiperazin-1-yl)methanone

To a solution of 1-(2-azidophenyl)piperazine (209 mg, 1.03 mmol) in EtOH (5 mL) was added 1,2-dimethyl-5-(4-phenylpiperazine-1-carbonyl)-1H-pyrrole-3-carbaldehyde (320 mg, 1.03 mmol), NaBH₃CN (192 mg, 3.08 mmol) and two drops of acetic acid. The reaction mixture was stirred at rt for 16 hrs, then purified by prep-HPLC to afford (4-((4-(2-azidophenyl)piperazin-1-yl)methyl)-1,5-dimethyl-1H-pyrrol-2-yl)(4-phenylpiperazin-1-yl)methanone I-97 (100 mg, 0.2 mmol, 20%) as a white solid. ESI-MS (EI+, m/z): 499.2 [M+H]⁺. ¹H NMR (400 MHz, DMSO-d₆) δ 7.23 (t, J=7.9 Hz, 2H), 7.16-7.03 (m, 4H), 6.97 (d, J=8.0 Hz, 2H), 6.81 (t, J=7.2 Hz, 1H), 6.25 (s, 1H), 3.76 (d, J=4.6 Hz, 4H), 3.55 (s, 3H), 3.33 (s, 2H), 3.19-3.13 (m, 4H), 2.93 (s, 4H), 2.61-2.40 (m, 4H), 2.19 (s, 3H).

### Example 12: (S)-(4-((4-(2-azidophenyl)piperazin-1-yl)methyl)-1,5-dimethyl-1H-pyrrol-2-yl)(2-methyl-4-phenylpiperazin-1-yl)methanone, I-83

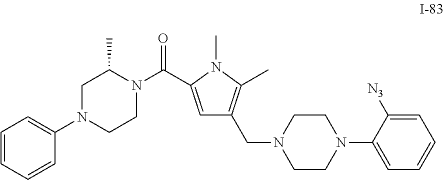 [SMILES: Cc1c(CN2CCN(c3ccccc3N=[N+]=[N-])CC2)cc(C(=O)N2CCN(c3ccccc3)C[C@@H]2C)n1C]

### Synthetic Scheme:

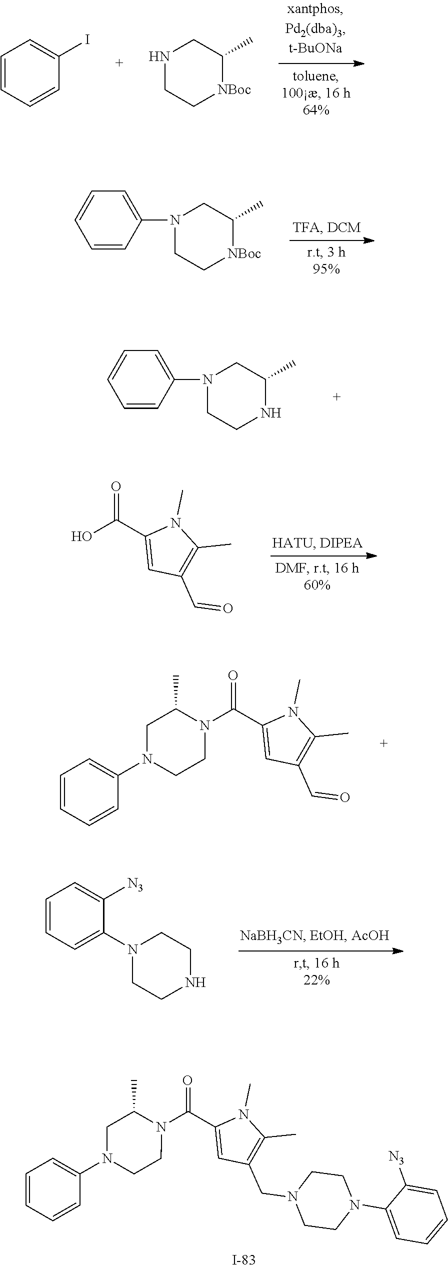

### Procedures and Characterization:

Analysis was performed following Method A. Separation was performed following Method D.

### Step 1: (S)-tert-butyl-2-methyl-4-phenylpiperazine-1-carboxylate

To a solution of iodobenzene (2.08 g, 10.19 mmol) in toluene (60 mL) at rt was added (S)-tert-butyl-2-methylpiperazine-1-carboxylate (1.7 g, 8.49 mmol), Pd₂(dba)₃ (778 mg, 0.85 mmol), t-BuONa (2.44 g, 25.46 mmol), XantPhos (982 mg, 1.70 mmol) under nitrogen. The reaction mixture was stirred at 100° C. for 16 h, then cooled to rt and diluted with EtOAc (60 mL×3). The combined organic layers were washed with brine, dried over Na₂SO₄, filtered, concentrated, and purified by chromatography (silica, EtOAc/PE=1/10) to afford (S)-tert-butyl-2-methyl-4-phenylpiperazine-1-carboxylate (1.8 g, 6.5 mmol, 64%) as an oil. ESI-MS (EI+, m/z): 277.2 [M+H]⁺.

### Step 2: (S)-3-methyl-1-phenylpiperazine

To art solution of (S)-tert-butyl 2-methyl-4-phenylpiperazine-1-carboxylate (1.8 g, 6.5 mmol) in DCM (20 mL) was added TFA (10 mL). The mixture was stirred at rt for 3 hrs, then concentrated and purified by chromatography (silica, EtOAc/PE=1/1) to afford (S)-3-methyl-1-phenylpiperazine (1.2 g, 6.17 mmol, 95%) as an oil. ESI-MS (EI+, m/z): 177.2 [M+H]⁺.

### Step 3: (S)-1,2-dimethyl-5-(2-methyl-4-phenylpiperazine-1-carbonyl)-1H-pyrrole-3-carbaldehyde

To art solution of (S)-3-methyl-1-phenylpiperazine (200 mg, 1.13 mmol) in DMF (20 mL) was added 4-formyl-1,5-dimethyl-1H-pyrrole-2-carboxylic acid (189 mg, 1.13 mmol), EDCI 326 mg, 1.7 mmol), HOBT (232 mg, 1.7 mmol), and DIPEA (584 mg, 4.52 mmol). The mixture was stirred at rt for 16 hrs, then diluted with H₂O (100 mL) and extracted with EtOAc (80 mL×3). The combined organic layers were washed with brine, dried over Na₂SO₄, filtered, concentrated, and purified by chromatography (silica, EtOAc/PE=1/1) to afford (S)-1,2-dimethyl-5-(2-methyl-4-phenylpiperazine-1-carbonyl)-1H-pyrrole-3-carbaldehyde (300 mg, 0.92 mmol, 82%) as a solid. ESI-MS (EI+, m/z): 326.3 [M+H]⁺.

### Step 4: (S)-(4-((4-(2-azidophenyl)piperazin-1-yl)methyl)-1,5-dimethyl-1H-pyrrol-2-yl)(2-methyl-4-phenylpiperazin-1-yl)methanone

To a solution of 1-(2-azidophenyl)piperazine (188 mg, 0.92 mmol) in EtOH (6 mL) at 0° C. was added (S)-1,2-dimethyl-5-(2-methyl-4-phenylpiperazine-1-carbonyl)-1H-pyrrole-3-carbaldehyde (300 mg, 0.92 mmol), NaBH₃CN (172 mg, 2.77 mmol), and two-drops of acetic acid. The mixture was stirred at rt for 16 hrs, then purified by prep-HPLC to afford (S)-(4-((4-(2-azidophenyl)piperazin-1-yl)methyl)-1,5-dimethyl-1H-pyrrol-2-yl)(2-methyl-4-phenylpiperazin-1-yl)methanone I-83 (105 mg, 0.2 mmol, 22%) as a white solid. ESI-MS (EI+, m/z): 513.2 [M+H]⁺. ¹H NMR (400 MHz, DMSO) δ 7.28-7.18 (m, 2H), 7.15-7.02 (m, 4H), 6.94 (d, J=8.0 Hz, 2H), 6.80 (t, J=7.3 Hz, 1H), 6.24 (d, J=15.3 Hz, 1H), 4.66 (s, 1H), 4.20 (d, J=13.4 Hz, 1H), 3.80-3.49 (m, 5H), 3.37 (s, 1H), 2.87 (dt, J=12.0, 11.4 Hz, 6H), 2.65 (td, J=11.8, 3.2 Hz, 1H), 2.57-2.36 (m, 5H), 2.19 (s, 3H), 1.31 (d, J=6.7 Hz, 3H).

### Example 13: (S)-(4-(1H-indol-7-yl)-2-methylpiperazin-1-yl)(4-((4-(2-chlorophenyl)piperazin-1-yl)methyl)-1,5-dimethyl-1H-pyrrol-2-yl)methanone, I-31

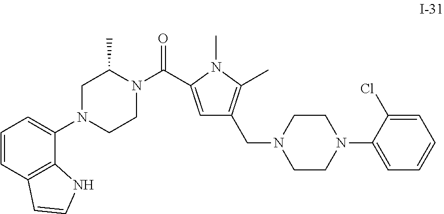 [SMILES: Cc1c(CN2CCN(c3ccccc3Cl)CC2)cc(C(=O)N2CCN(c3cccc4cc[nH]c34)C[C@@H]2C)n1C]

### Synthetic Scheme:

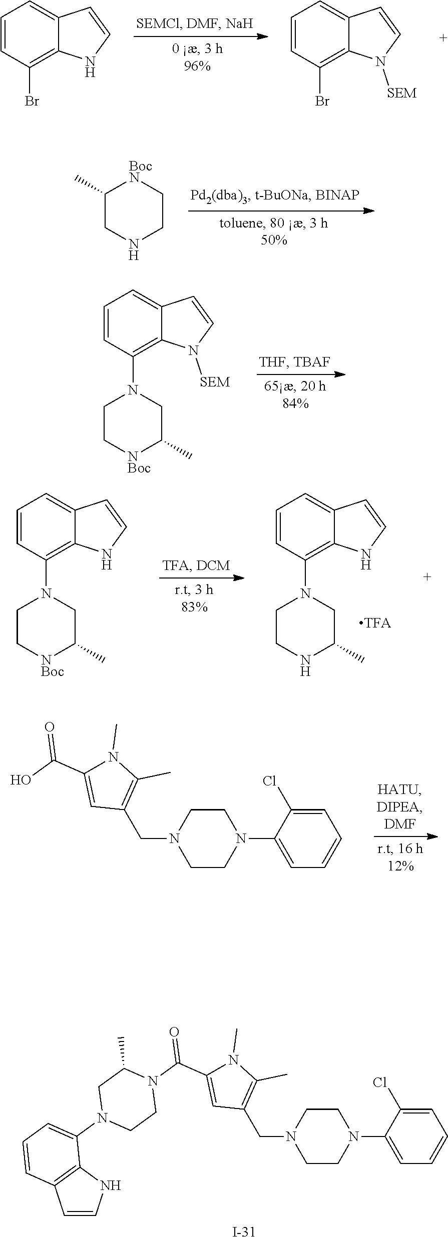

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid. Analysis was performed following Method A. Separation was performed following Method C.

### Step 1: 7-bromo-1-((2-(trimethylsilyl)ethoxy)methyl)-1H-indole

To a solution of 7-bromo-1H-indole (2.5 g, 12.75 mmol) in anhydrous DMF (30 mL) at rt was added NaH (765 mg, 19.13 mmol). The reaction mixture was stirred at 0° C. for 1 hr, then (2-(chloromethoxy)ethyl)trimethylsilane (2.76 g, 16.58 mmol) was added, and the reaction was stirred at 0° C. for 3 hrs. The mixture was poured into ice water and extracted with EtOAc (100 mL×2). The combined organic layers were washed with brine (100 mL), dried over Na₂SO₄, filtered, and concentrated in vacuo. The crude product was purified by chromatography (silica, EtOAc/PE=1/20) to afford 7-bromo-1-((2-(trimethylsilyl)ethoxy)methyl)-1H-indole (4.0 g, 12.26 mmol, 96%) as a white solid. ESI-MS (EI+, m/z): 326.1 [M+H]⁺.

### Step 2: (S)-tert-butyl-2-methyl-4-(1-((2-(trimethylsilyl)ethoxy)methyl)-1H-indol-7-yl)piperazine-1-carboxylate

To a rt solution of 7-bromo-1-((2-(trimethylsilyl)ethoxy)methyl)-1H-indole (3.8 g, 11.64 mmol) in anhydrous toluene (100 mL) was added (S)-tert-butyl-2-methylpiperazine-1-carboxylate (2.79 g, 13.98 mmol), Pd₂(dba)₃ (1.07 g, 1.17 mmol), t-BuONa (2.24 g, 23.29 mmol), and BINAP (1.45 g, 2.33 mmol) under nitrogen. The reaction mixture was stirred at 80° C. for 3 hrs, then cooled to rt, diluted with H₂O (200 mL), and extracted with EtOAc (100 mL×2). The combined organic layers were washed with brine (100 mL), dried over Na₂SO₄, filtered, and concentrated in vacuo. The crude product was purified by chromatography (silica, EtOAc/PE=1/10) to afford (S)-tert-butyl-2-methyl-4-(1-((2-(trimethylsilyl)ethoxy)methyl)-1H-indol-7-yl)piperazine-1-carboxylate (2.6 g, 5.83 mmol, 50.1%) as a yellow oil. ESI-MS (EI+, m/z): 446.3 [M+H]⁺.

### Step 3: (S)-tert-butyl-4-(1H-indol-7-yl)-2-methylpiperazine-1-carboxylate

To a rt solution of (S)-tert-butyl-2-methyl-4-(1-((2-(trimethylsilyl)ethoxy)methyl)-1H-indol-7-yl)piperazine-1-carboxylate (2.5 g, 5.61 mmol) in anhydrous THF (6.0 mL) was added TBAF (6.0 mL, 1M in THF). The reaction mixture was stirred at 65° C. for 20 hrs. The mixture was then cooled to rt, concentrated, and purified by chromatography (silica, EtOAc/PE=1/5) to afford (S)-tert-butyl-4-(1H-indol-7-yl)-2-methylpiperazine-1-carboxylate (1.5 g, 4.76 mmol, 84%) as a white oil. ESI-MS (EI+, m/z): 338.3 [M+Na].

Step 4: (S)-7-(3-methylpiperazin-1-yl)-1H-indole trifluoroacetate

To a solution of (S)-tert-butyl-4-(1H-indol-7-yl)-2-methylpiperazine-1-carboxylate (1.5 g, 4.76 mmol) in DCM (5.0 mL) at 0° C. was added TFA (2.31 g, 23.78 mmol). The reaction mixture was stirred at rt for 3 hrs, then concentrated to afford (S)-7-(3-methylpiperazin-1-yl)-1H-indole trifluoroacetate (1.25 g, 4.52 mmol, 87%) as a yellow oil. ESI-MS (EI+, m/z): 238.3 [M+Na]⁺

### Step 5: (S)-(4-(1H-indol-7-yl)-2-methylpiperazin-1-yl)(4-((4-(2-chlorophenyl)piperazin-1-yl)methyl)-1,5-dimethyl-1H-pyrrol-2-yl)methanone trifluoroacetate

To a solution of (S)-7-(3-methylpiperazin-1-yl)-1H-indole trifluoroacetate (620 mg, 1.91 mmol) in DMF (10 mL) at 0° C. was added 4-((4-(2-chlorophenyl)piperazin-1-yl)methyl)-1,5-dimethyl-1H-pyrrole-2-carboxylic acid (655 mg, 1.91 mmol), HATU (1.09 g, 2.87 mmol), and DIPEA (740 mg, 5.73 mmol). The reaction mixture was stirred at rt for 16 hrs, then purified by prep-HPLC to afford (S)-(4-(1H-indol-7-yl)-2-methylpiperazin-1-yl)(4-((4-(2-chlorophenyl)piperazin-1-yl)methyl)-1,5-dimethyl-1H-pyrrol-2-yl)methanone trifluoroacetate I-31 (150 mg, 0.23 mmol, 12%) as a white solid. ESI-MS (EI+, m/z): 545.3 [M+H]⁺. ¹H NMR (500 MHz, CDCl3) δ 12.47 (s, 1H), 8.60 (s, 1H), 7.40 (dd, J=28.2, 7.8 Hz, 2H), 7.26-7.23 (m, 2H), 7.09-7.04 (m, 3H), 6.84 (d, J=7.5 Hz, 1H), 6.57 (s, 1H), 6.48 (s, 1H), 4.88 (s, 1H), 4.39 (s, 1H), 4.12 (q, J=13.6 Hz, 2H), 3.68 (s, 3H), 3.64 (d, J=11.0 Hz, 3H), 3.47-3.37 (m, 3H), 3.28 (t, J=12.2 Hz, 3H), 3.11-2.84 (m, 4H), 2.27 (s, 3H), 1.61 (d, J=6.7 Hz, 3H).

### Example 14: (4-((4-(2-Chlorophenyl)piperazin-1-yl)methyl)-1,5-dimethyl-1H-pyrrol-2-yl)(4-(3,4,5-trifluorophenyl)piperazin-1-yl)methanone, I-111

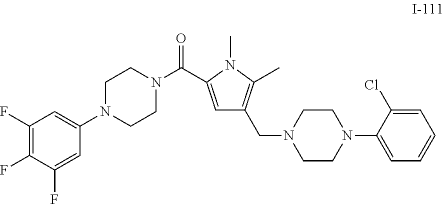 [SMILES: Cc1c(CN2CCN(c3ccccc3Cl)CC2)cc(C(=O)N2CCN(c3cc(F)c(F)c(F)c3)CC2)n1C]

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid. The synthesis of 1-(3,4,5-trifluorophenyl)piperazine was the same as Example 8 and the procedure for (4-((4-(2-chlorophenyl)piperazin-1-yl)methyl)-1,5-dimethyl-1H-pyrrol-2-yl)(4-(3,4,5-trifluorophenyl)piperazin-1-yl)methanone was the same as Example 1.

Analysis was performed following Method B. Separation was performed following Method D. ESI-MS (EI⁺, m/z): 546.3 [M+H]⁺. ¹H NMR (500 MHz, DMSO-d₆) δ 7.38 (d, J=8.0 Hz, 1H), 7.27 (d, J=7.5 Hz, 1H), 7.14 (d, J=8.0 Hz, 1H), 7.03 (d, J=7.5 Hz, 1H), 6.88 (dd, J=11.5, 6.0 Hz, 2H), 6.26 (s, 1H), 3.73 (m, 4H), 3.55 (s, 3H), 3.34 (s, 2H), 3.31 (m, 4H), 3.22 (m, 4H), 2.94 (m, 4H), 2.19 (s, 3H).

### Example 15: (4-((4-(2-Chlorophenyl)piperazin-1-yl)methyl)-1,5-dimethyl-1H-pyrrol-2-yl)(4-(4-fluorophenyl)-1,4-diazepan-1-yl)methanone, I-49

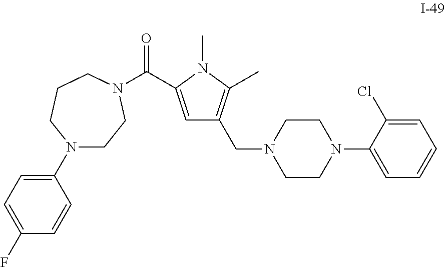 [SMILES: Cc1c(CN2CCN(c3ccccc3Cl)CC2)cc(C(=O)N2CCCN(c3ccc(F)cc3)CC2)n1C]

### Synthetic Scheme:

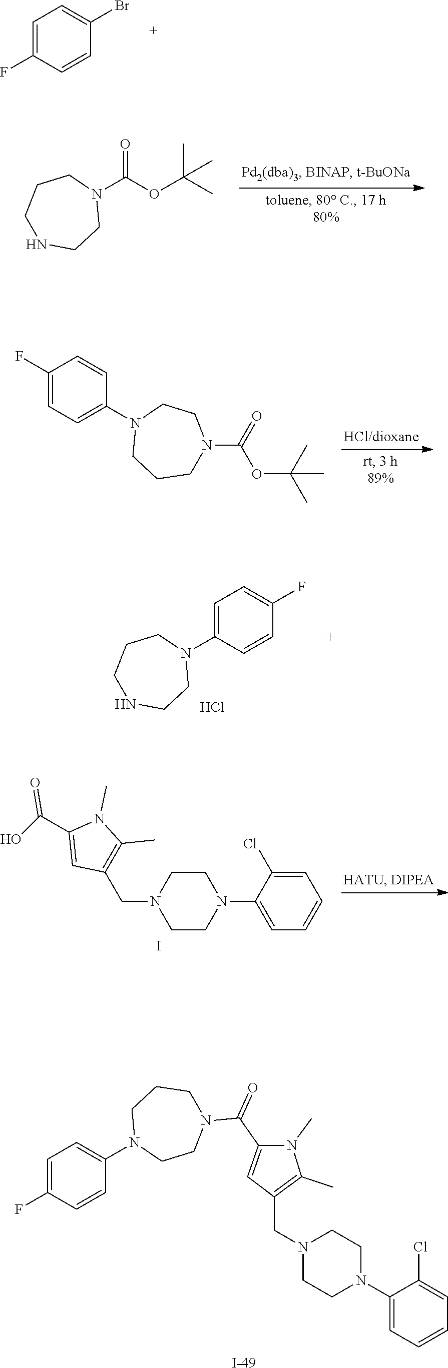

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: tert-butyl 4-(4-fluorophenyl)-1,4-diazepane-1-carboxylate

A solution of 1-bromo-4-fluorobenzene (875 mg, 5.0 mmol), tert-butyl-1,4-diazepane-1-carboxylate (1.0 g, 5.0 mmol), t-BuONa (720 mg, 7.5 mmol), BINAP (62 mg, 0.1 mmol), and Pd₂(dba)₃ (92 mg, 0.1 mmol) in dry toluene (20 mL) was stirred for 17 hrs at 80° C. The crude product was purified by chromatography (silica, EtOAc/PE=1/10) to afford tert-butyl-4-(4-fluorophenyl)-1,4-diazepane-1-carboxylate (1.0 g, 3.4 mmol, 80%) as a yellow solid. ESI-MS (EI⁺, m/z): 295.2 [M+H]⁺.

### Step 2: 1-(4-fluorophenyl)-1,4-diazepane hydrochloride salt

To a solution of tert-butyl 4-(4-fluorophenyl)-1,4-diazepane-1-carboxylate (1.0 g, 3.4 mmol) in Et₂O (15 mL) was added 4M HCl/dioxane (20 mL). The mixture was stirred for 3 hrs at rt. The solid was filtered to afford 1-(4-fluorophenyl)-1,4-diazepane hydrochloride salt (700 mg, 3.0 mmol, 89%) as a yellow solid. ESI-MS (EI⁺, m/z): 195.2 [M+H]⁺.

### (4-((4-(2-Chlorophenyl)piperazin-1-yl)methyl)-1,5-dimethyl-1H-pyrrol-2-yl)(4-(4-fluorophenyl)-1,4-diazepan-1-yl)methanone

The procedure for the synthesis of (4-((4-(2-Chlorophenyl)piperazin-1-yl)methyl)-1,5-dimethyl-1H-pyrrol-2-yl)(4-(4-fluorophenyl)-1,4-diazepan-1-yl)methanone I-49 was the same as Example 1.

ESI-MS (EI⁺, m/z): 524.3 [M+H]⁺. ¹H NMR (500 MHz, DMSO-d₆) δ 7.39 (d, J=7.5 Hz, 1H), 7.29 (t, J=7.5 Hz, 1H), 7.15 (d, J=7.5 Hz, 1H), 7.03 (t, J=7.5 Hz, 1H), 6.93 (t, J=8.5 Hz, 2H), 6.66 (s, 2H), 6.06 (s, 1H), 3.80 (m, 2H), 3.64 (m, 2H), 3.60-3.43 (m, 4H), 3.31 (m, 5H), 3.16 (m, 2H), 2.95 (m, 4H), 2.11 (s, 3H), 1.88 (m, 2H).

### Example 16: (S)-(4-((4-(2-Chlorophenyl)piperazin-1-yl)methyl)-1,5-dimethyl-1H-pyrrol-2-yl)(2-methyl-4-(3,4,5-trifluorophenyl)piperazin-1-yl)methanone, I-89

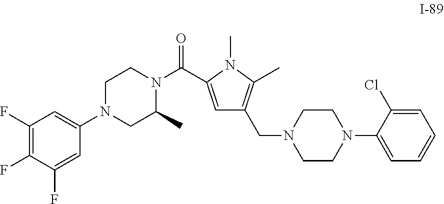 [SMILES: Cc1c(CN2CCN(c3ccccc3Cl)CC2)cc(C(=O)N2CCN(c3cc(F)c(F)c(F)c3)C[C@@H]2C)n1C]

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid. The synthesis of (S)-3-methyl-1-(3,4,5-trifluorophenyl)piperazine was the same as Example 8 and the procedure for (S)-(4-((4-(2-Chlorophenyl)piperazin-1-yl)methyl)-1,5-dimethyl-1H-pyrrol-2-yl)(2-methyl-4-(3,4,5-trifluorophenyl)piperazin-1-yl)methanone I-89 was the same as example 1.

Analysis was performed following Method B. Separation was performed following Method D. ESI-MS (EI+, m/z): 560.3 [M+H]⁺. 1H NMR (500 MHz, DMSO-d6) δ 7.39 (dd, J=8.0, 1.5 Hz, 1H), 7.28 (dd, J=11.5, 4.0 Hz, 1H), 7.14 (d, J=7.0 Hz, 1H), 7.03 (dd, J=11.0, 4.5 Hz, 1H), 6.86 (dd, J=11.5, 6.0 Hz, 2H), 6.22 (s, 1H), 4.62 (m, 1H), 4.17 (d, J=13.5 Hz, 1H), 3.63 (m, 2H), 3.53 (s, 3H), 3.33 (m, 7H), 2.93 (m, 5H), 2.73 (d, J=3.5 Hz, 1H), 2.19 (s, 3H), 1.25 (d, J=6.5 Hz, 3H).

### Example 17: (4-((4-(2-Chlorophenyl)piperazin-1-yl)methyl)-1,5-dimethyl-1H-pyrrol-2-yl)(3-phenyl-6,7-dihydropyrazolo[1,5-a]pyrazin-5(4H)-yl)methanone, I-35

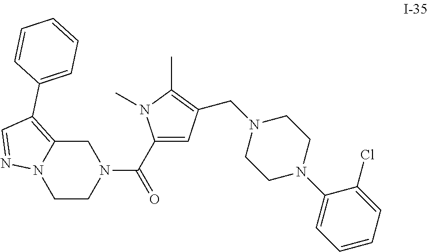 [SMILES: Cc1c(CN2CCN(c3ccccc3Cl)CC2)cc(C(=O)N2CCn3ncc(-c4ccccc4)c3C2)n1C]

### Synthetic Scheme:

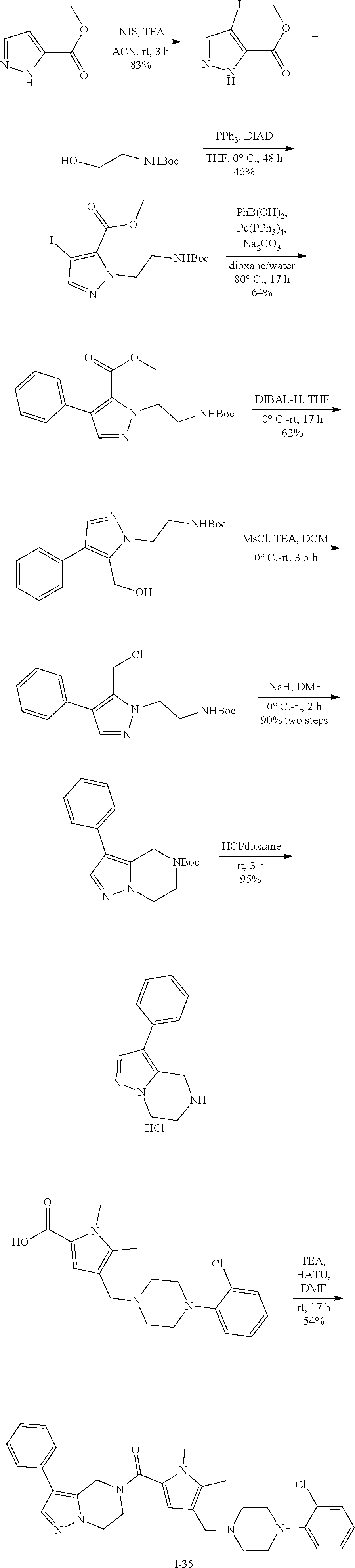

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: Methyl-4-iodo-1H-pyrazole-5-carboxylate

To a solution of methyl-1H-pyrazole-5-carboxylate (10.44 g, 83 mmol) and N-iodosuccinimide (20.46 g, 91 mmol) in CH₃CN (300 mL) was added TFA (1.92 mL) under N₂ atmosphere. The reaction was stirred at rt for 3 hrs. The reaction mixture was then quenched with saturated aq. NaHCO₃ (300 mL) and extracted with EtOAc (3×300 mL). The organic phases were combined, washed with brine, dried with Na₂SO₄, filtered, and concentrated. The product was obtained by recrystallation in EtOAc as a white solid (17.36 g, 68.9 mmol). ESI-MS (EI⁺, m/z): 252.7 [M+H]⁺.

### Step 2: Methyl-1-(2-(tert-butoxycarbonylamino)ethyl)-4-iodo-1H-pyrazole-5-carboxylate

To a solution of methyl-4-iodo-1H-pyrazole-5-carboxylate (17.36 g, 68.9 mmol), PPh₃ (18.05 g, 68.9 mmol) and tert-butyl-2-hydroxyethylcarbamate (11.09 g, 68.9 mmol) in THF (200 mL) was added DIAD (13.92 g, 68.9 mmol) slowly at 0° C. The reaction was warmed to rt and kept at this temperature for 48 hrs. The solvent was then removed and the crude mixture was purified by column chromatography (EtOAc/MeOH=100:1 to 1:1) to afford the product as a yellow solid (12.52 g, 31.7 mmol). ESI-MS (EI⁺, m/z): 340.0 [M−56]⁺.

### Step 3: Methyl-1-(2-(tert-butoxycarbonylamino)ethyl)-4-phenyl-1H-pyrazole-5-carboxylate

To a solution of methyl-1-(2-(tert-butoxycarbonylamino)ethyl)-4-phenyl-1H-pyrazole-5-carboxylate (2.0 g, 5.0 mmol), phenylboronic acid (740 mg, 6.0 mmol), Na₂CO₃ (1.6 g, 15 mmol) in dioxane (100 mL), and water (30 mL) was added Pd(PPh₃)₄ (200 mg, 0.17 mmol). The solution was stirred for 17 hrs at 80° C. under N₂, then diluted with water (50 mL) and extracted with DCM (100 mL). The organic phase was washed with water (100 mL×2) and brine (100 mL), dried (Na₂SO₄), filtered, and concentrated in vacuo. The crude product was purified by chromatography (silica, EtOAc/PE=1/5) to afford methyl-1-(2-(tert-butoxycarbonylamino)ethyl)-4-phenyl-1H-pyrazole-5-carboxylate (1.1 g, 3.1 mmol, 64%) as a white solid. ESI-MS (EI⁺, m/z): 346.2 [M+H]⁺.

### Step 4: tert-Butyl-2-(5-(hydroxymethyl)-4-phenyl-1H-pyrazol-1-yl)ethylcarbamate

A solution of methyl 1-(2-(tert-butoxycarbonylamino)ethyl)-4-phenyl-1H-pyrazole-5-carboxylate (280 mg, 0.81 mmol) in dry THF (5 mL) was cooled down to 0° C. and 1 M DIBAL-H (4 mL, 4.0 mmol) was added dropwise. The mixture was allowed to warm to rt and stirred for 17 hrs. The solution was quenched with MeOH and the mixture was concentrated. The residue was diluted with water (20 mL) and extracted with DCM (50 mL). The organic phase was washed with water (10 mL×2), and brine (10 mL), dried (Na₂SO₄), filtered, and concentrated in vacuo. The crude product was purified by chromatography (silica, EtOAc/PE=1/1) to afford tert-butyl-2-(5-(hydroxymethyl)-4-phenyl-1H-pyrazol-1-yl)ethylcarbamate (160 mg, 0.5 mmol, 62%) as a white solid. ESI-MS (EI⁺, m/z): 318.0 [M+H]⁺.

### Step 5: tert-Butyl-2-(5-(chloromethyl)-4-phenyl-1H-pyrazol-1-yl)ethylcarbamate

To a solution of tert-butyl-2-(5-(hydroxymethyl)-4-phenyl-1H-pyrazol-1-yl)ethylcarbamate (140 mg, 0.44 mmol) in dry DCM (5 mL) was added TEA (67 mg, 0.66 mmol) and methanesulfonyl chloride (76 mg, 0.66 mmol) at 0° C. The mixture was allowed to warm to rt and stirred for 3.5 hrs, then aq. NaHCO₃ was added and the mixture was extracted with DCM (20 mL). The organic phase was washed with water (10 mL×2) and brine (10 mL), dried (Na₂SO₄), filtered, and concentrated to afford crude tert-butyl 2-(5-(chloromethyl)-4-phenyl-1H-pyrazol-1-yl)ethylcarbamate (200 mg) as a yellow oil. ESI-MS (EI⁺, m/z): 336.2 [M+H]⁺.

### Step 6: tert-Butyl 3-phenyl-6,7-dihydropyrazolo[1,5-a]pyrazine-5(4H)-carboxylate

To a solution of crude tert-butyl-2-(5-(chloromethyl)-4-phenyl-1H-pyrazol-1-yl)ethylcarbamate (200 mg) in dry DMF (5 mL) was added 60% NaH (71 mg, 1.78 mmol) at 0° C. The mixture was allowed to warm to rt and stirred for 2 hrs, then aq. NH₄Cl was added and the mixture was extracted with EtOAc (20 mL). The organic phase was washed with water (10 mL×2) and brine (10 mL), dried (Na₂SO₄), filtered, and concentrated in vacuo. The crude product was purified by chromatography (silica, EtOAc/PE=1/1) to afford tert-butyl-3-phenyl-6,7-dihydropyrazolo[1,5-a]pyrazine-5(4H)-carboxylate (120 mg, 0.4 mmol, 90% for two steps) as a colorless oil. ESI-MS (EI⁺, m/z): 300.3 [M+H]⁺.

### Step 7: 3-Phenyl-4,5,6,7-tetrahydropyrazolo[1,5-a]pyrazine hydrochloride salt

To a solution of tert-butyl-3-phenyl-6,7-dihydropyrazolo[1,5-a]pyrazine-5 (4H)-carboxylate (120 mg, 0.4 mmol) was added 4 M HCl/dioxane (10 mL). The solution was stirred for 3 hrs at rt, then filtered to afford crude 3-phenyl-4,5,6,7-tetrahydropyrazolo[1,5-a]pyrazine hydrochloride salt (90 mg, 0.38 mmol, 95%) as a white solid. ESI-MS (EI⁺, m/z): 200.2 [M+H]⁺.

### Step 8: (4-((4-(2-Chlorophenyl)piperazin-1-yl)methyl)-1,5-dimethyl-1H-pyrrol-2-yl)(3-phenyl-6,7-dihydropyrazolo[1,5-a]pyrazin-5(4H)-yl)methanone

To a solution of crude 3-phenyl-4,5,6,7-tetrahydropyrazolo[1,5-a]pyrazine hydrochloride salt (90 mg, 0.38 mmol) and 4-((4-(2-chlorophenyl)piperazin-1-yl)methyl)-1,5-dimethyl-1H-pyrrole-2-carboxylic acid (80 mg, 0.23 mmol) in DMF (3 mL) was added HATU (900 mg, 2.36 mmol) and TEA (116 mg, 1.15 mmol). The mixture was stirred at rt for 17 hrs, then purified by prep-HPLC (Boston C18 21*250 mm 10 μm, Mobile phase: A: aqueous 0.1% trifluoroacetic acid; B: acetonitrile) to afford (4-((4-(2-chlorophenyl)piperazin-1-yl)methyl)-1,5-dimethyl-1H-pyrrol-2-yl)(3-phenyl-6,7-dihydropyrazolo[1,5-a]pyrazin-5(4H)-yl)methanone I-35 (66 mg, 0.12 mmol, 54%) as a white solid.

ESI-MS (EI⁺, m/z): 529.3 [M+H]⁺. ¹H NMR (500 MHz, DMSO-d₆) δ 7.85 (s, 1H), 7.45-7.35 (m, 5H), 7.32-7.25 (m, 1H), 7.19-7.10 (m, 2H), 7.07-7.01 (m, 1H), 6.41 (s, 1H), 5.10 (s, 2H), 4.26 (m, 2H), 4.15 (m, 2H), 3.56 (s, 3H), 3.33 (s, 2H), 3.32 (s, 4H), 2.90 (m, 4H), 2.18 (s, 3H).

### Example 18: (4-((4-(2-chlorophenyl)piperazin-1-yl)methyl)-1,5-dimethyl-1H-pyrrol-2-yl)(3,4-dihydrobenzo[4,5]imidazo[1,2-a]pyrazin-2(1H)-yl)methanone, I-91

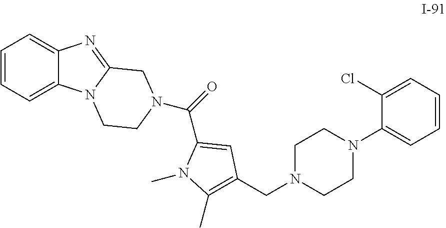 [SMILES: Cc1c(CN2CCN(c3ccccc3Cl)CC2)cc(C(=O)N2CCn3c(nc4ccccc43)C2)n1C]

### Synthetic Scheme:

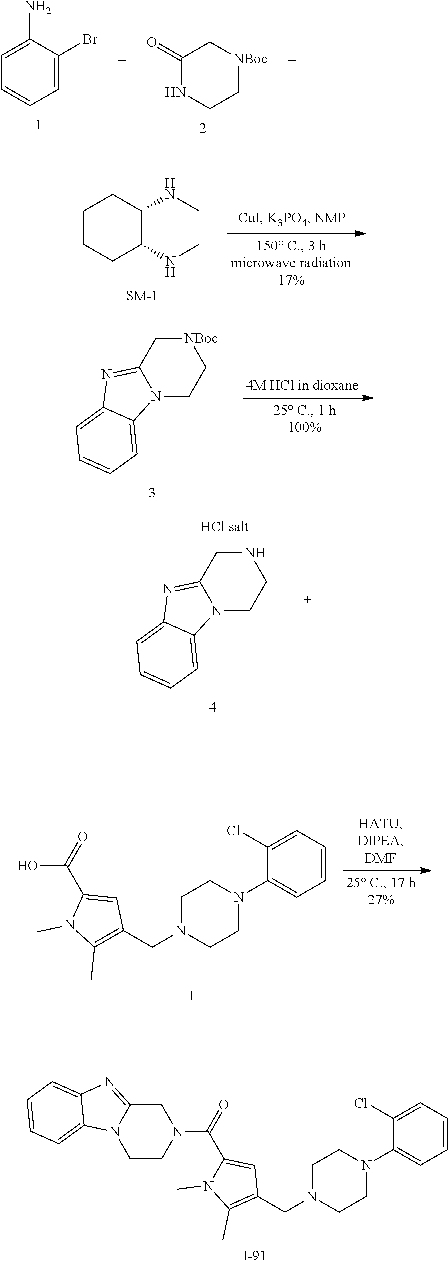

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: tert-Butyl-3,4-dihydropyrazino[1,2-a]benzimidazole-2(1H)-carboxylate

A mixture of 2-bromobenzenamine (430 mg, 2.5 mmol), tert-butyl-3-oxopiperazine-1-carboxylate (501 mg, 2.5 mmol), CuI (48 mg, 0.25 mmol), (1R,2S)—N¹,N²-dimethylcyclohexane-1,2-diamine (36 mg, 0.25 mmol), K₃PO₄ (1.592 g, 7.5 mmol) and NMP (9 mL) was stirred at 150° C. for 3 hrs under microwave radiation, then EtOAc (100 mL) was added. The EtOAc layer was washed with H₂O (50 mL) and brine (50 mL), dried (Na₂SO₄), filtered, and concentrated in vacuo. The residue was purified by chromatography (silica, MeOH/CH₂Cl_{2=1/10}) to afford tert-Butyl-3,4-dihydropyrazino[1,2-a]benzimidazole-2(1H)-carboxylate (120 mg, 0.44 mmol, 17%) as a yellow solid. MS (EI⁺, m/z): 274.1 [M+H]⁺.

### Step 2: 1,2,3,4-Tetrahydropyrazino[1,2-a]benzimidazole hydrochloride

To a mixture of tert-Butyl-3,4-dihydropyrazino[1,2-a]benzimidazole-2(1H)-carboxylate (120 mg, 0.44 mmol) was added HCl in dioxane (10 mL, 4M, 40 mmol). The mixture was stirred at 25° C. for 1 hr, the mixture was concentrated in vacuo to give 1,2,3,4-tetrahydropyrazino[1,2-a]benzimidazole hydrochloride (92 mg, 0.44 mmol, 100%) as a yellow solid. MS (EI⁺, m/z): 174.1 [M+H]⁺.

### Step 3: (4-((4-(2-chlorophenyl)piperazin-1-yl)methyl)-1,5-dimethyl-1H-pyrrol-2-yl)(3,4-dihydrobenzo[4,5]imidazo[1,2-a]pyrazin-2(1H)-yl)methanone

A mixture of 1,2,3,4-tetrahydropyrazino[1,2-a]benzimidazole hydrochloride (92 mg, 0.44 mmol), 4-((4-(2-chlorophenyl)piperazin-1-yl)methyl)-1,5-dimethyl-1H-pyrrole-2-carboxylic acid hydrochloride (80 mg, 0.21 mmol), HATU (104 mg, 0.27 mmol), DIPEA (272 mg, 2.1 mmol), and DMF (3 mL) was stirred at 25° C. for 17 hrs. The reaction mixture was purified by prep-HPLC to afford (4-((4-(2-chlorophenyl)piperazin-1-yl)methyl)-1,5-dimethyl-1H-pyrrol-2-yl)(3,4-dihydrobenzo[4,5]imidazo[1,2-a]pyrazin-2(1H)-yl)methanone I-91 (28.5 mg, 0.057 mmol, 27%) as a white solid. MS (EI⁺, m/z): 503.2 [M+H]⁺. ¹H NMR (500 MHz, CDCl₃) δ 7.73-7.71 (m, 1H), 7.37-7.34 (m, 2H), 7.32-7.27 (m, 2H), 7.23-7.20 (m, 1H), 7.05-7.04 (m, 1H), 6.97-6.94 (m, 1H), 6.47 (s, 1H), 5.27 (s, 2H), 4.29-4.26 (m, 4H), 3.67 (s, 3H), 3.42 (s, 2H), 3.06 (s, 4H), 2.62 (s, 4H), 2.23 (s, 3H).

### Example 19: (S)-(4-((4-(2-chlorophenyl)piperazin-1-yl)methyl)-1,5-dimethyl-1H-pyrrol-2-yl)(4-(3,4-difluorophenyl)-2-methylpiperazin-1-yl)methanone, I-116

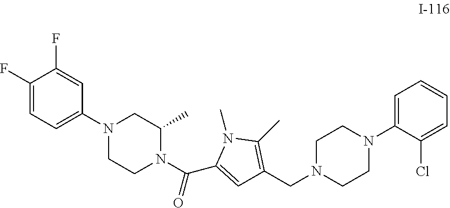 [SMILES: Cc1c(CN2CCN(c3ccccc3Cl)CC2)cc(C(=O)N2CCN(c3ccc(F)c(F)c3)C[C@@H]2C)n1C]

### Synthetic Scheme:

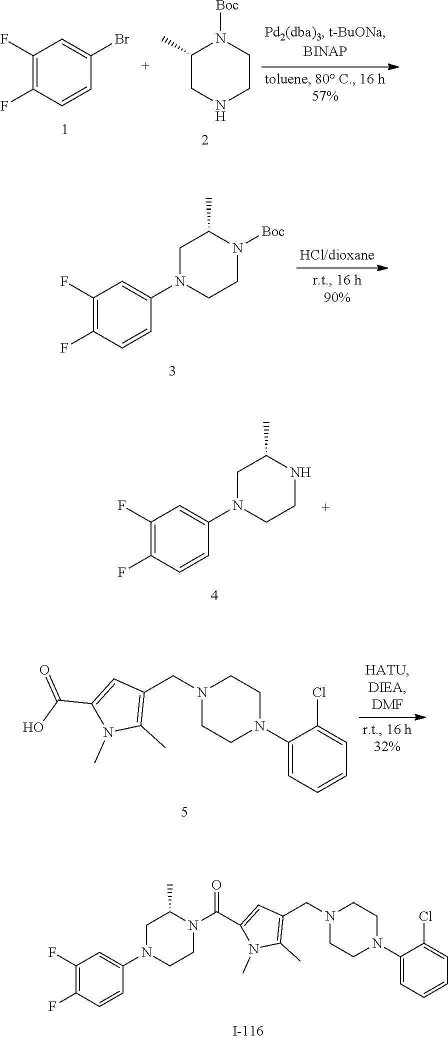

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: (S)-tert-butyl 4-(3,4-difluorophenyl)-2-methylpiperazine-1-carboxylate

A mixture of 4-bromo-1,2-difluorobenzene (1 g, 5.18 mmol), (S)-tert-butyl 2-methylpiperazine-1-carboxylate (1.04 g, 5.18 mmol), t-BuONa (747 mg, 7.77 mmol), BINAP (40 mg, 0.06 mmol) and Pd₂(dba)₃ (20 mg, 0.02 mmol) in toluene (15 mL) was stirred at 80° C. for 3 hrs. The mixture was then purified by chromatography (silica, EtOAc/PE=1/8) to afford (S)-tert-butyl-4-(3,4-difluorophenyl)-2-methylpiperazine-1-carboxylate (921 mg, 2.95 mmol, 57%) as product. ESI-MS (EI⁺, m/z): 257.1 [M−55]⁺.

### Step 2: (S)-1-(3,4-difluorophenyl)-3-methylpiperazine

To a solution of HCl (4 N, dioxane, 15 mL) in an ice bath was added (S)-tert-butyl-4-(3,4-difluorophenyl)-2-methylpiperazine-1-carboxylate (500 mg, 1.6 mmol). The solution was stirred at 20° C. for 16 hrs, then concentrated to afford (S)-1-(3,4-difluorophenyl)-3-methylpiperazine (302 mg, 1.44 mmol, 90%). ESI-MS (EI⁺, m/z): 213.2 [M+H]⁺. The synthesis of (S)-(4-((4-(2-Chlorophenyl)piperazin-1-yl)methyl)-1,5-dimethyl-1H-pyrrol-2-yl)(4-(3,4-difluorophenyl)-2-methylpiperazin-1-yl)methanone I-116 was the same as Example 1: ESI-MS (EI⁺, m/z): 542.3 [M+H]⁺. ¹H NMR (500 MHz, CDCl₃) δ 7.35 (dd, J=7.9, 1.3 Hz, 1H), 7.21 (t, J=7.7 Hz, 1H), 7.11-7.00 (m, 2H), 6.96 (t, J=7.6 Hz, 1H), 6.70 (ddd, J=13.0, 6.8, 2.9 Hz, 1H), 6.63-6.55 (m, 1H), 6.31 (s, 1H), 4.87 (s, 1H), 4.42 (d, J=13.7 Hz, 1H), 3.65 (s, 3H), 3.44 (d, J=10.7 Hz, 4H), 3.32 (d, J=11.8 Hz, 1H), 3.06 (s, 4H), 2.93 (dd, J=11.9, 3.6 Hz, 1H), 2.84-2.49 (m, 5H), 2.23 (s, 3H), 1.43 (d, J=6.8 Hz, 3H).

### Example 20: 3-(4-(4-((4-(2-chlorophenyl)piperazin-1-yl)methyl)-1,5-dimethyl-1H-pyrrole-2-carbonyl)piperazin-1-yl)benzonitrile, I-110

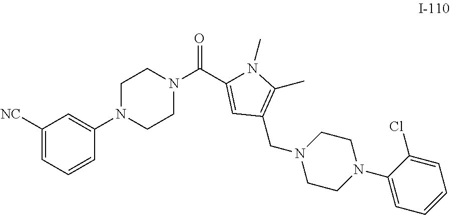 [SMILES: Cc1c(CN2CCN(c3ccccc3Cl)CC2)cc(C(=O)N2CCN(c3cccc(C#N)c3)CC2)n1C]

### Synthetic Scheme:

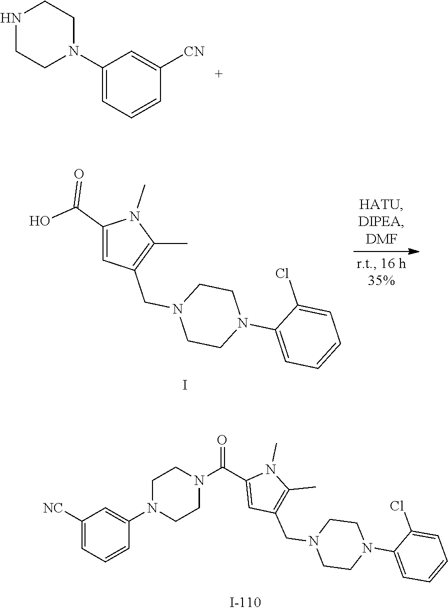

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method A. Separation was performed following Method D. The synthesis of 3-(4-(4-((4-(2-chlorophenyl)piperazin-1-yl)methyl)-1,5-dimethyl-1H-pyrrole-2-carbonyl)piperazin-1-yl)benzonitrile I-110 was the same as Example 1:

ESI-MS (EI+, m/z): 517.4 [M+H]⁺. ¹H NMR (400 MHz, CDCl3) δ 7.38-7.32 (m, 2H), 7.21 (td, J=8.0, 1.5 Hz, 1H), 7.16-7.12 (m, 3H), 7.04 (dd, J=8.1, 1.4 Hz, 1H), 6.96 (td, J=7.8, 1.5 Hz, 1H), 6.34 (s, 1H), 4.01-3.86 (m, 4H), 3.67 (s, 3H), 3.43 (s, 2H), 3.29-3.19 (m, 4H), 3.06 (s, 4H), 2.62 (s, 4H), 2.24 (s, 3H).

### Example 21: 2-(4-((5-(4-(4-Fluorophenyl)-1,4-diazepane-1-carbonyl)-1,2-dimethyl-1H-pyrrol-3-yl)methyl)piperazin-1-yl)benzonitrile, I-50

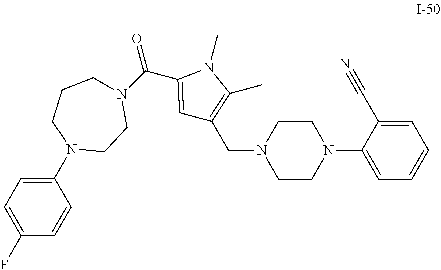 [SMILES: Cc1c(CN2CCN(c3ccccc3C#N)CC2)cc(C(=O)N2CCCN(c3ccc(F)cc3)CC2)n1C]

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid. Analysis was performed following Method B. Separation was performed following Method D. ESI-MS (EI⁺, m/z): 515.2 [M+H]⁺. ¹H NMR (500 MHz, DMSO-d₆) δ 7.69 (d, J=7.5 Hz, 1H), 7.59 (t, J=7.5 Hz, 1H), 7.15 (d, J=8.0 Hz, 1H), 7.08 (t, J=7.5 Hz, 1H), 6.93 (t, J=8.5 Hz, 2H), 6.66 (s, 2H), 6.07 (s, 1H), 3.79 (m, 2H), 3.64 (m, 2H), 3.57-3.42 (m, 4H), 3.35 (s, 2H), 3.33-3.30 (m, 3H), 3.23-3.05 (m, 6H), 2.53 (s, 2H), 2.11 (s, 3H), 1.88 (s, 2H).

### Example 22: (S)-(4-((4-(2-chlorophenyl)piperazin-1-yl)methyl)-1,5-dimethyl-1H-pyrrol-2-yl)(2-methyl-4-(pyrazin-2-yl)piperazin-1-yl)methanone, I-51

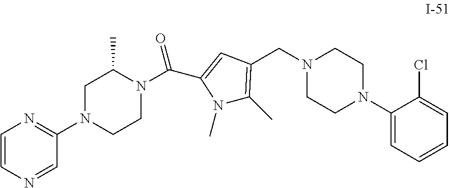 [SMILES: Cc1c(CN2CCN(c3ccccc3Cl)CC2)cc(C(=O)N2CCN(c3cnccn3)C[C@@H]2C)n1C]

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid. The synthesis of (S)-(4-((4-(2-chlorophenyl)piperazin-1-yl)methyl)-1,5-dimethyl-1H-pyrrol-2-yl)(2-methyl-4-(pyrazin-2-yl)piperazin-1-yl)methanone I-51 was the same as example 1. Analysis was performed following Method B. Separation was performed following Method D. ESI-MS (EI+, m/z): 508.3 [M+H]⁺. ¹H NMR (500 MHz, CDCl3) δ 8.15 (s, 1H), 8.07 (s, 1H), 7.87 (d, J=2.5 Hz, 1H), 7.35 (d, J=7.8 Hz, 1H), 7.21 (t, J=7.1 Hz, 1H), 7.04 (d, J=8.2 Hz, 1H), 6.96 (t, J=7.6 Hz, 1H), 6.32 (s, 1H), 4.89 (s, 1H), 4.43 (d, J=13.9 Hz, 1H), 4.25 (d, J=12.4 Hz, 1H), 4.11 (d, J=12.9 Hz, 1H), 3.66 (s, 3H), 3.44 (s, 3H), 3.26 (d, J=13.0 Hz, 1H), 3.05 (m, 5H), 2.63 (s, 4H), 2.22 (s, 3H), 1.34 (d, J=6.7 Hz, 3H).

### Example 23: (S)-2-(4-(4-((4-(2-chlorophenyl)piperazin-1-yl)methyl)-1,5-dimethyl-1H-pyrrole-2-carbonyl)-3-methylpiperazin-1-yl)-4-fluorobenzonitrile, I-86

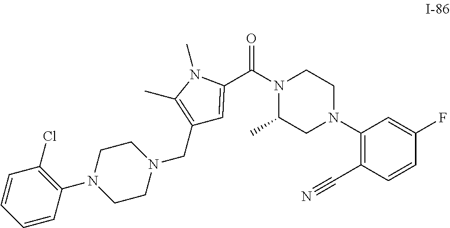 [SMILES: Cc1c(CN2CCN(c3ccccc3Cl)CC2)cc(C(=O)N2CCN(c3cc(F)ccc3C#N)C[C@@H]2C)n1C]

### Synthetic Scheme:

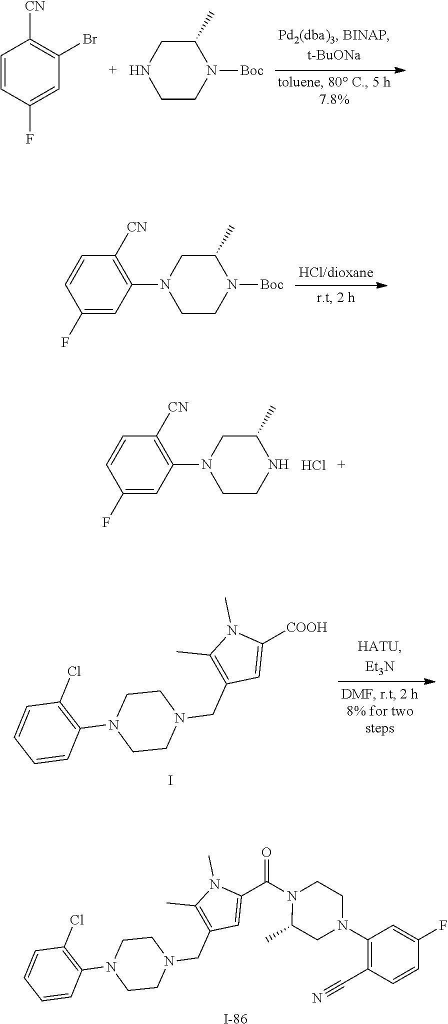

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I).

The synthesis of (S)-4-fluoro-2-(3-methylpiperazin-1-yl)benzonitrile hydrochloride was the same as Example 19.

Analysis was performed following Method B. Separation was performed following Method D. The procedure for (S)-2-(4-(4-((4-(2-chlorophenyl)piperazin-1-yl)methyl)-1,5-dimethyl-1H-pyrrole-2-carbonyl)-3-methylpiperazin-1-yl)-4-fluorobenzonitrile I-86 was the same as Example 1. ESI-MS (EI⁺, m/z): 549.3 [M+H]⁺. ¹H NMR (500 MHz, DMSO-d₆)) δ: 7.81-7.84 (dd, J₁=6.5 Hz, J₂=8.0 Hz, 1H), 7.39 (d, J=7.0 Hz, 1H), 7.27-7.30 (t, J=7.5 Hz, 1H), 7.14 (d, J=8.0 Hz, 1H), 6.96-7.07 (m, 3H), 6.24 (s, 1H), 4.72 (s, 1H), 4.25 (d, J=13.5 Hz, 1H), 3.54 (s, 3H), 3.38-3.51 (m, 8H), 2.88-3.02 (m, 7H), 2.20 (s, 3H), 1.40 (d, J=7.0 Hz, 3H).

### Example 24: 2-(4-(4-((4-(2-Chlorophenyl)piperazin-1-yl)methyl)-1,5-dimethyl-1H-pyrrole-2-carbonyl)piperazin-1-yl)-4-fluorobenzonitrile, I-85

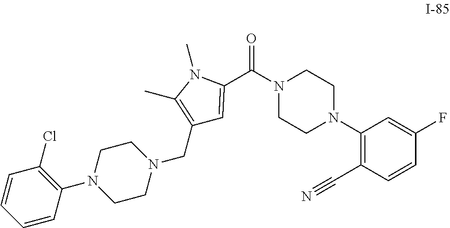 [SMILES: Cc1c(CN2CCN(c3ccccc3Cl)CC2)cc(C(=O)N2CCN(c3cc(F)ccc3C#N)CC2)n1C]

### Synthetic Scheme:

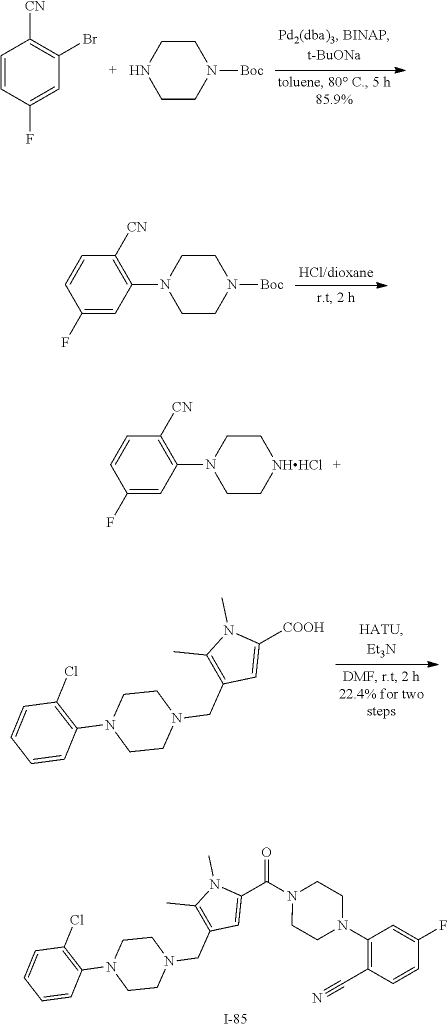

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). The synthesis of 4-fluoro-2-(3-piperazin-1-yl)benzonitrile hydrochloride was the same as Example 19.

Analysis was performed following Method B. Separation was performed following Method D. The procedure for 2-(4-(4-((4-(2-chlorophenyl)piperazin-1-yl)methyl)-1,5-dimethyl-1H-pyrrole-2-carbonyl)piperazin-1-yl)-4-fluorobenzonitrile I-85 was the same as Example 1. ESI-MS (EI⁺, m/z): 535.2 [M+H]⁺. ¹H NMR (500 MHz, CDCl₃) δ: 7.56-7.59 (dd, J₁=6.5 Hz, J₂=8.5 Hz, 1H), 7.34 (d, J=8.0 Hz, 1H), 7.18-7.21 (t, J=8.0 Hz, 1H), 7.03 (d, J=8.0 Hz, 1H), 6.94-6.97 (t, J=7.0 Hz, 1H), 6.73-6.76 (m, 1H), 6.67-6.70 (m, 1H), 6.34 (s, 1H), 3.96-3.98 (m, 4H), 3.66 (s, 3H), 3.47 (s, 2H), 3.23-3.25 (m, 4H), 3.07 (s, 4H), 2.65 (s, 4H), 2.34 (s, 3H),

### Example 25: (S)-(4-((4-(2-chlorophenyl)piperazin-1-yl)methyl)-1,5-dimethyl-1H-pyrrol-2-yl)(4-(5-fluoropyrimidin-2-yl)-2-methylpiperazin-1-yl)methanone, I-77

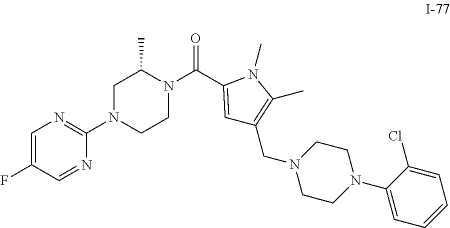 [SMILES: Cc1c(CN2CCN(c3ccccc3Cl)CC2)cc(C(=O)N2CCN(c3ncc(F)cn3)C[C@@H]2C)n1C]

### Synthetic Scheme:

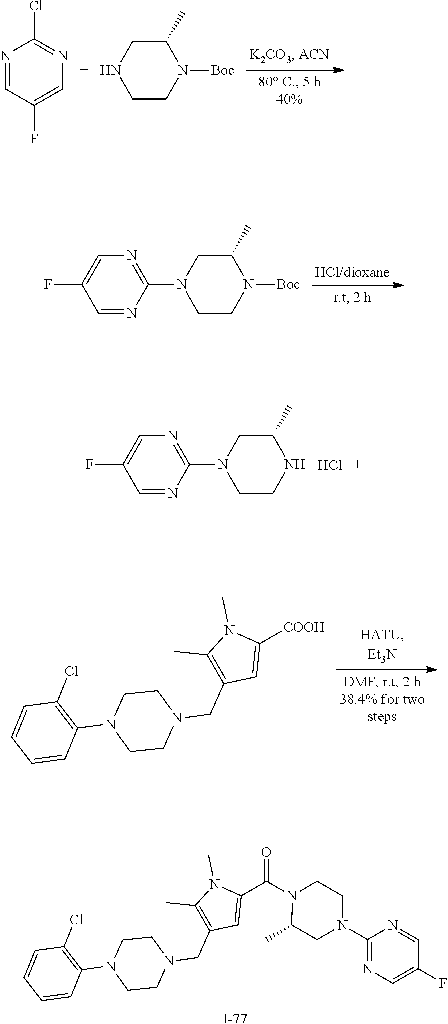

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). The synthesis of (S)-5-fluoro-2-(3-methylpiperazin-1-yl)pyrimidine hydrochloride was the same as Example 19.

Analysis was performed following Method A. Separation was performed following Method D. The procedure for (S)-(4-((4-(2-chlorophenyl)piperazin-1-yl)methyl)-1,5-dimethyl-1H-pyrrol-2-yl)(4-(5-fluoropyrimidin-2-yl)-2-methylpiperazin-1-yl)methanone I-77 was the same as Example 1. ESI-MS (EI⁺, m/z): 526.2 [M+H]⁺. ¹H NMR (500 MHz, DMSO-d₆)) δ: 8.46 (s, 2H), 7.37-7.40 (dd, J₁=2.0 Hz, J₂=9.5 Hz, 1H), 7.26-7.30 (m, 1H), 7.13-7.15 (dd, J₁=1.0 Hz, J₂=10.0 Hz, 1H), 7.00-7.04 (m, 1H), 6.23 (s, 1H), 4.65 (d, J=3.5 Hz, 1H), 4.39-4.49 (m, 2H), 4.19 (d, J=16.5 Hz, 1H), 3.53 (s, 3H), 3.48 (s, 4H), 3.16-3.20 (m, 2H), 2.95-3.03 (m, 5H), 2.50-2.53 (m, 2H), 2.19 (s, 3H), 1.11-1.16 (m, 3H).

### Example 26: (S)-(4-(1H-indol-5-yl)-2-methylpiperazin-1-yl)(4-((4-(2-chlorophenyl)piperazin-1-yl)methyl)-1,5-dimethyl-1H-pyrrol-2-yl)methanone trifluoroacetate salt, I-33

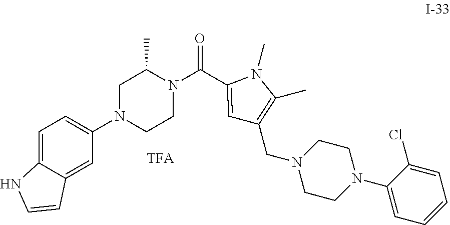 [SMILES: Cc1c(CN2CCN(c3ccccc3Cl)CC2)cc(C(=O)N2CCN(c3ccc4[nH]ccc4c3)C[C@@H]2C)n1C]

### Synthetic Scheme:

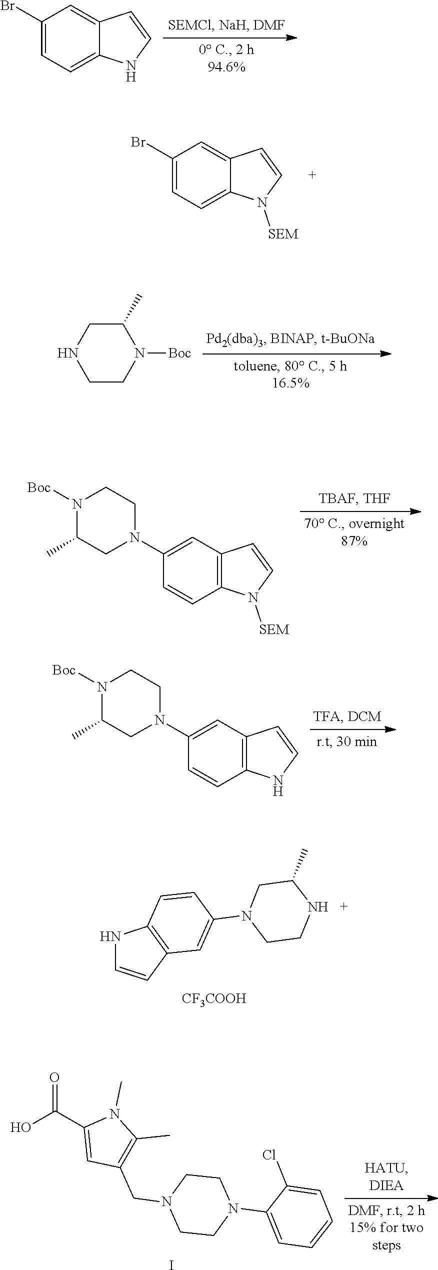

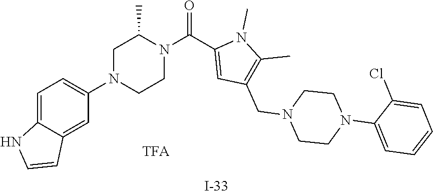

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method A. Separation was performed following Method C.

### Step 1: 5-Bromo-1-((2-(trimethylsilyl)ethoxy)methyl)-1H-indole

To a solution of 5-bromo-1H-indole (3.0 g, 15.3 mmol) in DMF (20 mL) at 0° C. was slowly added NaH (60% in mineral oil, 920 mg, 22.9 mmol). After the mixture was stirred at 0° C. for 1 hr, SEMCl (3.3 mL, 18.3 mmol) was added and the mixture was stirred continuely at 0° C. for 1 hr. The reaction mixture was then quenched with saturated aq. NH₄Cl (5 mL), diluted with EtOAc (200 mL), and washed with water (50 mL×2) and brine (50 mL). The organic phase was dried over Na₂SO₄, filtered, and concentrated. The residue was purified by column chromatography on silica to give 5-bromo-1-((2-(trimethylsilyl)ethoxy)methyl)-1H-indole (4.71 g, 14.44 mmol, 90%) as a yellow liquid. ESI-MS (EI⁺, m/z): 326.2 [M+H]⁺.

### Step 2: (S)-tert-butyl 2-methyl-4-(1-((2-(trimethylsilyl)ethoxy)methyl)-1H-indol-5-yl)piperazine-1-carboxylate

To a solution of 5-bromo-1-((2-(trimethylsilyl)ethoxy)methyl)-1H-indole (2 g, 6.13 mmol) and (S)-tert-butyl-2-methylpiperazine-1-carboxylate (1.23 g, 6.13 mmol) in toluene (20 mL) was added Pd₂(dba)₃ (281 mg, 0.31 mmol), BINAP (381 mg, 0.613 mmol), and t-BuONa (1.17 g, 12.26 mmol). The mixture was stirred at 80° C. for 5 hrs under nitrogen. The resulting mixture was concentrated in vacuo and the residue was purified by column chromatography on silica gel to give (S)-tert-butyl-2-methyl-4-(1-((2-(trimethylsilyl)ethoxy)methyl)-1H-indol-5-yl)piperazine-1-carboxylate (645 mg, 1.45 mmol, 16.5%) as a yellow liquid. ESI-MS (EI⁺, m/z): 446.4 [M+H]⁺.

### Step 3: (S)-tert-butyl 4-(1H-indol-5-yl)-2-methylpiperazine-1-carboxylate

To a solution of (R)-tert-butyl-2-methyl-4-(1-((2-(trimethylsilyl)ethoxy)methyl)-1H-indol-5-yl)piperazine-1-carboxylate (645 mg, 1.45 mmol) in THF (2 mL) was added TBAF (1 M in THF, 7.25 mL). The mixture was stirred at 70° C. overnight. The resulting reaction mixture was diluted with EtOAc (100 mL), then washed with water (50 mL×2) and brine (50 mL). The organic phase was dried over Na₂SO₄, filtered, and concentrated. The residue was purified by column chromatography on silica to give (S)-tert-butyl-4-(1H-indol-5-yl)-2-methylpiperazine-1-carboxylate (270 mg, 0.85 mmol, 87%) as a yellow liquid. ESI-MS (EI⁺, m/z): 316.4 [M+H]⁺.

### Step 4: (S)-5-(3-methylpiperazin-1-yl)-1H-indole trifluoroacetate salt

To a solution of (R)-tert-butyl-4-(1H-indol-5-yl)-2-methylpiperazine-1-carboxylate (170 mg, 0.54 mmol) in DCM (2 mL) was added TFA (1.2 mL, 16.2 mmol). The mixture was stirred at rt for 30 min. The resulting reaction mixture was concentrated to give (S)-5-(3-methylpiperazin-1-yl)-1H-indole trifluoroacetate (190 mg, crude) which was used without further purification. ESI-MS (EI⁺, m/z): 216.3 [M+H]⁺.

### Step 5: (S)-2-(4-(5-(4-(1H-indol-5-yl)-2-methylpiperazine-1-carbonyl)-2,4-di-methylbenzyl)piperazin-1-yl)benzonitrile trifluoroacetate salt

To a solution of (S)-5-(3-methylpiperazin-1-yl)-1H-indole trifluoroacetate (160 mg, crude) and 4-((4-(2-chlorophenyl)piperazin-1-yl)methyl)-1,5-dimethyl-1H-pyrrole-2-carboxylic acid (80 mg, 0.24 mmol) in DMF (2 mL) was added HATU (112 mg, 0.29 mmol) and DIEA (0.2 mL, 1.22 mmol). The mixture was stirred at rt for 2 hrs. The resulting reaction mixture was purified by prep-HPLC to afford (S)-(4-(1H-indol-5-yl)-2-methylpiperazin-1-yl)(4-((4-(2-chlorophenyl)piperazin-1-yl)methyl)-1,5-dimethyl-1H-pyrrol-2-yl)methanone trifluoroacetate I-33 (40 mg, 0.06 mmol, 15% for two steps). ESI-MS (EI⁺, m/z): 545.2 [M+H]⁺. ¹H NMR (500 MHz, CDCl₃) δ: 12.13-12.19 (m, 1H), 8.63 (s, 1H), 7.63 (s, 1H), 7.37 (d, J=9.0 Hz, 2H), 7.23-7.26 (m, 2H), 7.04-7.06 (m, 2H), 6.56 (d, J=12.5 Hz, 2H), 4.91 (s, 1H), 4.45 (d, J=15.0 Hz, 1H), 4.08-4.15 (m, 2H), 3.81-3.86 (m, 1H), 3.72-3.78 (m, 1H), 3.66 (s, 3H), 3.58-3.64 (m, 2H), 3.24-3.52 (m, 7H), 2.99-3.03 (m, 2H), 2.27 (s, 3H), 1.59 (d, J=7.0 Hz, 3H).

### Example 27: (S)-(4-(1H-indol-6-yl)-2-methylpiperazin-1-yl)(4-((4-(2-chlorophenyl)piperazin-1-yl)methyl)-1,5-dimethyl-1H-pyrrol-2-yl)methanone trifluoroacetate salt, I-34

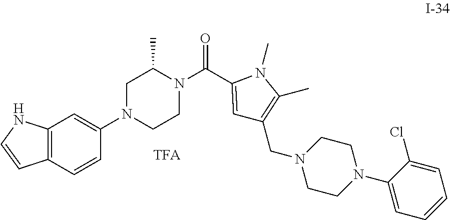 [SMILES: Cc1c(CN2CCN(c3ccccc3Cl)CC2)cc(C(=O)N2CCN(c3ccc4cc[nH]c4c3)C[C@@H]2C)n1C]

### Synthetic Scheme:

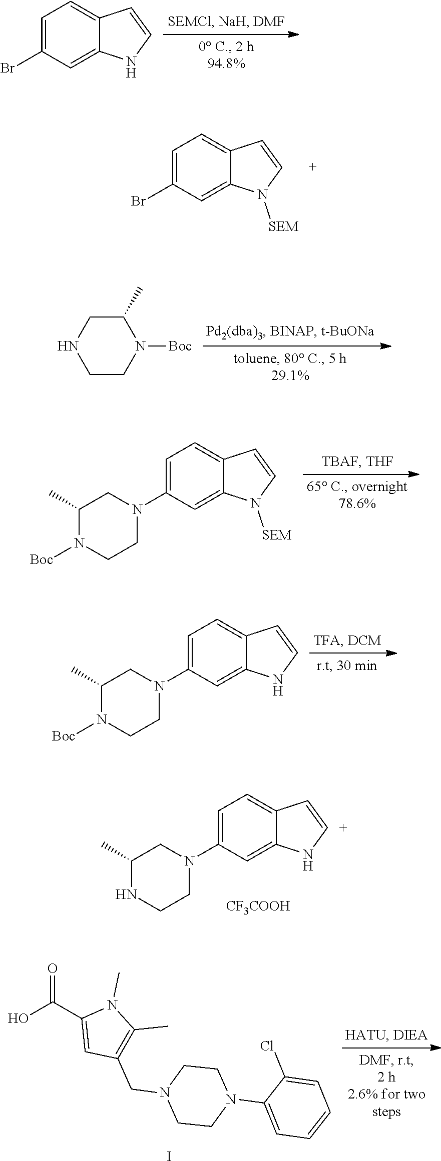

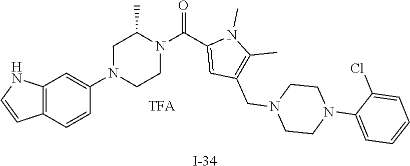

### Procedures and Characterization:

The procedure for (S)-(4-(1H-indol-6-yl)-2-methylpiperazin-1-yl)(4-((4-(2-chlorophenyl)piperazin-1-yl)methyl)-1,5-dimethyl-1H-pyrrol-2-yl)methanone trifluoroacetate salt was the same as Example 26.

Analysis was performed following Method A. Separation was performed following Method C. ESI-MS (EI⁺, m/z): 545.1 [M+H]⁺. ¹H NMR (500 MHz, CDCl₃) δ: 12.08-12.11 (m, 1H), 8.53 (s, 1H), 7.60 (d, J=8.5 Hz, 1H), 7.36-7.38 (m, 2H), 7.23-7.25 (m, 1H), 7.19-7.20 (t, J=2.5 Hz, 1H), 7.03-7.07 (m, 3H), 6.54 (s, 1H), 6.50 (s, 1H), 4.88 (s, 1H), 4.42 (d, J=14.0 Hz, 1H), 4.08-4.16 (m, 2H), 3.61-3.74 (m, 7H), 3.13-3.52 (m, 7H), 2.99-3.01 (m, 2H), 2.26 (s, 3H), 1.55 (d, J=6.5 Hz, 3H).

### Example 28: (S)-(4-(3-(3-aminopropyl)phenyl)-2-methylpiperazin-1-yl)(4-((4-(2-chlorophenyl)piperazin-1-yl)methyl)-1,5-dimethyl-1H-pyrrol-2-yl)methanone trifluoroacetate salt, I-30

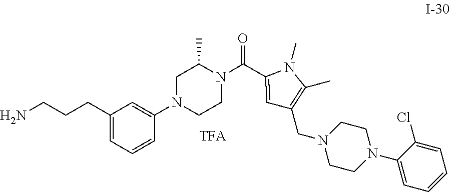 [SMILES: Cc1c(CN2CCN(c3ccccc3Cl)CC2)cc(C(=O)N2CCN(c3cccc(CCCN)c3)C[C@@H]2C)n1C]

### Synthetic Scheme:

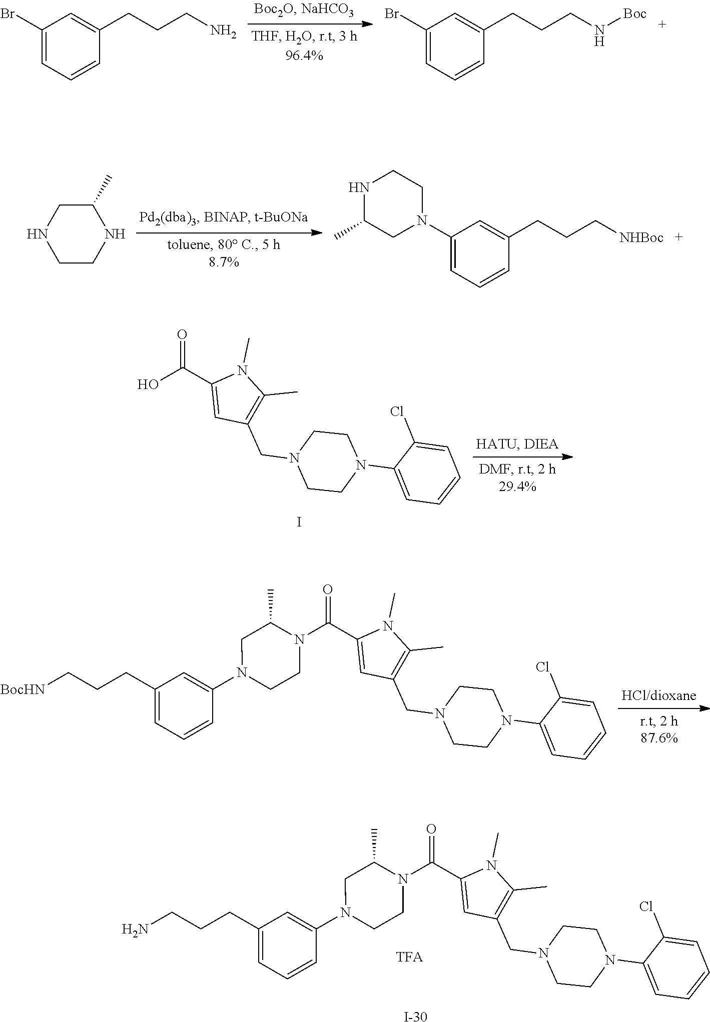

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method A. Separation was performed following Method C.

### Step 1: tert-Butyl 3-(3-bromophenyl)propylcarbamate

A mixture of 3-(3-bromophenyl)propan-1-amine (2 g, 13.3 mmol), Boc₂O (4.3 g, 19.95 mmol) and NaHCO₃ (2.2 g, 26.6 mmol) in THF (20 mL) and water (5 mL) was stirred at rt for 3 hrs. The resulting mixture was concentrated and the residue was diluted with EtOAc (200 mL), then washed with water (50 mL×2) and brine (50 mL). The organic phase was dried, filtered, and concentrated. The residue was purified by column chromatography on silca gel to give tert-butyl-3-(3-bromophenyl)propylcarbamate (2.8 g, 8.91 mmol, 96%) as a white solid. ESI-MS (EI⁺, m/z): 214.1 [M-Boc+H]⁺.

### Step 2: (S)-tert-butyl 3-(3-(3-methylpiperazin-1-yl)phenyl)propylcarbamate

The procedure for (S)-tert-butyl-3-(3-(3-methylpiperazin-1-yl)phenyl)propylcarbamate was the same as Example 21. ESI-MS (EI+, m/z): 334.4 [M+H]⁺.

### Step 3: (S)-tert-butyl-3-(3-(4-(4-((4-(2-chlorophenyl)piperazin-1-yl)methyl)-1,5-dimethyl-1H-pyrrole-2-carbonyl)-3-methylpiperazin-1-yl)phenyl)propylcarbamate

The procedure for ((S)-tert-butyl 3-(3-(4-(4-((4-(2-chlorophenyl)piperazin-1-yl)methyl)-1,5-dimethyl-1H-pyrrole-2-carbonyl)-3-methylpiperazin-1-yl)phenyl)propylcarbamate was the same as Example 21. ESI-MS (EI⁺, m/z): 663.4 [M+H]⁺.

### Step 4: (S)-(4-(3-(3-aminopropyl)phenyl)-2-methylpiperazin-1-yl)(4-((4-(2-chlorophenyl)piperazin-1-yl)methyl)-1,5-dimethyl-1H-pyrrol-2-yl)methanone trifluoroacetate salt

To a solution of ((S)-tert-butyl-3-(3-(4-(4-((4-(2-chlorophenyl)piperazin-1-yl)methyl)-1,5-dimethyl-1H-pyrrole-2-carbonyl)-3-methylpiperazin-1-yl)phenyl)propylcarbamate (50 mg, 0.0.76 mmol) in MeOH (1 mL) was added HCl (4 M in dioxane, 2 mL). The mixture was stirred at rt for 2 hrs, then the resulting mixture was concentrated and purified by prep-HPLC to give (S)-(4-(3-(3-aminopropyl)phenyl)-2-methylpiperazin-1-yl)(4-((4-(2-chlorophenyl)piperazin-1-yl)methyl)-1,5-dimethyl-1H-pyrrol-2-yl)methanone trifluoroacetate salt I-30 (44.7 mg, 0.066 mmol, 88%) as a white solid. ESI-MS (EI⁺, m/z): 563.4 [M+H]⁺. ¹H NMR (500 MHz, CDCl₃) δ: 11.46-11.49 (m, 1H), 7.92-8.03 (m, 2H), 7.35 (d, J=7.5 Hz, 1H), 7.15-7.23 (m, 2H), 6.98-7.04 (m, 2H), 6.83 (d, J=7.5 Hz, 2H), 6.72 (d, J=5.5 Hz, 1H), 6.42 (s, 1H), 4.75 (s, 1H), 3.98-4.29 (m, 6H), 3.38-3.61 (m, 7H), 3.18 (s, 2H), 2.82-3.00 (m, 6H), 2.63 (s, 2H), 2.24 (s, 3H), 1.99-2.01 (t, J=5.0 Hz, 2H), 1.41 (s, 3H).

### Example 29: (R)-(4-(3-(3-aminopropyl)phenyl)-2-methylpiperazin-1-yl)(4-((4-(2-chlorophenyl)piperazin-1-yl)methyl)-1,5-dimethyl-1H-pyrrol-2-yl)methanone trifluoroacetate salt, I-27

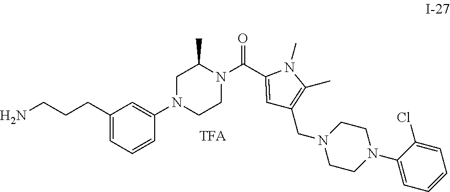 [SMILES: Cc1c(CN2CCN(c3ccccc3Cl)CC2)cc(C(=O)N2CCN(c3cccc(CCCN)c3)C[C@H]2C)n1C]

### Synthetic Scheme:

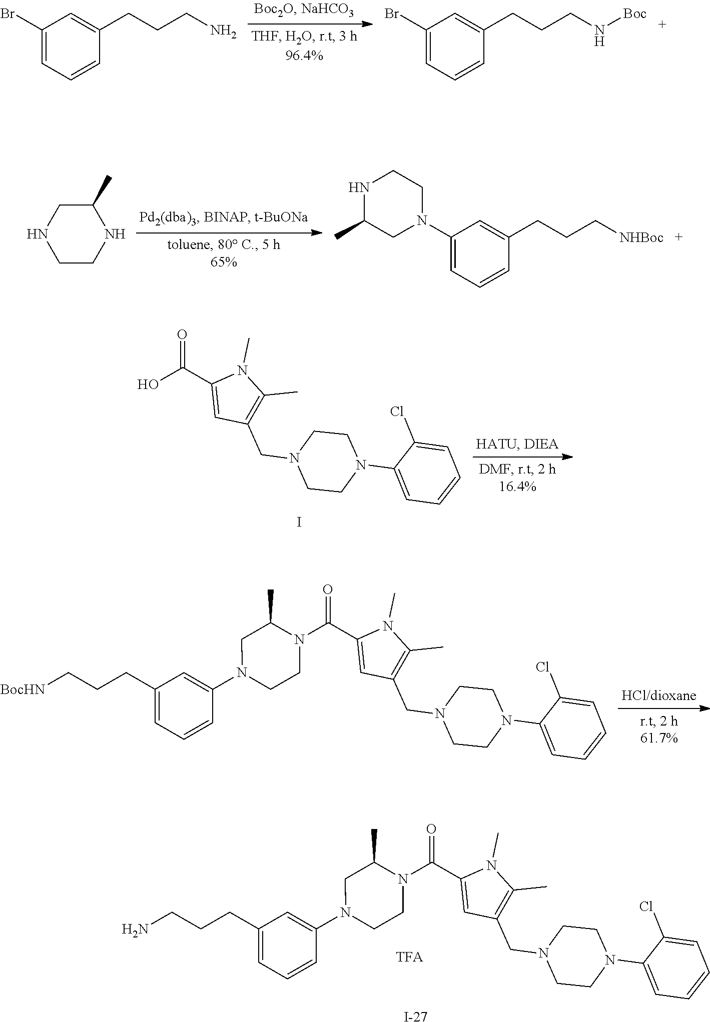

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). The procedure for (R)-(4-(3-(3-aminopropyl)phenyl)-2-methylpiperazin-1-yl)(4-((4-(2-chlorophenyl)piperazin-1-yl)methyl)-1,5-dimethyl-1H-pyrrol-2-yl)methanone trifluoroacetate salt I-27 was the same as Example 23.

Analysis was performed following Method A. Separation was performed following Method C. ESI-MS (EI⁺, m/z): 563.3 [M+H]⁺. ¹H NMR (500 MHz, CDCl₃) δ: 11.73-11.74 (m, 1H), 8.01-8.06 (m, 3H), 7.35 (d, J=8.0 Hz, 1H), 7.19-7.22 (t, J=7.0 Hz, 1H), 7.13-7.16 (t, J=8.0 Hz, 1H), 7.01-7.04 (m, 2H), 6.77-6.78 (m, 2H), 6.67 (d, J=7.5 Hz, 1H), 6.41 (s, 1H), 4.74 (s, 1H), 4.26 (d, J=5.0 Hz, 1H), 4.08 (s, 2H), 3.54-3.61 (m, 6H), 3.36-346 (m, 4H), 3.17-2.21 (t, J=11.5 Hz, 2H), 2.69-2.98 (m, 6H), 2.61 (s, 2H), 2.23 (s, 3H), 1.98 (s, 2H), 1.40 (d, J=7.0 Hz, 3H).

### Example 30: (R)-(4-((4-(2-(3-aminopropyl)phenyl)piperazin-1-yl)methyl)-1,5-dimethyl-1H-pyrrol-2-yl)(2-methyl-4-phenylpiperazin-1-yl)methanone ditrifluoroacetate salt, I-18

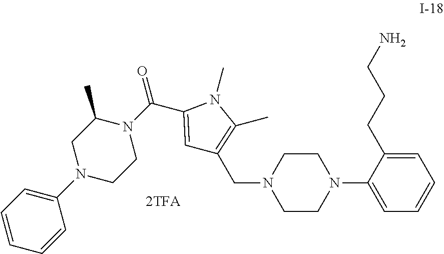 [SMILES: Cc1c(CN2CCN(c3ccccc3CCCN)CC2)cc(C(=O)N2CCN(c3ccccc3)C[C@H]2C)n1C]

### Synthetic Scheme:

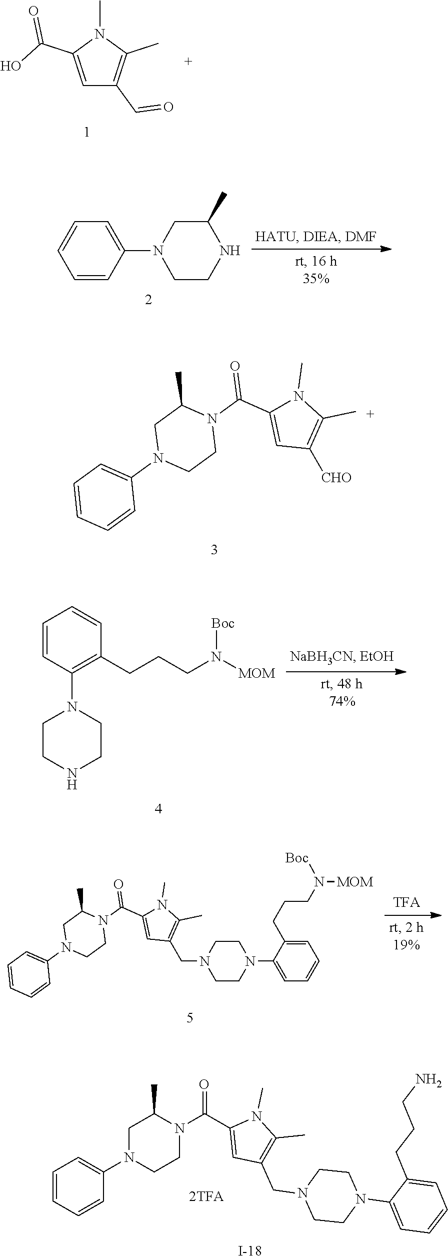

### Procedures and Characterization:

Analysis was performed following Method A. Separation was performed following Method C.

### Step 1: (R)-1,2-dimethyl-5-(2-methyl-4-phenylpiperazine-1-carbonyl)-1H-pyrrole-3-carbaldehyde

A mixture of 4-formyl-1,5-dimethyl-1H-pyrrole-2-carboxylic acid (400 mg, 2.4 mmol), (R)-3-methyl-1-phenylpiperazine (508 mg, 2.88 mmol), HATU (1.186 g, 3.12 mmol), DIPEA (1.55 g, 12 mmol) in DMF (10 mL) was stirred at rt for 16 hrs. The mixture was purified by chromatography (silica, EtOAc/PE=1/5) to give crude product. The crude product was purified by prep-HPLC to afford (R)-1,2-dimethyl-5-(2-methyl-4-phenylpiperazine-1-carbonyl)-1H-pyrrole-3-carbaldehyde (270 mg, 0.83 mmol, 35%) as yellow oil. ESI-MS (EI⁺, m/z): 326.3 [M+H]⁺.

### Step 2: (R)-tert-butyl 3-(2-(4-((1,2-dimethyl-5-(2-methyl-4-phenylpiperazine-1-carbonyl)-1H-pyrrol-3-yl)methyl)piperazin-1-yl)phenyl)propyl(methoxymethyl) carbamate

To a solution of (R)-1,2-dimethyl-5-(2-methyl-4-phenylpiperazine-1-carbonyl)-1H-pyrrole-3-carbaldehyde (98 mg, 0.3 mmol), tert-butyl-methoxymethyl(3-(2-(piperazin-1-yl)phenyl)propyl)carbamate (110 mg, 0.3 mmol) in EtOH (3 mL) and HOAc (1 drop) was stirred at rt for 2 hrs. Then NaBH₃CN (38 mg, 0.6 mmol) was added and stirred at rt for 14 hrs. The mixture was purified by chromatography (silica, EtOAc/PE=1/5) to afford (R)-tert-butyl 34244-((1,2-dimethyl-5-(2-methyl-4-phenylpiperazine-1-carbonyl)-1H-pyrrol-3-yl)methyl)piperazin-1-yl)phenyl)propyl (methoxymethyl) carbamate (150 mg, 0.223 mmol, 74%) as a yellow oil. ESI-MS (EI⁺, m/z): 673.5 [M+H]⁺.

### Step 3: (R)-(4-((4-(2-(3-aminopropyl)phenyl)piperazin-1-yl)methyl)-1,5-dimethyl-1H-pyrrol-2-yl)(2-methyl-4-phenylpiperazin-1-yl)methanone ditrifluoroacetate salt

The mixture of (R)-tert-butyl-3-(2-(4-((1,2-dimethyl-5-(2-methyl-4-phenylpiperazine-1-carbonyl)-1H-pyrrol-3-yl)methyl)piperazin-1-yl)phenyl)propyl(methoxy methyl) carbamate (180 mg, 0.27 mmol) in TFA (4 mL) was stirred at rt for 2 hrs then concentrated to give crude. The crude product was purified by prep-HPLC to afford (R)-(4-((4-(2-(3-aminopropyl)phenyl)piperazin-1-yl)methyl)-1,5-dimethyl-1H-pyrrol-2-yl)(2-methyl-4-phenylpiperazin-1-yl)methanone ditrifluoroacetate salt I-18 (38 mg, 0.05 mmol, 19%) as a white solid. ESI-MS (EI⁺, m/z): 529.4 [M+H]⁺. ¹H NMR (500 MHz, DMSO) δ 9.95 (s, 1H), 8.70 (s, 1H), 7.92 (s, 2H), 7.24 (t, J=7.2 Hz, 4H), 7.16-7.11 (m, 2H), 6.96 (d, J=8.2 Hz, 2H), 6.82 (t, J=7.2 Hz, 1H), 6.48 (s, 1H), 4.67 (s, 1H), 4.36-4.11 (m, 3H), 3.67-3.52 (m, 5H), 3.48-3.33 (m, 3H), 3.27-3.15 (m, 2H), 3.13-2.98 (m, 4H), 2.96-2.78 (m, 3H), 2.73-2.62 (m, 3H), 2.58 (t, J=5.4 Hz, 1H), 2.51 (s, 2H), 2.30 (s, 3H), 1.96-1.82 (m, 2H), 1.34 (d, J=6.7 Hz, 3H).

### Example 31: Synthesis of 2-(4-(4-((4-(2-chlorophenyl)piperazin-1-yl)methyl)-1,5-dimethyl-1H-pyrrole-2-carbonyl)piperazin-1-yl)-6-fluorobenzonitrile, I-107

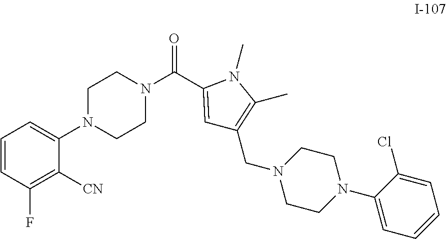 [SMILES: Cc1c(CN2CCN(c3ccccc3Cl)CC2)cc(C(=O)N2CCN(c3cccc(F)c3C#N)CC2)n1C]

### Synthetic Scheme:

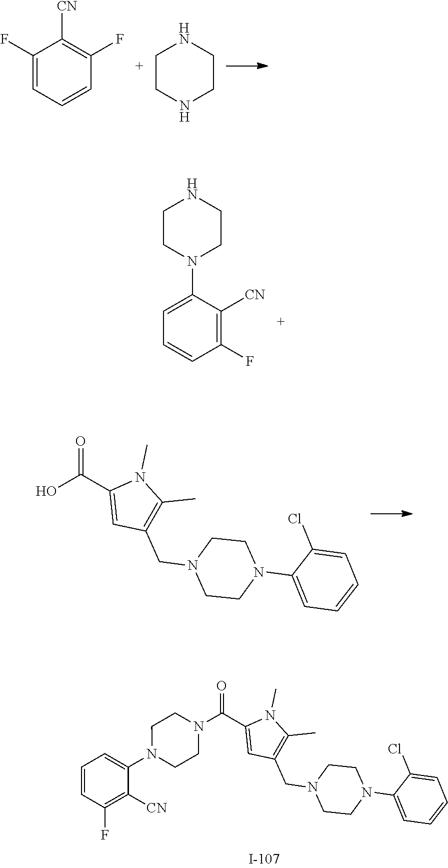

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid. Analysis was performed following Method B. Separation was performed following Method D.

### Step 1: 2-fluoro-6-(piperazin-1-yl)benzonitrile

A solution of 2,6-Difluorobenzonitrile (2 g, 14.4 mmol) and piperazine (6.2 g, 72 mmol) in DMF (20 mL) was heated at 65° C. for 3.5 hrs. Upon cooling, the reaction mixture was diluted with H₂O and extracted with EtOAc (4×80 mL). The combined organic layers were washed with H₂O (100 mL×3), then dried, filtered, and concentrated in vacuo to afford the desired product (2.1 g, 72%) with 86% purity as a light yellow oil. MS (EI⁺, m/z): 206 [M+H]⁺.

### Step 2: 2-(4-(4-((4-(2-chlorophenyl)piperazin-1-yl)methyl)-1,5-dimethyl-1H-pyrrole-2-carbonyl)piperazin-1-yl)-6-fluorobenzonitrile

To a solution of 4-((4-(2-chlorophenyl)piperazin-1-yl)methyl)-1,5-dimethyl-1H-pyrrole-2-carboxylic acid (100 mg, 0.29 mmol), 2-fluoro-6-(piperazin-1-yl)benzonitrile (119 mg, 0.58 mmol), and HATU (163 mg, 0.43 mmol) in DMF (4 mL) was added DIPEA (112 mg, 0.87 mmol). The mixture was stirred for 3 hrs, followed by filtration. The filtrate was purified by prep-HPLC to afford 2-(4-(4-((4-(2-chlorophenyl)piperazin-1-yl)methyl)-1,5-dimethyl-1H-pyrrole-2-carbonyl)piperazin-1-yl)-6-fluorobenzonitrile I-107 (79.8 mg, 0.15 mmol, 52%) as a white solid. MS (EI⁺, m/z): 535 [M+H]⁺. ¹H NMR (500 MHz, DMSO-d₆): δ 7.62 (q, J=8.5 Hz, 1H), 7.38 (d, J=8 Hz, 1H), 7.26 (t, J=7.5 Hz, 1H). 7.13 (d, J=7.5 Hz, 1H), 7.01-7.06 (m, 3H), 6.28 (s, 1H), 3.81 (s, 4H), 3.56 (s, 3H), 3.34 (s, 2H), 3.25 (t, J=5 Hz, 4H), 2.94 (s, 4H), 2.50 (s, 4H), 2.20 (s, 3H).

### Example 32: Synthesis of (S)-(4-((4-(2-chlorophenyl)piperazin-1-yl)methyl)-1,5-dimethyl-1H-pyrrol-2-yl)(2-methyl-4-(pyrimidin-2-yl)piperazin-1-yl)methanone, I-74

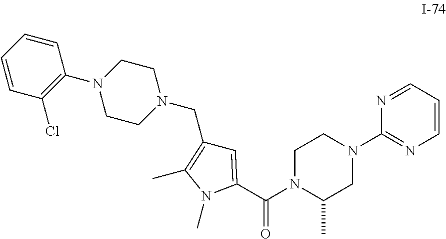 [SMILES: Cc1c(CN2CCN(c3ccccc3Cl)CC2)cc(C(=O)N2CCN(c3ncccn3)C[C@@H]2C)n1C]

### Synthetic Scheme:

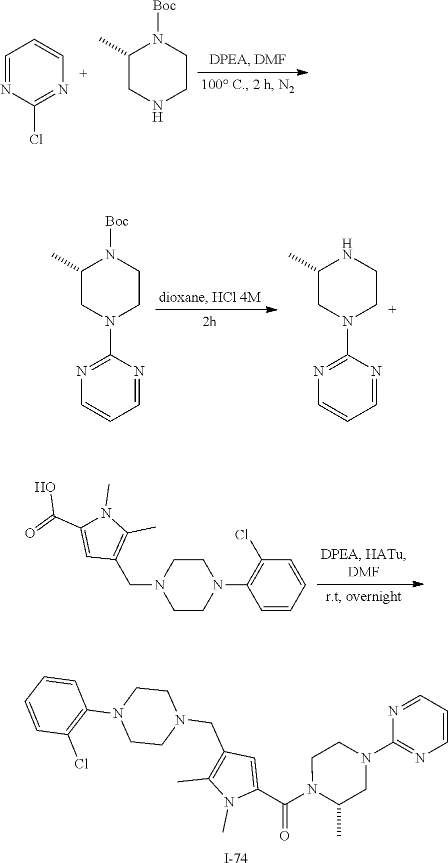

### Procedures and Characterization:

Analysis was performed following Method A. Separation was performed following Method D.

The general procedure for Intermediate I was followed to obtain 4-((4-(2-chlorophenyl)piperazin-1-yl)methyl)-1,5-dimethyl-1H-pyrrole-2-carboxylic acid.

### Step 1: Synthesis of (S)-tert-butyl-2-methyl-4-(pyrimidin-2-yl)piperazine-1-carboxylate

To the mixture of 2-chloropyrimidine (200 mg, 1.75 mmol), (S)-tert-butyl-2-methylpiperazine-1-carboxylate (350 mg, 1.75 mmol), and DIPEA (677 mg, 5.24 mmol) was added DMF (4 mL). The mixture was stirred at 100° C. for 3 hrs, then extracted with EtOAc/H₂O (50 mL/50 mL). The organic phase was dried over anhydrous Na₂SO₄ and concentrated in vacuo. The residue was purified by chromatography (silica, PE/EtOAc=0-10%) to afford (S)-tert-butyl-2-methyl-4-(pyrimidin-2-yl)-piperazine-1-carboxylate (330 mg, 67.8%). MS (EI+, m/z): 279 [M+H]⁺.

### Step 2: Synthesis of (S)-2-(3-methylpiperazin-1-yl)pyrimidine

A solution of 4N HCl dioxane (5 mL) was added to (S)-tert-butyl-2-methyl-4-(pyrimidin-2-yl)piperazine-1-carboxylate (250 mg, 0.898 mmol). The solution was stirred at rt for 2 hrs then concentrated in vacuo. MS (EI+, m/z): 179 [M+H]⁺.

### Step 3: Synthesis of (S)-(4-((4-(2-chlorophenyl)piperazin-1-yl)methyl)-1,5-dimethyl-1H-pyrrol-2-yl)(2-methyl-4-(pyrimidin-2-yl)piperazin-1-yl)methanone

To a solution of (S)-2-(3-methylpiperazin-1-yl)pyrimidine (100 mg, 0.561 mmol), 4-((4-(2-chlorophenyl)piperazin-1-yl)methyl)-1,5-dimethyl-1H-pyrrole-2-carboxylic acid (177.42 mg, 0.0.51 mmol), HATU (315 mg, 0.765 mmol), and DIPEA (131.84 mg, 1.02 mmol) was added DMF (4 mL). The mixture was stirred at rt overnight and then extracted with EtOAc/H₂O (20 ml/20 ml). The organic phase was dried over anhydrous Na₂SO₄, and concentrated in vacuo. The residue was purified by prep-HPLC to afford (S)-(4-((4-(2-chlorophenyl)piperazin-1-yl)methyl)-1,5-dimethyl-1H-pyrrol-2-yl)(2-methyl-4-(pyrimidin-2-yl)piperazin-1-yl)methanone I-74 (128 mg, 49.4%).

MS (EI+, m/z): 508 [M]⁺. ¹H NMR (500 MHz, DMSO-d₆) δ 8.38 (d, J=4.0 Hz, 2H), 7.46 (d, J=7.5 Hz, 1H), 7.35 (t, J=7.5 Hz, 1H), 7.22 (d, J=7.0 Hz, 1H), 7.13-7.11 (m, 1H), 6.66 (t, J=5.0 Hz, 1H), 6.45 (s, 1H), 5.76 (s, 1H), 4.65-4.51 (m, 3H), 4.27-4.18 (m, 3H), 3.57 (s, 3H), 3.47 (s, 3H), 3.21-3.19 (m, 3H), 3.01 (t, J=12.0 Hz, 3H), 2.29 (s, 3H), 1.18 (d, J=6.5 Hz, 3H).

### Example 33: (S)-(4-((4-(2-chlorophenyl)piperazin-1-yl)methyl)-1,5-dimethyl-1H-pyrrol-2-yl)(4-(3-fluorophenyl)-2-methylpiperazin-1-yl)methanone 2,2,2-trifluoroacetate, I-75

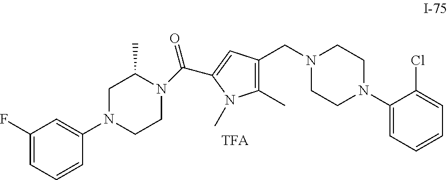 [SMILES: Cc1c(CN2CCN(c3ccccc3Cl)CC2)cc(C(=O)N2CCN(c3cccc(F)c3)C[C@@H]2C)n1C]

### Procedures and Characterization:

The procedure was the same as Example 19.

Analysis was performed following Method A. Separation was performed following Method C. (S)-(4-((4-(2-chlorophenyl)piperazin-1-yl)methyl)-1,5-dimethyl-1H-pyrrol-2-yl)(4-(3-fluorophenyl)-2-methylpiperazin-1-yl)methanone-2,2,2-trifluoroacetate I-75: ESI-MS (EI⁺, m/z): 524.3 [M+H]⁺. ¹H NMR (500 MHz, CDCl₃) δ 7.37 (d, J=7.9 Hz, 1H), 7.26-7.16 (m, 2H), 7.04 (dd, J=18.4, 10.9 Hz, 2H), 6.72-6.63 (m, 1H), 6.57 (dd, J=15.3, 7.4 Hz, 2H), 6.42 (s, 1H), 4.80 (s, 1H), 4.34 (d, J=11.1 Hz, 1H), 4.20-4.06 (m, 2H), 3.94-3.62 (m, 5H), 3.58 (d, J=11.4 Hz, 1H), 3.43 (m, 4H), 3.28 (t, J=12.0 Hz, 2H), 3.02 (m, 3H), 2.81 (td, J=11.9, 3.1 Hz, 1H), 2.27 (s, 3H), 1.42 (d, J=6.7 Hz, 3H).

### Example 34: (S)-(4-(3-azidophenyl)-2-methylpiperazin-1-yl)(4-((4-(2-chlorophenyl)piperazin-1-yl)methyl)-1,5-dimethyl-1H-pyrrol-2-yl)methanone, I-104

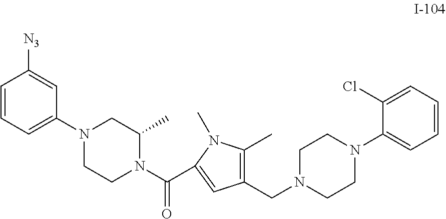 [SMILES: Cc1c(CN2CCN(c3ccccc3Cl)CC2)cc(C(=O)N2CCN(c3cccc(N=[N+]=[N-])c3)C[C@@H]2C)n1C]

### Synthetic Scheme:

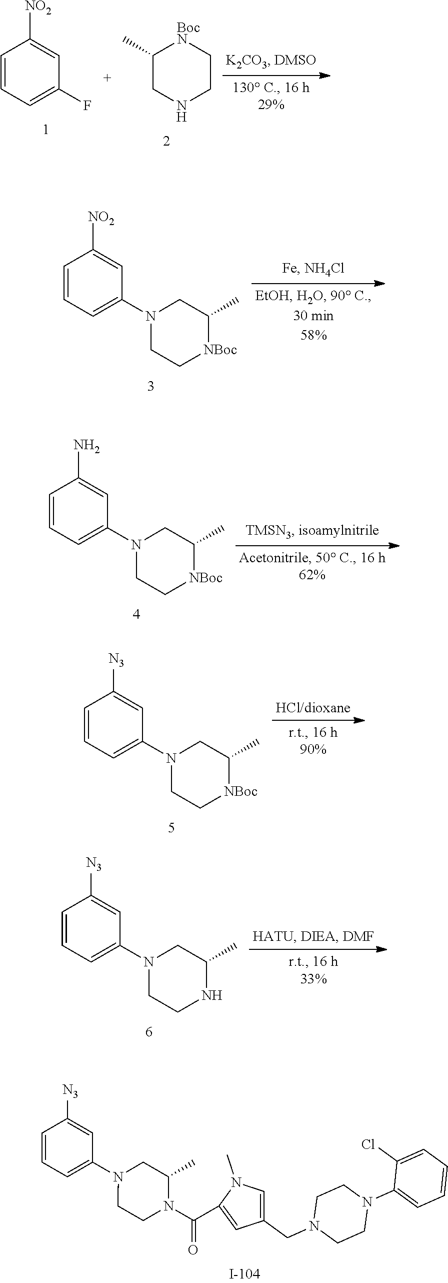

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid. Analysis was performed following Method B. Separation was performed following Method D.

### Step 1: (S)-tert-butyl 2-methyl-4-(3-nitrophenyl)piperazine-1-carboxylate

A mixture of K₂CO₃ (8.8 g, 63.8 mmol), 1-fluoro-3-nitrobenzene (3 g, 21.3 mmol), and (S)-tert-butyl-2-methylpiperazine-1-carboxylate (4.26 g, 21.3 mmol) in DMSO (80 mL) was stirred at 130° C. for 16 hrs. The mixture was then filtered and the filtrate was washed with water, extracted with EtOAc, and purified by chromatography (silica, EtOAc/PE=1/10) to afford (S)-tert-butyl-2-methyl-4-(3-nitrophenyl)piperazine-1-carboxylate (1.98 g, 6.18 mmol, 29%). ESI-MS (EI⁺, m/z): 222.2 [M-99]⁺.

### Step 2: (S)-tert-butyl 4-(3-aminophenyl)-2-methylpiperazine-1-carboxylate

A mixture of (S)-tert-butyl-2-methyl-4-(3-nitrophenyl)piperazine-1-carboxylate (1.5 g, 4.67 mmol), Fe (1.05 g, 18.69 mmol) and NH₄Cl (1.24 g, 23.36 mmol) in EtOH (30 mL) and H₂O (12 mL) was stirred at 90° C. for 30 min. The mixture was filtered, concentrated, and purified by chromatography (silica, EtOAc/PE=1/4) to afford (S)-tert-butyl 4-(3-aminophenyl)-2-methylpiperazine-1-carboxylate (788 mg, 2.71 mmol, 58%). ESI-MS (EI⁺, m/z): 292.3 [M+H]⁺.

### Step 3: (S)-tert-butyl-4-(3-azidophenyl)-2-methylpiperazine-1-carboxylate

A solution of acetonitrile (20 mL), (S)-tert-butyl-4-(3-aminophenyl)-2-methylpiperazine-1-carboxylate (1.3 g, 4.47 mmol), TMSN₃ (1.03 g, 8.93 mmol), and isoamylnitrile (1.04 g, 8.93 mmol) was stirred for 16 hrs at 50° C. The mixture was then concentrated and purified by chromatography (silica, EtOAc/PE=1/4) to afford (S)-tert-butyl-4-(3-azidophenyl)-2-methylpiperazine-1-carboxylate (879 mg, 2.77 mmol, 62%). ESI-MS (EI⁺, m/z): 318.2 [M+H]⁺.

The synthesis of (S)-(4-(3-azidophenyl)-2-methylpiperazin-1-yl)(4-((4-(2-chlorophenyl)piperazin-1-yl)methyl)-1,5-dimethyl-1H-pyrrol-2-yl)methanone I-104 was the same as Example 1.

ESI-MS (EI⁺, m/z): 548.2 [M+H]⁺. ¹H NMR (500 MHz, CDCl₃) δ 7.34 (d, J=7.8 Hz, 1H), 7.28-7.18 (m, 2H), 7.04 (d, J=7.7 Hz, 1H), 6.96 (t, J=7.6 Hz, 1H), 6.68 (dd, J=8.3, 2.0 Hz, 1H), 6.60-6.54 (m, 1H), 6.49 (s, 1H), 6.31 (s, 1H), 4.86 (s, 1H), 4.41 (d, J=13.3 Hz, 1H), 3.65 (s, 3H), 3.56 (d, J=11.4 Hz, 1H), 3.44 (m, 4H), 3.15-2.94 (m, 5H), 2.82 (td, J=11.8, 3.3 Hz, 1H), 2.62 (s, 4H), 2.23 (s, 3H), 1.42 (d, J=6.7 Hz, 3H).

### Example 35: (4-((4-(2-Chlorophenyl)piperazin-1-yl)methyl)-1,5-dimethyl-1H-pyrrol-2-yl)(2-phenyl-6,7-dihydropyrazolo[1,5-a]pyrazin-5(4H)-yl)methanone 2,2,2-trifluoroacetate, I-81

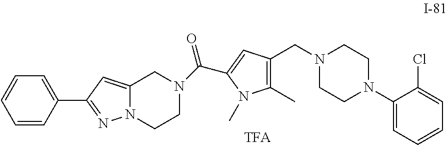 [SMILES: Cc1c(CN2CCN(c3ccccc3Cl)CC2)cc(C(=O)N2CCn3nc(-c4ccccc4)cc3C2)n1C]

### Synthetic Scheme:

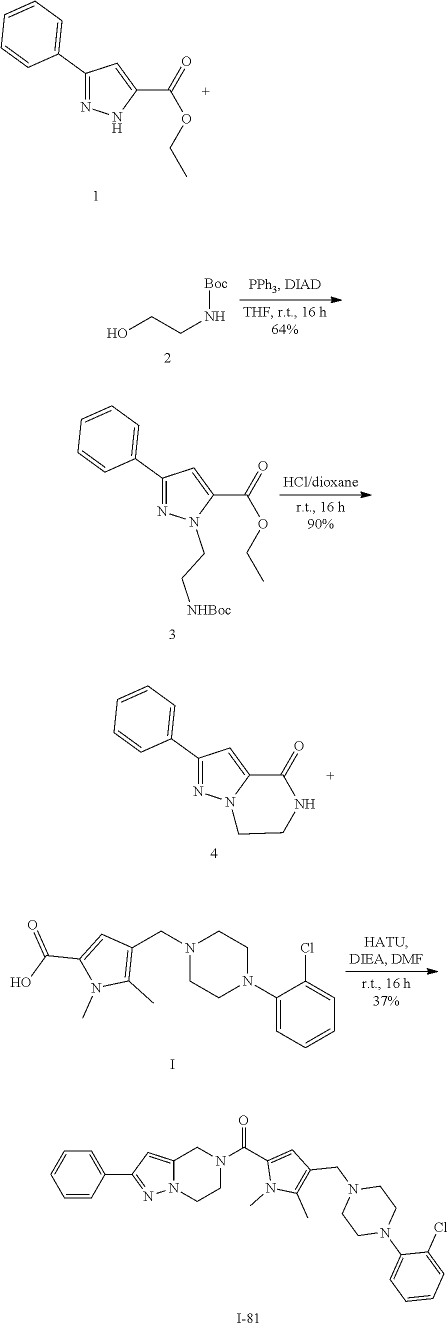

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method C.

### Step 1: Ethyl-1-(2-(tert-butoxycarbonylamino)ethyl)-3-phenyl-1H-pyrazole-5-carboxylate

A mixture of ethyl 3-phenyl-1H-pyrazole-5-carboxylate (2.16 g, 10 mmol), tert-butyl-2-hydroxyethylcarbamate (3.22 g, 20 mmol), DIAD (4.04 g, 20 mmol), and PPh₃ (5.24 g, 20 mmol) in THF (70 mL) was stirred at rt for 16 hrs. The mixture was then concentrated and purified by chromatography (silica, EtOAc/PE=1/7) to afford ethyl 1-(2-(tert-butoxycarbonylamino)ethyl)-3-phenyl-1H-pyrazole-5-carboxylate (2.3 g, 6.4 mmol, 64%). ESI-MS (EI⁺, m/z): 360.3 [M+H]⁺.

### Step 2: 2-Phenyl-6,7-dihydropyrazolo[1,5-a]pyrazin-4(5H)-one

To a solution of HCl (4 N, dioxane) (30 mL) in an ice bath was added ethyl-1-(2-(tert-butoxycarbonylamino)ethyl)-3-phenyl-1H-pyrazole-5-carboxylate (1.55 g, 4.3 mmol). The solution was stirred for 16 hrs at 20° C., then concentrated to afford 2-phenyl-6,7-dihydropyrazolo[1,5-a]pyrazin-4(5H)-one (828 mg, 3.87 mmol, 90%). ESI-MS (EI⁺, m/z): 214.1 [M+H]⁺.

The synthesis of (4-((4-(2-Chlorophenyl)piperazin-1-yl)methyl)-1,5-dimethyl-1H-pyrrol-2-yl)(2-phenyl-6,7-dihydropyrazolo[1,5-a]pyrazin-5(4H)-yl)methanone-2,2,2-trifluoroacetate I-81 was the same as Example 1.

ESI-MS (EI⁺, m/z): 529.3 [M+H]⁺. ¹H NMR (500 MHz, CDCl₃) δ 7.76 (d, J=7.8 Hz, 2H), 7.39 (t, J=7.7 Hz, 2H), 7.30 (m, 2H), 7.07 (m, 2H), 6.59 (s, 1H), 6.39 (s, 1H), 5.01 (s, 2H), 4.32 (d, J=5.3 Hz, 2H), 4.23 (d, J=5.2 Hz, 2H), 4.14 (s, 2H), 3.73-3.62 (m, 5H), 3.51-3.22 (m, 4H), 3.00 (t, J=9.7 Hz, 2H), 2.28 (s, 3H).

### Example 36: (S)-(4-((4-(3-chloropyridin-2-yl)piperazin-1-yl)methyl)-1,5-dimethyl-1H-pyrrol-2-yl)(4-(4-fluorophenyl)-2-methylpiperazin-1-yl)methanone, I-92

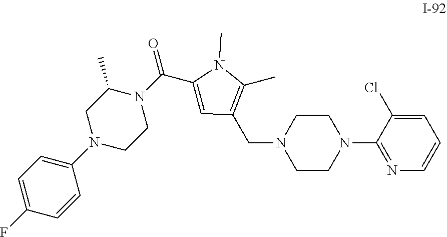 [SMILES: Cc1c(CN2CCN(c3ncccc3Cl)CC2)cc(C(=O)N2CCN(c3ccc(F)cc3)C[C@@H]2C)n1C]

### Synthetic Scheme:

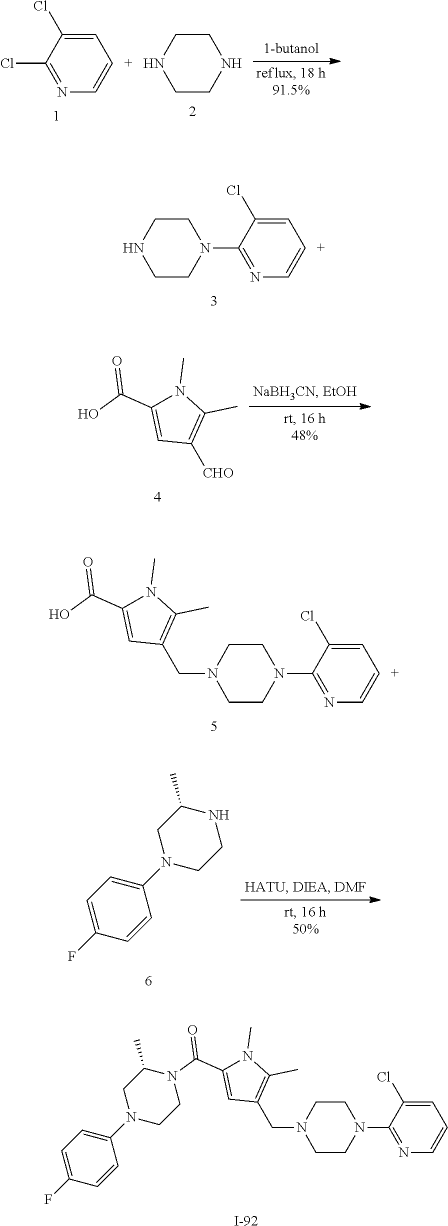

### Procedures and Characterization:

Analysis was performed following Method B. Separation was performed following Method D.

### Step 1: 1-(3-Chloropyridin-2-yl)piperazine

A mixture of 2,3-dichloropyridine (0.9 g, 6.1 mmol) and piperazine (5.5 g, 64.1 mmol) in n-butanol (25 mL) was refluxed for 18 hrs. The mixture was then concentrated, poured into water (100 mL), and extracted with EtOAc (100 mL×2). The combined organic layers were dried over Na₂SO₄, filtered, and concentrated in vacuo to give crude 1-(3-chloropyridin-2-yl)piperazine (1.1 g, 92%) as a yellow oil. ESI-MS (EI⁺, m/z): 198.2 [M+H]⁺.

### Step 2: 4-((4-(3-Chloropyridin-2-yl)piperazin-1-yl)methyl)-1,5-dimethyl-1H-pyrrole-2-carboxylic acid

To a mixture of 4-formyl-1,5-dimethyl-1H-pyrrole-2-carboxylic acid (1.17 g, 7.0 mmol) and 1-(3-chloropyridin-2-yl)piperazine (1.66 g, 8.4 mmol) in EtOH (20 mL) was added NaBH₃CN (880 g, 14.0 mmol). The mixture was stirred at rt for 16 hrs, then concentrated and poured into H₂O (100 mL). The mixture was adjusted pH=3 with aq. HCl (3 M) at 0° C. and filtered to afford 4-((4-(3-chloropyridin-2-yl)piperazin-1-yl)methyl)-1,5-dimethyl-1H-pyrrole-2-carboxylic acid (1.17 g, 48%) as a yellow solid. ESI-MS (EI⁺, m/z): 349.1 [M+H]⁺.

### Step 3: (S)-(4-((4-(3-chloropyridin-2-yl)piperazin-1-yl)methyl)-1,5-dimethyl-1H-pyrrol-2-yl)(4-(4-fluorophenyl)-2-methylpiperazin-1-yl)methanone

A mixture of 4-((4-(3-chloropyridin-2-yl)piperazin-1-yl)methyl)-1,5-dimethyl-1H-pyrrole-2-carboxylic acid (90 mg, 0.258 mmol), (S)-1-(4-fluorophenyl)-3-methylpiperazine (55 mg, 0.284 mmol), HATU (127 mg, 0.335 mmol), and DIPEA (67 mg, 0.516 mmol) in DMF (3 mL) was stirred at rt for 16 hrs. The mixture was purified by prep-HPLC to afford (S)-(4-((4-(3-chloropyridin-2-yl)piperazin-1-yl)methyl)-1,5-dimethyl-1H-pyrrol-2-yl)(4-(4-fluorophenyl)-2-methylpiperazin-1-yl)methanone I-92 (67 mg, 50%) as a white solid. ESI-MS (EI⁺, m/z): 525.3 [M+H]⁺. ¹H NMR (500 MHz, CDCl₃) δ 8.17 (dd, J=4.7, 1.4 Hz, 1H), 7.56 (dd, J=7.7, 1.4 Hz, 1H), 6.98 (t, J=8.7 Hz, 2H), 6.90-6.85 (m, 2H), 6.81 (dd, J=7.7, 4.8 Hz, 1H), 6.30 (s, 1H), 4.86 (s, 1H), 4.41 (d, J=13.6 Hz, 1H), 3.65 (s, 3H), 3.53-3.24 (m, 9H), 2.91 (dd, J=11.8, 3.6 Hz, 1H), 2.74 (td, J=11.8, 3.1 Hz, 1H), 2.60 (s, 4H), 2.22 (s, 3H), 1.45 (d, J=6.8 Hz, 3H).

### Example 37: Synthesis of (4-((4-(3-chloropyridin-2-yl)piperazin-1-yl)methyl)-1,5-dimethyl-1H-pyrrol-2-yl)(4-(3,4-difluorophenyl) piperazin-1-yl)methanone, I-84

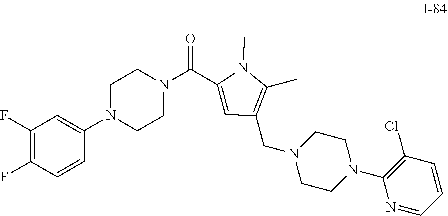 [SMILES: Cc1c(CN2CCN(c3ncccc3Cl)CC2)cc(C(=O)N2CCN(c3ccc(F)c(F)c3)CC2)n1C]

### Synthetic Scheme

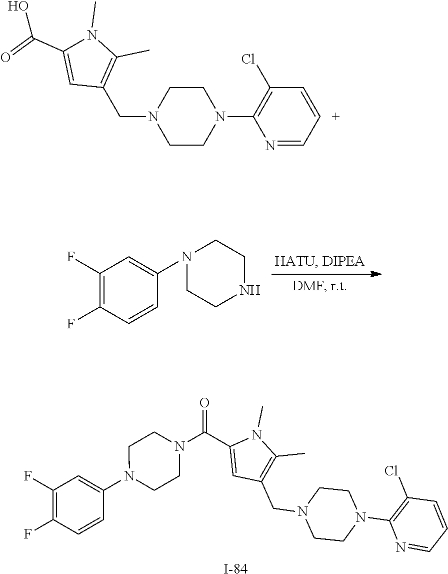

### Procedures and Characterization:

The synthesis of 4-((4-(3-chloropyridin-2-yl)piperazin-1-yl)methyl)-1,5-dimethyl-1H-pyrrole-2-carboxylic acid was the same as Example 36.

Analysis was performed following Method A. Separation was performed following Method C.

### Step 1: Synthesis of (4-((4-(3-chloropyridin-2-yl) piperazin-1-yl) methyl)-1,5-dimethyl-1H-pyrrol-2-yl)(4-(3,4-difluorophenyl) piperazin-1-yl)methanone

To a mixture of 4-((4-(3-chloropyridin-2-yl)piperazin-1-yl)methyl)-1,5-dimethyl-1H-pyrrole-2-carboxylic acid (82 mg, 0.229 mmol), 1-(3,4-difluorophenyl)piperazine (50 mg, 0.252 mmol), HATU (131 mg, 0.344 mmol), and DIPEA (29.64 mg, 0.229 mmol) was added DMF (3 mL). This mixture was stirred at rt overnight. The mixture was extracted with EtOAc/H₂O (20 ml/20 ml). The organic phase was dried over anhydrous Na₂SO₄ and concentrated in vacuo. The residue was purified by HPLC to afford (4-((4-(3-chloropyridin-2-yl)piperazin-1-yl)methyl)-1,5-dimethyl-1H-pyrrol-2-yl)(4-(3,4-difluorophenyl)piperazin-1-yl)methanone I-84 (62 mg, 51.2%). MS (EI+, m/z): 530 [M+H]⁺. ¹H NMR (500 MHz, DMSO-d₆) δ 8.27 (d, J=4.0 Hz, 1H), 7.88 (d, J=8.0 Hz, 1H), 7.29 (q, J=9.5, 19.0 Hz, 1H), 7.06-7.02 (m, 1H), 6.77 (d, J=8.5 Hz, 1H), 6.48 (s, 1H), 4.25 (s, 2H), 3.92-3.88 (m, 8H), 3.76 (s, 4H), 3.58 (s, 3H), 3.50-3.48 (m, 2H), 3.14 (s, 2H), 2.29 (s, 3H).

### Example 38: 3-(4-(4-((4-(3-Chloropyridin-2-yl)piperazin-1-yl)methyl)-1,5-dimethyl-1H-pyrrole-2-carbonyl)piperazin-1-yl)benzonitrile, I-79

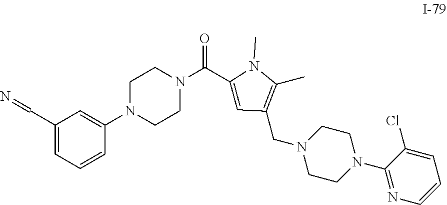 [SMILES: Cc1c(CN2CCN(c3ncccc3Cl)CC2)cc(C(=O)N2CCN(c3cccc(C=N)c3)CC2)n1C]

### Procedures and Characterization:

The synthesis of 4-((4-(3-chloropyridin-2-yl)piperazin-1-yl)methyl)-1,5-dimethyl-1H-pyrrole-2-carboxylic acid was the same as Example 36. The procedure for 1-79 was the same as Example 1.

Analysis was performed following Method A. Separation was performed following Method D. ESI-MS (EI⁺, m/z): 518.1 [M+H]⁺. ¹H NMR (500 MHz, DMSO-d₆) δ 8.21 (dd, J=4.5, 1.0 Hz, 1H), 7.78 (dd, J=7.5, 1.5 Hz, 1H), 7.41 (t, J=8.0 Hz, 1H), 7.37 (s, 1H), 7.31 (d, J=8.5 Hz, 1H), 7.20 (d, J=7.5 Hz, 1H), 6.99 (dd, J=7.5, 4.5 Hz, 1H), 6.28 (s, 1H), 3.76 (m, 4H), 3.56 (s, 3H), 3.35 (s, 2H), 3.30 (m, 8H), 3.23 (m, 4H), 2.20 (s, 3H).

### Example 39: (4-((4-(3-Chloropyridin-2-yl)piperazin-1-yl)methyl)-1,5-dimethyl-1H-pyrrol-2-yl)(4-(4-fluorophenyl)piperazin-1-yl)methanone, I-80

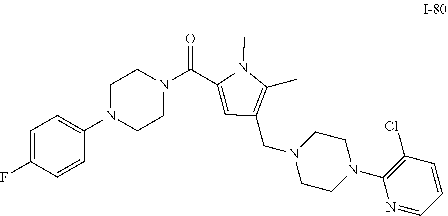 [SMILES: Cc1c(CN2CCN(c3ncccc3Cl)CC2)cc(C(=O)N2CCN(c3ccc(F)cc3)CC2)n1C]

### Procedures and Characterization:

The procedure was the same as Example 36.

Analysis was performed following Method A. Separation was performed following Method D. ESI-MS (EI⁺, m/z): 511.1 [M+H]⁺. ¹H NMR (500 MHz, DMSO-d₆) δ 8.25-8.16 (m, 1H), 7.78 (d, J=6.5 Hz, 1H), 7.08 (t, J=8.5 Hz, 2H), 7.02-6.96 (m, 3H), 6.26 (s, 1H), 3.77 (m, 4H), 3.56 (s, 3H), 3.34 (m, 2H), 3.34 (m, 4H), 3.23 (m, 4H), 3.12 (m, 4H), 2.19 (s, 3H).

### Example 40: (S)-(4-((4-(3-chloropyridin-2-yl)piperazin-1-yl)methyl)-1,5-dimethyl-1H-pyrrol-2-yl)(4-(3,4-difluorophenyl)-2-methylpiperazin-1-yl)methanone, I-78

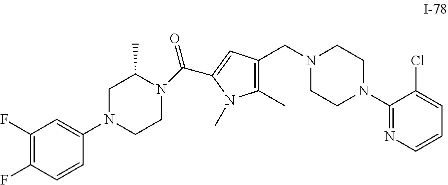 [SMILES: Cc1c(CN2CCN(c3ncccc3Cl)CC2)cc(C(=O)N2CCN(c3ccc(F)c(F)c3)C[C@@H]2C)n1C]

### Procedures and Characterization:

The procedure was the same as Examples 36 and 19.

Analysis was performed following Method B. Separation was performed following Method D. (S)-(4-((4-(3-chloropyridin-2-yl)piperazin-1-yl)methyl)-1,5-dimethyl-1H-pyrrol-2-yl)(4-(3,4-difluorophenyl)-2-methylpiperazin-1-yl)methanone I-78: ESI-MS (EI⁺, m/z): 543.2 [M+H]⁺. ¹H NMR (500 MHz, CDCl₃) δ 8.17 (d, J=4.3 Hz, 1H), 7.57 (d, J=7.7 Hz, 1H), 7.05 (q, J=9.4 Hz, 1H), 6.81 (dd, J=7.7, 4.8 Hz, 1H), 6.70 (ddd, J=13.0, 6.7, 2.8 Hz, 1H), 6.58 (d, J=9.1 Hz, 1H), 6.30 (s, 1H), 4.86 (s, 1H), 4.41 (d, J=13.6 Hz, 1H), 3.65 (s, 3H), 3.55-3.23 (m, 9H), 2.93 (dd, J=11.9, 3.5 Hz, 1H), 2.81-2.71 (m, 1H), 2.60 (s, 4H), 2.22 (s, 3H), 1.43 (d, J=6.8 Hz, 3H).

### Example 41: (S)-(4-(3-chlorophenyl)-2-methylpiperazin-1-yl)(4-((4-(2-chlorophenyl)piperazin-1-yl)methyl)-1,5-dimethyl-1H-pyrrol-2-yl)methanone, I-112

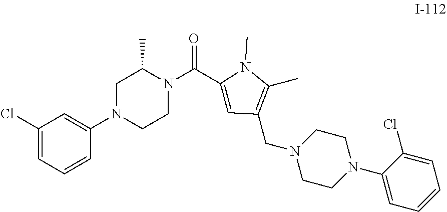 [SMILES: Cc1c(CN2CCN(c3ccccc3Cl)CC2)cc(C(=O)N2CCN(c3cccc(Cl)c3)C[C@@H]2C)n1C]

### Synthetic Scheme:

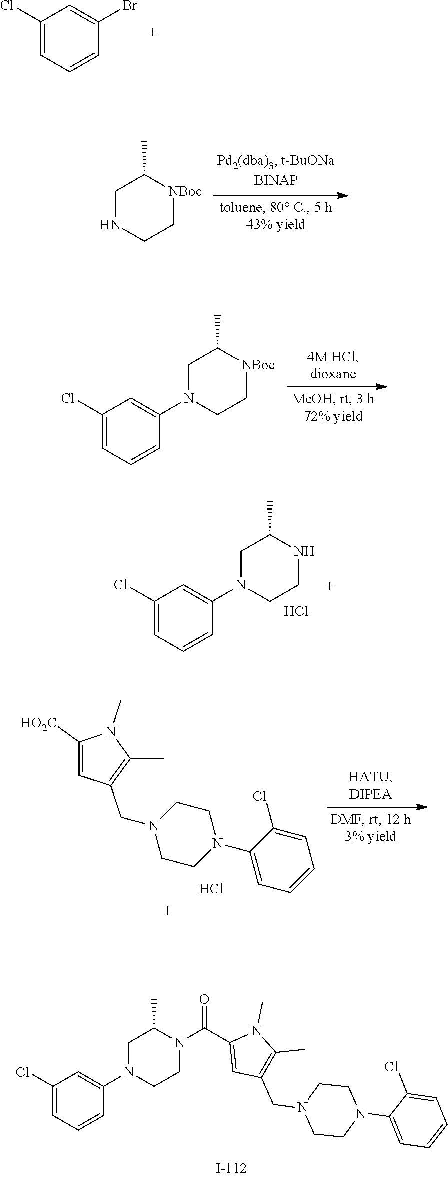

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: (S)-tert-butyl-4-(3-chlorophenyl)-2-methylpiperazine-1-carboxylate

To a rt solution of (S)-tert-butyl-2-methylpiperazine-1-carboxylate (1.0 g, 5.0 mmol), Pd₂(dba)₃ (91.6 mg, 0.1 mmol), t-BuONa (720 mg, 7.5 mmol), and BINAP (124.4 mg, 0.2 mmol) in toluene (15 mL) under N₂ atmosphere was added 1-bromo-3-chlorobenzene (1.42 g, 7.5 mmol). The solution was then heated to 80° C. for 5 hrs. The reaction was monitored by TLC and following completion was cooled to rt. The solvent was filtered and concentrated to yield a crude brown mixture that was purified by column chromatography (EtOAc/PE=1:20 to 1:5) to afford (S)-tert-butyl-4-(3-chlorophenyl)-2-methylpiperazine-1-carboxylate (666 mg, 2.15 mmol) as a pale yellow oil. ESI-MS (EI⁺, m/z): 311.0 [M+H]⁺.

### Step 2: (S)-1-(3-chlorophenyl)-3-methylpiperazine hydrochloride

To a solution of (S)-tert-butyl-4-(3-chlorophenyl)-2-methylpiperazine-1-carboxylate (666 mg, 2.15 mmol) in MeOH (5 mL) was added 4 M HCl/dioxane (15 mL). After stirring for 3 hrs at rt, the solvent was removed in vacuo to yield (S)-1-(3-chlorophenyl)-3-methylpiperazine hydrochloride (381 mg, 1.55 mmol) as a yellow solid which was used without further purification. ESI-MS (EI⁺, m/z): 211.1 [M+H]⁺.

### Step 3: (S)-(4-(3-chlorophenyl)-2-methylpiperazin-1-yl)(4-((4-(2-chlorophenyl)piperazin-1-yl)methyl)-1,5-dimethyl-1H-pyrrol-2-yl)methanone

To a rt solution of 4-((4-(2-chlorophenyl)piperazin-1-yl)methyl)-1,5-dimethyl-1H-pyrrole-2-carboxylic acid hydrochloride (1.19 g, 3.1 mmol), HATU (766 mg, 2.0 mmol), and DIPEA (1.20 g, 9.3 mmol) in DMF (4 mL) was added (S)-1-(3-chlorophenyl)-3-methylpiperazine hydrochloride (381 mg, 1.55 mmol). The reaction was stirred at rt overnight. The solution was then purified by prep-HPLC (Boston C18 21*250 mm 10 μm, Mobile phase: A: 0.1% ammonium hydrogen carbonate; B: acetonitrile) to afford (S)-(4-(3-chlorophenyl)-2-methylpiperazin-1-yl)(4-((4-(2-chlorophenyl)piperazin-1-yl)methyl)-1,5-dimethyl-1H-pyrrol-2-yl)methanone I-112 (25.6 mg, 0.047 mmol) as a white solid. MS (EI⁺, m/z): 540.2 [M+H]⁺. ¹H NMR (500 MHz, CDCl₃) δ 7.34 (d, J=8.0 Hz, 1H), 7.26-7.16 (m, 2H), 7.04 (d, J=8.0 Hz, 1H), 6.96 (t, J=7.5 Hz, 1H), 6.87-6.83 (m, 2H), 6.77 (dd, J=8.5, 2.0 Hz, 1H), 6.32 (s, 1H), 4.86 (s, 1H), 4.41 (d, J=13.0 Hz, 1H), 3.65 (s, 3H), 3.55 (d, J=12.0 Hz, 1H), 3.48-3.42 (m, 4H), 3.07-2.98 (m, 5H), 2.85-2.80 (m, 1H), 2.64 (s, 4H), 2.23 (s, 3H), 1.42 (d, J=7.0 Hz, 4H).

### Example 42: 5-(4-(4-((4-(2-Chlorophenyl)piperazin-1-yl)methyl)-1,5-dimethyl-1H-pyrrole-2-carbonyl)piperazin-1-yl)-2-fluorobenzonitrile, I-105

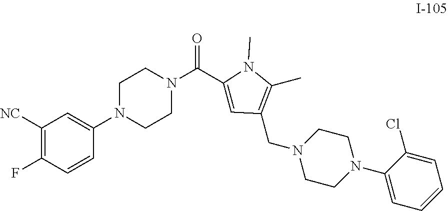 [SMILES: Cc1c(CN2CCN(c3ccccc3Cl)CC2)cc(C(=O)N2CCN(c3ccc(F)c(C#N)c3)CC2)n1C]

### Synthetic Scheme:

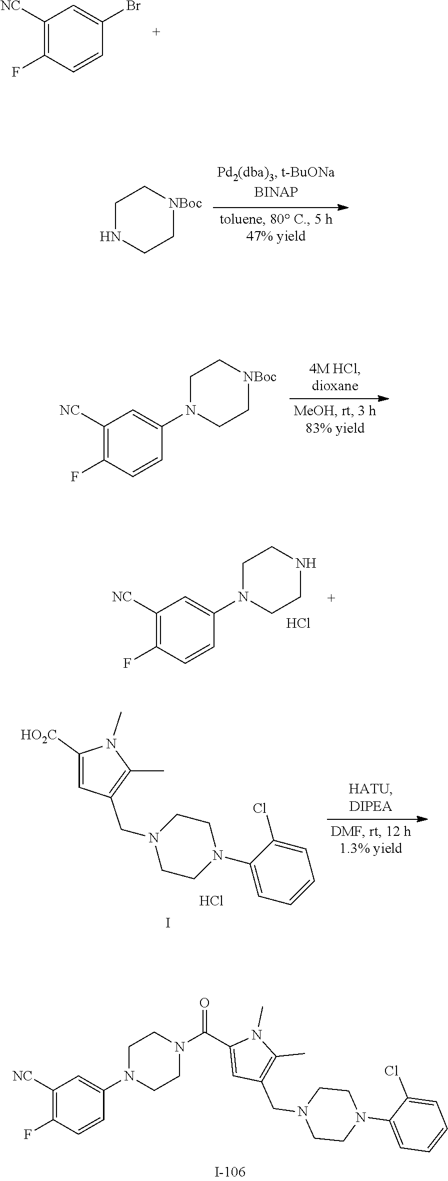

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid (I). Analysis was performed following Method B. Separation was performed following Method D.

### Step 1: tert-Butyl 4-(3-cyano-4-fluorophenyl)piperazine-1-carboxylate

To a rt solution of tert-butyl-piperazine-1-carboxylate (930 mg, 5.0 mmol), Pd₂(dba)₃ (91.6 mg, 0.1 mmol), t-BuONa (720 mg, 7.5 mmol), and BINAP (124.4 mg, 0.2 mmol) in toluene (15 mL) under N₂ atmosphere was added 5-bromo-2-fluorobenzonitrile (1.5 g, 7.5 mmol). The solution was then heated to 80° C. and stirred for 5 hrs. The reaction was monitored by TLC and following reaction completion was cooled to rt. The solution was filters then concentrated to yield a crude brown mixture which was then purified by column chromatography (EtOAc/PE=1:20 to 1:5) to afford tert-butyl-4-(3-cyano-4-fluorophenyl)piperazine-1-carboxylate (717 mg, 2.35 mmol) as a pale yellow oil. ESI-MS (EI⁺, m/z): 250.1 [M-56]⁺.

### Step 2: 2-Fluoro-5-(piperazin-1-yl)benzonitrile hydrochloride

To a solution of tert-butyl-4-(3-cyano-4-fluorophenyl)piperazine-1-carboxylate (717 mg, 2.35 mmol) in MeOH (5 mL) was added 4 M HCl/dioxane (15 mL). After stirring for 3 hrs at rt, the solvent was removed in vacuo to yield 2-fluoro-5-(piperazin-1-yl)benzonitrile hydrochloride (472 mg, 1.95 mmol) as yellow solid, which was used without further purification. ESI-MS (EI⁺, m/z): 206.1 [M+H]⁺.

### Step 3: 5-(4-(4-((4-(2-Chlorophenyl)piperazin-1-yl)methyl)-1,5-dimethyl-1H-pyrrole-2-carbonyl)piperazin-1-yl)-2-fluorobenzonitrile

To a rt solution of 4-((4-(2-chlorophenyl)piperazin-1-yl)methyl)-1,5-dimethyl-1H-pyrrole-2-carboxylic acid hydrochloride (1.50 g, 3.9 mmol), HATU (963 mg, 2.5 mmol), and DIPEA (1.51 g, 11.7 mmol) in DMF (4 mL) was added 2-fluoro-5-(piperazin-1-yl)benzonitrile hydrochloride (472 mg, 1.95 mmol). The reaction was stirred at rt overnight. The solution was then purified by prep-HPLC (Boston C18 21*250 mm 10 μm, Mobile phase: A: 0.1% ammonium hydrogen carbonate; B: acetonitrile) to afford 5-(4-(4-((4-(2-chlorophenyl)piperazin-1-yl)methyl)-1,5-dimethyl-1H-pyrrole-2-carbonyl)piperazin-1-yl)-2-fluorobenzonitrile I-105 (13.6 mg, 0.025 mmol) as a white solid. MS (EI⁺, m/z): 535.3 [M+H]⁺. ¹H NMR (500 MHz, CDCl₃) δ 7.35 (dd, J=8.0, 1.0 Hz, 1H), 7.22-7.19 (m, 1H), 7.15-7.10 (m, 2H), 7.07-7.03 (m, 2H), 6.96 (t, J=7.5 Hz, 1H), 6.35 (s, 1H), 3.93-3.91 (m, 4H), 3.67 (s, 3H), 3.44 (s, 2H), 3.18-3.16 (m, 4H), 3.07 (s, 4H), 2.64 (s, 4H), 2.23 (s, 3H).

### Example 43: 2-(8-((1,2-dimethyl-5-(4-(pyridin-2-yl)piperazine-1-carbonyl)-1H-pyrrol-3-yl)methyl)-3,8-diazabicyclo[3.2.1]octan-3-yl)benzonitrile, I-52

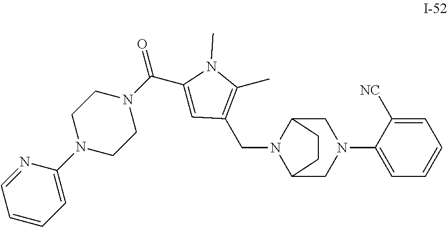 [SMILES: Cc1c(CN2C3CCC2CN(c2ccccc2C#N)C3)cc(C(=O)N2CCN(c3ccccn3)CC2)n1C]

### Synthetic Scheme:

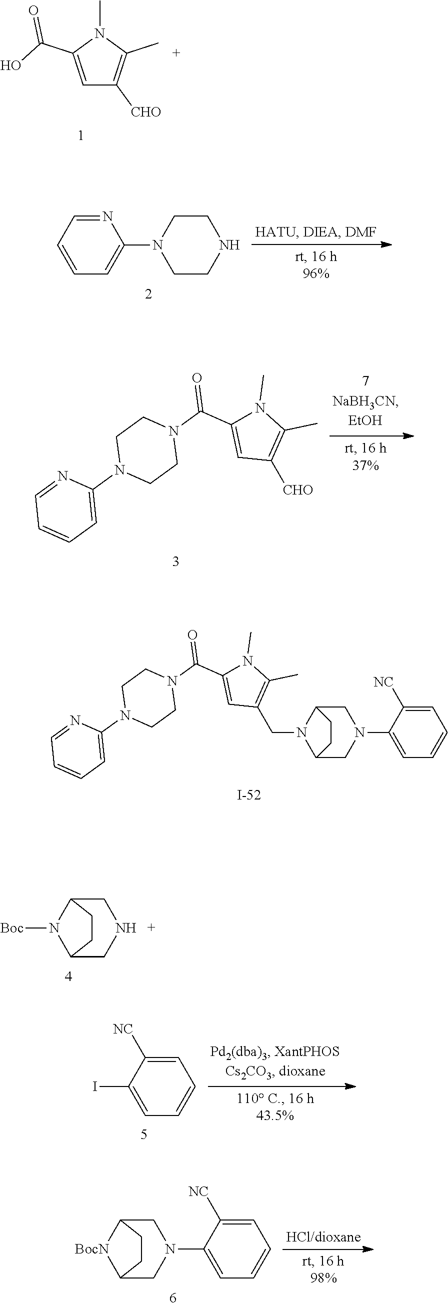

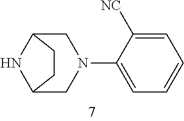 [SMILES: N#Cc1ccccc1N1CC2CCC(C1)N2]

### Procedures and Characterization:

The general procedure for Intermediate I was followed to obtain 4-formyl-1,5-dimethyl-1H-pyrrole-2-carboxylic acid.

Analysis was performed following Method B. Separation was performed following Method D.

### Step 1: 1,2-Dimethyl-5-(4-(pyridin-2-yl)piperazine-1-carbonyl)-1H-pyrrole-3-carbaldehyde

A mixture of 4-formyl-1,5-dimethyl-1H-pyrrole-2-carboxylic acid (500 mg, 3.0 mmol), 1-(pyridin-2-yl)piperazine (538 mg, 3.3 mmol), HATU (1.48 g, 3.9 mmol), DIPEA (775 mg, 6.0 mmol) in DMF (10 mL) was stirred at rt for 16 hrs. The mixture was purified by chromatography (silica, EtOAc/PE=1/10) to afford 1,2-dimethyl-5-(4-(pyridin-2-yl)piperazine-1-carbonyl)-1H-pyrrole-3-carbaldehyde (900 mg, 2.88 mmol, 96%) as a white solid. ESI-MS (EI⁺, m/z): 313.2 [M+H]⁺.

### Step 2: tert-Butyl 3-(2-cyanophenyl)-3,8-diazabicyclo[3.2.1]octane-8-carboxylate

A solution of tert-butyl 3,8-diazabicyclo[3.2.1]octane-8-carboxylate (106 mg, 0.5 mmol), 2-iodobenzonitrile (229 mg, 1 mmol), Cs₂CO₃ (326 mg, 1 mmol), XantPhos (23 mg, 0.04 mmol), Pd₂(dba)₃ (18.3 mg, 0.02 mmol) in dioxane (10 mL) was stirred under N₂ at 110° C. for 16 hrs. The reaction mixture was concentrated and purified by chromatography (silica, EtOAc/PE=1/5) to afford tert-butyl-3-(2-cyanophenyl)-3,8-diazabicyclo[3.2.1]octane-8-carboxylate (68 mg, 0.217 mmol, 43.5%) as a yellow oil. ESI-MS (EI⁺, m/z): 314.2 [M+H]⁺.

### Step 3: 2-(3,8-Diazabicyclo[3.2.1]octan-3-yl)benzonitrile

A mixture of tert-butyl-3-(2-cyanophenyl)-3,8-diazabicyclo[3.2.1]octane-8-carboxylate (150 mg, 0.479 mmol) in HCl/dioxane (10 mL) was stirred at rt for 16 hrs. The reaction mixture was concentrated, neutralized with aq. NaHCO₃ to pH=8, then extracted with EtOAc (50 mL×2). The organic layers were concentrated to give product 1-(3-chloro-4-fluorophenyl)piperazine (100 mg, 0.469 mmol, 98%) as a yellow solid. ESI-MS (EI⁺, m/z): 214.1 [M+H]⁺.

### Step 4: 2-(8-((1,2-Dimethyl-5-(4-(pyridin-2-yl)piperazine-1-carbonyl)-1H-pyrrol-3-yl)methyl)-3,8-diazabicyclo[3.2.1]octan-3-yl)benzonitrile

To a solution of 2-(3,8-diazabicyclo[3.2.1]octan-3-yl)benzonitrile (85 mg, 0.4 mmol), tert-butyl-3-(2-cyanophenyl)-3,8-diazabicyclo[3.2.1]octane-8-carboxylate (150 mg, 0.48 mmol) in EtOH (10 mL) and HOAc (1 drop) was stirred at rt for 2 hrs. Then NaBH₃CN (50 mg, 0.8 mmol) was added and the mixture was stirred at rt for 14 hrs. The mixture was then purified by prep-HPLC to afford 2-(8-((1,2-dimethyl-5-(4-(pyridin-2-yl)piperazine-1-carbonyl)-1H-pyrrol-3-yl)methyl)-3,8-diazabicyclo[3.2.1]octan-3-yl)benzonitrile I-52 (75.5 mg, 0.148 mmol, 37%) as a white solid. ESI-MS (EI⁺, m/z): 510.3 [M+H]⁺. ¹H NMR (500 MHz, CDCl₃) δ 8.20 (d, J=4.6 Hz, 1H), 7.51 (dd, J=11.4, 4.4 Hz, 2H), 7.43 (t, J=7.8 Hz, 1H), 6.93 (dd, J=7.9, 4.4 Hz, 2H), 6.67 (dd, J=10.4, 5.5 Hz, 2H), 6.37 (s, 1H), 3.89 (t, J=5.0 Hz, 4H), 3.67 (s, 3H), 3.60 (m, J=5.0 Hz, 4H), 3.37-3.34 (m, 4H), 3.28 (s, 2H), 3.04 (d, J=10.3 Hz, 2H), 2.24 (s, 3H), 2.10-1.94 (m, 4H).

### Example 44: (4-(1-(4-(2-Chlorophenyl)piperazin-1-yl)ethyl)-1,5-dimethyl-1H-pyrrol-2-yl)((S)-4-(4-fluorophenyl)-2-methylpiperazin-1-yl)methanone, I-46

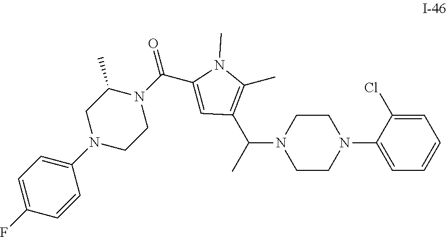 [SMILES: Cc1c(C(C)N2CCN(c3ccccc3Cl)CC2)cc(C(=O)N2CCN(c3ccc(F)cc3)C[C@@H]2C)n1C]

### Synthetic Scheme:

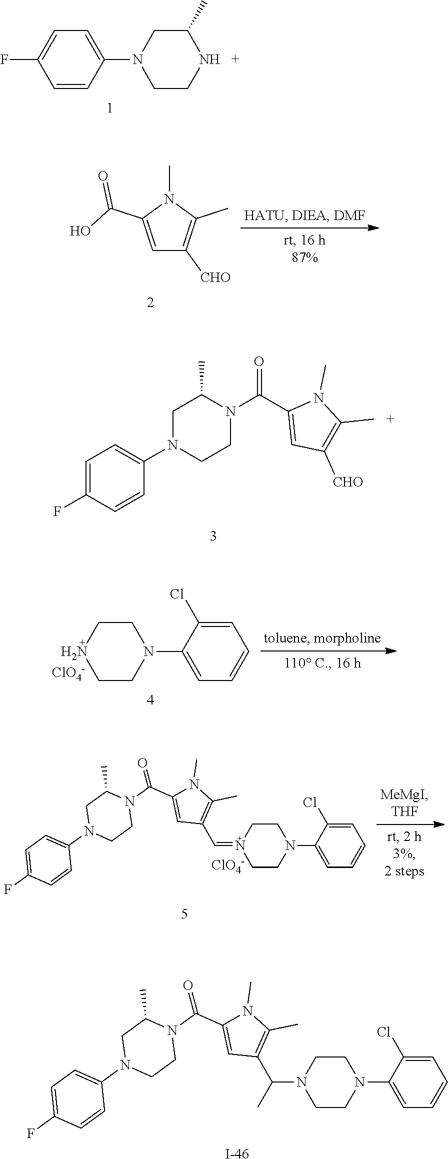

### Procedures and Characterization:

The general procedure for Intermediate I was followed to obtain 4-formyl-1,5-dimethyl-1H-pyrrole-2-carboxylic acid.

Analysis was performed following Method B. Separation was performed following Method D.

### Step 1: (S)-5-(4-(4-fluorophenyl)-2-methylpiperazine-1-carbonyl)-1,2-dimethyl-1H-pyrrole-3-carbaldehyde

A mixture of 4-formyl-1,5-dimethyl-1H-pyrrole-2-carboxylic acid (334 mg, 2.0 mmol), (S)-1-(4-fluorophenyl)-3-methylpiperazine (427 mg, 2.2 mmol), HATU (989 mg, 2.6 mmol), DIPEA (517 mg, 4 mmol) in DMF (10 mL) was stirred at rt for 16 hrs. The mixture was purified by chromatography (silica, EtOAc/PE=1/4) to afford (S)-5-(4-(4-fluorophenyl)-2-methylpiperazine-1-carbonyl)-1,2-dimethyl-1H-pyrrole-3-carbaldehyde (600 mg, 1.75 mmol, 87%) as a yellow solid. ESI-MS (EI+, m/z): 344.1 [M+H]⁺.

### Step 2: (S)-4-(2-chlorophenyl)-1-((5-(4-(4-fluorophenyl)-2-methylpiperazine-1-carbonyl)-1,2-dimethyl-1H-pyrrol-3-yl)methylene)piperazin-1-ium perchlorate

A mixture of (S)-5-(4-(4-fluorophenyl)-2-methylpiperazine-1-carbonyl)-1,2-dimethyl-1H-pyrrole-3-carbaldehyde (408 mg, 1.17 mmol), 4-(2-chlorophenyl)piperazin-1-ium perchlorate (300 mg, 0.9 mmol) and morpholine (1 drop) in toluene (15 mL) was stirred under N₂ at 110° C. for 16 hrs and concentrated. The residue was washed with EtOH (20 mL) and Et₂O (20 mL) to yield crude (S)-4-(2-chlorophenyl)-1-((5-(4-(4-fluorophenyl)-2-methylpiperazine-1-carbonyl)-1,2-dimethyl-1H-pyrrol-3-yl)methylene)piperazin-1-ium perchlorate as a yellow solid that was used without further purification.

### Step 3: (4-(1-(4-(2-Chlorophenyl)piperazin-1-yl)ethyl)-1,5-dimethyl-1H-pyrrol-2-yl)((S)-4-(4-fluorophenyl)-2-methylpiperazin-1-yl)methanone

To a solution of (S)-4-(2-chlorophenyl)-1-((5-(4-(4-fluorophenyl)-2-methylpiperazine-1-carbonyl)-1,2-dimethyl-1H-pyrrol-3-yl)methylene)piperazin-1-ium perchlorate (700 mg, crude, 1.1 mmol) in THF (50 mL) at 0° C. was added methylmagnesium iodide (914 mg, 5.5 mmol). The mixture was stirred at rt for 2 hrs then purified by prep-HPLC and chiral-HPLC to afford (4-(1-(4-(2-chlorophenyl)piperazin-1-yl)ethyl)-1,5-dimethyl-1H-pyrrol-2-yl)((S)-4-(4-fluorophenyl)-2-methylpiperazin-1-yl)methanone I-46 (P2: 18 mg, 0.033 mmol, 3%) as a white solid. ESI-MS (EI⁺, m/z): 342.1 [M−195]⁺. ¹H NMR (500 MHz, CDCl₃) δ 7.34 (d, J=7.9 Hz, 1H), 7.21 (t, J=7.7 Hz, 1H), 7.03 (d, J=8.0 Hz, 1H), 6.97 (dd, J=18.2, 9.5 Hz, 4H), 6.87 (dd, J=8.9, 4.5 Hz, 2H), 6.28 (s, 1H), 4.86 (s, 1H), 4.40 (d, J=12.0 Hz, 1H), 3.64 (s, 3H), 3.56-3.42 (m, 3H), 3.31 (d, J=10.8 Hz, 1H), 3.05 (s, 4H), 2.93-2.89 (m, 1H), 2.77-2.70 (m, 1H), 2.64 (s, 4H), 1.47-1.43 (m, 3H), 1.39 (d, J=6.7 Hz, 3H).

### Example 45: (S)-(4-((4-(2-chlorophenyl)-1,4-diazepan-1-yl)methyl)-1,5-dimethyl-1H-pyrrol-2-yl)(4-(4-fluorophenyl)-2-methylpiperazin-1-yl)methanone, I-47

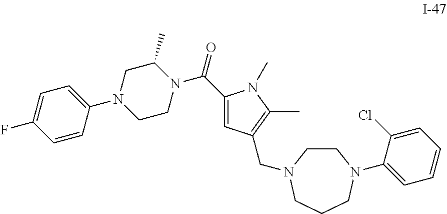 [SMILES: Cc1c(CN2CCCN(c3ccccc3Cl)CC2)cc(C(=O)N2CCN(c3ccc(F)cc3)C[C@@H]2C)n1C]

### Synthetic Scheme:

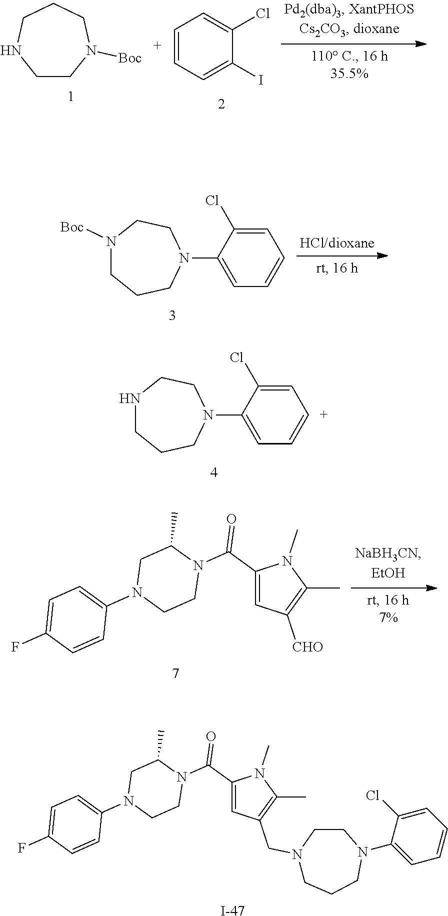

### Procedures and Characterization:

Analysis was performed following Method A. Separation was performed following Method D.

### Step 1: tert-Butyl 4-(2-chlorophenyl)-1,4-diazepane-1-carboxylate

A solution of tert-butyl 1,4-diazepane-1-carboxylate (1 g, 5 mmol), 1-chloro-2-iodobenzene (2.38 g, 10 mmol), Cs₂CO₃ (3.26 g, 10 mmol), XantPhos (289 mg, 0.5 mmol), Pd₂(dba)₃ (229 mg, 0.25 mmol) in dioxane (20 mL) was stirred under N₂ at 110° C. for 16 hrs. The reaction mixture was concentrated and then purified by chromatography (silica, EtOAc/PE=1/10) to afford tert-butyl-4-(2-chlorophenyl)-1,4-diazepane-1-carboxylate (550 mg, 1.77 mmol, 36%) as a yellow oil. ESI-MS (EI⁺, m/z): 311.1 [M+H]⁺.

### Step 2: 1-(2-Chlorophenyl)-1,4-diazepane

A mixture of tert-butyl 4-(2-chlorophenyl)-1,4-diazepane-1-carboxylate (550 mg, 1.77 mmol) in HCl/dioxane (20 mL) was stirred at rt for 16 hrs. The reaction mixture was concentrated to give crude product 1-(3-chloro-4-fluorophenyl)piperazine (400 mg, crude) as a yellow solid. ESI-MS (EI⁺, m/z): 211.1 [M+H]⁺.

### Step 3: (S)-(4-((4-(2-chlorophenyl)-1,4-diazepan-1-yl)methyl)-1,5-dimethyl-1H-pyrrol-2-yl)(4-(4-fluorophenyl)-2-methylpiperazin-1-yl)methanone

To a solution of (S)-5-(4-(4-fluorophenyl)-2-methylpiperazine-1-carbonyl)-1,2-dimethyl-1H-pyrrole-3-carbaldehyde (206 mg, 0.6 mmol) and 1-(2-chlorophenyl)-1,4-diazepane (105 mg, 0.5 mmol) in EtOH (5 mL) was added HOAc (1 drop). The mixture was stirred at rt for 2 hrs, then NaBH₃CN (63 mg, 1 mmol) was added and the mixture was stirred at rt for 14 hrs. The mixture was purified by prep-HPLC to afford (S)-(4-((4-(2-chlorophenyl)-1,4-diazepan-1-yl)methyl)-1,5-dimethyl-1H-pyrrol-2-yl)(4-(4-fluorophenyl)-2-methylpiperazin-1-yl)methanone I-47 (23.7 mg, 0.044 mmol, 7%) as a white solid. ESI-MS (EI⁺, m/z): 538.0 [M+H]⁺. ¹H NMR (500 MHz, CDCl₃) δ 7.32 (dd, J=7.9, 1.5 Hz, 1H), 7.19-7.12 (m, 1H), 7.07 (dd, J=8.1, 1.4 Hz, 1H), 6.97 (dt, J=12.0, 2.9 Hz, 2H), 6.89-6.85 (m, 3H), 6.30 (s, 1H), 4.86 (s, 1H), 4.41 (d, J=13.7 Hz, 1H), 3.64 (s, 3H), 3.51 (s, 2H), 3.44 (t, J=11.7 Hz, 2H), 3.33-3.28 (m, 5H), 2.90 (dd, J=11.8, 3.6 Hz, 1H), 2.81-2.71 (m, 5H), 2.21 (d, J=8.8 Hz, 3H), 1.97-1.92 (m, 2H), 1.45 (d, J=6.8 Hz, 3H).

### Example 46: (S)-3-(4-(4-((4-(2-chlorophenyl)-1,4-diazepan-1-yl)methyl)-1,5-dimethyl-1H-pyrrole-2-carbonyl)-3-methylpiperazin-1-yl)benzonitrile, I-41

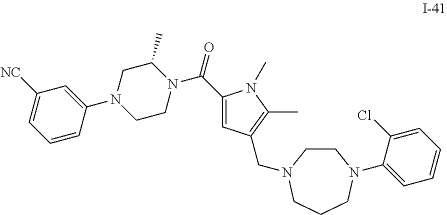 [SMILES: Cc1c(CN2CCCN(c3ccccc3Cl)CC2)cc(C(=O)N2CCN(c3cccc(C#N)c3)C[C@@H]2C)n1C]

### Synthetic Scheme:

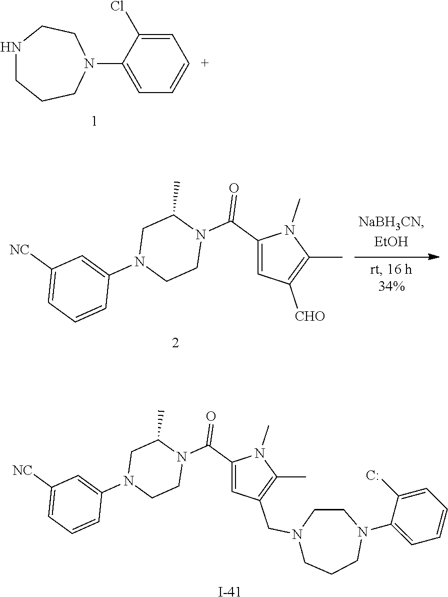

### Procedures and Characterization:

The synthesis of (S)-3-(4-(4-((4-(2-chlorophenyl)-1,4-diazepan-1-yl)methyl)-1,5-dimethyl-1H-pyrrole-2-carbonyl)-3-methylpiperazin-1-yl)benzonitrile I-41 was the same as Example 45.

Analysis was performed following Method A. Separation was performed following Method D. ESI-MS (EI⁺, m/z): 545.3 [M+H]⁺. ¹H NMR (500 MHz, CDCl₃) δ 7.33 (t, J=8.8 Hz, 2H), 7.12 (m, 5H), 6.88 (t, J=7.4 Hz, 1H), 6.31 (s, 1H), 4.89 (s, 1H), 4.44 (d, J=13.2 Hz, 1H), 3.65 (s, 3H), 3.58-3.44 (m, 5H), 3.33-3.28 (m, 4H), 3.05 (dd, J=12.1, 3.3 Hz, 1H), 2.89-2.76 (m, 5H), 2.21 (d, J=11.6 Hz, 3H), 1.98-1.93 (quartet, J=5.5 Hz, 2H), 1.42 (d, J=6.7 Hz, 3H).

### Example 47: 1-({5-[4-(3-Chlorophenyl)piperazine-1-carbonyl]-1,2-dimethyl-1H-pyrrol-3-yl}methyl)-4-(3-chloropyridin-2-yl)piperazine, I-98

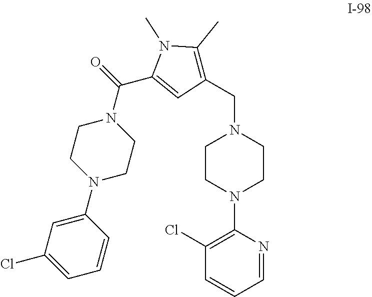 [SMILES: Cc1c(CN2CCN(c3ncccc3Cl)CC2)cc(C(=O)N2CCN(c3cccc(Cl)c3)CC2)n1C]

### Procedures and Characterization:

Analysis was performed following Method A. Separation was performed following Method D.

ESI-MS (EI+, m/z): 527.1 [M+H]+. 1H NMR (500 MHz, CDCl3) δ 8.17 (dd, J=5.0 Hz, 1.2 Hz, 1H), 7.57 (dd, J=7.5 Hz, 1.2 Hz, 1H), 7.19 (t, J=8.0 Hz, 1H), 6.90 (s, 1H), 6.86-6.79 (m, 3H), 6.33 (s, 1H), 3.92-3.89 (m, 4H), 3.67 (s, 3H), 3.42-3.36 (m, 6H), 3.24-3.21 (m, 4H), 2.59 (br, 4H), 2.23 (s, 3H).

### Example 48: (S)-(4-(3-chlorophenyl)-2-methylpiperazin-1-yl)(4-((4-(3-chloropyridin-2-yl)piperazin-1-yl)methyl)-1,5-dimethyl-1H-pyrrol-2-yl)methanone, I-90

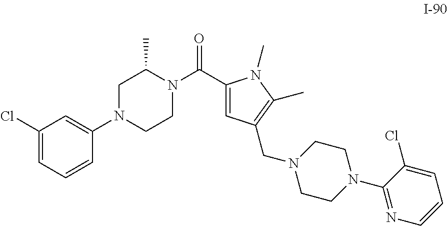 [SMILES: Cc1c(CN2CCN(c3ncccc3Cl)CC2)cc(C(=O)N2CCN(c3cccc(Cl)c3)C[C@@H]2C)n1C]

### Synthetic Scheme:

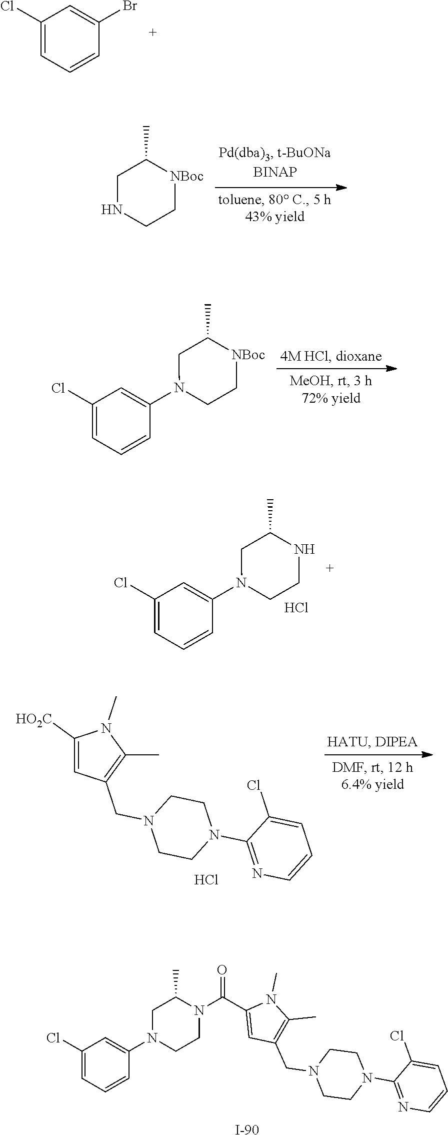

### Procedures and Characterization:

The synthesis of 4-((4-(3-chloropyridin-2-yl)piperazin-1-yl)methyl)-1,5-dimethyl-1H-pyrrole-2-carboxylic acid was the same as Example 36.

Analysis was performed following Method B. Separation was performed following Method D.

### Step 1: (S)-tert-butyl 4-(3-chlorophenyl)-2-methylpiperazine-1-carboxylate

To a rt solution of (S)-tert-butyl 2-methylpiperazine-1-carboxylate (1.0 g, 5.0 mmol), Pd₂(dba)₃ (91.6 mg, 0.1 mmol), t-BuONa (720 mg, 7.5 mmol) and BINAP (124.4 mg, 0.2 mmol) in toluene (15 mL) under N₂ atmosphere was added 1-bromo-3-chlorobenzene (1.42 g, 7.5 mmol). Then the solution was heated to 80° C. and stirred for 5 hrs. The reaction was monitored by TLC and following reaction completion was cooled to rt. The solvent was filtered and concentrated to yield a crude brown mixture which was purified by column chromatography (EtOAc/PE=1:20 to 1:5) to afford (S)-tert-butyl-4-(3-chlorophenyl)-2-methylpiperazine-1-carboxylate (666 mg, 2.15 mmol) as a pale yellow oil. ESI-MS (EI⁺, m/z): 311.0 [M+H]⁺.

### Step 2: (S)-1-(3-chlorophenyl)-3-methylpiperazine hydrochloride

To a solution of (S)-tert-butyl-4-(3-chlorophenyl)-2-methylpiperazine-1-carboxylate (666 mg, 2.15 mmol) in MeOH (5 mL) was added 4 M HCl/dioxane (15 mL). After stirring at rt for 3 hrs the solvent was removed in vacuo to yield (S)-1-(3-chlorophenyl)-3-methylpiperazine hydrochloride (381 mg, 1.55 mmol) as a yellow solid, which was used without further purification. ESI-MS (EI⁺, m/z): 211.1 [M+H]⁺.

### Step 3: (S)-(4-(3-chlorophenyl)-2-methylpiperazin-1-yl)(4-((4-(3-chloropyridin-2-yl)piperazin-1-yl)methyl)-1,5-dimethyl-1H-pyrrol-2-yl)methanone

To a rt solution of 4-((4-(3-chloropyridin-2-yl)piperazin-1-yl)methyl)-1,5-dimethyl-1H-pyrrole-2-carboxylic acid hydrochloride (1.19 g, 3.1 mmol), HATU (766 mg, 2.0 mmol), and DIPEA (1.20 g, 9.3 mmol) in DMF (4 mL) was added (S)-1-(3-chlorophenyl)-3-methylpiperazine hydrochloride (381 mg, 1.55 mmol). The reaction was stirred at rt overnight, then purified by prep-HPLC (Boston C18 21*250 mm 10 μm, Mobile phase: A: 0.1% ammonium hydrogen carbonate; B: acetonitrile) to afford (S)-(4-(3-chlorophenyl)-2-methylpiperazin-1-yl)(4-((4-(3-chloropyridin-2-yl)piperazin-1-yl)methyl)-1,5-dimethyl-1H-pyrrol-2-yl)methanone I-90 (54.2 mg, 0.1 mmol) as a white solid. MS (EI⁺, m/z): 541.3 [M+H]⁺. ¹H NMR (500 MHz, CDCl3) δ 8.17 (dd, J=4.5, 1.0 Hz, 1H), 7.57 (dd, J=7.5, 1.0 Hz, 1H), 7.18 (t, J=8.0 Hz, 1H), 6.87-6.77 (m, 4H), 6.33 (s, 1H), 4.86 (s, 1H), 4.41 (d, J=13.5 Hz, 1H), 3.65 (s, 3H), 3.56 (d, J=12.0 Hz, 1H), 3.46-3.40 (m, 8H), 3.00 (dd, J=12.0, 3.5 Hz, 1H), 2.85-2.79 (tdm, 1H), 2.63 (s, 4H), 2.23 (s, 3H), 1.42 (d, J=6.5 Hz, 3H).

### Example 49: (S)-(4-((4-(2-(3-aminopropyl)phenyl)piperazin-1-yl)methyl)-1,5-dimethyl-1H-pyrrol-2-yl)(2-methyl-4-phenylpiperazin-1-yl)methanone ditrifluoroacetate salt, I-24

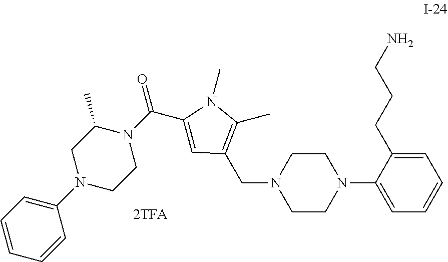 [SMILES: Cc1c(CN2CCN(c3ccccc3CCCN)CC2)cc(C(=O)N2CCN(c3ccccc3)C[C@@H]2C)n1C]

### Synthetic Scheme:

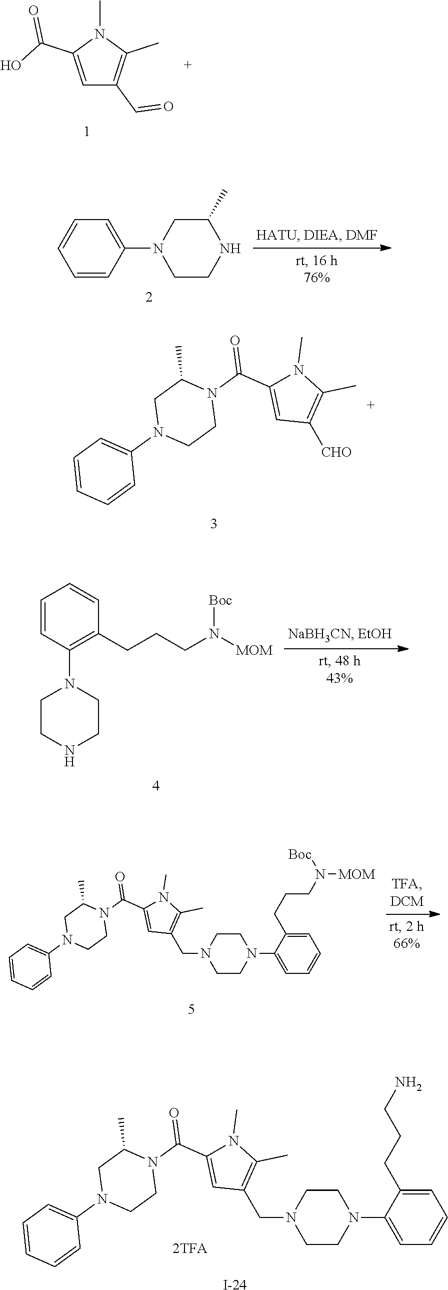

### Procedures and Characterization:

The general procedure for Intermediate I was followed to obtain 4-formyl-1,5-dimethyl-1H-pyrrole-2-carboxylic acid.

Analysis was performed following Method A. Separation was performed following Method C.

### Step 1: (S)-1,2-Dimethyl-5-(2-methyl-4-phenylpiperazine-1-carbonyl)-1H-pyrrole-3-carbaldehyde

A mixture of 4-formyl-1,5-dimethyl-1H-pyrrole-2-carboxylic acid (400 mg, 2.4 mmol), (S)-3-methyl-1-phenylpiperazine (508 mg, 2.88 mmol), HATU (1.186 g, 3.12 mmol), DIPEA (1.55 g, 12 mmol) in DMF (10 mL) was stirred at rt for 16 hrs. The mixture was purified by chromatography (silica, EtOAc/PE=1/5) to afford (S)-1,2-dimethyl-5-(2-methyl-4-phenylpiperazine-1-carbonyl)-1H-pyrrole-3-carbaldehyde (590 mg, 1.813 mmol, 76%) as a yellow oil. ESI-MS (EI⁺, m/z): 326.2 [M+H]⁺.

### Step 2: (S)-tert-Butyl-3-(2-(4-((1,2-dimethyl-5-(2-methyl-4-phenylpiperazine-1-carbonyl)-1H-pyrrol-3-yl)methyl)piperazin-1-yl)phenyl)propyl(methoxymethyl) carbamate

A solution of (S)-1,2-dimethyl-5-(2-methyl-4-phenylpiperazine-1-carbonyl)-1H-pyrrole-3-carbaldehyde (228 mg, 0.7 mmol), tert-butyl-methoxymethyl(3-(2-(piperazin-1-yl)phenyl)propyl)carbamate (254 mg, 0.7 mmol) in EtOH (10 mL), and HOAc (1 drop) was stirred at rt for 2 hrs. Then NaBH₃CN (88 mg, 1.4 mmol) was added and the mixture was stirred at rt for 14 hrs. The mixture was purified by chromatography (silica, EtOAc/PE=1/2) to afford (S)-tert-butyl-3-(2-(4-((1,2-dimethyl-5-(2-methyl-4-phenylpiperazine-1-carbonyl)-1H-pyrrol-3-yl)methyl)piperazin-1-yl)phenyl)propyl (methoxymethyl) carbamate (200 mg, 0.30 mmol, 43%) as a yellow oil. ESI-MS (EI⁺, m/z): 673.5 [M+H]⁺.

### Step 3: (S)-(4-((4-(2-(3-aminopropyl)phenyl)piperazin-1-yl)methyl)-1,5-dimethyl-1H-pyrrol-2-yl)(2-methyl-4-phenylpiperazin-1-yl)methanone ditrifluoroacetate salt

A mixture of (S)-tert-butyl 3-(2-(4-((1,2-dimethyl-5-(2-methyl-4-phenylpiperazine-1-carbonyl)-1H-pyrrol-3-yl)methyl)piperazin-1-yl)phenyl)propyl (methoxy methyl) carbamate (180 mg, 0.27 mmol) in TFA (3 mL) and DCM (10 mL) was stirred at rt for 16 hrs. The reaction mixture was concentrated to give the crude product which was purified by prep-HPLC to afford (S)-(4-((4-(2-(3-aminopropyl)phenyl)piperazin-1-yl)methyl)-1,5-dimethyl-1H-pyrrol-2-yl)(2-methyl-4-phenylpiperazin-1-yl)methanone ditrifluoroacetate salt I-24 (135 mg, 0.178 mmol, 66%) as a white solid. ESI-MS (EI⁺, m/z): 529.5 [M+H]⁺. ¹H NMR (500 MHz, DMSO) δ 10.05 (s, 1H), 7.95 (s, 3H), 7.24 (t, J=7.3 Hz, 4H), 7.13 (m, 2H), 6.95 (d, J=8.2 Hz, 2H), 6.81 (t, J=7.3 Hz, 1H), 6.47 (s, 1H), 4.66 (s, 1H), 4.26-4.19 (m, 3H), 3.66-3.52 (m, 5H), 3.46 (d, J=11.1 Hz, 2H), 3.39 (s, 1H), 3.21 (d, J=9.5 Hz, 2H), 3.13-2.97 (m, 4H), 2.89-2.75 (m, 3H), 2.74-2.60 (m, 3H), 2.30 (s, 3H), 1.92-1.80 (m, 2H), 1.33 (d, J=6.7 Hz, 3H).

### Example 50: (R)-(4-((4-(2-chlorophenyl)piperazin-1-yl)methyl)-1-methyl-1H-pyrrol-2-yl)(2-methyl-4-phenylpiperazin-1-yl)methanone, I-137

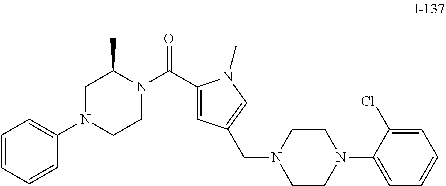 [SMILES: C[C@@H]1CN(c2ccccc2)CCN1C(=O)c1cc(CN2CCN(c3ccccc3Cl)CC2)cn1C]

### Synthetic Scheme:

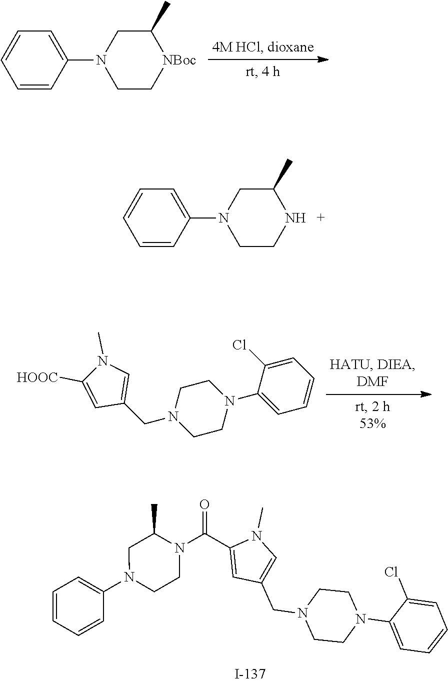

### Procedures and Characterization:

The general procedure for Intermediate II was followed to obtain 4-((4-(2-chlorophenyl)piperazin-1-yl)methyl)-1-methyl-1H-pyrrole-2-carboxylic acid.

Analysis was performed following Method A. Separation was performed following Method D.

### Step 1: (R)-3-methyl-1-phenylpiperazine

A mixture of (R)-tert-butyl-2-methyl-4-phenylpiperazine-1-carboxylate (500 mg, 1.81 mmol) in HCl/dioxane (5 mL) and MeOH (10 mL) was stirred at rt for 4 hrs. The reaction mixture was concentrated to give crude product (R)-3-methyl-1-phenylpiperazine (400 mg) as a yellow solid. ESI-MS (EI⁺, m/z): 177.2 [M+H]⁺.

### Step 2: (R)-(4-((4-(2-chlorophenyl) piperazin-1-yl)methyl)-1-methyl-1H-pyrrol-2-yl)(2-methyl-4-phenylpiperazin-1-yl)methanone

A mixture of (R)-3-methyl-1-phenylpiperazine (crude 97 mg), 4-((4-(2-chlorophenyl)piperazin-1-yl)methyl)-1-methyl-1H-pyrrole-2-carboxylic acid (176 mg, 0.5 mmol), HATU (247 mg, 0.65 mmol), DIPEA (129 mg, 1.0 mmol) in DMF (5 mL) was stirred at rt for 2 hrs. The mixture was purified by prep-HPLC to afford (R)-(4-((4-(2-chlorophenyl)piperazin-1-yl)methyl)-1-methyl-1H-pyrrol-2-yl)(2-methyl-4-phenyl piperazin-1-yl)methanone I-137 (130.5 mg, 0.265 mmol, 53%) as a white solid. ESI-MS (EI⁺, m/z): 492.3 [M+H]⁺. ¹H NMR (500 MHz, CDCl₃) δ 7.35 (dd, J=7.9, 1.4 Hz, 1H), 7.28 (t, J=7.8 Hz, 2H), 7.21 (dt, J=7.8, 2 Hz, 1H), 7.05 (dd, J=8.0, 1.3 Hz, 1H), 6.99-6.86 (m, 4H), 6.67 (s, 1H), 6.33 (d, J=1.6 Hz, 1H), 4.85 (s, 1H), 4.40 (d, J=13.2 Hz, 1H), 3.76 (s, 3H), 3.57 (d, J=12.1 Hz, 1H), 3.45 (d, J=6.3 Hz, 4H), 3.08 (s, 4H), 2.96 (dd, J=12.0, 3.5 Hz, 1H), 2.79 (td, J=11.8, 3.3 Hz, 1H), 2.65 (s, 4H), 1.45 (d, J=6.7 Hz, 3H).

### Example 51: (S)-(4-((4-(2-chlorophenyl)piperazin-1-yl)methyl)-1-methyl-1H-pyrrol-2-yl)(2-methyl-4-phenylpiperazin-1-yl)methanone, I-136

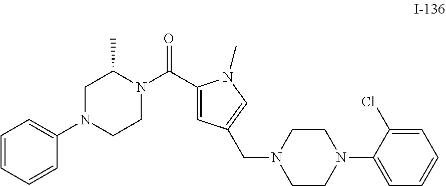 [SMILES: C[C@H]1CN(c2ccccc2)CCN1C(=O)c1cc(CN2CCN(c3ccccc3Cl)CC2)cn1C]

### Synthetic Scheme:

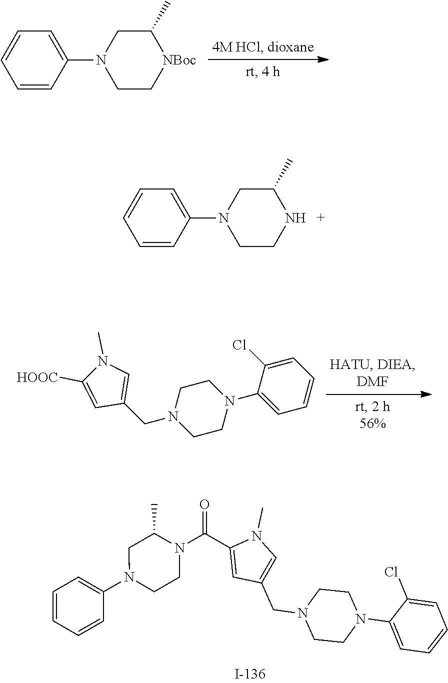

### Procedures and Characterization:

The general procedure for Intermediate II was followed to obtain 4-((4-(2-chlorophenyl)piperazin-1-yl)methyl)-1-methyl-1H-pyrrole-2-carboxylic acid.

Analysis was performed following Method A. Separation was performed following Method D.

### Step 1: (S)-3-Methyl-1-phenylpiperazine

A mixture of (S)-tert-butyl 2-methyl-4-phenylpiperazine-1-carboxylate (360 mg, 1.3 mmol) in HCl/dioxane (5 mL) and MeOH (10 mL) was stirred at rt for 4 hrs. The reaction mixture was then concentrated to give crude (S)-3-methyl-1-phenylpiperazine (250 mg) as a yellow solid. ESI-MS (EI⁺, m/z): 177.2 [M+H]⁺.

### Step 2: (S)-(4-((4-(2-Chlorophenyl) piperazin-1-yl)methyl)-1-methyl-1H-pyrrol-2-yl)(2-methyl-4-phenylpiperazin-1-yl)methanone

A mixture of (S)-3-methyl-1-phenylpiperazine (crude 97 mg), 4-((4-(2-chlorophenyl)piperazin-1-yl)methyl)-1-methyl-1H-pyrrole-2-carboxylic acid (176 mg, 0.5 mmol), HATU (247 mg, 0.65 mmol), DIPEA (129 mg, 1.0 mmol) in DMF (5 mL) was stirred at rt for 2 hrs. The mixture was purified by prep-HPLC to afford (S)-(4-((4-(2-chlorophenyl)piperazin-1-yl)methyl)-1-methyl-1H-pyrrol-2-yl)(2-methyl-4-phenyl piperazin-1-yl)methanone I-136 (135.2 mg, 0.275 mmol, 55%) as a white solid. ESI-MS (EI⁺, m/z): 492.2 [M+H]⁺. ¹H NMR (500 MHz, CDCl₃) δ 7.35 (dd, J=7.9, 1.4 Hz, 1H), 7.28 (t, J=8.0 Hz, 2H), 7.21 (td, J=8.0, 1.4 Hz, 1H), 7.05 (dd, J=8.0, 1.2 Hz, 1H), 6.98-6.85 (m, 4H), 6.67 (s, 1H), 6.33 (d, J=1.5 Hz, 1H), 4.85 (s, 1H), 4.40 (d, J=13.1 Hz, 1H), 3.76 (s, 3H), 3.57 (d, J=11.4 Hz, 1H), 3.45 (d, J=8.1 Hz, 4H), 3.09 (s, 4H), 2.96 (dd, J=12.0, 3.5 Hz, 1H), 2.79 (dt, J=12.0, 3.5 Hz, 1H), 2.65 (s, 4H), 1.45 (d, J=6.7 Hz, 3H).

### Example 52: (S)-(4-((4-(2-chlorophenyl)piperazin-1-yl)methyl)-1-methyl-1H-pyrrol-2-yl)(4-(3,5-difluorophenyl)-2-methylpiperazin-1-yl)methanone, I-94

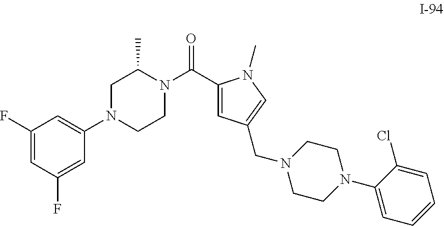 [SMILES: C[C@H]1CN(c2cc(F)cc(F)c2)CCN1C(=O)c1cc(CN2CCN(c3ccccc3Cl)CC2)cn1C]

### Synthetic Scheme:

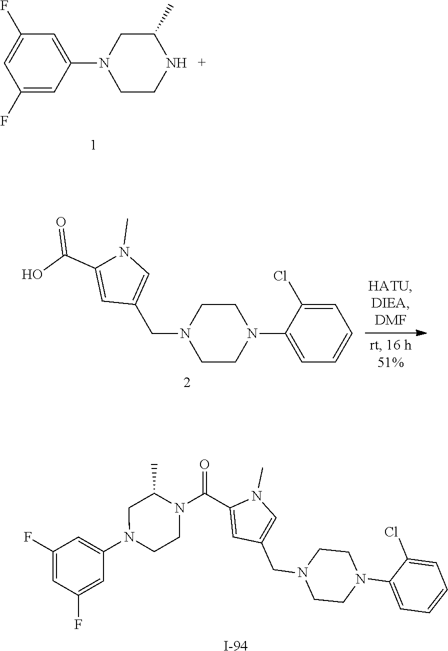

### Procedures and Characterization:

The general procedure for Intermediate II was followed to obtain 4-((4-(2-chlorophenyl)piperazin-1-yl)methyl)-1-methyl-1H-pyrrole-2-carboxylic acid.

The procedure for (S)-(4-((4-(2-chlorophenyl)piperazin-1-yl)methyl)-1-methyl-1H-pyrrol-2-yl)(4-(3,5-difluorophenyl)-2-methylpiperazin-1-yl)methanone I-94 was the same as Example 1. Analysis was performed following Method B. Separation was performed following Method D. ESI-MS (EI⁺, m/z): 528.3 [M+H]⁺. ¹H NMR (500 MHz, CDCl₃) δ 7.35 (d, J=7.5 Hz, 1H), 7.21 (t, J=7.7 Hz, 1H), 7.05 (d, J=7.6 Hz, 1H), 6.96 (t, J=7.6 Hz, 1H), 6.68 (s, 1H), 6.39-6.31 (m, 3H), 6.28 (t, J=8.8 Hz, 1H), 4.85 (s, 1H), 4.40 (d, J=13.4 Hz, 1H), 3.76 (s, 3H), 3.56 (d, J=11.9 Hz, 1H), 3.46-3.42 (m, 4H), 3.20-2.94 (m, 5H), 2.86 (td, J=11.8, 3.4 Hz, 1H), 2.65 (s, 4H), 1.40 (d, J=6.7 Hz, 3H).

### Example 53: (S)-(4-((4-(2-chlorophenyl)piperazin-1-yl)methyl)-1-methyl-1H-pyrrol-2-yl)(2-methyl-4-(3,4,5-trifluorophenyl)piperazin-1-yl)methanone, I-88

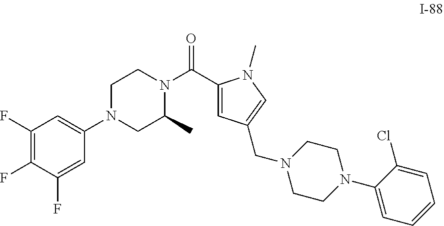 [SMILES: C[C@H]1CN(c2cc(F)c(F)c(F)c2)CCN1C(=O)c1cc(CN2CCN(c3ccccc3Cl)CC2)cn1C]

### Synthetic Scheme:

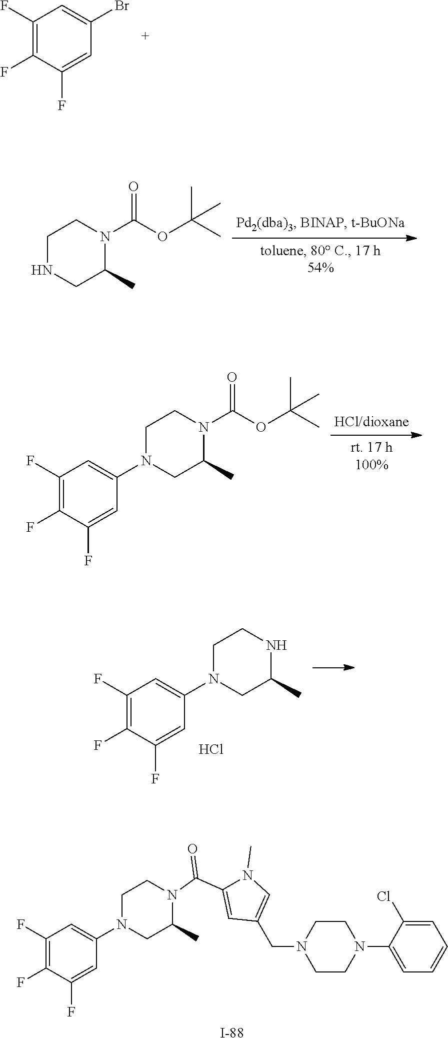

### Procedures and Characterization:

The general procedure for Intermediate II was followed to obtain 4-((4-(2-chlorophenyl)piperazin-1-yl)methyl)-1-methyl-1H-pyrrole-2-carboxylic acid.

Analysis was performed following Method B. Separation was performed following Method D.

### Step 1: (S)-tert-butyl 2-methyl-4-(3,4,5-trifluorophenyl)piperazine-1-carboxylate

A solution of 5-bromo-1,2,3-trifluorobenzene (1.05 g, 5.0 mmol), (S)-tert-butyl 2-methylpiperazine-1-carboxylate (1.0 g, 5.0 mmol), t-BuONa (720 mg, 7.5 mmol), BINAP (62 mg, 0.1 mmol), and Pd₂(dba)₃ (92 mg, 0.1 mmol) in dry toluene (20 mL) was stirred for 17 hrs at 80° C. The crude product was purified by chromatography (silica, EtOAc/PE=1/30) to afford (S)-tert-butyl-2-methyl-4-(3,4,5-trifluorophenyl)piperazine-1-carboxylate (0.9 g, 2.7 mmol, 54%) as a yellow oil. ESI-MS (EI⁺, m/z): 275.0 [M−56]⁺.

### Step 2: (S)-3-methyl-1-(3,4,5-trifluorophenyl)piperazine hydrochloride salt

To a solution of (S)-tert-butyl 2-methyl-4-(3,4,5-trifluorophenyl)piperazine-1-carboxylate (0.9 g, 2.7 mmol) was added 4M HCl/dioxane (20 mL). The mixture was stirred for 3 hrs at rt, then concentrated to afford crude (S)-3-methyl-1-(3,4,5-trifluorophenyl)piperazine hydrochloride salt (700 mg, 2.7 mmol, 100%) as a yellow solid. ESI-MS (EI⁺, m/z): 231.2 [M+H]⁺. (S)-(4-((4-(2-chlorophenyl)piperazin-1-yl)methyl)-1-methyl-1H-pyrrol-2-yl)(2-methyl-4-(3,4,5-trifluorophenyl)piperazin-1-yl)methanone I-88: ESI-MS (EI⁺, m/z): 546.3 [M+H]⁺. ¹H NMR (500 MHz, DMSO-d₆) δ 7.39 (d, J=8.0 Hz, 1H), 7.28 (t, J=7.0 Hz, 1H), 7.14 (d, J=7.0 Hz, 1H), 7.02 (t, J=7.5 Hz, 1H), 6.86 (dd, J=11.5, 6.0 Hz, 3H), 6.27 (s, 1H), 4.61 (s, 1H), 4.16 (d, J=12.5 Hz, 1H), 3.61 (m, 5H), 3.36 (s, 2H), 3.31 (m, 6H), 2.94 (m, 5H), 1.26 (d, J=6.8 Hz, 3H).

### Example 54: (4-((4-(2-Chlorophenyl)piperazin-1-yl)methyl)-1-methyl-1H-pyrrol-2-yl)(4-(3,4,5-trifluorophenyl)piperazin-1-yl)methanone trifluoroacetate salt, I-132

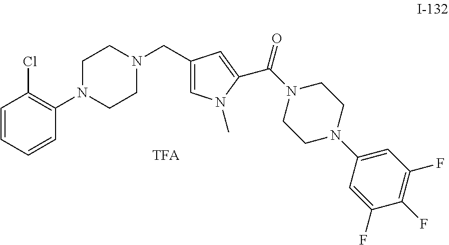 [SMILES: Cn1cc(CN2CCN(c3ccccc3Cl)CC2)cc1C(=O)N1CCN(c2cc(F)c(F)c(F)c2)CC1]

### Synthetic Scheme:

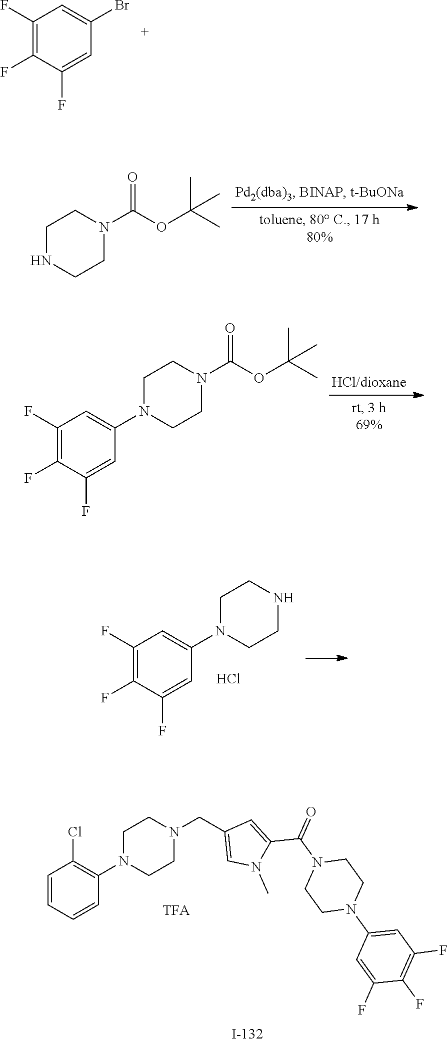

### Procedures and Characterization:

The general procedure for Intermediate II was followed to obtain 4-((4-(2-chlorophenyl)piperazin-1-yl)methyl)-1-methyl-1H-pyrrole-2-carboxylic acid.

Analysis was performed following Method A. Separation was performed following Method C.

### Step 1: tert-Butyl-4-(3,4,5-trifluorophenyl)piperazine-1-carboxylate

A solution of 5-bromo-1,2,3-trifluorobenzene (5.0 g, 23.7 mmol), tert-butyl piperazine-1-carboxylate (5.3 g, 28.4 mmol), t-BuONa (3.4 g, 35.5 mmol), BINAP (300 mg, 0.5 mmol), and Pd₂(dba)₃ (458 mg, 0.5 mmol) in dry toluene (100 mL) was stirred for 17 hrs at 80° C. The crude product was purified by chromatography (silica, EtOAc/PE=1/10) to afford tert-butyl-4-(3,4,5-trifluorophenyl)piperazine-1-carboxylate (6.0 g, 18.9 mmol, 80%) as a yellow solid. ESI-MS (EI⁺, m/z): 261.1 [M-56]⁺.

### Step 2: 1-(3,4,5-trifluorophenyl)piperazine hydrochloride salt

To a solution of tert-butyl piperazine-1-carboxylate (200 mg, 0.63 mmol) in Et₂O (5 mL) was added 4M HCl/dioxane (10 mL). The mixture was stirred for 3 hrs at rt. The mixture was filtered to afford 1-(3,4,5-trifluorophenyl)piperazine hydrochloride salt (110 mg, 0.43 mmol, 69%) as a white solid. ESI-MS (EI⁺, m/z): 217.1 [M+H]⁺.

The procedure for (4-((4-(2-Chlorophenyl)piperazin-1-yl)methyl)-1-methyl-1H-pyrrol-2-yl)(4-(3,4,5-trifluorophenyl)piperazin-1-yl)methanone trifluoroacetate salt I-132 was the same as Example 1.

ESI-MS (EI⁺, m/z): 532.0 [M+H]⁺. ¹H NMR (500 MHz, DMSO-d₆) δ 9.76 (s, 1H), 7.46 (d, J=8.0 Hz, 1H), 7.34 (t, J=7.5 Hz, 1H), 7.21 (d, J=8.0 Hz, 1H), 7.16-7.05 (m, 2H), 6.90 (m, 2H), 6.52 (s, 1H), 4.25 (m, 2H), 3.73 (m, 7H), 3.45 (m, 4H), 3.24 (m, 4H), 3.20-3.11 (m, 2H), 2.99 (m, 2H).

### Example 55: (S)-(1-(3-aminopropyl)-4-((4-(2-chlorophenyl)piperazin-1-yl)methyl)-5-methyl-1H-pyrrol-2-yl)(2-methyl-4-phenylpiperazin-1-yl)methanone dihydrochloride salt, I-29

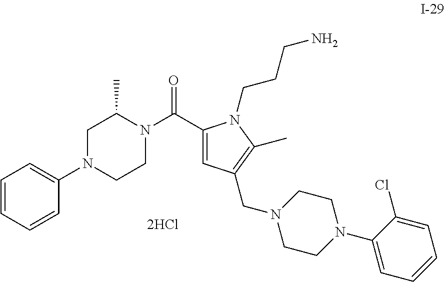 [SMILES: Cc1c(CN2CCN(c3ccccc3Cl)CC2)cc(C(=O)N2CCN(c3ccccc3)C[C@@H]2C)n1CCCN]

### Scheme:

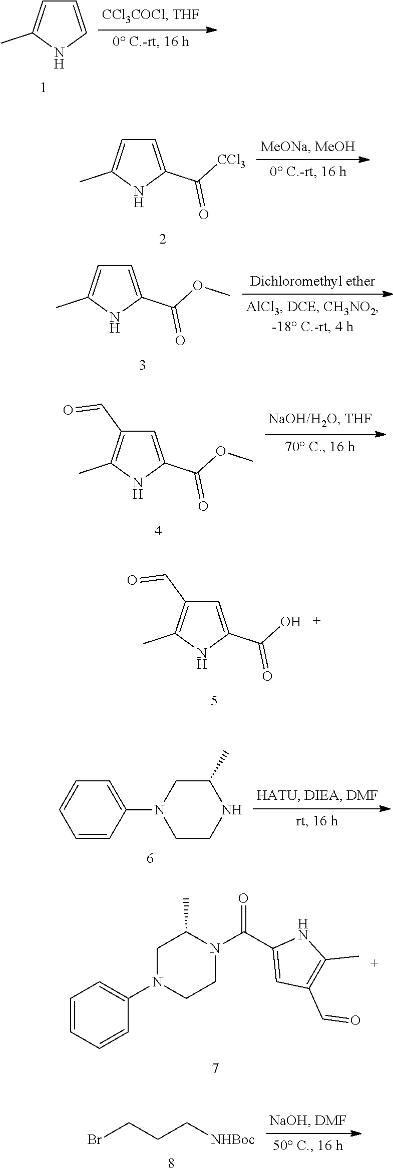

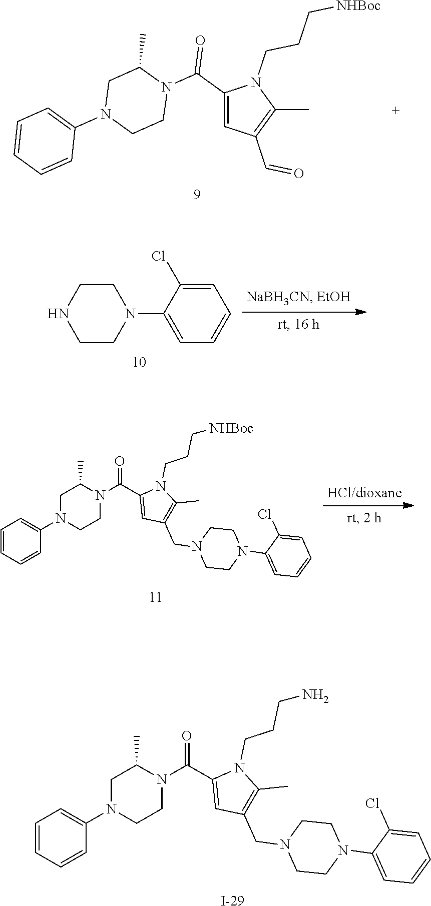

### Procedures and Characterization:

The general procedure for Intermediate I was followed to obtain (S)-2-methyl-5-(2-methyl-4-phenylpiperazine-1-carbonyl)-1H-pyrrole-3-carbaldehyde. The procedure for (S)-(1-(3-aminopropyl)-4-((4-(2-chlorophenyl)piperazin-1-yl)methyl)-5-methyl-1H-pyrrol-2-yl)(2-methyl-4-phenylpiperazin-1-yl)methanone dihydrochloride salt I-29 was the same as Examples 32 and 49. Analysis was performed following Method A.

ESI-MS (EI⁺, m/z): 549.4 [M+H]+. ¹H NMR (500 MHz, DMSO-d6) δ 11.16 (s, 1H), 8.12 (s, 3H), 7.49-7.43 (m, 1H), 7.34 (t, J=7.0 Hz, 1H), 7.25 (t, J=7.5 Hz, 2H), 7.19 (d, J=7.5 Hz, 1H), 7.11 (t, J=7.5 Hz, 1H), 7.02 (s, 2H), 6.86 (d, J=6.5 Hz, 1H), 6.75 (s, 1H), 4.68 (s, 1H), 4.35-4.03 (m, 5H), 3.63 (d, J=11.5 Hz, 1H), 3.55 (d, J=13.0 Hz, 1H), 3.42 (m, 5H), 3.28-3.10 (m, 5H), 2.92 (m, 1H), 2.75 (m, 3H), 2.35 (s, 3H), 2.03-1.90 (m, 2H), 1.38 (d, J=6.6 Hz, 3H).

### Example 56: Synthesis of (4-((4-(3-chloropyridin-2-yl)piperazin-1-yl)methyl)-1-methyl-1H-pyrrol-2-yl)(4-phenylpiperazin-1-yl)methanone, I-119

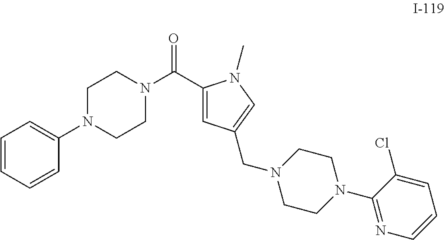 [SMILES: Cn1cc(CN2CCN(c3ncccc3Cl)CC2)cc1C(=O)N1CCN(c2ccccc2)CC1]

### Synthetic Scheme:

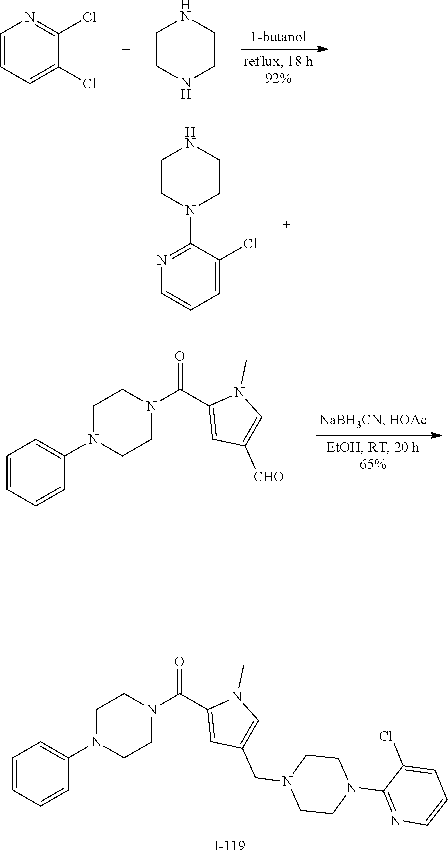 [SMILES: Clc1cccnc1Cl ; C1CNCCN1 ; Clc1cccnc1N1CCNCC1 ; Cn1cc(C=O)cc1C(=O)N1CCN(c2ccccc2)CC1 ; Cn1cc(CN2CCN(c3ncccc3Cl)CC2)cc1C(=O)N1CCN(c2ccccc2)CC1]

### Procedures and Characterization:

Analysis was performed following Method B. Separation was performed following Method D.

### Step 1: 1-(3-chloropyridin-2-yl)piperazine

A solution of 2,3-dichloropyridine (900 mg, 6.1 mmol) and piperazine (5.5 g, 64.1 mmol) was dissolved in n-butanol (25 mL) and reflux for 18 hrs. After concentrating the mixture by evaporation in vacuo, the residue was taken up in EtOAc and washed with H₂O. The combined organic phases were dried and concentrated to afford 1-(3-chloropyridin-2-yl)piperazine (1.1 mg, 5.6 mmol, 92%) as a yellow oil. MS (EI⁺, m/z): 198 [M+H]⁺.

### Step 2: (4-((4-(3-chloropyridin-2-yl)piperazin-1-yl)methyl)-1-methyl-1H-pyrrol-2-yl)(4-phenylpiperazin-1-yl)methanone

To a mixture of 1-methyl-5-(4-phenylpiperazine-1-carbonyl)-1H-pyrrole-3-carbaldehyde (180 mg, 0.6 mmol) and 1-(3-chloropyridin-2-yl)piperazine (237 mg, 1.2 mmol) in EtOH (10 mL) was added HOAc (5 drops) and NaBH₃CN (188 mg, 3.0 mmol). The resulting solution was then stirred for 20 hrs. The solution was concentrated to give a residue, which was then quenched with aqueous NH₄Cl (30 mL) and extracted with EtOAc (3×25 mL). The combined organic phases were washed with water (50 mL) and brine (30 mL), dried (Na₂SO₄), filtered, concentrated in vacuo, and purified by prep-HPLC to afford (4-((4-(3-chloropyridin-2-yl)piperazin-1-yl)methyl)-1-methyl-1H-pyrrol-2-yl)(4-phenylpiperazin-1-yl)methanone I-119 (187 mg, 0.39 mmol, 65%) as a white solid. MS (EI⁺, m/z): 479 [M+H]⁺. ¹H NMR (500 MHz, DMSO-d₆): δ 8.20 (dd, J=1.5 Hz, 4.5 Hz, 1H), 7.77 (dd, J=1.5 Hz, 7.5 Hz, 1H), 7.23 (t, J=7.5 Hz, 2H), 6.96-6.99 (m, 3H), 6.81-6.84 (m, 2H), 6.31 (d, J=1 Hz, 1H), 3.76 (d, J=4 Hz, 4H), 3.66 (s, 3H), 3.36 (s, 2H), 3.24 (s, 4H), 3.17 (t, J=5 Hz, 4H), 2.50 (s, 4H).

### Example 57: Synthesis of (1-methyl-4-((4-o-tolylpiperazin-1-yl)methyl)-1H-pyrrol-2-yl)(4-phenylpiperazin-1-yl)methanone, I-134

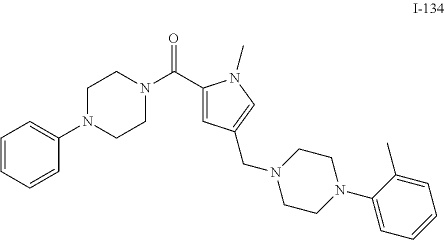 [SMILES: Cc1ccccc1N1CCN(Cc2cc(C(=O)N3CCN(c4ccccc4)CC3)n(C)c2)CC1]

### Synthetic Scheme:

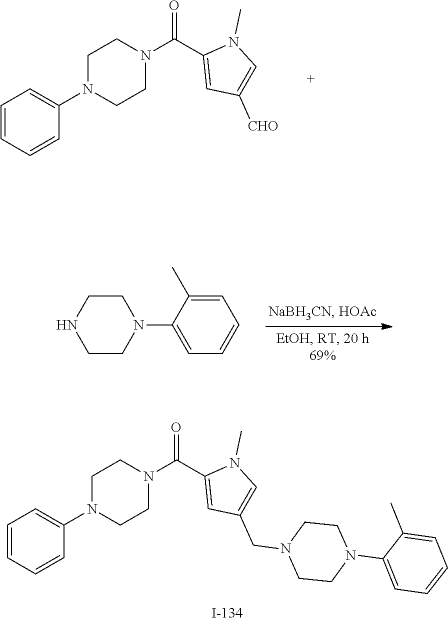 [SMILES: Cn1cc(C=O)cc1C(=O)N1CCN(c2ccccc2)CC1 ; Cc1ccccc1N1CCNCC1 ; Cc1ccccc1N1CCN(Cc2cc(C(=O)N3CCN(c4ccccc4)CC3)n(C)c2)CC1]

### Procedures and Characterization:

Analysis was performed following Method B. Separation was performed following Method D.

### Step 1: (1-methyl-4-((4-o-tolylpiperazin-1-yl)methyl)-1H-pyrrol-2-yl)(4-phenylpiperazin-1-yl)methanone

To a mixture of 1-methyl-5-(4-phenylpiperazine-1-carbonyl)-1H-pyrrole-3-carbaldehyde (150 mg, 0.5 mmol) and 1-o-tolylpiperazine (176 mg, 1 mmol) in EtOH (6 mL) was added HOAc (3 drops) and NaBH₃CN (157 mg, 2.5 mmol). The resulting solution was then stirred for 20 hrs. The solvent was removed in vacuo and the residue was quenched with aq. NH₄Cl solution (30 mL), then extracted with EtOAc (3×20 mL). The combined organic phases were washed with water (50 mL) and brine (30 mL), then dried (Na₂SO₄), filtered, concentrated in vacuo, and purified by prep-HPLC to afford (1-methyl-4-((4-o-tolylpiperazin-1-yl)methyl)-1H-pyrrol-2-yl)(4-phenylpiperazin-1-yl)methanone I-134 (157.2 mg, 0.34 mmol, 69%) as a white solid.

MS (EI⁺, m/z): 458 [M+H]⁺. ¹H NMR (500 MHz, DMSO-d₆): δ 7.23 (dd, J=7.5 Hz, 8.5 Hz, 2H), 7.12 (dd, J=7.5 Hz, 9.5 Hz, 2H), 6.93-7.00 (m, 4H), 6.80-6.85 (m, 2H), 6.30 (d, J=1.5 Hz, 1H), 3.77 (t, J=5 Hz, 4H), 3.66 (s, 3H), 3.36 (s, 2H), 3.17 (t, J=5 Hz, 4H), 2.82 (s, 4H), 2.50 (s, 4H), 2.22 (s, 3H).

### Example 58: (S)-(4-((4-(2-chlorophenyl)piperazin-1-yl)methyl)-1-methyl-1H-pyrrol-2-yl)(4-(3,4-difluorophenyl)-2-methylpiperazin-1-yl)methanone, I-117

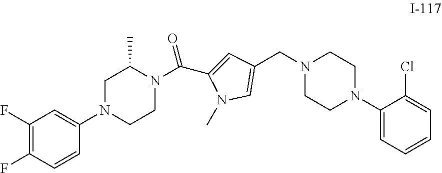 [SMILES: C[C@H]1CN(c2ccc(F)c(F)c2)CCN1C(=O)c1cc(CN2CCN(c3ccccc3Cl)CC2)cn1C]

### Synthetic Scheme:

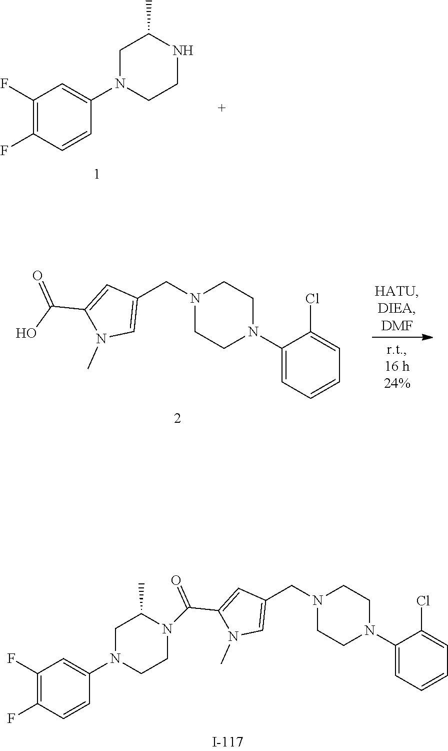 [SMILES: C[C@H]1CN(c2ccc(F)c(F)c2)CCN1 ; Cn1cc(CN2CCN(c3ccccc3Cl)CC2)cc1C(=O)O ; C[C@H]1CN(c2ccc(F)c(F)c2)CCN1C(=O)c1cc(CN2CCN(c3ccccc3Cl)CC2)cn1C]

### Procedures and Characterization:

The general procedure for Intermediate II was followed to obtain 4-((4-(2-chlorophenyl)piperazin-1-yl)methyl)-1-methyl-1H-pyrrole-2-carboxylic acid.

Analysis was performed following Method B. Separation was performed following Method D. The procedure was the same as general procedure II and Example 1. (S)-(4-((4-(2-chlorophenyl)piperazin-1-yl)methyl)-1-methyl-1H-pyrrol-2-yl)(4-(3,4-difluorophenyl)-2-methylpiperazin-1-yl)methanone I-117: ESI-MS (EI⁺, m/z): 528.3 [M+H]⁺. ¹H NMR (500 MHz, CDCl₃) δ 7.35 (dd, J=7.9, 1.2 Hz, 1H), 7.25-7.18 (m, 1H), 7.10-7.01 (m, 2H), 7.00-6.93 (m, 1H), 6.76-6.65 (m, 2H), 6.62-6.55 (m, 1H), 6.33 (d, J=1.5 Hz, 1H), 4.85 (s, 1H), 4.41 (d, J=13.3 Hz, 1H), 3.76 (s, 3H), 3.45 (m, 4H), 3.32 (d, J=11.9 Hz, 1H), 3.08 (s, 4H), 2.93 (dd, J=11.9, 3.6 Hz, 1H), 2.86-2.53 (m, 5H), 1.45 (t, J=13.8 Hz, 3H).

### Example 59: (4-((4-(2-Chlorophenyl)piperazin-1-yl)methyl)-1-(2,2,2-trifluoroethyl)-1H-pyrrol-2-yl)(4-phenylpiperazin-1-yl)methanone trifluoroacetate salt, I-114

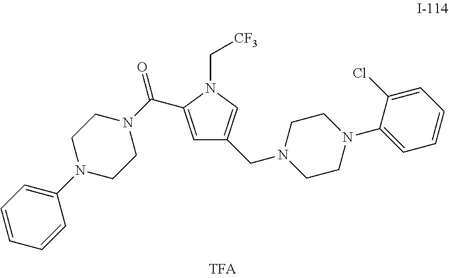 [SMILES: O=C(c1cc(CN2CCN(c3ccccc3Cl)CC2)cn1CC(F)(F)F)N1CCN(c2ccccc2)CC1]

### Synthetic Scheme:

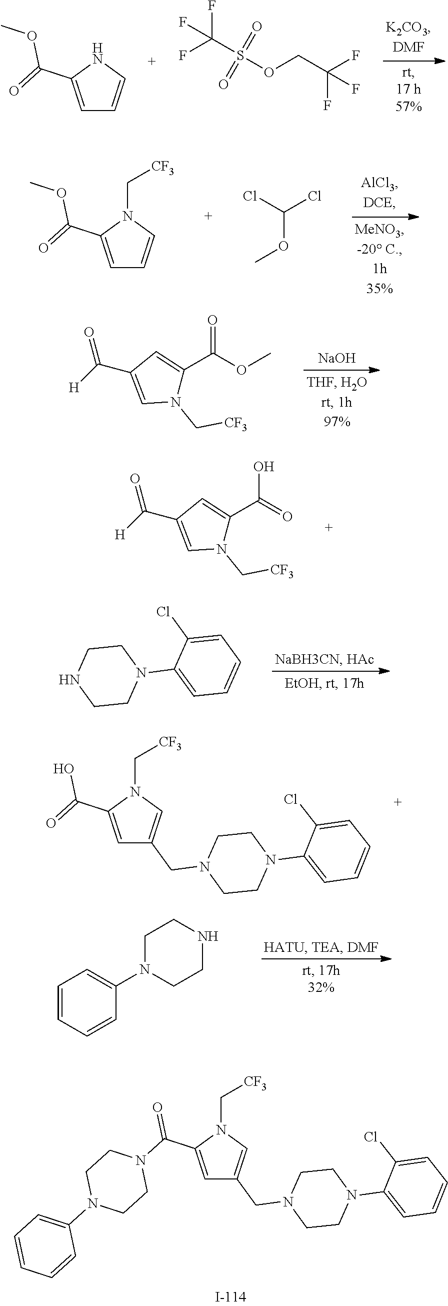

### Procedures and Characterization:

Analysis was performed following Method A. Separation was performed following Method C.

### Step 1: Methyl-1-(2,2,2-trifluoroethyl)-1H-pyrrole-2-carboxylate

To a solution of methyl-1H-pyrrole-2-carboxylate (1.27 g, 10.1 mmol), K₂CO₃ (3.5 g, 25.2 mmol) in DMF (60 mL) was added 2,2,2-trifluoroethyl-trifluoromethanesulfonate (2.6 g, 11.2 mmol). The mixture was stirred for 17 hrs at rt. The mixture was extracted with EtOAc, the combines organic layers were washed with aq. NH₄Cl and brine, and concentrated. The crude product was purified by chromatography (silica, EtOAc/PE=1/10) to afford methyl 1-(2,2,2-trifluoroethyl)-1H-pyrrole-2-carboxylate as a colorless oil (1.2 g, 5.8 mmol, 57%).

### Step 2: Methyl-4-formyl-1-(2,2,2-trifluoroethyl)-1H-pyrrole-2-carboxylate

To a solution of methyl-1-(2,2,2-trifluoroethyl)-1H-pyrrole-2-carboxylate (0.55 g, 2.65 mmol), AlCl₃ (777 mg, 5.8 mmol) in 1,2-DCE (5 mL), and MeNO₃ (5 mL) was added a solution of dichloro(methoxy)methane (305 mg, 2.65 mmol) at −20° C. The mixture was stirred for 1 hr at −20° C. The mixture was then poured onto ice-water and extracted with DCM. The organic layer was washed with aq. NH₄Cl and brine, and then concentrated. The crude product was purified by chromatography (silica, EtOAc/PE=1/5) to afford methyl 4-formyl-1-(2,2,2-trifluoroethyl)-1H-pyrrole-2-carboxylate as a yellow solid (220 mg, 0.93 mmol, 35%). ESI-MS (EI⁺, m/z): 250.2 [M+H]⁺.

### Step 3: 4-Formyl-1-(2,2,2-trifluoroethyl)-1H-pyrrole-2-carboxylic acid

To a solution of methyl 4-formyl-1-(2,2,2-trifluoroethyl)-1H-pyrrole-2-carboxylate (220 mg, 0.93 mmol) in THF (5 mL), and water (5 mL) was added NaOH (112 mg, 2.8 mmol). The mixture was stirred at rt for 1 hr. The solvent was removed in vacuo and the mixture was extracted with Et₂O (10 mL) The aqueous layer was then acidified with 1 M HCl and extracted with EtOAc (20 mL). The combined organic layers were washed with aq. NH₄Cl and brine, dried over MgSO₄, and concentrated to afford 4-formyl-1-(2,2,2-trifluoroethyl)-1H-pyrrole-2-carboxylic acid as a yellow solid (200 mg, 0.9 mmol, 97%). ESI-MS (EI⁺, m/z): 222.1 [M+H]⁺.

### Step 4: 4-((4-(2-Chlorophenyl)piperazin-1-yl)methyl)-1-(2,2,2-trifluoroethyl)-1H-pyrrole-2-carboxylic acid

A solution of 4-formyl-1-(2,2,2-trifluoroethyl)-1H-pyrrole-2-carboxylic acid (200 mg, 0.9 mmol) and 1-(2-chlorophenyl)piperazine (267 mg, 1.35 mmol) in EtOH (5 mL) was stirred at rt for 1 hr. Then cat. HOAc and NaBH₃CN (113 mg, 1.8 mmol) were added and the mixture was stirred at rt for 17 hrs. The solvent was reduced in vacuo and the mixture was extracted with EtOAc (20 mL). The organic layer was washed with aq. NH₄Cl and brine, dried over MgSO₄ and concentrated to afford crude 4-((4-(2-chlorophenyl)piperazin-1-yl)methyl)-1-(2,2,2-trifluoroethyl)-1H-pyrrole-2-carboxylic acid as a brown oil (270 mg). ESI-MS (EI⁺, m/z): 402.0 [M+H]⁺.

### Step 5: (4-((4-(2-Chlorophenyl)piperazin-1-yl)methyl)-1-(2,2,2-trifluoroethyl)-1H-pyrrol-2-yl)(4-phenylpiperazin-1-yl)methanone

To a solution of crude 4-((4-(2-chlorophenyl)piperazin-1-yl)methyl)-1-(2,2,2-trifluoroethyl)-1H-pyrrole-2-carboxylic acid (270 mg, 0.67 mmol) and 1-phenylpiperazine (110 mg, 0.67 mmol) in DMF (5 mL) was added HATU (250 mg, 0.67 mmol) and TEA (0.3 mL, 2.0 mmol). The mixture was stirred at rt for 17 hrs. The solution was purified by prep-HPLC (Boston C18 21*250 mm 10 μm, Mobile phase: A: 0.1% trifluoroacetic acid; B: acetonitrile) to afford (4-((4-(2-chlorophenyl)piperazin-1-yl)methyl)-1-(2,2,2-trifluoroethyl)-1H-pyrrol-2-yl)(4-phenylpiperazin-1-yl)methanone I-114 (138 mg, 0.25 mmol, 32%) as a white solid. ESI-MS (EI⁺, m/z): 546.1 [M+H]⁺. ¹H NMR (500 MHz, DMSO-d₆+D₂O) δ 7.46 (dd, J=8.0, 1.5 Hz, 1H), 7.34 (m, 2H), 7.29-7.19 (m, 3H), 7.13 (t, J=7.5 Hz, 1H), 6.98 (d, J=8.0 Hz, 2H), 6.84 (t, J=7.0 Hz, 1H), 6.71 (d, J=1.5 Hz, 1H), 5.25-5.16 (m, 2H), 4.31 (m, 2H), 3.81 (m, 4H), 3.51 (m, 2H), 3.45 (m, 2H), 3.19 (m, 6H), 3.00 (m, 2H).

### Example 60: (S)-2-(4-(4-((4-(2-chlorophenyl)piperazin-1-yl)methyl)-1-methyl-1H-pyrrole-2-carbonyl)-3-methylpiperazin-1-yl)-4-fluorobenzonitrile, I-87

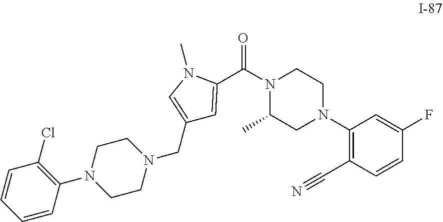 [SMILES: C[C@H]1CN(c2cc(F)ccc2C#N)CCN1C(=O)c1cc(CN2CCN(c3ccccc3Cl)CC2)cn1C]

### Synthetic Scheme:

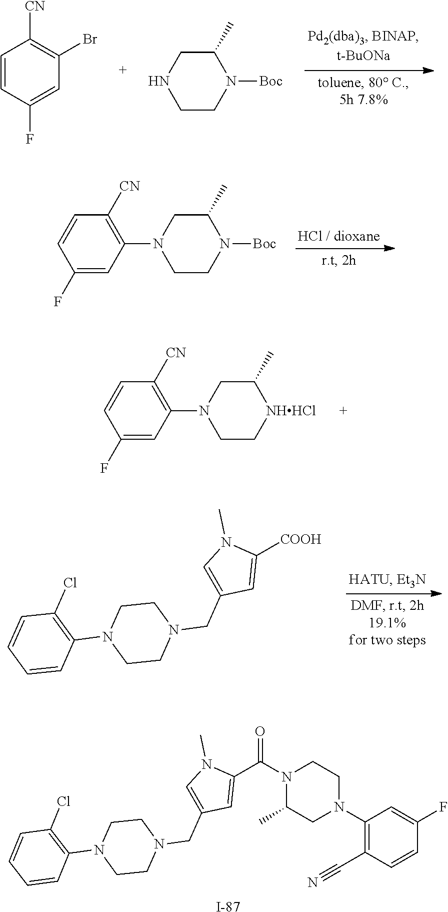

### Procedures and Characterization:

The general procedure for Intermediate II was followed to obtain 4-((4-(2-chlorophenyl)piperazin-1-yl)methyl)-1-methyl-1H-pyrrole-2-carboxylic acid.

Analysis was performed following Method B. Separation was performed following Method D.

### Step 1: (S)-tert-butyl 4-(2-cyano-5-fluorophenyl)-2-methylpiperazine-1-carboxylate

To a solution of 2-bromo-4-fluorobenzonitrile (1 g, 5.03 mmol) and (S)-tert-butyl 2-methylpiperazine-1-carboxylate (1.01 g, 5.03 mmol) in toluene (20 mL) was added Pd₂(dba)₃ (229 mg, 0.25 mmol), BINAP (313 mg, 0.503 mmol), and t-BuONa (986 mg, 10.06 mmol). The mixture was stirred at 80° C. for 5 hrs under nitrogen. The resulting reaction mixture was concentrated in vacuo and the residue was purified by column chromatography on silica to give (S)-tert-butyl-4-(2-cyano-5-fluorophenyl)-2-methylpiperazine-1-carboxylate (125 mg, 0.39 mmol, 7.8%) as a yellow liquid. ESI-MS (EI⁺, m/z): 342.0 [M+Na]⁺.

### Step 2: (S)-4-fluoro-2-(3-methylpiperazin-1-yl)benzonitrile hydrochloride salt

To a solution of (S)-tert-butyl-4-(2-cyano-5-fluorophenyl)-2-methylpiperazine-1-carboxylate (125 mg, 0.39 mmol) in MeOH (1 mL) was added HCl (4 M in dioxane, 5 mL). The mixture was stirred at rt for 2 hrs, then the mixture was concentrated to give (S)-4-fluoro-2-(3-methylpiperazin-1-yl)benzonitrile hydrochloride (123 mg, crude) which was used without further purification. ESI-MS (EI⁺, m/z): 220.1 [M+H]⁺.

### Step 3: (S)-2-(4-(4-((4-(2-chlorophenyl)piperazin-1-yl)methyl)-1-methyl-1H-pyrrole-2-carbonyl)-3-methylpiperazin-1-yl)-4-fluorobenzonitrile

To a solution of (S)-4-fluoro-2-(3-methylpiperazin-1-yl)benzonitrile hydrochloride (60 mg, crude) and 4-((4-(2-chlorophenyl)piperazin-1-yl)methyl)-1-methyl-1H-pyrrole-2-carboxylic acid (92.5 mg, 0.25 mmol) in DMF (1 mL) was added HATU (100 mg, 0.27 mmol) and TEA (0.17 mL, 1.25 mmol). The mixture was stirred at rt for 2 hrs. The mixture was then purified by prep-HPLC to afford (S)-2-(4-(4-((4-(2-chlorophenyl)piperazin-1-yl)methyl)-1-methyl-1H-pyrrole-2-carbonyl)-3-methylpiperazin-1-yl)-4-fluorobenzonitrile I-87 (25.4 mg, 0.072 mmol, 19.1% for two steps). ESI-MS (EI⁺, m/z): 535.2 [M+H]⁺. ¹H NMR (500 MHz, DMSO-d6) δ: 7.81-7.84 (dd, J₁=6.5 Hz, J₂=8.5 Hz, 1H), 7.38-7.40 (dd, J₁=1.0 Hz, J₂=8.0 Hz, 1H), 7.27-7.30 (t, J=7.0 Hz, 1H), 7.15 (d, J=8.0 Hz, 1H), 6.96-7.07 (m, 3H), 6.85 (s, 1H), 6.30 (s, 1H), 4.71 (s, 1H), 4.24 (d, J=12.5 Hz, 1H), 3.65 (s, 3H), 3.39-3.52 (m, 9H), 2.89-3.03 (m, 6H), 1.38-1.41 (m, 3H).

### Example 61: 1-(2-Chlorophenyl)-4-{1-[1-methyl-5-(4-phenylpiperazine-1-carbonyl)-1H-pyrrol-3-yl]ethyl}piperazine, I-106

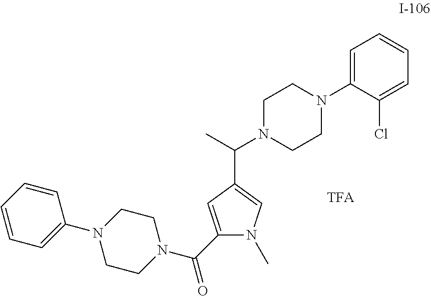 [SMILES: CC(c1cc(C(=O)N2CCN(c3ccccc3)CC2)n(C)c1)N1CCN(c2ccccc2Cl)CC1]

### Synthetic Scheme:

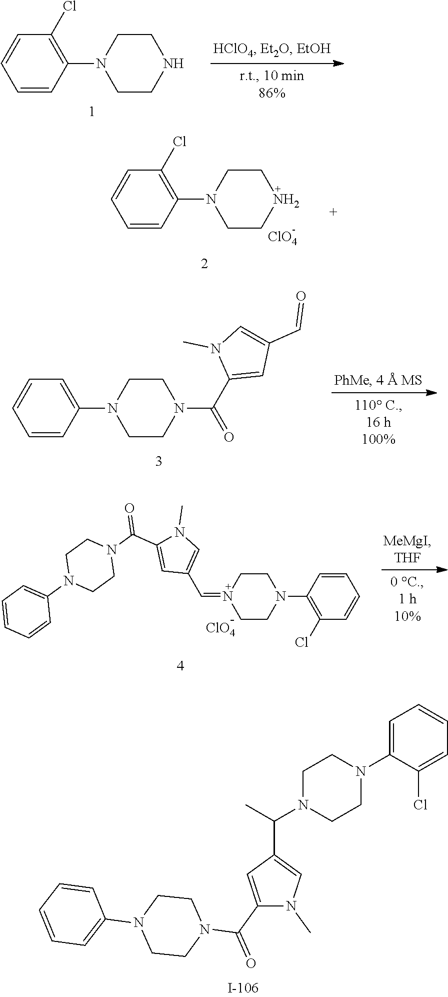

### Procedures and Characterization:

Analysis was performed following Method A. Separation was performed following Method C.

### Step 1: 4-(2-Chlorophenyl)piperazin-1-ium perchlorate

To a solution of 1-(2-chlorophenyl)piperazine (1.0 g, 5.1 mmol) in ethyl ether (5 mL) was added perchloric acid (0.5 mL, 5.1 mmol, in 1 mL ethanol) for 10 mins. The reaction mixture was then concentrated and recrystallization from MTBE to afford 4-(2-chlorophenyl)piperazin-1-ium perchlorate (1.3 g, 4.4 mmol, 86%) as a white solid.

### Step 2: 4-(2-Chlorophenyl)-1-01-methyl-5-(4-phenylpiperazine-1-carbonyl)-1H-pyrrol-3-yl)methylene)piperazin-1-ium perchlorate

A mixture of 4-(2-chlorophenyl)piperazin-1-ium perchlorate (500 mg, 1.69 mmol), 1-methyl-5-(4-phenylpiperazine-1-carbonyl)-1H-pyrrole-3-carb aldehyde (602 mg, 2.03 mmol), 4 Å MS (1 g) in PhMe (10 mL) was heated at 110° C. for 16 hrs. The mixture was then concentrated and washed with EtOH (15 mL) to afford the crude 4-(2-chlorophenyl)-1-((1-methyl-5-(4-phenylpiperazine-1-carbonyl)-1H-pyrrol-3-yl)methylene)piperazin-1-ium perchlorate (1 g, 1.69 mmol, 100%) which was used without further purification.

### Step 3: 1-(2-Chlorophenyl)-4-{1-[1-methyl-5-(4-phenylpiperazine-1-carbonyl)-1H-pyrrol-3-yl]ethyl}piperazine

To a solution of 4-(2-chlorophenyl)-1-((1-methyl-5-(4-phenylpiperazine-1-carbonyl)-1H-pyrrol-3-yl)methylene)piperazin-1-ium perchlorate (300 mg, 0.52 mmol) in THF (10 mL) was added MeMgI (3 M in Et₂O, 0.52 mL, 1.56 mmol) under ice-bath, and stirred at 0° C. for 1 hr. The mixture was then filtered and purified by prep-HPLC to afford 1-(2-chlorophenyl)-4-{1-[1-methyl-5-(4-phenylpiperazine-1-carbonyl)-1H-pyrrol-3-yl]ethyl}piperazine I-106 (25.7 mg, 0.052 mmol, 10%) as a white solid. ESI-MS (EI⁺, m/z): 492.1 [M+H]⁺. ¹H NMR (500 MHz, CDCl₃) δ 12.62 (br, 1H), 7.36 (d, J=8.0 Hz, 1H), 7.30 (t, J=7.5 Hz, 2H), 7.25 (t, J=7.5 Hz, 1H), 7.05 (dd, J=8.0 Hz, 15.5 Hz, 2H), 6.98-6.91 (m, 3H), 6.84 (s, 1H), 6.46 (s, 1H), 4.47-4.42 (m, 1H), 3.91 (t, J=4.5 Hz, 4H), 3.81 (s, 3H), 3.63 (d, J=10.5 Hz, 1H), 3.54 (d, J=11.5 Hz, 1H), 3.42-3.32 (m, 4H), 3.24 (t, J=4.5 Hz, 4H), 3.04-2.95 (m, 2H), 1.73 (d, J=7.0 Hz, 3H).

### Example 62: 1-{[1-Methyl-5-(4-phenylpiperazine-1-carbonyl)-1H-pyrrol-3-yl]methyl}-4-[2-(trifluoromethyl)phenyl]piperazine, I-126

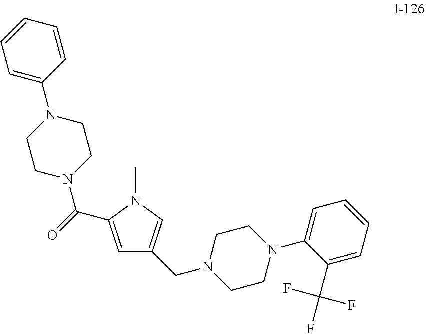 [SMILES: Cn1cc(CN2CCN(c3ccccc3C(F)(F)F)CC2)cc1C(=O)N1CCN(c2ccccc2)CC1]

### Procedures and Characterization:

Analysis was performed following Method A. Separation was performed following Method D.

ESI-MS (EI⁺, m/z): 512.3 [M+H]⁺. ¹H NMR (500 MHz, CDCl₃) δ 7.61 (d, J=7.0 Hz, 1H), 7.50 (t, J=8.0 Hz, 1H), 7.39 (d, J=8.0 Hz, 1H), 7.32-7.27 (m, 2H), 7.21 (t, J=7.5 Hz, 1H), 6.96 (d, J=8.0 Hz, 2H), 6.91 (t, J=7.0 Hz, 1H), 6.69 (d, J=1.5 Hz, 1H), 6.37 (d, J=1.5 Hz, 1H), 3.92 (t, J=5.0 Hz, 4H), 3.77 (s, 3H), 3.46 (s, 2H), 3.22 (t, J=5.0 Hz, 4H), 2.97 (t, J=5.0 Hz, 4H), 2.62 (br, 4H).

### Example 63: 3-(4-(4-((4-(2-chlorophenyl)piperazin-1-yl)methyl)-1-methyl-1H-pyrrole-2-carbonyl)piperazin-1-yl)benzonitrile, I-138

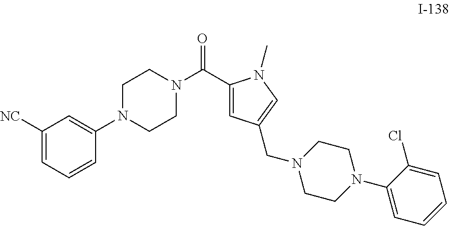 [SMILES: Cn1cc(CN2CCN(c3ccccc3Cl)CC2)cc1C(=O)N1CCN(c2cccc(C#N)c2)CC1]

### Synthetic Scheme:

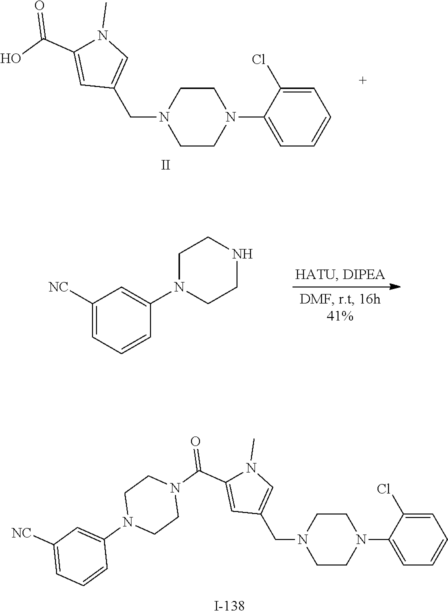

### Procedures and Characterization:

The general procedure II was followed to obtain Intermediate II. The procedure for 3-(4-(4-((4-(2-chlorophenyl)piperazin-1-yl)methyl)-1-methyl-1H-pyrrole-2-carbonyl)piperazin-1-yl)benzonitrile I-138 was the same as Example 1.

Analysis was performed following Method A. Separation was performed following Method C. ESI-MS (EI+, m/z): 503.1 [M+H]⁺. ¹H NMR (500 MHz, DMSO) δ 9.89 (s, 1H), 7.47-7.40 (m, 2H), 7.38-7.30 (m, 3H), 7.21 (d, J=7.7 Hz, 2H), 7.15-7.09 (m, 2H), 6.54 (s, 1H), 4.25 (s, 2H), 3.74 (d, J=26.3 Hz, 7H), 3.48 (dd, J=27.5, 12.1 Hz, 4H), 3.31 (s, 4H), 3.16 (d, J=10.8 Hz, 2H), 3.01 (s, 2H).

### Example 64: (4-((4-(2-fluorophenyl)piperazin-1-yl)methyl)-1-methyl-1H-pyrrol-2-yl)(4-phenylpiperazin-1-yl)methanone, I-125

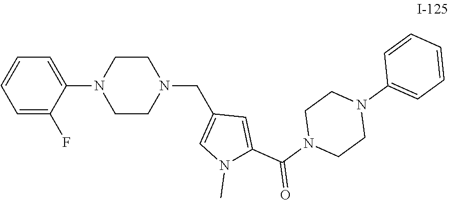 [SMILES: Cn1cc(CN2CCN(c3ccccc3F)CC2)cc1C(=O)N1CCN(c2ccccc2)CC1]

### Synthetic Scheme:

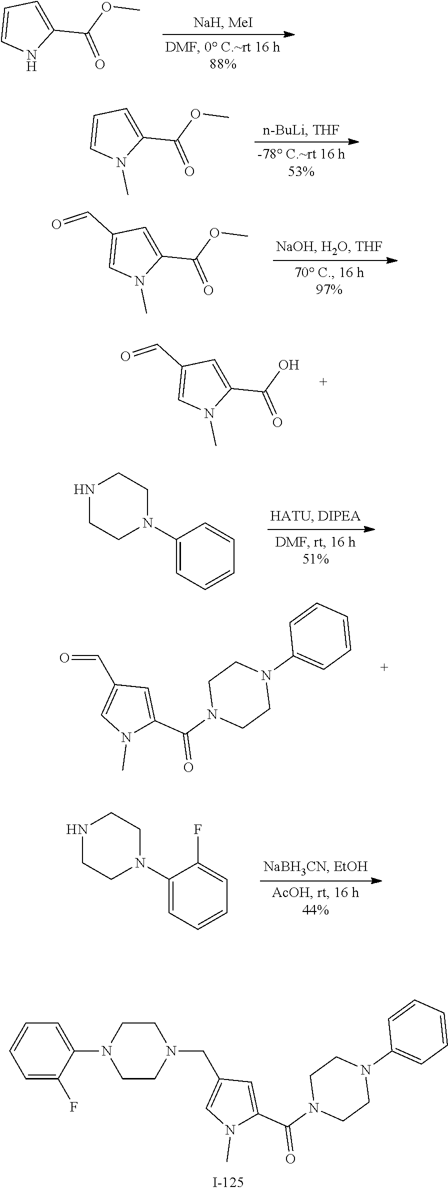

### Procedures and Characterization:

Analysis was performed following Method B. Separation was performed following Method D.

### Step 1: methyl-1-methyl-1H-pyrrole-2-carboxylate

To a solution of methyl-1H-pyrrole-2-carboxylate (25.0 g, 199.79 mmol) in DMF (100 ml) at 0° C. was added NaH (11.18 g, 279.21 mmol). The reaction mixture was stirred at 0° C. for 1 hr, then MeI (36.88 g, 259.73 mmol) was added. The reaction mixture was stirred for 16 hrs and allowed to warm from 0° C. to rt, then the mixture was diluted with H₂O (500 mL) and extracted with EtOAc (150 mL×3). The combined organic layers were washed with brine, dried over Na₂SO₄, filtered, concentrated, and purified by chromatography (silica, EtOAc/PE=1/10) to afford methyl-1-methyl-1H-pyrrole-2-carboxylate (24.7 g, 177.51 mmol, 88%) as an oil. ESI-MS (EI+, m/z): 140.2 [M+H]⁺.

### Step 2: methyl 4-formyl-1-methyl-1H-pyrrole-2-carboxylate

To a solution of methyl-1-methyl-1H-pyrrole-2-carboxylate (10.0 g, 71.86 mmol) in THF (200 mL) at −78° C. was added n-BuLi (31.6 mL, 79.05 mmol) under nitrogen. The mixture was stirred at −78° C. for 2 hrs. DMF (10.49 g, 143.72 mmol) was then added and the mixture was stirred for an additional 16 hrs while allowed to warm to rt. The mixture was then quenched with H₂O (500 mL) and extracted with EtOAc (200 mL×3). The combined organic layers were washed with brine, dried over Na₂SO₄, filtered, concentrated, and purified by chromatography (silica, EtOAc/PE=1/8) to afford methyl 4-formyl-1-methyl-1H-pyrrole-2-carboxylate (6.37 g, 38.09 mmol, 53%) as an oil. ESI-MS (EI+, m/z): 168.3 [M+H]⁺.

### Step 3: 4-formyl-1-methyl-1H-pyrrole-2-carboxylic acid

To a rt solution of methyl-4-formyl-1-methyl-1H-pyrrole-2-carboxylate (4.6 g, 27.52 mmol) in THF (34 mL) was added NaOH (5.5 g, 137.59 mmol) and H₂O (34 mL). The mixture was stirred at 70° C. for 16 hrs, then cooled to rt and diluted with H₂O (200 mL). The mixture was adjusted pH to 3-4 with 2N HCl and extracted with EtOAc (100 mL×3). The combined organic layers were washed with brine, dried over Na₂SO₄, filtered, and concentrated to afford 4-formyl-1-methyl-1H-pyrrole-2-carboxylic acid (4.0 g, 26.70 mmol, 97%) as a solid. ESI-MS (EI+, m/z): 154.4 [M+H]⁺.

### Step 4: 1-methyl-5-(4-phenylpiperazine-1-carbonyl)-1H-pyrrole-3-carbaldehyde

To a solution of 4-formyl-1-methyl-1H-pyrrole-2-carboxylic acid (3.8 g, 24.81 mmol) in DMF (60 mL) was added 1-phenylpiperazine (4.42 g, 27.30 mmol), HATU (15.56 g, 40.94 mmol), and DIPEA (6.41 g, 49.63 mmol). The reaction mixture was stirred at rt for 16 hrs, then diluted with H₂O (300 mL) and extracted with EtOAc (100 mL×3). The combined organic layers were washed with brine, dried over Na₂SO₄, filtered, concentrated, and purified by chromatography (silica, EtOAc/PE=1/1) to afford 1-methyl-5-(4-phenylpiperazine-1-carbonyl)-1H-pyrrole-3-carbaldehyde (3.8 g, 12.78 mmol, 51%) as an oil. ESI-MS (EI+, m/z): 298.4 [M+H]⁺.

### Step 5: (4-((4-(2-fluorophenyl)piperazin-1-yl)methyl)-1-methyl-1H-pyrrol-2-yl)(4-phenylpiperazin-1-yl)methanone

To a solution of 1-methyl-5-(4-phenylpiperazine-1-carbonyl)-1H-pyrrole-3-carbaldehyde (130 mg, 0.44 mmol) in EtOH (6 mL) was added 1-(2-fluorophenyl)piperazine (87 mg, 0.48 mmol), NaBH₃CN (82 mg, 1.31 mmol), and acetic acid (2 drops). The reaction mixture was stirred at rt for 16 hrs, then purified by prep-HPLC to afford (4-((4-(2-fluorophenyl)piperazin-1-yl)methyl)-1-methyl-1H-pyrrol-2-yl)(4-phenylpiperazin-1-yl)methanone I-125 (96 mg, 0.21 mmol, 44%) as a white solid. ESI-MS (EI+, m/z): 462.3 [M+H]⁺. ¹H NMR (500 MHz, DMSO) δ 7.24 (t, J=7.9 Hz, 2H), 7.09 (d, J=7.3 Hz, 2H), 7.02-6.93 (m, 4H), 6.83 (d, J=16.2 Hz, 2H), 6.31 (d, J=1.4 Hz, 1H), 3.77 (s, 4H), 3.66 (s, 3H), 3.34 (s, 6H), 3.22-3.14 (m, 4H), 3.00 (s, 2H), 2.51-2.49 (m, 2H).

### Example 65: (S)-2-(4-(4-((4-(2-chlorophenyl)piperazin-1-yl)methyl)-1-methyl-1H-pyrrole-2-carbonyl)-3-methylpiperazin-1-yl)-6-fluorobenzonitrile, I-101

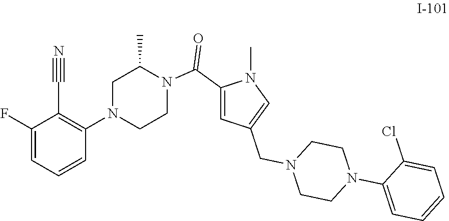 [SMILES: C[C@H]1CN(c2cccc(F)c2CN)CCN1C(=O)c1cc(CN2CCN(c3ccccc3Cl)CC2)cn1C]

### Synthetic Scheme:

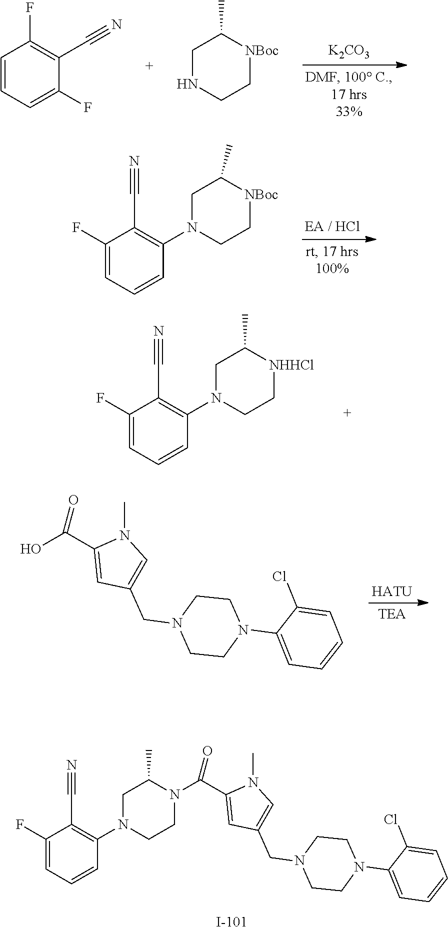

### Procedures and Characterization:

General procedure II was followed to obtain 4-((4-(2-chlorophenyl)piperazin-1-yl)methyl)-1-methyl-1H-pyrrole-2-carboxylic acid.

Analysis was performed following Method B. Separation was performed following Method D.

### Step 1: (S)-tert-butyl-4-(2-cyano-3-fluorophenyl)-2-methylpiperazine-1-carboxylate

To a solution of 2,6-difluorobenzonitrile (0.8 g, 5.7 mmol) in DMF (10 mL) was added (S)-tert-butyl 2-methylpiperazine-1-carboxylate (1.14 g, 5.7 mmol) and K₂CO₃ (2.35 g, 17.1 mmol). The solution was stirred for 17 hrs at 100° C., then concentrated to give the crude. The crude was purified by chromatography (silica, EtOAc/PE=1/10) to afford (S)-tert-butyl-4-(2-cyano-3-fluorophenyl)-2-methylpiperazine-1-carboxylate (0.6 g, 1.88 mmol, 33%) as a white solid. MS (EI⁺, m/z): 320 [M+H]⁺.

### Step 2: (S)-2-fluoro-6-(3-methylpiperazin-1-yl)benzonitrile hydrochloride

To a solution of (S)-tert-butyl 4-(2-cyano-3-fluorophenyl)-2-methylpiperazine-1-carboxylate (0.6 g, 1.88 mmol) in EtOAc (20 mL) was added EtOAc/HCl (20 ml, 3 M). The mixture was stirred for 17 hrs at rt, then diluted, filtered, and concentrated in vacuo to afford (S)-2-fluoro-6-(3-methylpiperazin-1-yl)benzonitrile hydrochloride (0.5 g, 100%). ESI-MS (EI⁺, m/z): 220 [M+H]⁺.

### Step 3: (S)-2-(4-(4-((4-(2-chlorophenyl)piperazin-1-yl)methyl)-1-methyl-1H-pyrrole-2-carbonyl)-3-methylpiperazin-1-yl)-6-fluorobenzonitrile

To a solution of (S)-2-fluoro-6-(3-methylpiperazin-1-yl)benzonitrile hydrochloride (0.1 g, 0.39 mmol) in DMF (2 mL) was added HATU (148 mg, 0.39 mmol) and DIPEA (117 mg. 1.17 mmol). The mixture was stirred at rt for 17 hrs, then diluted with water (20 mL) and extracted with EtOAc (50×3 mL). The organic phases were dried (Na₂SO₄), filtered, and concentrated in vacuo. The crude product was purified by prep-HPLC to afford (S)-2-(4-(4-((4-(2-chlorophenyl)piperazin-1-yl)methyl)-1-methyl-1H-pyrrole-2-carbonyl)-3-methylpiperazin-1-yl)-6-fluorobenzonitrile I-101 (21 mg, 10%) as a white solid. ESI-MS (EI⁺, m/z): 535 [M+1]⁺. ¹H NMR (500 MHz, CD₃OD) δ 7.49-7.51 (m, 1H), 7.37-7.39 (m, 2H), 7.13-7.15 (m, 3H), 6.78-6.87 (m, 2H), 6.33 (s, 1H), 4.74 (s, 1H), 4.47-4.49 (m, 1H), 3.61-3.69 (m, 5H), 3.47-3.55 (m, 6H), 3.06-3.08 (m, 1H), 2.88-2.90 (m, 2H), 2.70 (s, 1H), 2.22 (s, 3H), 2.04-2.07 (m, 2H), 1.43-1.44 (m, 3H), 1.28 (s, 3H).

### Example 66: 3-(4-(4-((4-(2-chlorophenyl)piperazin-1-yl)methyl)-1,5-dimethyl-1H-pyrrole-2-carbonyl)-1,4-diazepan-1-yl)benzonitrile, I-40

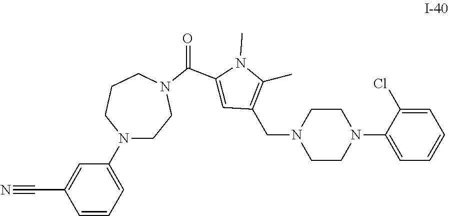 [SMILES: Cc1c(CN2CCN(c3ccccc3Cl)CC2)cc(C(=O)N2CCCN(c3cccc(C#N)c3)CC2)n1C]

### Synthetic Scheme:

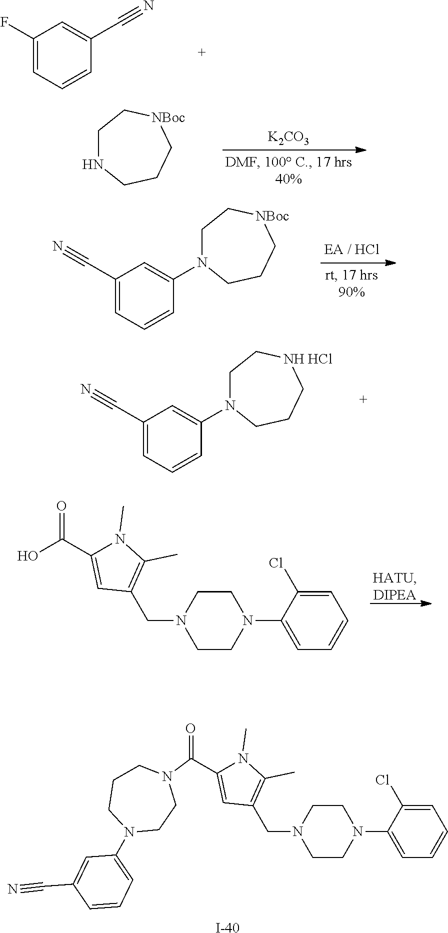

### Procedures and Characterization:

General procedure I was followed to obtain 4-((4-(2-chlorophenyl)piperazin-1-yl)methyl)-1-methyl-1H-pyrrole-2-carboxylic acid.

Analysis was performed following Method B. Separation was performed following Method D.

### Step 1: tert-butyl-4-(3-cyanophenyl)-1,4-diazepane-1-carboxylate

To a solution of 2-fluorobenzonitrile (0.5 g, 4.13 mmol) in DMF (10 mL) was added tert-butyl-1,4-diazepane-1-carboxylate (0.83 g, 4.13 mmol) and K₂CO₃ (1.7 g, 12.1 mmol). The solution was stirred for 17 hrs at 100° C., concentrated to give the crude, and then purified by chromatography (silica, EtOAc/PE=1/10) to afford tert-butyl-4-(3-cyanophenyl)-1,4-diazepane-1-carboxylate (0.30 g, 40%) as a white solid. MS (EI⁺, m/z): 302 [M+H]⁺.

### Step 2: 3-(1,4-diazepan-1-yl)benzonitrile hydrochloride

To a solution of tert-butyl-4-(3-cyanophenyl)-1,4-diazepane-1-carboxylate (0.30 g, 0.5 mmol) in EtOAc (10 mL) was added EtOAc/HCl (10 ml, 3 M). The mixture was stirred for 17 hrs at rt, then diluted, filtered, and concentrated in vacuo to afford 3-(1,4-diazepan-1-yl)benzonitrile hydrochloride (0.2 g, 90%). ESI-MS (EI⁺, m/z): 202 [M+H]⁺.

### Step 3: 3-(4-(4-((4-(2-chlorophenyl)piperazin-1-yl)methyl)-1,5-dimethyl-1H-pyrrole-2-carbonyl)-1,4-diazepan-1-yl)benzonitrile

To a solution of 3-(1,4-diazepan-1-yl)benzonitrile hydrochloride (0.1 g, 0.36 mmol) in DMF (2 mL) was added HATU (136.8 mg, 0.36 mmol) and DIPEA (117 mg, 1.17 mmol). The mixture was stirred at rt for 17 hrs, then diluted with water (20 mL) and extracted with EtOAc (50×3 mL). The organic phases were dried (Na₂SO₄), filtered, and concentrated in vacuo. The crude product was purified by prep-HPLC to afford 3-(4-(4-((4-(2-chlorophenyl)piperazin-1-yl)methyl)-1,5-dimethyl-1H-pyrrole-2-carbonyl)-1,4-diazepan-1-yl)benzonitrile I-40 (20.4 mg, 18%) as a white solid. ESI-MS (EI⁺, m/z): 531 [M+1]⁺. ¹H NMR (500 MHz, CDCl₃) δ 7.21-7.37 (m, 3H), 6.85-7.07 (m, 5H), 6.27 (s, 1H), 3.97 (s, 2H), 3.63-3.70 (s, 6H), 3.35-3.44 (m, 5H), 3.08 (s, 4H), 2.64 (s, 4H), 2.20 (s, 3H), 1.99-2.06 (m, 2H).

### Example 67: 2-(4-((5-(4-(3-cyanophenyl)-1,4-diazepane-1-carbonyl)-1,2-dimethyl-1H-pyrrol-3-yl)methyl)piperazin-1-yl)benzonitrile, I-45

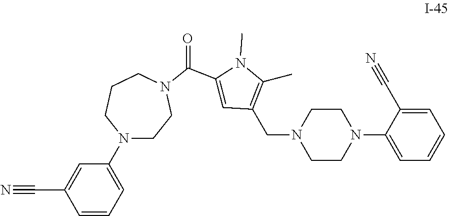 [SMILES: Cc1c(CN2CCN(c3ccccc3C#N)CC2)cc(C(=O)N2CCCN(c3cccc(C#N)c3)CC2)n1C]

### Procedures and Characterization:

The procedure for 2-(4-((5-(4-(3-cyanophenyl)-1,4-diazepane-1-carbonyl)-1,2-dimethyl-1H-pyrrol-3-yl)methyl)piperazin-1-yl)benzonitrile I-45 was the same as Example 66. Analysis was performed following Method B. Separation was performed following Method D. ESI-MS (EI⁺, m/z): 522 [M+1]⁺. ¹H NMR (500 MHz, CD₃OD) δ 7.67-7.69 (m, 1H), 7.58-7.59 (m, 1H), 6.94-7.15 (m, 6H), 6.06 (s, 1H), 3.51-3.83 (m, 8H), 3.25 (s, 3H), 3.03 (s, 8H), 3.49-3.64 (m, 7H), 3.32-3.33 (m, 2H), 3.27-3.30 (m, 3H), 2.95-2.97 (m, 1H), 1.57-1.59 (m, 3H), 2.11 (s, 3H), 1.86 (s, 2H).

### Example 68: (4-((4-(2-chlorophenyl)piperazin-1-yl)methyl)-1,5-dimethyl-1H-pyrrol-2-yl)(3,4-dihydropyrazino[1,2-a]indol-2(1H)-yl)methanone, I-76

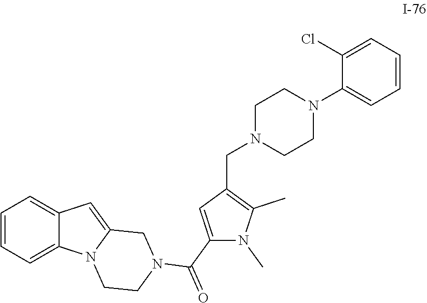 [SMILES: Cc1c(CN2CCN(c3ccccc3Cl)CC2)cc(C(=O)N2CCn3c(cc4ccccc43)C2)n1C]

### Synthetic Scheme:

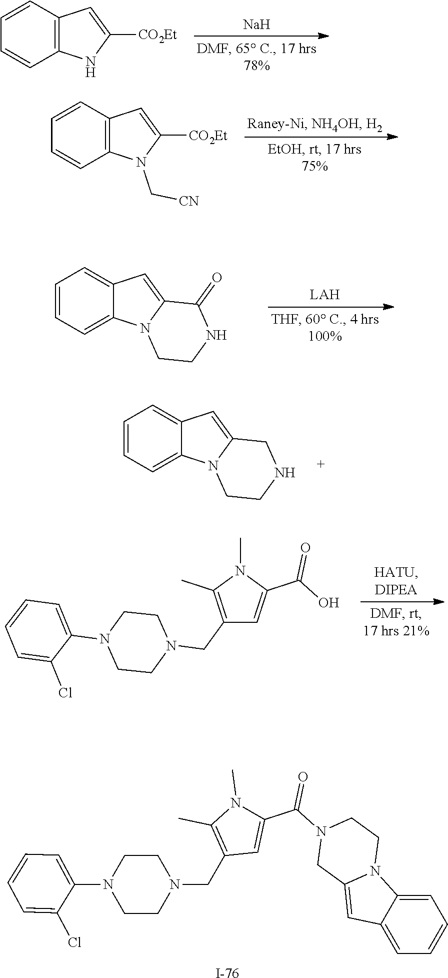

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid. Analysis was performed following Method A. Separation was performed following Method C.

### Step 1: ethyl-1-(cyanomethyl)-1H-indole-2-carboxylate

To a solution of ethyl-1H-indole-2-carboxylate (1.89 g, 10 mmol) in DMF (20 mL) was added NaH (0.46 g, 20 mmol) and 2-bromoacetonitrile (1.18 g, 10 mmol) and the solution was stirred for 17 hrs at 65° C., then concentrated to give the crude. The crude was purified by chromatography (silica, EtOAc/PE=1/5) to afford ethyl-1-(cyanomethyl)-1H-indole-2-carboxylate (1.8 g, 7.8 mmol, 78%) as a yellow solid. MS (EI⁺, m/z): 229 [M+H]⁺.

### Step 2: 3,4-dihydropyrazino[1,2-a]indol-1(2H)-one

To a solution of ethyl-1-(cyanomethyl)-1H-indole-2-carboxylate (1.8 g, 7.8 mmol) in EtOH (20 mL) was added Raney-Ni (2 g), and NH₄OH (0.5 ml). The mixture was stirred for 17 hrs at rt under H₂. The mixture was then filtered and concentrated in vacuo to afford 3,4-dihydropyrazino[1,2-a]indol-1(2H)-one (1.1 g, 75%). ESI-MS (EI⁺, m/z): 187 [M+H]⁺.

### Step 3: 1,2,3,4-tetrahydropyrazino[1,2-a]indole

To a solution of 3,4-dihydropyrazino[1,2-a]indol-1(2H)-one (0.2 g, 1.1 mmol) in THF (2 mL) was added LiAlH₄ (83 mg, 2.2 mmol) at 0° C. The mixture was stirred at 60° C. for 4 hrs, then diluted with water (1 mL) and extracted with EtOAc (20×3 mL). The organic phases were dried (Na₂SO₄), filtered, and concentrated in vacuo. The crude product was purified by chromatography (silica, EtOAc/PE=1/5) to afford 1,2,3,4-tetrahydropyrazino[1,2-a]indole (190 mg, 100%) as a yellow solid. ESI-MS (EI⁺, m/z): 173 [M+1]⁺.

### Step 3: (4-((4-(2-chlorophenyl)piperazin-1-yl)methyl)-1,5-dimethyl-1H-pyrrol-2-yl)(3,4-dihydropyrazino[1,2-a]indol-2(1H)-yl)methanone

To a solution of 1,2,3,4-tetrahydropyrazino[1,2-a]indole (0.1 g, 0.19 mmol) in DMF (2 mL) was added HATU (72.2 mg, 0.19 mmol) and DIPEA (117 mg, 0.57 mmol). The mixture was stirred at rt for 17 hrs, then diluted with water (20 mL) and extracted with EtOAc (50×3 mL). The organic phases were dried (Na₂SO₄), filtered, and concentrated in vacuo. The crude product was purified by prep-HPLC to afford (4-((4-(2-chlorophenyl)piperazin-1-yl)methyl)-1,5-dimethyl-1H-pyrrol-2-yl)(3,4-dihydropyrazino[1,2-a]indol-2(1H)-yl)methanone I-76 (20 mg, 21%) as a white solid. ESI-MS (EI⁺, m/z): 502 [M+1]⁺. ¹H NMR (500 MHz, CD₃OD) δ 7.49-7.51 (m, 1H), 7.36-7.38 (m, 2H), 7.25-7.27 (m, 1H), 7.16-7.17 (m, 2H), 7.01-7.08 (m, 2H), 6.53 (s, 1H), 6.29 (s, 1H), 5.14 (s, 2H), 4.22-4.28 (m, 4H), 3.65 (s, 3H), 3.54 (s, 2H), 3.09 (s, 4H), 2.72 (s, 4H), 2.29 (s, 3H).

### Example 69: (S)-3-(4-(4-((4-(3-chloropyridin-2-yl)piperazin-1-yl)methyl)-1,5-dimethyl-1H-pyrrole-2-carbonyl)-3-methylpiperazin-1-yl)benzonitrile, I-99

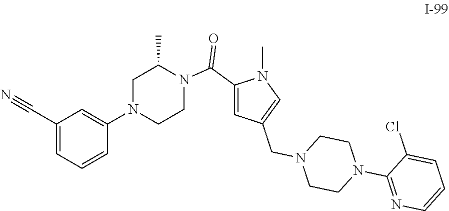 [SMILES: C[C@H]1CN(c2cccc(C#N)c2)CCN1C(=O)c1cc(CN2CCN(c3ncccc3Cl)CC2)cn1C]

### Synthetic Scheme:

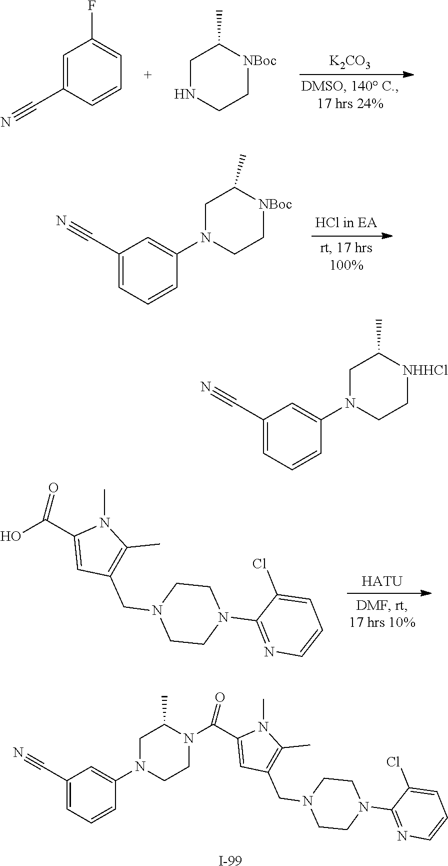

### Procedures and Characterization:

The procedure for 4-((4-(3-chloropyridin-2-yl)piperazin-1-yl)methyl)-1,5-dimethyl-1H-pyrrole-2-carboxylic acid was the same as Example 36.

Analysis was performed following Method B. Separation was performed following Method D.

### Step 1: ethyl-1-(cyanomethyl)-1H-indole-2-carboxylate

To a solution of ethyl-1H-indole-2-carboxylate (1.0, 8.26 mmol) in DMSO (10 mL) was added (S)-tert-butyl 2-methylpiperazine-1-carboxylate (1.7 g, 8.26 mmol) and K₂CO₃ (3.4 g, 24.7 mmol). The solution was stirred for 17 hrs at 140° C. then the solution was concentrated to give the crude. The crude was purified by chromatography (silica, EtOAc/PE=1/10) to afford (S)-tert-butyl-4-(3-cyanophenyl)-2-methylpiperazine-1-carboxylate (0.6 g, 1.99 mmol, 24%) as a white solid. MS (EI⁺, m/z): 302 [M+H]⁺.

### Step 2: (S)-3-(3-methylpiperazin-1-yl)benzonitrile hydrochloride

To a solution of (S)-tert-butyl-4-(3-cyanophenyl)-2-methylpiperazine-1-carboxylate (0.6 g, 1.99 mmol) in EtOAc (20 mL) was added EtOAc/HCl (20 ml, 3 M). The mixture was stirred for 17 hrs at rt, then the mixture was diluted, filtered, and concentrated in vacuo to afford (S)-3-(3-methylpiperazin-1-yl)benzonitrile hydrochloride (0.5 g, 100%). ESI-MS (EI⁺, m/z): 202 [M+H]⁺.

### Step 3: (S)-3-(4-(4-((4-(3-chloropyridin-2-yl)piperazin-1-yl)methyl)-1,5-dimethyl-1H-pyrrole-2-carbonyl)-3-methylpiperazin-1-yl)benzonitrile

To a solution of (S)-3-(3-methylpiperazin-1-yl)benzonitrile hydrochloride (0.1 g, 0.45 mmol) in DMF (2 mL) was added HATU (171 mg, 0.45 mmol) and DIPEA (136 mg, 1.35 mmol). The mixture was stirred at rt for 17 hrs, then the solution was diluted with water (20 mL) and extracted with EtOAc (50×3 mL). The organic phases were dried (Na₂SO₄), filtered, and concentrated in vacuo. The crude product was purified by prep-HPLC to afford (S)-3-(4-(4-((4-(3-chloropyridin-2-yl)piperazin-1-yl)methyl)-1,5-dimethyl-1H-pyrrole-2-carbonyl)-3-methylpiperazin-1-yl)benzonitrile I-99 (29.2 mg, 10%) as a white solid. ESI-MS (EI⁺, m/z): 532 [M+1]⁺. ¹H NMR (500 MHz, CD₃OD) δ 8.16-8.18 (m, 1H), 7.72-7.73 (m, 1H), 7.39-7.41 (m, 1H), 7.28-7.29 (m, 2H), 7.14-7.16 (m, 1H), 6.97-6.98 (m, 1H), 6.44 (s, 1H), 4.49-4.50 (m, 1H), 3.53-3.66 (m, 9H), 3.32-3.38 (m, 4H), 3.05-3.06 (m, 1H), 2.86-2.88 (m, 1H), 2.70 (s, 4H), 2.28 (s, 3H), 1.45 (d, J=7.0 Hz, 3H).

### Example 70: (S)-3-(4-(4-((4-(2-chlorophenyl)piperazin-1-yl)methyl)-1,5-dimethyl-1H-pyrrole-2-carbonyl)-3-methylpiperazin-1-yl)benzonitrile, I-120

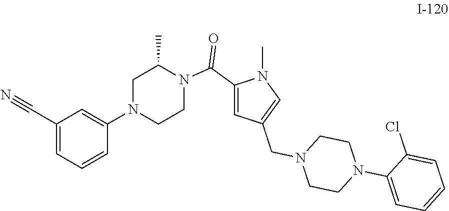 [SMILES: C[C@H]1CN(c2cccc(C#N)c2)CCN1C(=O)c1cc(CN2CCN(c3ccccc3Cl)CC2)cn1C]

### Procedures and Characterization:

The procedure for (S)-3-(4-(4-((4-(2-chlorophenyl)piperazin-1-yl)methyl)-1,5-dimethyl-1H-pyrrole-2-carbonyl)-3-methylpiperazin-1-yl)benzonitrile was the same as Example 69.

Analysis was performed following Method A. Separation was performed following Method C.

### (S)-3-(4-(4-((4-(2-chlorophenyl)piperazin-1-yl)methyl)-1,5-dimethyl-1H-pyrrole-2-carbonyl)-3-methylpiperazin-1-yl)benzonitrile, I-120

ESI-MS (EI⁺, m/z): 531 [M+1]⁺. ¹H NMR (500 MHz, CDCL₃) δ 7.38-7.40 (m, 1H), 7.34-7.36 (m, 2H), 7.08-7.14 (m, 5H), 6.47 (s, 1H), 4.85 (s, 2H), 4.20 (s, 1H), 4.13-4.15 (m, 2H), 3.67-3.69 (m, 4H), 3.31-3.41 (m, 7H), 3.02-3.07 (m, 3H), 2.87-2.90 (m, 1H), 2.88 (s, 3H), 1.43-1.44 (m, 3H).

### Example 71: (S)-2-(4-(4-((4-(2-chlorophenyl)piperazin-1-yl)methyl)-1-methyl-1H-pyrrole-2-carbonyl)-3-methylpiperazin-1-yl)benzonitrile, I-121

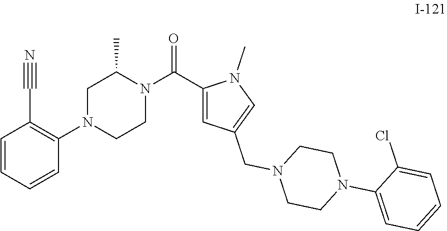 [SMILES: C[C@H]1CN(c2ccccc2CN)CCN1C(=O)c1cc(CN2CCN(c3ccccc3Cl)CC2)cn1C]

### Procedures and Characterization:

The procedure for (S)-2-(4-(4-((4-(2-chlorophenyl)piperazin-1-yl)methyl)-1-methyl-1H-pyrrole-2-carbonyl)-3-methylpiperazin-1-yl)benzonitrile I-121 was the same as Example 69. Analysis was performed following Method A. Separation was performed following Method C. ESI-MS (EI⁺, m/z): 535 [M+1]⁺. ¹H NMR (500 MHz, CD₃OD) δ 7.49-7.51 (m, 1H), 7.37-7.39 (m, 2H), 7.13-7.15 (m, 3H), 6.78-6.87 (m, 2H), 6.33 (s, 1H), 4.74 (s, 1H), 4.47-4.49 (m, 1H), 3.61-3.69 (m, 5H), 3.47-3.55 (m, 6H), 3.06-3.08 (m, 1H), 2.88-2.90 (m, 2H), 2.70 (s, 1H), 2.22 (s, 3H), 2.04-2.07 (m, 2H), 1.43-1.44 (m, 3H), 1.28 (s, 3H).

### Example 72: (S)-2-(4-(4-((4-(2-chlorophenyl)piperazin-1-yl)methyl)-1,5-dimethyl-1H-pyrrole-2-carbonyl)-3-methylpiperazin-1-yl)-6-fluorobenzonitrile, I-102

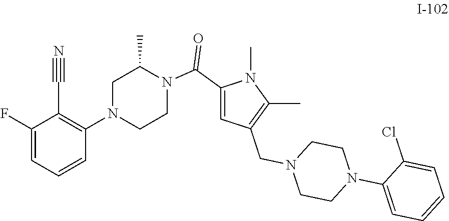 [SMILES: Cc1c(CN2CCN(c3ccccc3Cl)CC2)cc(C(=O)N2CCN(c3cccc(F)c3CN)C[C@@H]2C)n1C]

### Synthetic Scheme:

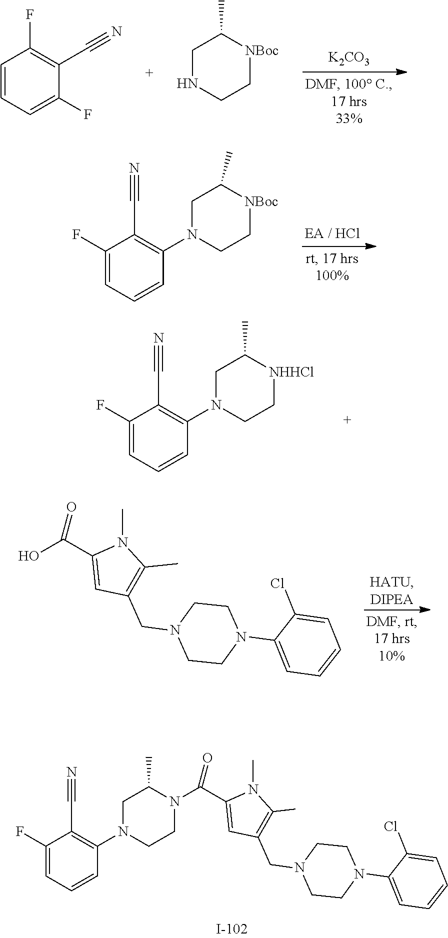

### Procedures and Characterization:

The general procedure for Intermediate I was followed to obtain 4-((4-(2-chlorophenyl)piperazin-1-yl)methyl)-1,5-dimethyl-1H-pyrrole-2-carboxylic acid. Analysis was performed following Method B. Separation was performed following Method D.

### Step 1: (S)-tert-butyl 4-(2-cyano-3-fluorophenyl)-2-methylpiperazine-1-carboxylate

To a solution of 2,6-difluorobenzonitrile (0.8 g, 5.7 mmol) in DMF (10 mL) was added (S)-tert-butyl 2-methylpiperazine-1-carboxylate (1.14 g, 5.7 mmol) and K₂CO₃ (2.35 g, 17.1 mmol). The solution was stirred for 17 hrs at 100° C., then concentrated to give the crude. The crude was purified by chromatography (silica, EtOAc/PE=1/10) to afford (S)-tert-butyl 4-(2-cyano-3-fluorophenyl)-2-methylpiperazine-1-carboxylate (0.6 g, 1.88 mmol, 33%) as a white solid. MS (EI⁺, m/z): 320 [M+H]⁺.

### Step 2: (S)-2-fluoro-6-(3-methylpiperazin-1-yl)benzonitrile hydrochloride

To a solution of (S)-tert-butyl 4-(2-cyano-3-fluorophenyl)-2-methylpiperazine-1-carboxylate (0.6 g, 1.88 mmol) in EtOAc (20 mL) was added EtOAc/HCl (20 ml, 3 M). The mixture was stirred for 17 hrs at rt, then diluted, filtered, and concentrated in vacuo to afford (S)-2-fluoro-6-(3-methylpiperazin-1-yl)benzonitrile hydrochloride (0.5 g, 100%). ESI-MS (EI⁺, m/z): 220 [M+H]⁺.

### Step 3: (S)-2-(4-(4-((4-(2-chlorophenyl)piperazin-1-yl)methyl)-1-methyl-1H-pyrrole-2-carbonyl)-3-methylpiperazin-1-yl)-6-fluorobenzonitrile

To a solution of (S)-2-fluoro-6-(3-methylpiperazin-1-yl)benzonitrile hydrochloride (0.1 g, 0.39 mmol) in DMF (2 mL) was added HATU (148 mg, 0.39 mmol) and DIPEA (117 mg, 1.17 mmol). The mixture was stirred at rt for 17 hrs, then diluted with water (20 mL) and extracted with EtOAc (50×3 mL). The organic phases were dried (Na₂SO₄), filtered, and concentrated in vacuo. The crude product was purified by prep-HPLC to afford (S)-2-(4-(4-((4-(2-chlorophenyl)piperazin-1-yl)methyl)-1-methyl-1H-pyrrole-2-carbonyl)-3-methylpiperazin-1-yl)-6-fluorobenzonitrile I-102 (21 mg, 10%) as a white solid. ESI-MS (EI⁺, m/z): 549 [M+1]⁺. ¹H NMR (500 MHz, CD₃OD) δ 7.65-7.66 (m, 1H), 7.40-7.42 (m, 1H), 7.38-7.39 (m, 1H), 7.28-7.30 (m, 1H). 7.04-7.05 (m, 3H), 6.25-6.27 (m, 1H), 4.74 (s, 1H), 4.24-4.25 (m, 1H), 3.54 (s, 3H), 3.51-3.52 (m, 2H), 3.33-3.34 (m, 2H), 2.95-2.96 (m, 1H), 2.20 (s, 3H), 1.40-1.41 (m, 3H).

### Example 73: (S)-3-(4-(4-((4-(2-chlorophenyl)piperazin-1-yl)methyl)-1-methyl-1H-pyrrole-2-carbonyl)-3-methylpiperazin-1-yl)benzonitrile, I-123

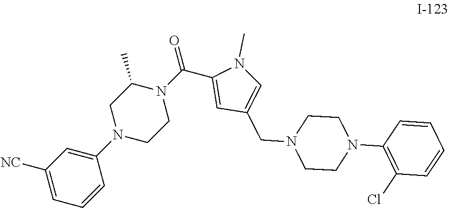 [SMILES: C[C@H]1CN(c2cccc(C#N)c2)CCN1C(=O)c1cc(CN2CCN(c3ccccc3Cl)CC2)cn1C]

### Procedures and Characterization:

The procedure for (S)-3-(4-(4-((4-(2-chlorophenyl)piperazin-1-yl)methyl)-1-methyl-1H-pyrrole-2-carbonyl)-3-methylpiperazin-1-yl)benzonitrile I-123 was the same as Example 69. Analysis was performed following Method B. Separation was performed following Method C. ESI-MS (EI⁺, m/z): 517.3 [M+H]⁺. ¹H NMR (500 MHz, MeOD-d₄) δ 7.Lis45-7.41 (m, 2H), 7.35-7.29 (m, 3H), 7.21-7.11 (m, 4H), 6.59 (d, J=1 Hz, 1H), 4.79 (s, 1H), 4.35 (s, 1H), 4.31 (s, 2H), 3.78 (s, 3H), 3.77 (s, 1H), 3.68-3.55 (m, 6H), 3.29 (s, 2H), 3.13-3.05 (m, 3H), 2.92-2.87 (m, 1H), 1.46 (d, J=6.5 Hz, 3H).

### Example 74: 2-(4-((1-methyl-5-(4-phenylpiperazine-1-carbonyl)-1H-pyrrol-3-yl)methyl)piperazin-1-yl)benzonitrile, I-124

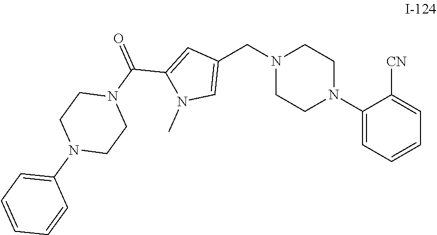 [SMILES: Cn1cc(CN2CCN(c3ccccc3C#N)CC2)cc1C(=O)N1CCN(c2ccccc2)CC1]

### Synthetic Scheme:

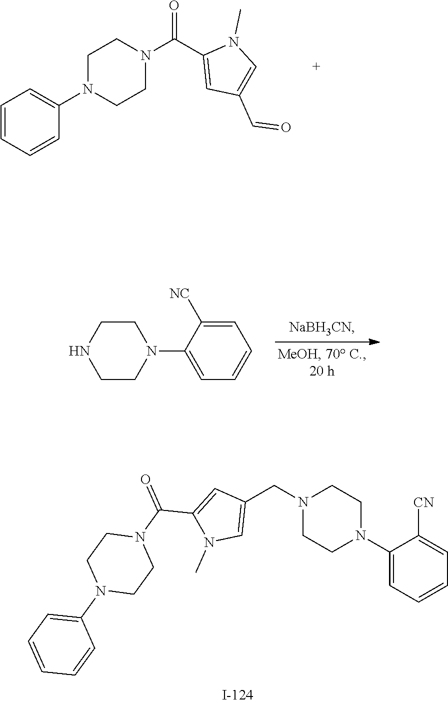 [SMILES: Cn1cc(C=O)cc1C(=O)N1CCN(c2ccccc2)CC1 ; N#Cc1ccccc1N1CCNCC1 ; Cn1cc(CN2CCN(c3ccccc3C#N)CC2)cc1C(=O)N1CCN(c2ccccc2)CC1]

### Procedures and Characterization:

The procedure for 1-methyl-5-(4-phenylpiperazine-1-carbonyl)-1H-pyrrole-3-carbaldehyde was the same as Example 64.

Analysis was performed following Method B. Separation was performed following Method D. ESI-MS (EI⁺, m/z): 469.3 [M+H]⁺. ¹H NMR (500 MHz, MeOD-d₄) δ 7.62 (d, J=8.0 Hz, 1H), 7.57 (t, J=6.0 Hz, 1H), 7.27 (t, J=8.5 Hz, 2H), 7.17 (d, J=8.0 Hz, 1H), 7.10 (q, J=1.0, 7.5 Hz, 1H), 7.02 (d, J=9.0 Hz, 2H), 6.90-6.87 (m, 2H), 6.48 (s, 1H), 3.93-3.91 (m, 4H), 3.75 (s, 3H), 3.54 (s, 2H), 3.25-3.22 (m, 8H), 2.72 (s, 4H).

### Example 75: AlphaLISA Ultra pS6K1 assay

TABLE 3
Key Reagents/Supplies

| Reagents/materials | Vendor | Cat. No. | Lot. No. |
|---|---|---|---|
| MCF-7 | ATCC | HTB-22 | 5105360 |
| DMEM | Invitrogen | 12430-054 | 1677193 |
| FBS | Invitrogen | 10099-141 | 1660516 |
| 0.25% Trypsin-EDTA | Invitrogen | 25200-072 | 1638603 |
| 384 well plate, tissue culture | Corning | CLS3701 | 29214010 |
| treated | | | |
| Corning 384 well storage | Corning | CLS3656 | 29514036 |
| plates | | | |
| Torin1 | Selleck | S2827 | 01 |
| Rapamycin | SELLECK | S1039 | 08 |
| OptiPlate-384, White Opaque | PerkinElmer | 6007299 | 8210-14501 |
| 384-well MicroPlate | | | |
| AlphaLISA SureFire Ultra p- | PerkinElmer | ALSU-PP70- | U0381 |
| p70 S6 Kinase (Thr389) | | A10K | |
| Assay Kit | | | |

Assay Protocol:
1. Seed MCF-7 cells in Corning 3701 plate and incubate for 20˜24 hour. 12,000˜16,000 cells will be seeded in 36 μL medium per well.
2. Change the culture medium with fresh medium and incubate for another 2 hours.
3. Add 12 μL (4λ) compounds into the cell plate by HAMILTON. Final DMSO concentration is 0.5%. Incubate for 2 hours.
4. Aspirate 38 μL by HAMILTON, 10 μL rest per well.
5. Add 10 μL 2× lysis buffer using HAMILTON; total volume in wells is 20 μL. Allow cells to shake for 30 min. Cover plate by plastic foil and store plate at −80° C. up to analysis.
6. Thaw cell lysate at RT and transfer 10 ul lysate to assay plate (Optiplate-384).
7. Add 5 ul acceptor beads into assay plate and incubation for 2 hours
8. Add 5 ul donor beads and incubation for 2 hours
9. Count the plate by EnSpire Multimode Plate Reader

### Example 76: AlphaLISA Ultra pAKT Assay

TABLE 4
Key Reagents/Supplies

| Reagents/materials | Vendor | Cat. No. | Lot. No. |
|---|---|---|---|
| MCF-7 | ATCC | HTB-22 | 5105360 |
| DMEM | Invitrogen | 12430-054 | 1677193 |
| FBS | Invitrogen | 10099-141 | 1660516 |
| 0.25% Trypsin-EDTA | Invitrogen | 25200-072 | 1638603 |
| 384 well plate, tissue | Corning | CLS3701 | 29214010 |
| culture treated | | | |
| Corning 384 well storage | Corning | CLS3656 | 29514036 |
| plates | | | |
| Torin1 | Selleck | S2827 | 01 |
| Rapamycin | SELLECK | S1039 | 08 |
| OptiPlate-384, White | PerkinElmer | 6007299 | 8210-14501 |
| Opaque 384-well | | | |
| MicroPlate | | | |
| AlphaLISA SureFire Ultra | PerkinElmer | ALSU-PAKT- | U0329 |
| p-Akt 1/2/3 (Ser473) Assay | | B10K | |
| Kits | | | |

Assay Protocol:
1. MCF-7 cells in Corning 3701 plate and incubate for 20˜24 hour. 16,000˜20,000 cells will be seeded in 36 μL medium per well.
2. Change the culture medium with fresh medium and incubate for another 2 hours.
3. Add 12 μL (4×) compounds into the cell plate by HAMILTON. Final DMSO concentration is 0.5%. Incubate for 2 hours.
4. Aspirate 38 μL by HAMILTON, 10 μL rest per well.
5. Add 10 μL 2× lysis buffer using HAMILTON; total volume in wells is 20 μL. Allow cells to shake for 30 min. Cover plate by plastic foil and store plate at −80° C. up to analysis.
6. Thaw cell lysate at RT and transfer 10 ul lysate to assay plate (Optiplate-384).
7. Add 5 ul acceptor beads into assay plate and incubation for 2 hours
8. Add 5 ul donor beads and incubation for 2 hours
9. Count the plate by EnSpire Multimode Plate Reader

### Example 157: Radioactive Glucose Uptake in MCF-7 Cells

Assay Protocol
1. Seed approximately 12,000 MCF-7 cells cultured without insulin per well in 96-well plates (BIOCOAT, #356690) and let the cells sit in hood for 30 min. (Cell culture medium: DMEM+10% FBS).
2. Put the cell culture plates in TC incubator (5% CO₂, 37° C.) and incubate overnight.
3. Take out the cell plates and wash with prewarmed KRH buffer (37° C.) three times using BioTek plate washer. (KRH buffer preparation: 136 mM NaCl, 4.7 mM KCl, 1.25 mM MgCl₂, 1.2 mM CaCl₂, 20 mM HEPES, 0.1 mg/ml sodium pyruvate, 0.1% BSA, pH 7.4).
4. Compound preparation and treatment:
   a) Add 12 ul compound stock in 100% DMSO to the first column of Echo LDV plate
   b) Serially dilute the compound solution in 100% DMSO (4 uL+8 uL DMSO) using Bravo for 9 doses
   c) Dispense 350 nL compounds using Echo to the working plate
   d) i) Without compound preincubation condition, dispense 116 uL KRH buffer containing tritiated 2-deoxy-D-glucose (3H-2DG) and 2-deoxy-D-glucose (2DG) to the working plate and mix.
   ii. With compound preincubation condition, dispense 110 uL KRH buffer only to the working plates and mix
   e) Keep the compound working plate at 37° C. incubator until use
5. Discard the KRH buffer (cells were kept in KRH buffer for 10-15 min before 2-DG addition) and transfer 100 uL compound working solution out to the cell culture plate using Bravo. Incubate for 20 mins at 37° C. (2DG final at 0.2 mM, 3H-2DG at 10 uCi/mL).
6. Add prewarmed 50 uL KRH buffer (37° C.) containing 60 mM cold 2DG to the wells to stop the 2DG uptake using Multidrop combi
7. Wash the cells with prewarmed PBS three times using BioTek plate washer
8. Add 50 uL lysis buffer (0.1 M NaOH) into the assay plate to lyse the cells using Bravo and shake at 800 rpm for 30 min.
9. Mix and transfer 20 uL lysate to a new white plate (Corning, #3610) and add 200 uL MicroScint-20 using Bravo
10. Shake the plate at 1000 rpm for 30 min and count in a MicroBeta plate reader.

### Example 158: ATP/Rotenone Assay

Assay Protocol

Day 1: Plate cells in Corning 96-Well Clear Bottom Black Polystyrene Microplates (corning 3340) in Glucose-free DMEM+10% dFBS
80 uL/well
25,000 cells/well for 293T
10,000 cells/well for MCF7

### Day 2:

### Glucose DR to Establish Glucose EC50

1. 1 h pre-treat with 10 uM Rotenone
   add 10 uL if 9× (90 uM) rotenone to each well (freshly made 10 mM stock diluted 1:111× in glucose-free DMEM.
2. Stimulate with glucose for 15 min
   Make glucose DR at 10× concentration in glucose-free DMEM
   Add 10 uL per well and incubate at 37° C. for 15 min
3. Reconstitute and Measure
   Remove plate, immediately add 100 uL of reconstituted Celltiter-Glo reagent (Promega; G7570) per well
   Shake plate covered for 10 min at room temperature
   Let plate sit for 2-5 min, read luminescence
   Calculate EC50 for glucose (usually ˜1 mM for 293T cells and ˜0.25 mM for MCF-7 cells).

### Determine Compound IC50s

1. 1 h pre-treat Rotenone/compound solution
   Make 9× (90 uM) rotenone: 10 mM stock of Rotenone fresh—dilute 1:111× in Glucose-free DMEM).
   Make compound dilution series in DMSO then dilute 1:111 in 90 uM rotenone in glucose-free media
   Add 10 uL of 9× rotenone/compound solutions per well
2. Stimulate with glucose for 15 min
   Make glucose DR at 10× concentration in glucose-free DMEM
   Add 10 uL per well and incubate at 37° C. for 15 min
3. Reconstitute and Measure
   Remove plate, immediately add 100 uL of reconstituted Celltiter-Glo reagent (Promega; G7570) per well
   Shake plate covered for 10 min at room temperature
   Let plate sit for 2-5 min, read luminescence
   Calculate IC50 for compounds

Table 5 shows the inhibitory activity (IC₅₀) of selected compounds of this invention in the pS6K1, pAKT, and glucose uptake assays. The compound numbers correspond to the compound numbers in Table 1. Compounds having an activity designated as “A” provided inhibitory activity (IC₅₀) of 0.01-1 μM; compounds designated as “B” provided inhibitory activity (IC₅₀) of 1-3 μM; compounds designated as “C” provided inhibitory activity (IC₅₀) of 3-10 μM; compounds designated as “D” provided inhibitory activity (IC₅₀)>0.3 μM; compounds designated as “E” provided inhibitory activity (IC₅₀)>1 μM; compounds designated as “F” provided inhibitory activity (IC₅₀)>3 μM; compounds designated as “G” provided inhibitory activity (IC₅₀)>5 μM and compounds designated as “H” provided inhibitory activity (IC₅₀)>10 μM.

TABLE 5
Assay Data for Exemplary Compounds

| | pS6K1 in | pAKT in | Glucose Uptake in |
|---|---|---|---|
| | MCF7 IC₅₀ | mCF7 IC₅₀ | MCF7 IC₅₀ |
| Compound Number | (μM) | (μM) | (μM) |
| I-1 | A | E | NA |
| I-2 | B | H | NA |
| I-3 | A | H | NA |
| I-4 | A | E | NA |
| I-14 | A | H | NA |
| I-15 | B | H | NA |
| I-16 | B | H | NA |
| I-17 | C | H | NA |
| I-18 | B | H | NA |
| I-21 | B | H | NA |
| I-22 | B | H | NA |
| I-23 | A | H | NA |
| I-24 | C | H | NA |
| I-27 | B | H | NA |
| I-29 | C | H | NA |
| I-30 | C | H | NA |
| I-31 | B | H | NA |
| I-33 | B | H | NA |
| I-34 | B | H | NA |
| I-35 | B | H | NA |
| I-36 | A | H | NA |
| I-37 | A | F | NA |
| I-38 | A | F | NA |
| I-39 | A | H | NA |
| I-40 | C | H | NA |
| I-41 | B | H | NA |
| I-42 | A | F | NA |
| I-43 | A | F | NA |
| I-44 | A | F | NA |
| I-45 | B | H | NA |
| I-46 | B | H | NA |
| I-47 | B | H | NA |
| I-48 | C | H | NA |
| I-49 | B | H | NA |
| I-50 | B | H | NA |
| I-51 | A | H | NA |
| I-52 | A | H | NA |
| I-53 | B | H | NA |
| I-54 | A | E | NA |
| I-55 | A | E | NA |
| I-56 | A | F | NA |
| I-57 | A | F | NA |
| I-58 | A | H | NA |
| I-59 | B | F | NA |
| I-60 | A | F | NA |
| I-61 | A | F | NA |
| I-62 | A | F | NA |
| I-63 | A | D | A |
| I-64 | A | F | NA |
| I-65 | A | F | NA |
| I-66 | A | F | NA |
| I-67 | A | H | NA |
| I-68 | A | E | NA |
| I-69 | A | F | NA |
| I-70 | A | F | NA |
| I-71 | A | F | NA |
| I-72 | A | D | A |
| I-73 | C | H | NA |
| I-74 | B | H | NA |
| I-75 | B | G | NA |
| I-76 | B | G | F |
| I-77 | B | G | NA |
| I-78 | A | F | NA |
| I-79 | A | F | NA |
| I-80 | A | F | NA |
| I-81 | B | H | NA |
| I-82 | B | H | NA |
| I-83 | B | H | NA |
| I-84 | A | F | NA |
| I-85 | A | F | NA |
| I-86 | A | H | NA |
| I-87 | A | G | NA |
| I-88 | B | H | NA |
| I-89 | C | H | NA |
| I-90 | B | F | NA |
| I-91 | A | G | NA |
| I-92 | A | E | NA |
| I-93 | A | G | NA |
| I-94 | A | G | NA |
| I-95 | A | F | NA |
| I-96 | A | F | NA |
| I-97 | B | G | NA |
| I-98 | A | F | NA |
| I-99 | A | F | NA |
| I-100 | A | H | NA |
| I-101 | A | G | NA |
| I-102 | A | H | NA |
| I-103 | B | H | NA |
| I-104 | B | G | NA |
| I-105 | A | F | NA |
| I-106 | B | F | NA |
| I-107 | A | F | NA |
| I-108 | A | E | NA |
| I-109 | A | E | NA |
| I-110 | A | E | NA |
| I-111 | A | F | NA |
| I-112 | A | F | NA |
| I-113 | A | F | NA |
| I-114 | B | H | NA |
| I-115 | A | F | NA |
| I-116 | A | E | NA |
| I-117 | A | F | NA |
| I-118 | A | F | NA |
| I-119 | B | F | NA |
| I-120 | A | E | NA |
| I-121 | A | E | NA |
| I-122 | A | E | NA |
| I-123 | A | E | NA |
| I-124 | A | F | NA |
| I-125 | B | H | NA |
| I-126 | C | H | NA |
| I-127 | B | H | NA |
| I-128 | B | H | NA |
| I-129 | B | H | NA |
| I-130 | B | H | NA |
| I-131 | C | H | NA |
| I-132 | C | H | NA |
| I-133 | A | E | NA |
| I-134 | B | F | NA |
| I-135 | B | F | NA |
| I-136 | B | F | NA |
| I-137 | C | H | NA |
| I-138 | B | F | NA |
| I-139 | C | H | NA |
| I-140 | B | H | NA |
| I-141 | C | H | NA |
| I-142 | B | F | NA |
| I-143 | A | F | NA |
| I-144 | B | F | NA |
| I-145 | C | H | NA |
| I-146 | C | H | NA |
| I-147 | C | H | NA |
| I-148 | B | H | NA |
| I-149 | C | H | NA |
| I-150 | C | H | NA |
| I-151 | B | H | NA |
| I-152 | B | H | NA |

We claim:

1. A compound of Formula I: 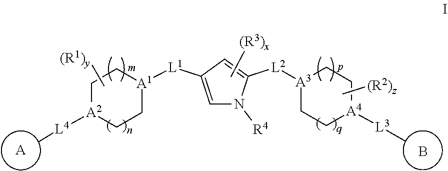
or a pharmaceutically acceptable salt thereof, wherein:
A¹ is N or CH;
A² is N or CR′, provided at least one of A¹ and A² comprises a nitrogen;
A³ is N or CH;
A⁴ is N or CH;
R′ is H or C₁₋₆ aliphatic; or
R′ and L⁴ are optionally taken together with their intervening atoms to form a 5-8 membered saturated spiro-fused ring having 0-2 heteroatoms independently selected from nitrogen, oxygen or sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each of m, n, p, q, and x is independently 0, 1, or 2;
each of y and z is independently 0, 1, 2, 3, or 4;
each of R¹ and R² is independently R, or:
two R¹ groups are optionally taken together to form ═O;
two R² groups are optionally taken together to form ═O;
two R¹ groups are optionally taken together to form a covalent bond or a bivalent C₁₋₄ alkylene chain;
two R² groups are optionally taken together to form a covalent bond or a bivalent C₁₋₄ alkylene chain;
an R¹ group and Ring A are optionally taken together with their intervening atoms to form a 5-8 membered aryl fused ring having 0-2 heteroatoms independently selected from nitrogen, oxygen or sulfur;
an R² group and Ring B are optionally taken together with their intervening atoms to form a 5-8 membered aryl fused ring having 0-2 heteroatoms independently selected from nitrogen, oxygen or sulfur; or
an R² group and L³ are optionally taken together with their intervening atoms to form a 5-8 membered aryl fused ring having 0-2 heteroatoms independently selected from nitrogen, oxygen or sulfur;
each of R³ is independently hydrogen, C₁₋₆ aliphatic, or —CN;
R⁴ is hydrogen or an optionally substituted C₁₋₆ aliphatic group;
Ring A is an optionally substituted ring selected from 6-membered aryl containing 0-2 nitrogen atoms, 5-membered heteroaryl with 1-4 heteroatoms independently selected from nitrogen, oxygen or sulfur, or 8-10 membered bicyclic aryl or heteroaryl with 1-4 heteroatoms independently selected from nitrogen, oxygen or sulfur;
Ring B is an optionally substituted ring selected from 6-membered aryl containing 0-2 nitrogen atoms, 5-membered heteroaryl with 1-4 heteroatoms independently selected from nitrogen, oxygen or sulfur, or 8-10 membered bicyclic aryl or heteroaryl with 1-4 heteroatoms independently selected from nitrogen, oxygen or sulfur;
L¹ is a covalent bond or a C₁₋₃ bivalent straight or branched saturated or unsaturated hydrocarbon chain wherein 1-2 methylene units of the chain are independently and optionally replaced with —C(O)—, —N(R)—, or —CH(R)—;
L² is a covalent bond or a C₁₋₃ bivalent straight or branched saturated or unsaturated hydrocarbon chain wherein 1-2 methylene units of the chain are independently and optionally replaced with —C(O)— or —CH(R)—;
L³ is a covalent bond or —N(R)—; and
L⁴ is a covalent bond or —N(R)—.

2. The compound according to claim 1, wherein A¹ is N.

3. The compound according to claim 2, wherein each of A², A³, and A⁴, is N, and wherein each of m, n, p, and q is independently 1 or 2.

4.-7. (canceled)

8. The compound according to claim 2, wherein, each of R¹ and R² is independently R, or: two R² groups are optionally taken together to form ═O; two R¹ groups are optionally taken together to form a covalent bond or a bivalent C₁₋₄ alkylene chain; an R² group and Ring B are optionally taken together with their intervening atoms to form a 5-8 membered fused ring having 0-2 heteroatoms independently selected from nitrogen, oxygen, or sulfur; or an R² group and L³ are optionally taken together with their intervening atoms to form a 5-8 membered aryl fused ring having 0-2 heteroatoms independently selected from nitrogen, oxygen or sulfur.

9. The compound according to claim 8, wherein each R¹ is independently hydrogen or —CH₃, or: two R¹ groups are optionally taken together to form a covalent bond or two R¹ groups are optionally taken together to form a bivalent C₁₋₄ alkylene chain.

10. The compound according to claim 8, wherein each R² is independently hydrogen or —CH₃, or: two R² groups are optionally taken together to form ═O; an R² group and Ring B are optionally taken together with their intervening atoms to form a 5-8 membered fused ring having 0-2 heteroatoms independently selected from nitrogen, oxygen, or sulfur; or an R² group and L³ are optionally taken together with their intervening atoms to form a 5-8 membered aryl fused ring having 0-2 heteroatoms independently selected from nitrogen, oxygen or sulfur.

11. The compound according to claim 2, wherein each of R³ is independently hydrogen, methyl, or —CN; R⁴ is an optionally substituted C₁₋₆ aliphatic group; and Ring A is an optionally substituted 6-membered aryl ring containing 0-2 nitrogen atoms.

12. (canceled)

13. The compound according to claim 11, wherein R⁴ is methyl.

14. (canceled)

15. The compound according to claim 11, wherein Ring A is phenyl,

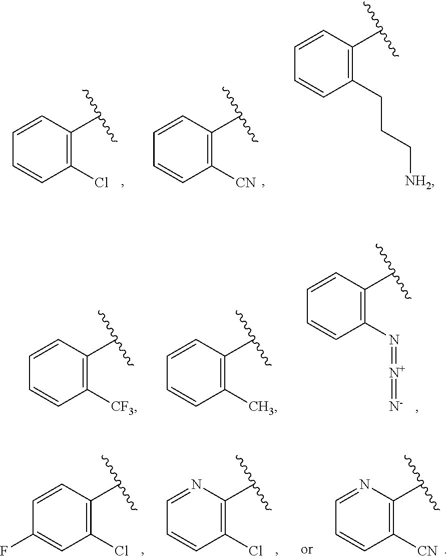

16. The compound according to claim 2, wherein Ring B is phenyl, pyridyl, pyrimidinyl, pyrazinyl, pyridazinyl, indolyl,

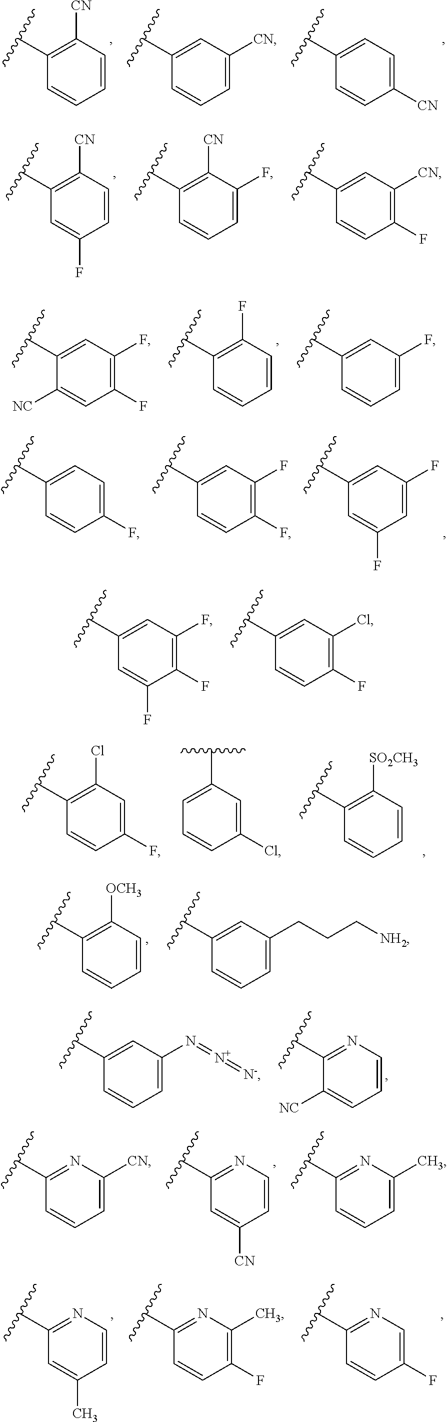

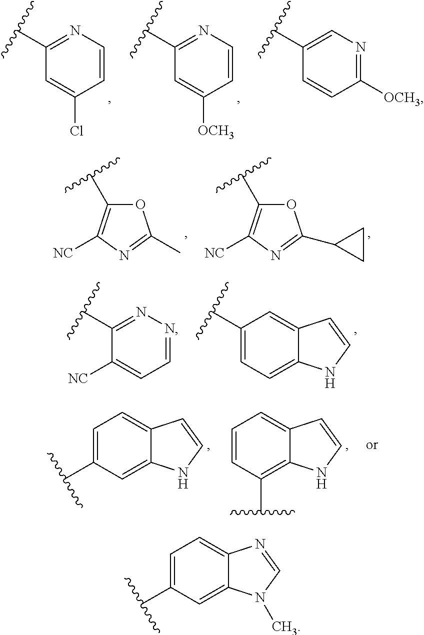

17. The compound according to claim 2, wherein L¹ is a C₁₋₃ bivalent straight or branched saturated or unsaturated hydrocarbon chain wherein 1-2 methylene units of the chain are independently and optionally replaced with —C(O)—, —N(R)—, or —CH(R)— and L² is a C₁₋₃ bivalent straight or branched saturated or unsaturated hydrocarbon chain wherein 1-2 methylene units of the chain are independently and optionally replaced with —C(O)— or —CH(R)—.

18. The compound according to claim 17, wherein L¹ is —CH₂—.

19. (canceled)

20. The compound according to claim 17, wherein L² is

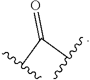 [SMILES: *C(*)=O]

21. The compound according to claim 2, wherein L³ is a covalent bond.

22. The compound according to claim 2, wherein L⁴ is a covalent bond.

23. The compound according to claim 1, wherein said compound is any of Formulae I-a, I-b, I-c, or I-d: 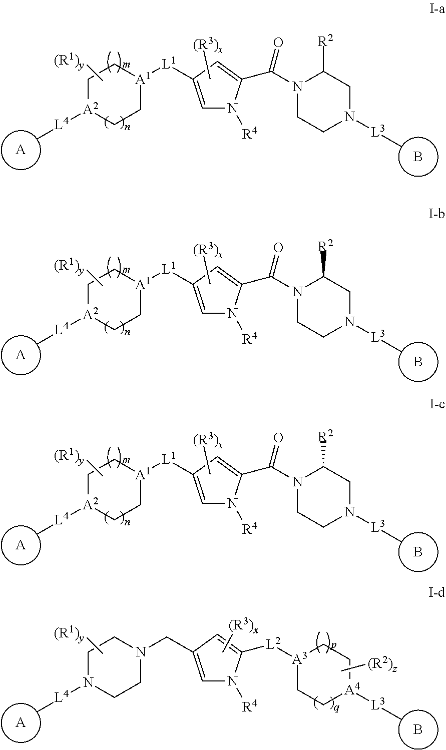
or a pharmaceutically acceptable salt thereof.

24. The compound according to claim 1, wherein said compound is selected from: 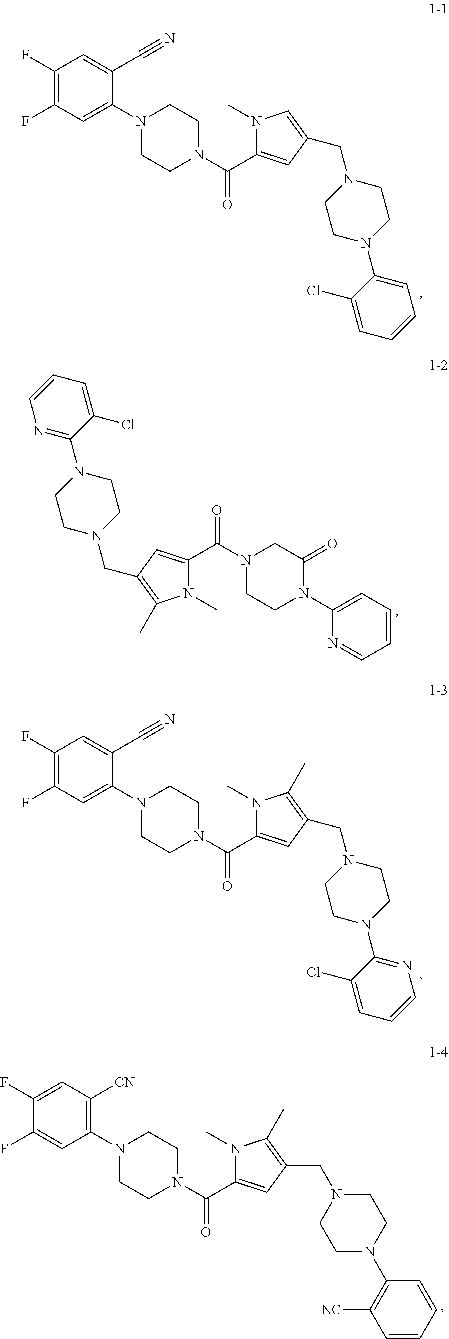 [SMILES: Cn1cc(CN2CCN(c3ccccc3Cl)CC2)cc1C(=O)N1CCN(c2cc(F)c(F)cc2C#N)CC1 ; Cc1c(CN2CCN(c3ncccc3Cl)CC2)cc(C(=O)N2CCN(c3ccccn3)C(=O)C2)n1C ; Cc1c(CN2CCN(c3ncccc3Cl)CC2)cc(C(=O)N2CCN(c3cc(F)c(F)cc3C#N)CC2)n1C ; Cc1c(CN2CCN(c3ccccc3C#N)CC2)cc(C(=O)N2CCN(c3cc(F)c(F)cc3C#N)CC2)n1C] 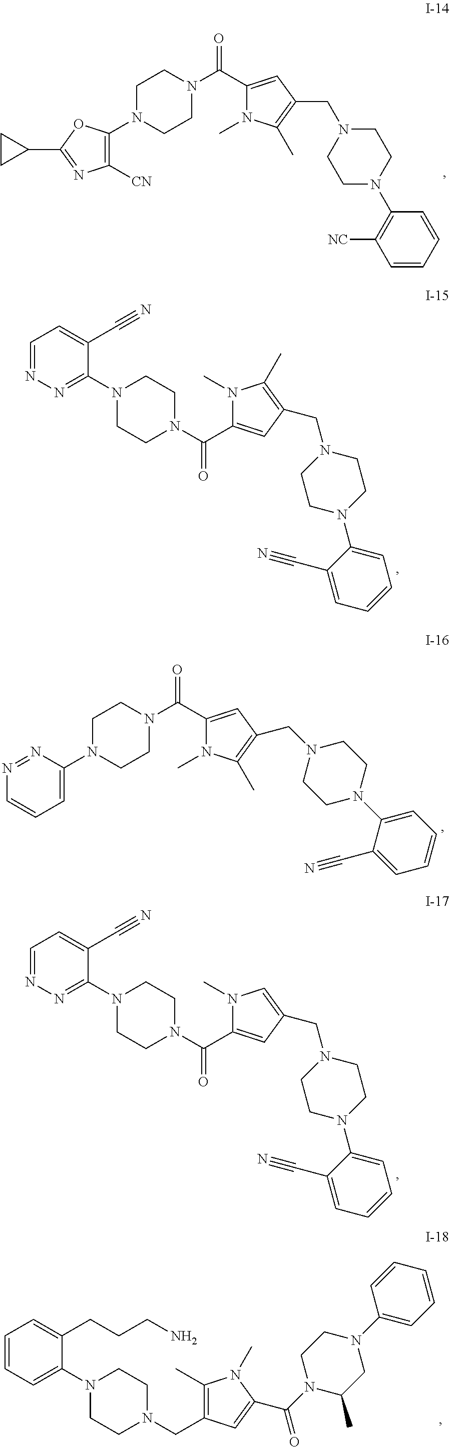 [SMILES: Cc1c(CN2CCN(c3ccccc3C#N)CC2)cc(C(=O)N2CCN(c3oc(C4CC4)nc3C#N)CC2)n1C ; Cc1c(CN2CCN(c3ccccc3C#N)CC2)cc(C(=O)N2CCN(c3nnccc3C#N)CC2)n1C ; Cc1c(CN2CCN(c3ccccc3C#N)CC2)cc(C(=O)N2CCN(c3cccnn3)CC2)n1C ; Cn1cc(CN2CCN(c3ccccc3C#N)CC2)cc1C(=O)N1CCN(c2nnccc2C#N)CC1 ; Cc1c(CN2CCN(c3ccccc3CCCN)CC2)cc(C(=O)N2CCN(c3ccccc3)C[C@H]2C)n1C] 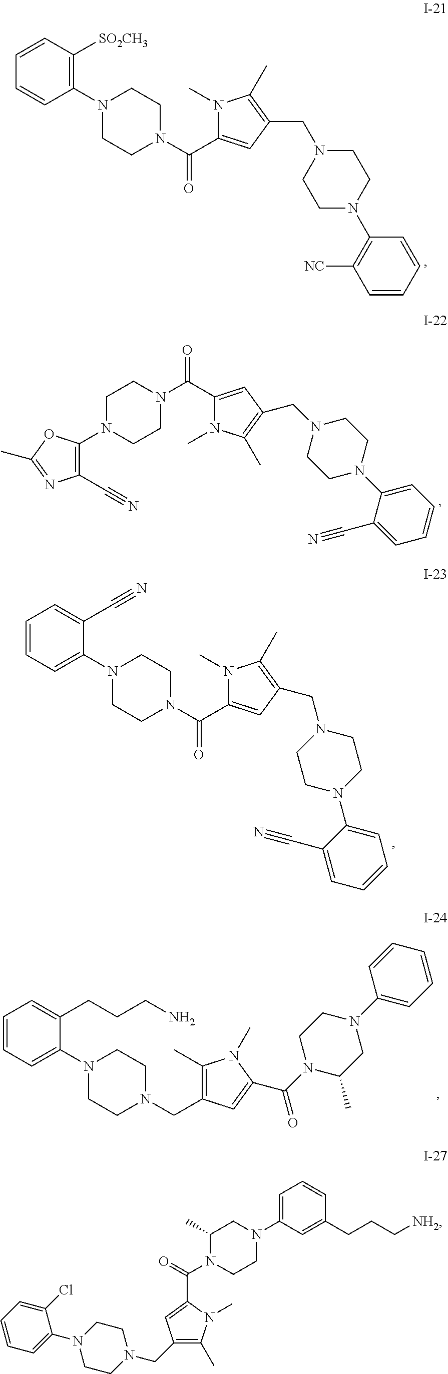 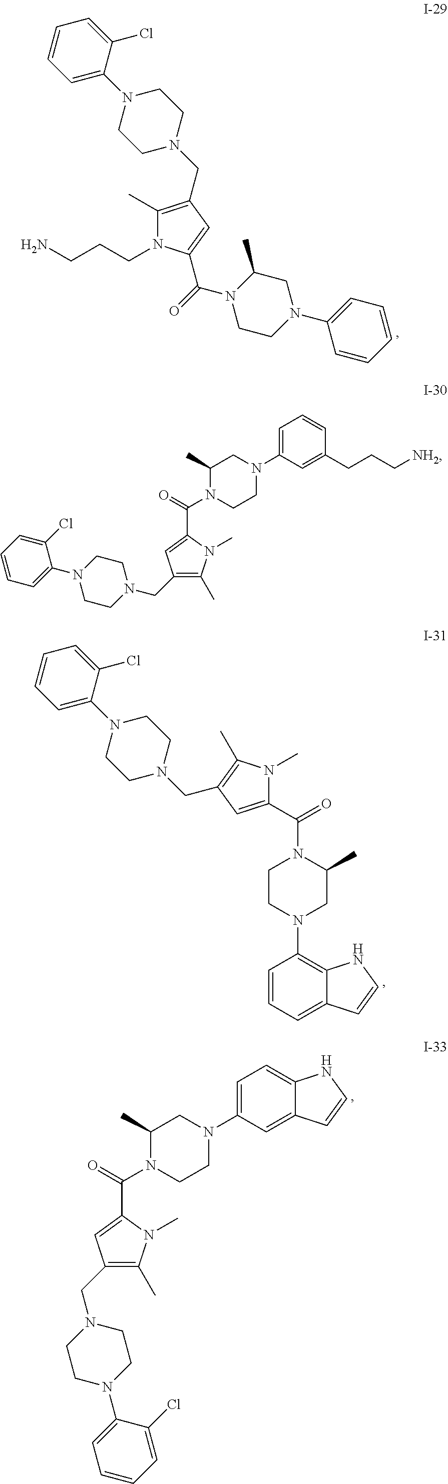 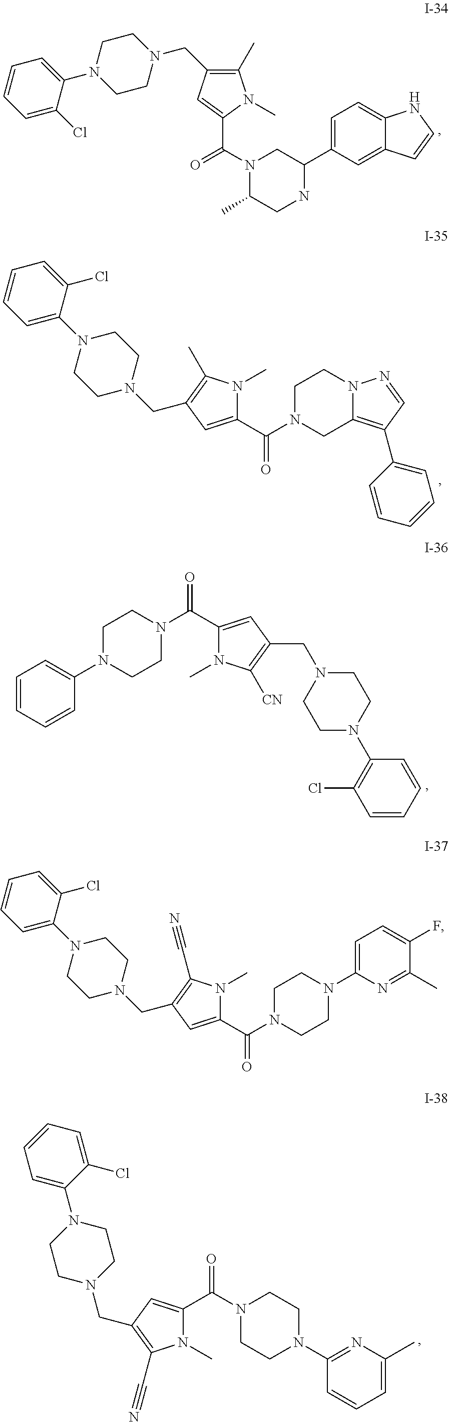 [SMILES: Cc1c(CN2CCN(c3ccccc3Cl)CC2)cc(C(=O)N2CC(c3ccc4[nH]ccc4c3)NC[C@@H]2C)n1C ; Cc1c(CN2CCN(c3ccccc3Cl)CC2)cc(C(=O)N2CCn3ncc(-c4ccccc4)c3C2)n1C ; Cn1c(C(=O)N2CCN(c3ccccc3)CC2)cc(CN2CCN(c3ccccc3Cl)CC2)c1C#N ; Cc1nc(N2CCN(C(=O)c3cc(CN4CCN(c5ccccc5Cl)CC4)c(C#N)n3C)CC2)ccc1F ; Cc1cccc(N2CCN(C(=O)c3cc(CN4CCN(c5ccccc5Cl)CC4)c(C#N)n3C)CC2)n1] 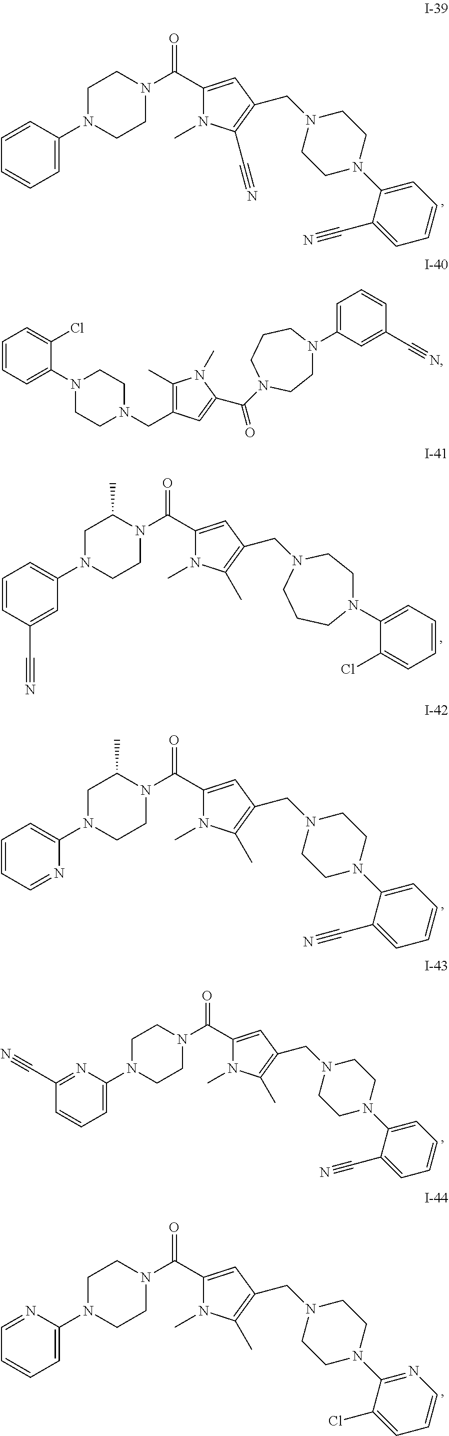 [SMILES: Cn1c(C(=O)N2CCN(c3ccccc3)CC2)cc(CN2CCN(c3ccccc3C#N)CC2)c1C#N ; Cc1c(CN2CCN(c3ccccc3Cl)CC2)cc(C(=O)N2CCCN(c3cccc(C#N)c3)CC2)n1C ; Cc1c(CN2CCCN(c3ccccc3Cl)CC2)cc(C(=O)N2CCN(c3cccc(C#N)c3)C[C@@H]2C)n1C ; Cc1c(CN2CCN(c3ccccc3C#N)CC2)cc(C(=O)N2CCN(c3ccccn3)C[C@@H]2C)n1C ; Cc1c(CN2CCN(c3ccccc3C#N)CC2)cc(C(=O)N2CCN(c3cccc(C#N)n3)CC2)n1C ; Cc1c(CN2CCN(c3ncccc3Cl)CC2)cc(C(=O)N2CCN(c3ccccn3)CC2)n1C] 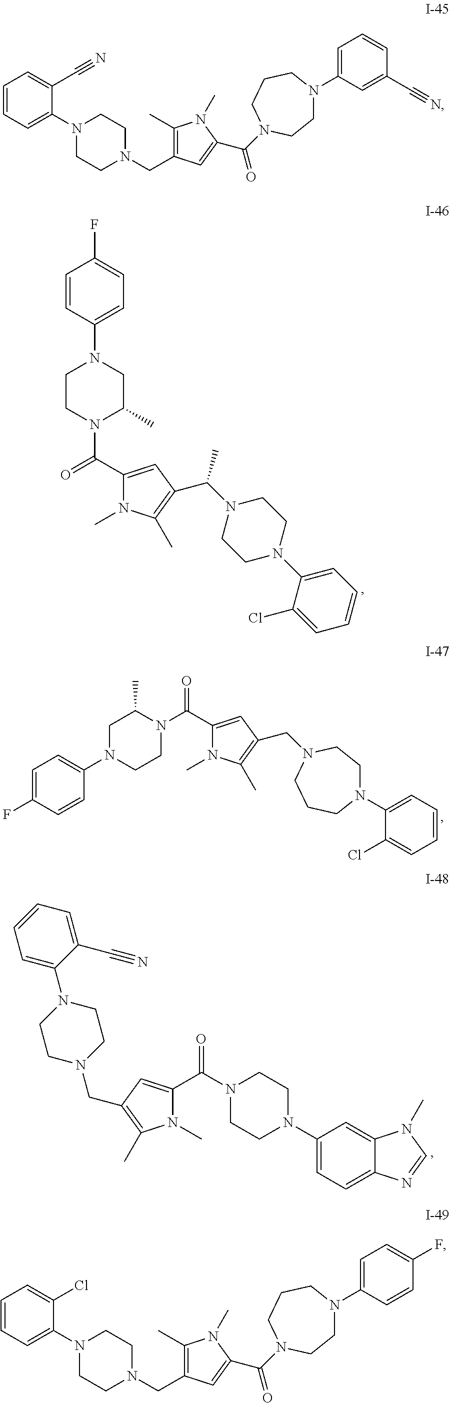 [SMILES: Cc1c(CN2CCN(c3ccccc3C#N)CC2)cc(C(=O)N2CCCN(c3cccc(C#N)c3)CC2)n1C ; Cc1c([C@H](C)N2CCN(c3ccccc3Cl)CC2)cc(C(=O)N2CCN(c3ccc(F)cc3)C[C@@H]2C)n1C ; Cc1c(CN2CCCN(c3ccccc3Cl)CC2)cc(C(=O)N2CCN(c3ccc(F)cc3)C[C@@H]2C)n1C ; Cc1c(CN2CCN(c3ccccc3C#N)CC2)cc(C(=O)N2CCN(c3ccc4ncn(C)c4c3)CC2)n1C ; Cc1c(CN2CCN(c3ccccc3Cl)CC2)cc(C(=O)N2CCCN(c3ccc(F)cc3)CC2)n1C] 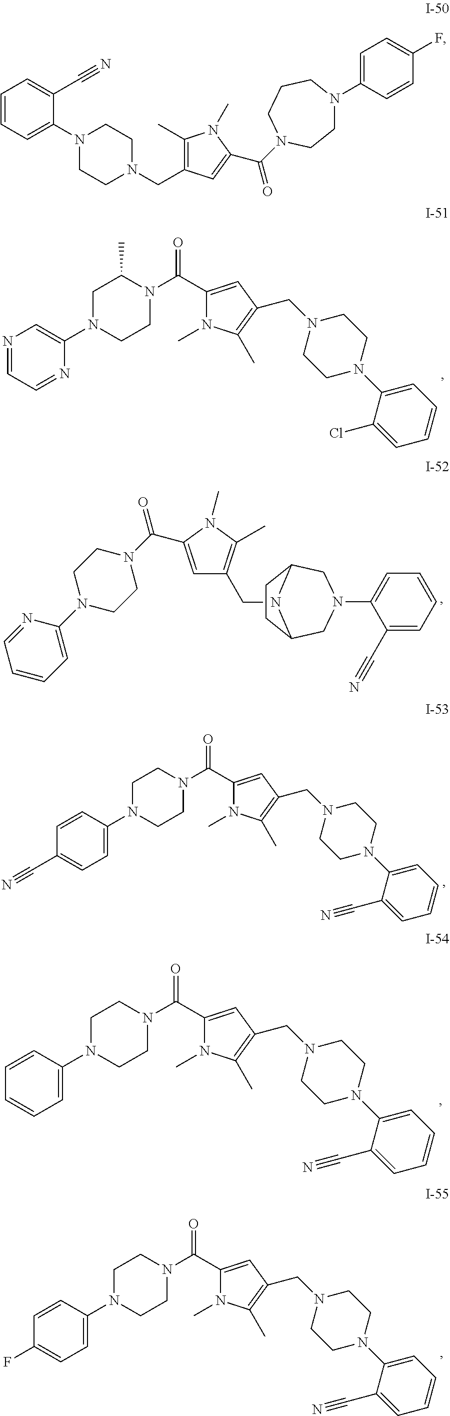 [SMILES: Cc1c(CN2CCN(c3ccccc3C#N)CC2)cc(C(=O)N2CCCN(c3ccc(F)cc3)CC2)n1C ; Cc1c(CN2CCN(c3ccccc3Cl)CC2)cc(C(=O)N2CCN(c3cnccn3)C[C@@H]2C)n1C ; Cc1c(CN2C3CCC2CN(c2ccccc2C#N)C3)cc(C(=O)N2CCN(c3ccccn3)CC2)n1C ; Cc1c(CN2CCN(c3ccccc3C#N)CC2)cc(C(=O)N2CCN(c3ccc(C#N)cc3)CC2)n1C ; Cc1c(CN2CCN(c3ccccc3C#N)CC2)cc(C(=O)N2CCN(c3ccccc3)CC2)n1C ; Cc1c(CN2CCN(c3ccccc3C#N)CC2)cc(C(=O)N2CCN(c3ccc(F)cc3)CC2)n1C] 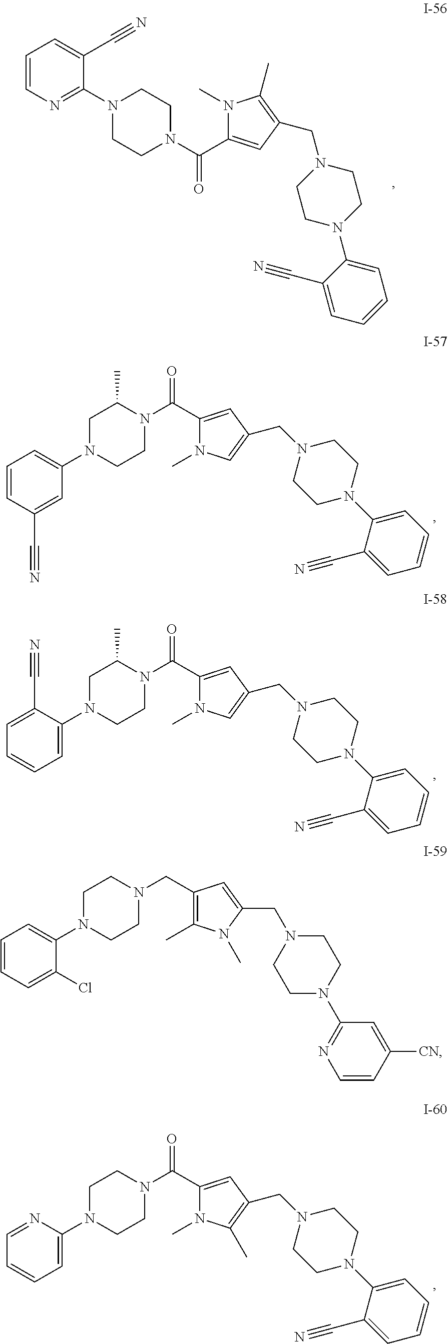 [SMILES: Cc1c(CN2CCN(c3ccccc3C#N)CC2)cc(C(=O)N2CCN(c3ncccc3C#N)CC2)n1C ; C[C@H]1CN(c2cccc(C#N)c2)CCN1C(=O)c1cc(CN2CCN(c3ccccc3C#N)CC2)cn1C ; C[C@H]1CN(c2ccccc2C#N)CCN1C(=O)c1cc(CN2CCN(c3ccccc3C#N)CC2)cn1C ; Cc1c(CN2CCN(c3ccccc3Cl)CC2)cc(CN2CCN(c3cc(C#N)ccn3)CC2)n1C ; Cc1c(CN2CCN(c3ccccc3C#N)CC2)cc(C(=O)N2CCN(c3ccccn3)CC2)n1C] 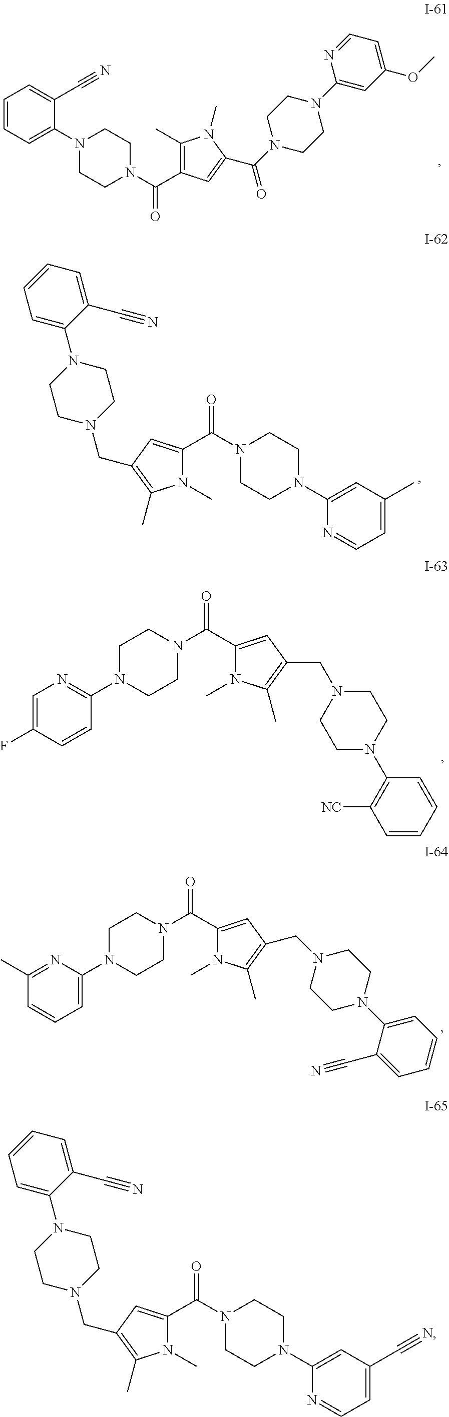 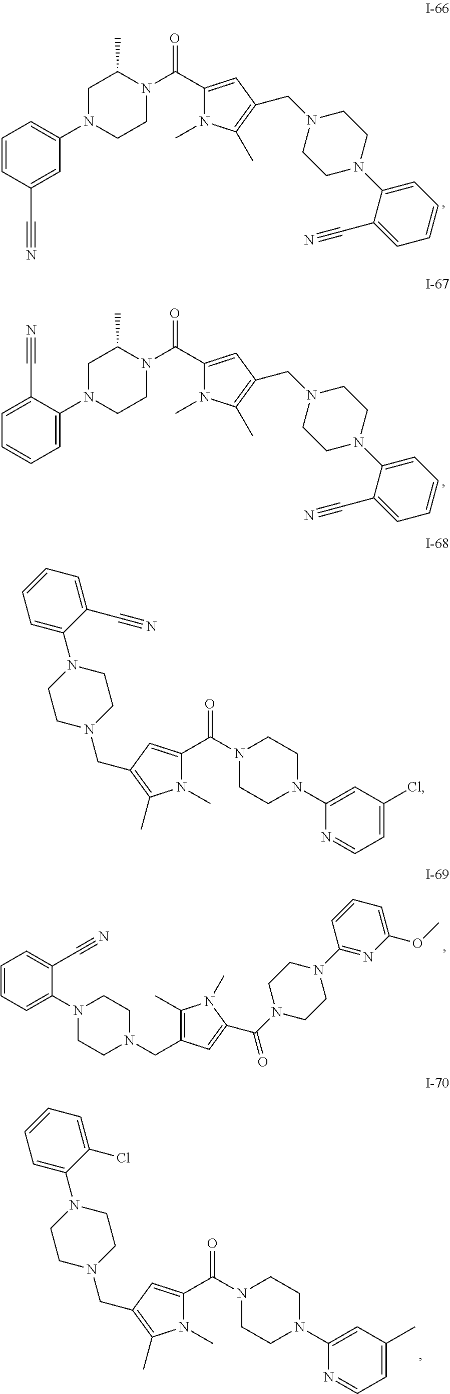 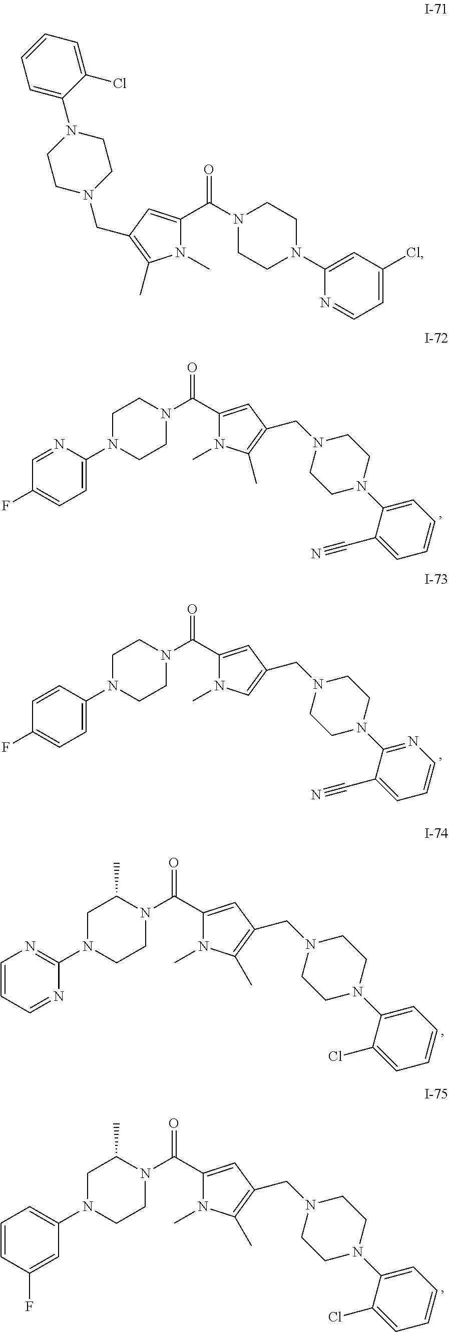 [SMILES: Cc1c(CN2CCN(c3ccccc3Cl)CC2)cc(C(=O)N2CCN(c3cc(Cl)ccn3)CC2)n1C ; Cc1c(CN2CCN(c3ccccc3C#N)CC2)cc(C(=O)N2CCN(c3ccc(F)cn3)CC2)n1C ; Cn1cc(CN2CCN(c3ncccc3C#N)CC2)cc1C(=O)N1CCN(c2ccc(F)cc2)CC1 ; Cc1c(CN2CCN(c3ccccc3Cl)CC2)cc(C(=O)N2CCN(c3ncccn3)C[C@@H]2C)n1C ; Cc1c(CN2CCN(c3ccccc3Cl)CC2)cc(C(=O)N2CCN(c3cccc(F)c3)C[C@@H]2C)n1C] 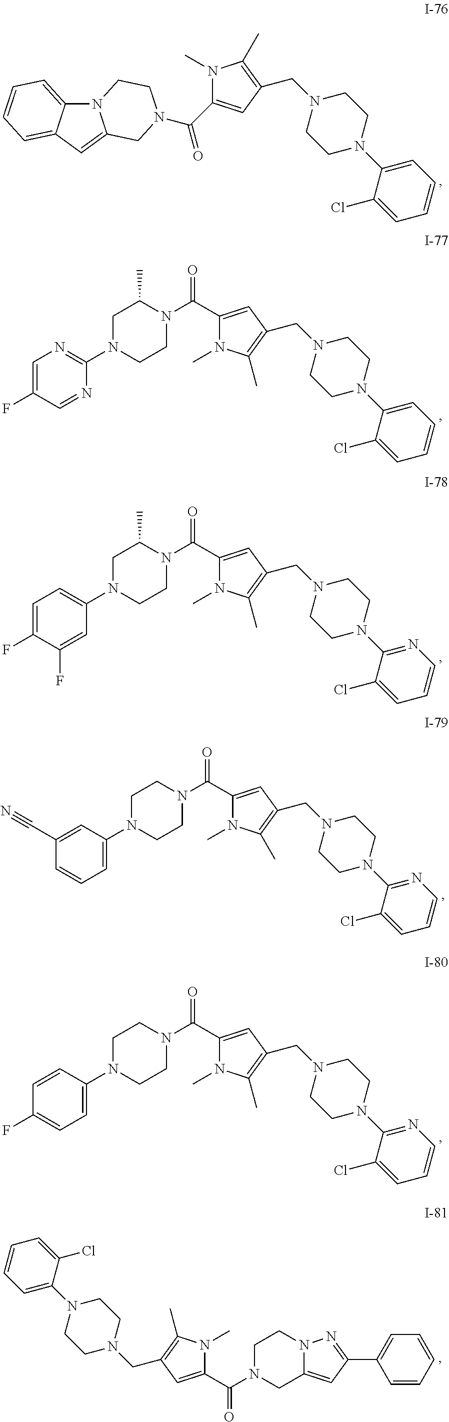 [SMILES: Cc1c(CN2CCN(c3ccccc3Cl)CC2)cc(C(=O)N2CCn3c(cc4ccccc43)C2)n1C ; Cc1c(CN2CCN(c3ccccc3Cl)CC2)cc(C(=O)N2CCN(c3ncc(F)cn3)C[C@@H]2C)n1C ; Cc1c(CN2CCN(c3ncccc3Cl)CC2)cc(C(=O)N2CCN(c3ccc(F)c(F)c3)C[C@@H]2C)n1C ; Cc1c(CN2CCN(c3ncccc3Cl)CC2)cc(C(=O)N2CCN(c3cccc(C#N)c3)CC2)n1C ; Cc1c(CN2CCN(c3ncccc3Cl)CC2)cc(C(=O)N2CCN(c3ccc(F)cc3)CC2)n1C ; Cc1c(CN2CCN(c3ccccc3Cl)CC2)cc(C(=O)N2CCn3nc(-c4ccccc4)cc3C2)n1C] 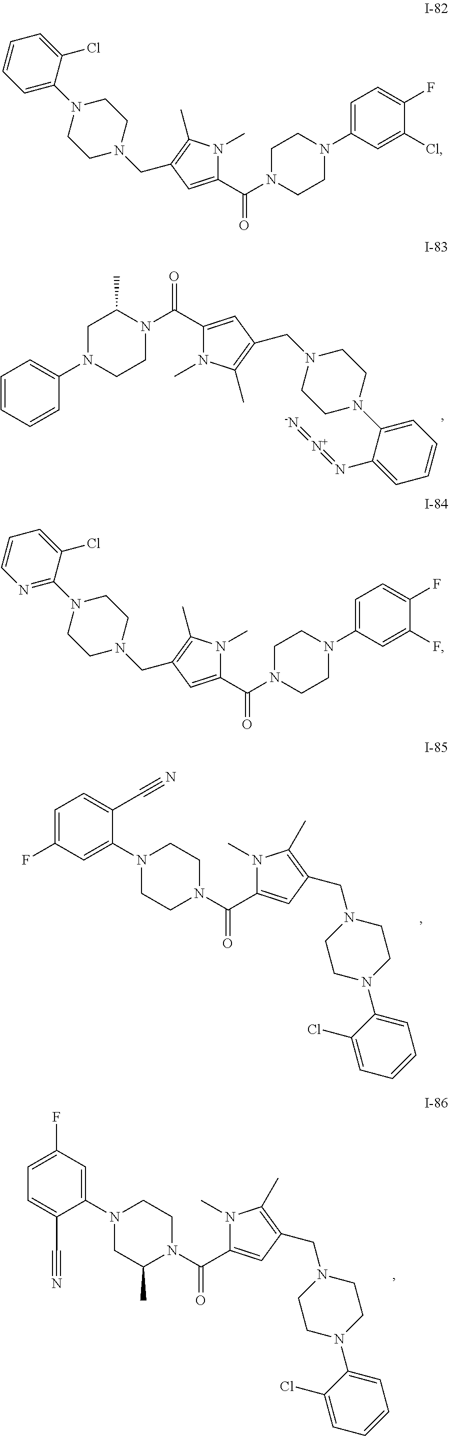 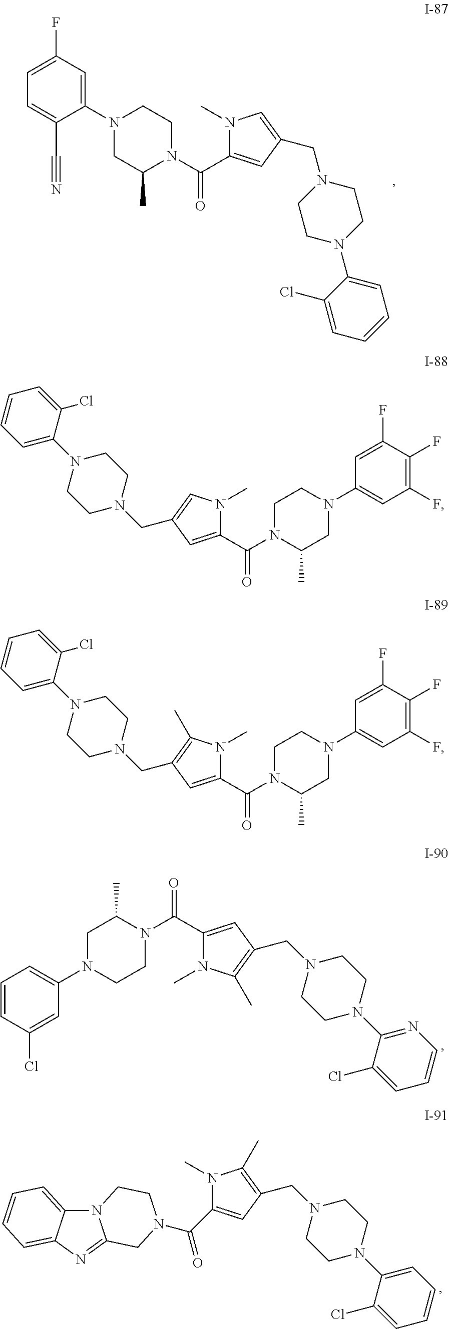 [SMILES: C[C@H]1CN(c2cc(F)ccc2C#N)CCN1C(=O)c1cc(CN2CCN(c3ccccc3Cl)CC2)cn1C ; C[C@H]1CN(c2cc(F)c(F)c(F)c2)CCN1C(=O)c1cc(CN2CCN(c3ccccc3Cl)CC2)cn1C ; Cc1c(CN2CCN(c3ccccc3Cl)CC2)cc(C(=O)N2CCN(c3cc(F)c(F)c(F)c3)C[C@@H]2C)n1C ; Cc1c(CN2CCN(c3ncccc3Cl)CC2)cc(C(=O)N2CCN(c3cccc(Cl)c3)C[C@@H]2C)n1C ; Cc1c(CN2CCN(c3ccccc3Cl)CC2)cc(C(=O)N2CCn3c(nc4ccccc43)C2)n1C] 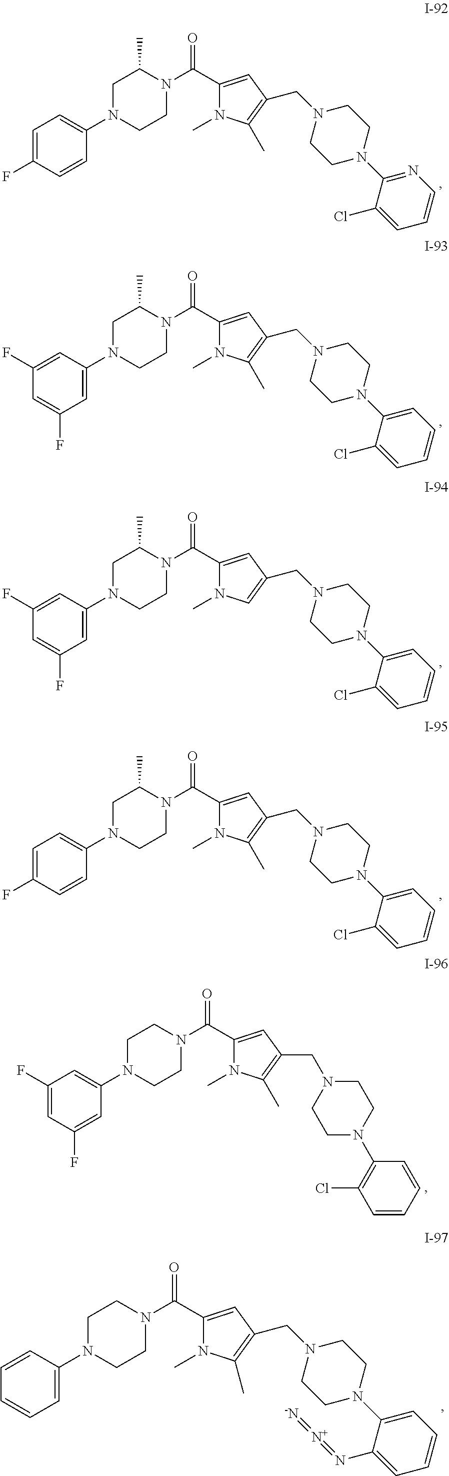 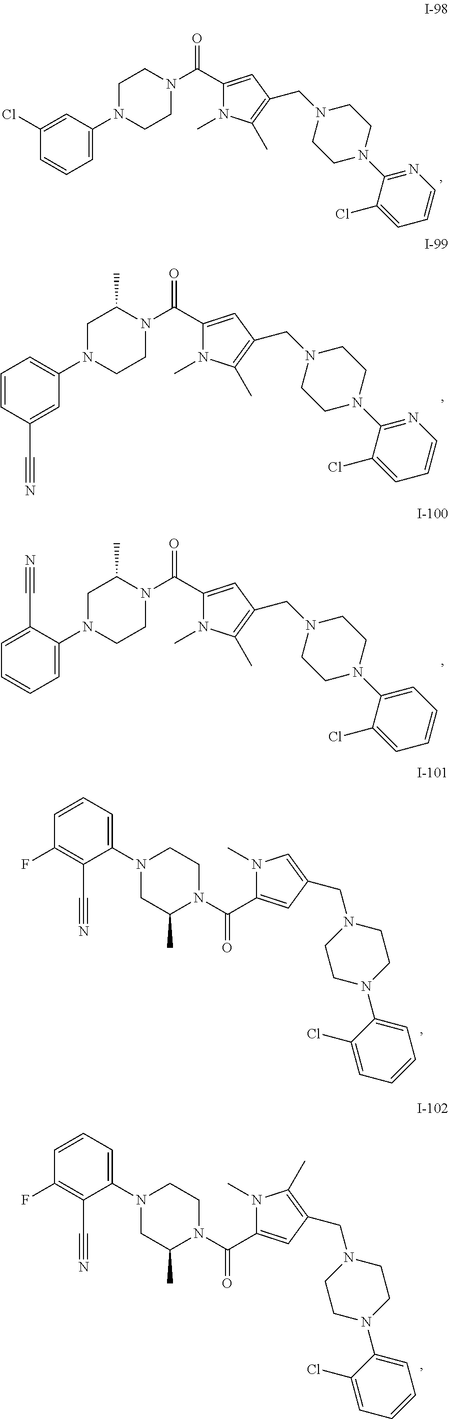 [SMILES: Cc1c(CN2CCN(c3ncccc3Cl)CC2)cc(C(=O)N2CCN(c3cccc(Cl)c3)CC2)n1C ; Cc1c(CN2CCN(c3ncccc3Cl)CC2)cc(C(=O)N2CCN(c3cccc(C#N)c3)C[C@@H]2C)n1C ; Cc1c(CN2CCN(c3ccccc3Cl)CC2)cc(C(=O)N2CCN(c3ccccc3C#N)C[C@@H]2C)n1C ; C[C@H]1CN(c2cccc(F)c2C#N)CCN1C(=O)c1cc(CN2CCN(c3ccccc3Cl)CC2)cn1C ; Cc1c(CN2CCN(c3ccccc3Cl)CC2)cc(C(=O)N2CCN(c3cccc(F)c3C#N)C[C@@H]2C)n1C] 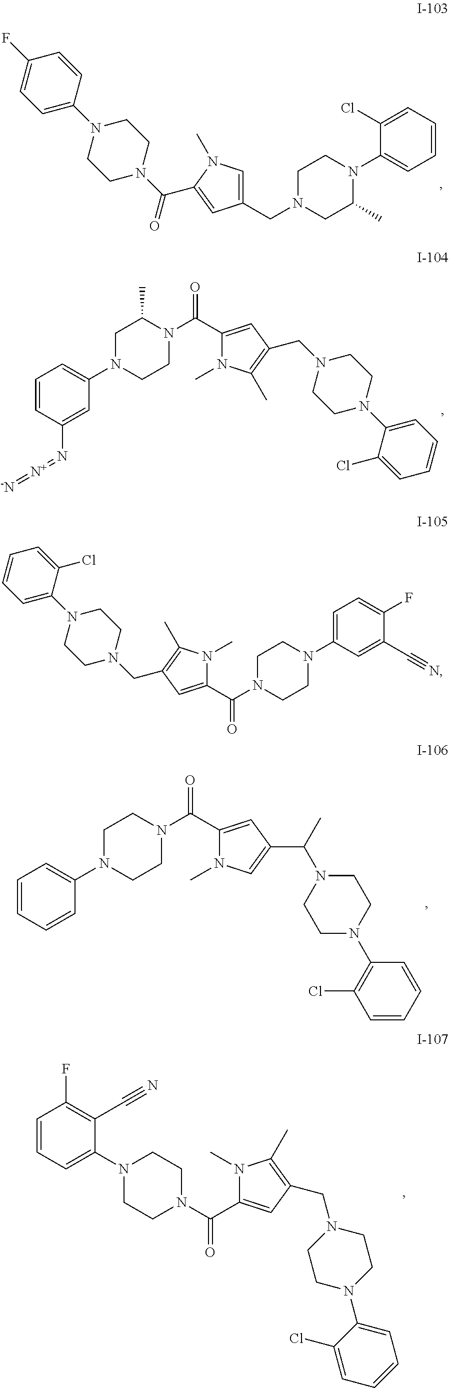 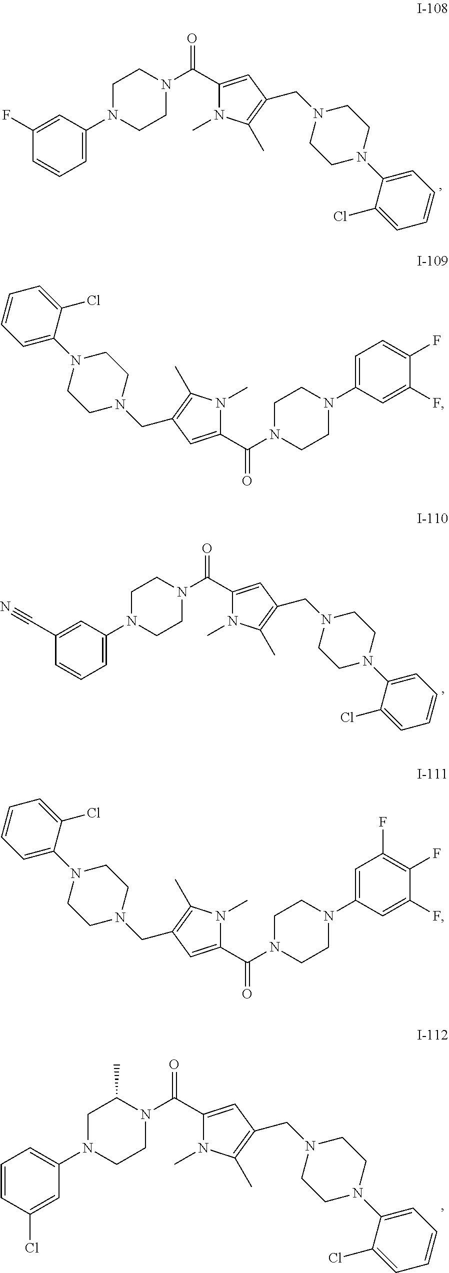 [SMILES: Cc1c(CN2CCN(c3ccccc3Cl)CC2)cc(C(=O)N2CCN(c3cccc(F)c3)CC2)n1C ; Cc1c(CN2CCN(c3ccccc3Cl)CC2)cc(C(=O)N2CCN(c3ccc(F)c(F)c3)CC2)n1C ; Cc1c(CN2CCN(c3ccccc3Cl)CC2)cc(C(=O)N2CCN(c3cccc(C#N)c3)CC2)n1C ; Cc1c(CN2CCN(c3ccccc3Cl)CC2)cc(C(=O)N2CCN(c3cc(F)c(F)c(F)c3)CC2)n1C ; Cc1c(CN2CCN(c3ccccc3Cl)CC2)cc(C(=O)N2CCN(c3cccc(Cl)c3)C[C@@H]2C)n1C] 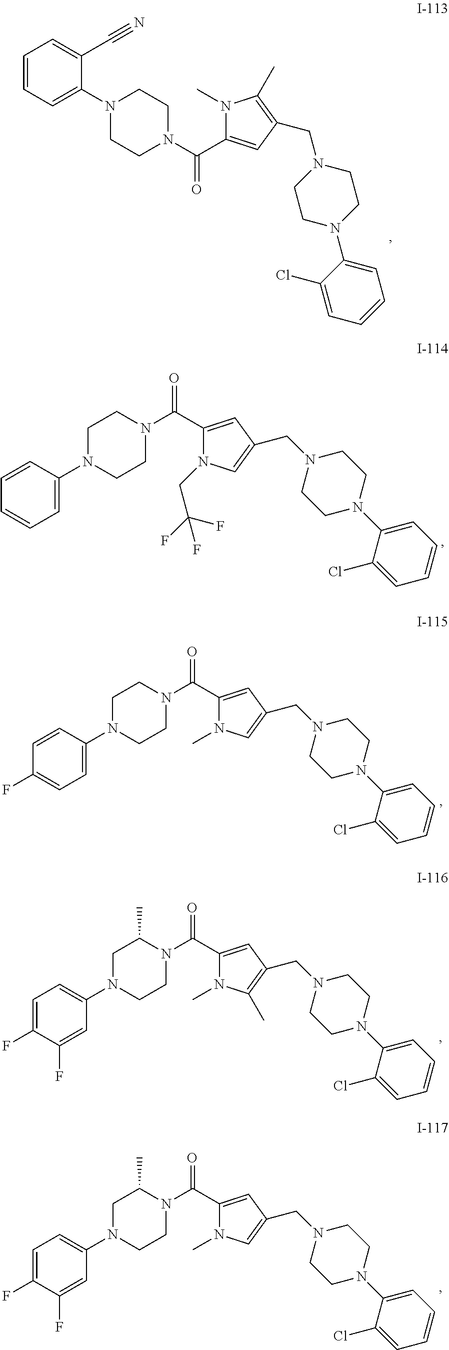 [SMILES: Cc1c(CN2CCN(c3ccccc3Cl)CC2)cc(C(=O)N2CCN(c3ccccc3C#N)CC2)n1C ; O=C(c1cc(CN2CCN(c3ccccc3Cl)CC2)cn1CC(F)(F)F)N1CCN(c2ccccc2)CC1 ; Cn1cc(CN2CCN(c3ccccc3Cl)CC2)cc1C(=O)N1CCN(c2ccc(F)cc2)CC1 ; Cc1c(CN2CCN(c3ccccc3Cl)CC2)cc(C(=O)N2CCN(c3ccc(F)c(F)c3)C[C@@H]2C)n1C ; C[C@H]1CN(c2ccc(F)c(F)c2)CCN1C(=O)c1cc(CN2CCN(c3ccccc3Cl)CC2)cn1C] 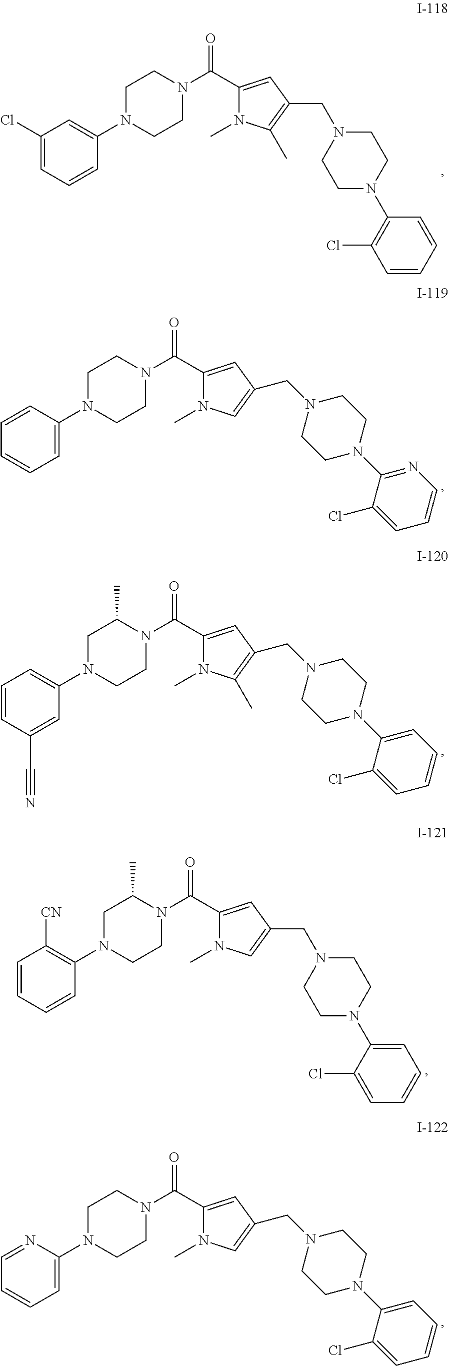 [SMILES: Cc1c(CN2CCN(c3ccccc3Cl)CC2)cc(C(=O)N2CCN(c3cccc(Cl)c3)CC2)n1C ; Cn1cc(CN2CCN(c3ncccc3Cl)CC2)cc1C(=O)N1CCN(c2ccccc2)CC1 ; Cc1c(CN2CCN(c3ccccc3Cl)CC2)cc(C(=O)N2CCN(c3cccc(C#N)c3)C[C@@H]2C)n1C ; C[C@H]1CN(c2ccccc2C#N)CCN1C(=O)c1cc(CN2CCN(c3ccccc3Cl)CC2)cn1C ; Cn1cc(CN2CCN(c3ccccc3Cl)CC2)cc1C(=O)N1CCN(c2ccccn2)CC1] 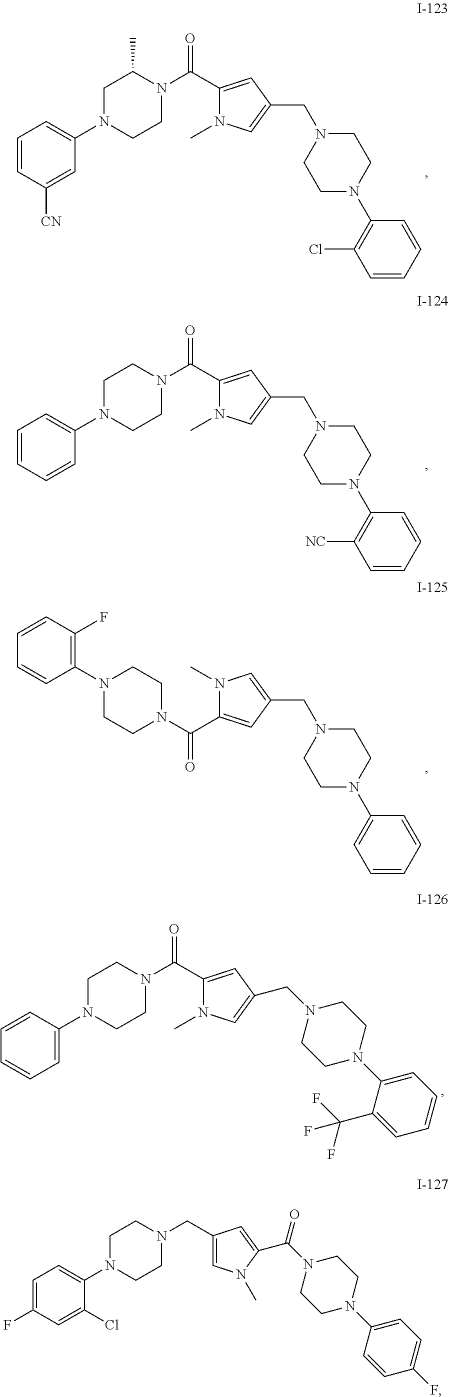 [SMILES: C[C@H]1CN(c2cccc(C#N)c2)CCN1C(=O)c1cc(CN2CCN(c3ccccc3Cl)CC2)cn1C ; Cn1cc(CN2CCN(c3ccccc3C#N)CC2)cc1C(=O)N1CCN(c2ccccc2)CC1 ; Cn1cc(CN2CCN(c3ccccc3)CC2)cc1C(=O)N1CCN(c2ccccc2F)CC1 ; Cn1cc(CN2CCN(c3ccccc3C(F)(F)F)CC2)cc1C(=O)N1CCN(c2ccccc2)CC1 ; Cn1cc(CN2CCN(c3ccc(F)cc3Cl)CC2)cc1C(=O)N1CCN(c2ccc(F)cc2)CC1] 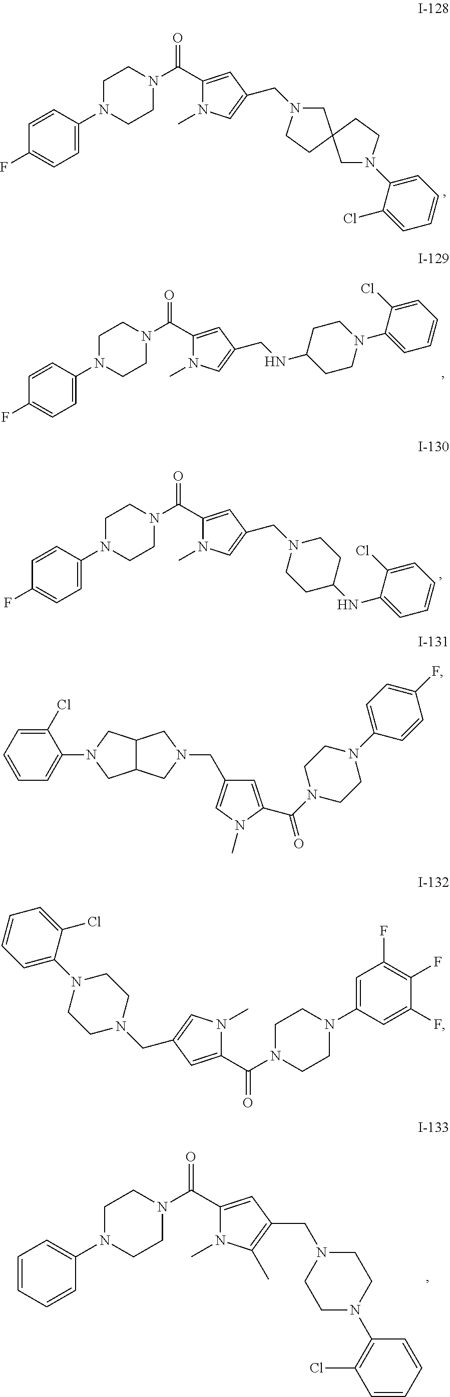 [SMILES: Cn1cc(CN2CCC3(CCN(c4ccccc4Cl)C3)C2)cc1C(=O)N1CCN(c2ccc(F)cc2)CC1 ; Cn1cc(CNC2CCN(c3ccccc3Cl)CC2)cc1C(=O)N1CCN(c2ccc(F)cc2)CC1 ; Cn1cc(CN2CCC(Nc3ccccc3Cl)CC2)cc1C(=O)N1CCN(c2ccc(F)cc2)CC1 ; Cn1cc(CN2CC3CN(c4ccccc4Cl)CC3C2)cc1C(=O)N1CCN(c2ccc(F)cc2)CC1 ; Cn1cc(CN2CCN(c3ccccc3Cl)CC2)cc1C(=O)N1CCN(c2cc(F)c(F)c(F)c2)CC1 ; Cc1c(CN2CCN(c3ccccc3Cl)CC2)cc(C(=O)N2CCN(c3ccccc3)CC2)n1C] 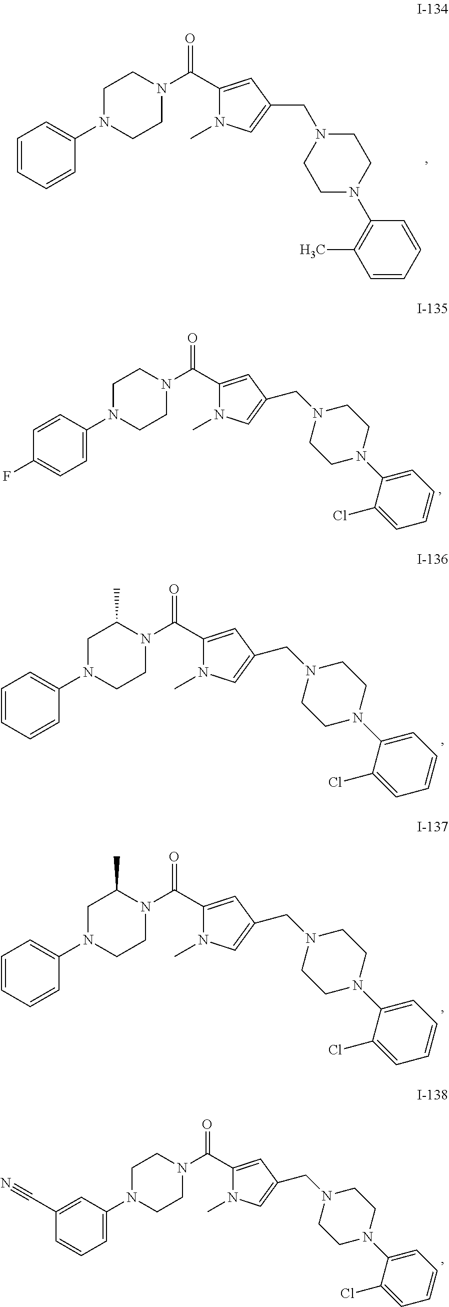 [SMILES: Cc1ccccc1N1CCN(Cc2cc(C(=O)N3CCN(c4ccccc4)CC3)n(C)c2)CC1 ; Cn1cc(CN2CCN(c3ccccc3Cl)CC2)cc1C(=O)N1CCN(c2ccc(F)cc2)CC1 ; C[C@H]1CN(c2ccccc2)CCN1C(=O)c1cc(CN2CCN(c3ccccc3Cl)CC2)cn1C ; C[C@@H]1CN(c2ccccc2)CCN1C(=O)c1cc(CN2CCN(c3ccccc3Cl)CC2)cn1C ; Cn1cc(CN2CCN(c3ccccc3Cl)CC2)cc1C(=O)N1CCN(c2cccc(C#N)c2)CC1] 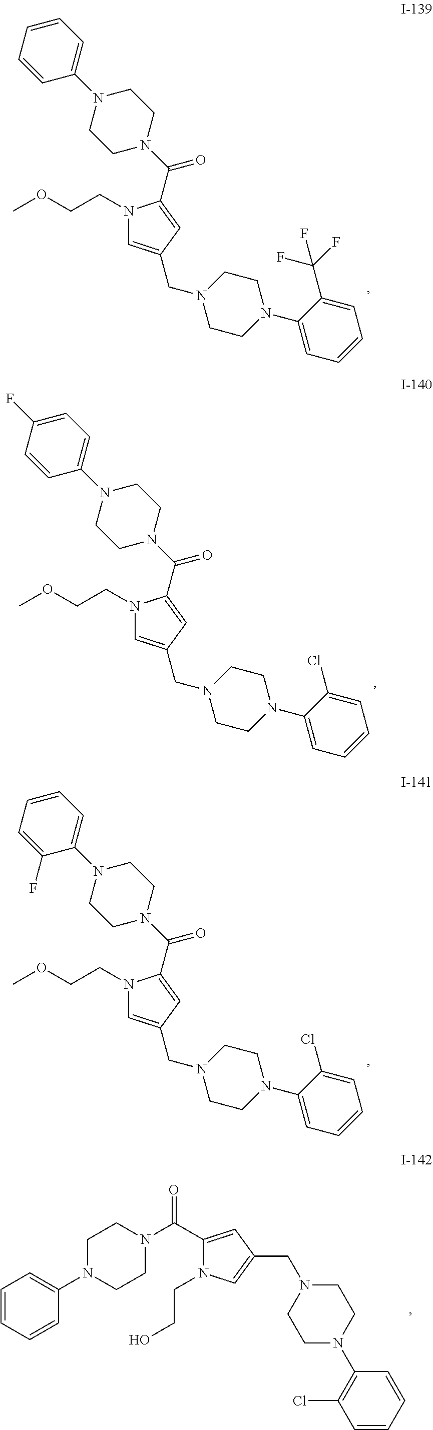 [SMILES: COCCn1cc(CN2CCN(c3ccccc3C(F)(F)F)CC2)cc1C(=O)N1CCN(c2ccccc2)CC1 ; COCCn1cc(CN2CCN(c3ccccc3Cl)CC2)cc1C(=O)N1CCN(c2ccc(F)cc2)CC1 ; COCCn1cc(CN2CCN(c3ccccc3Cl)CC2)cc1C(=O)N1CCN(c2ccccc2F)CC1 ; O=C(c1cc(CN2CCN(c3ccccc3Cl)CC2)cn1CCO)N1CCN(c2ccccc2)CC1] 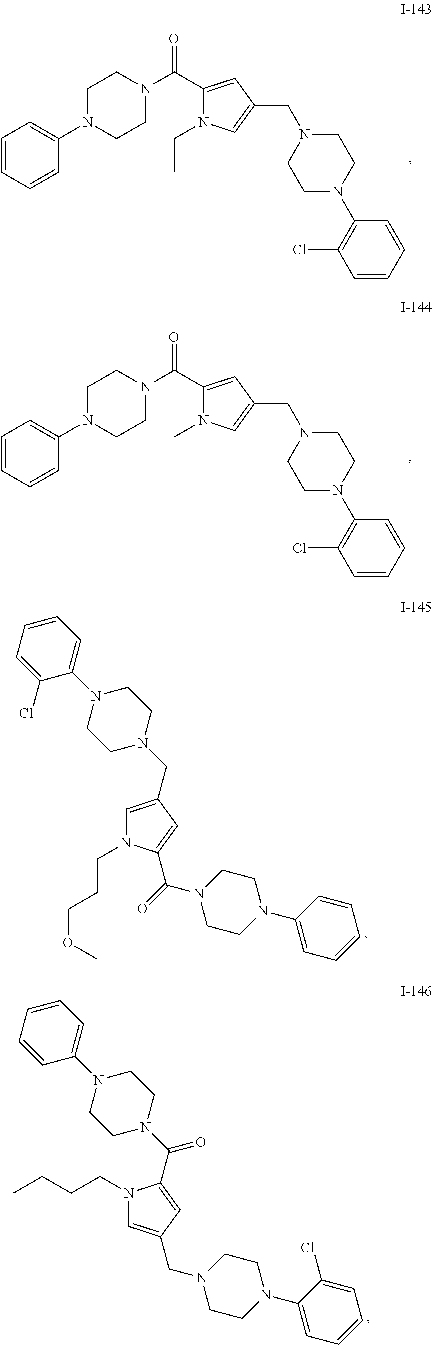 [SMILES: CCn1cc(CN2CCN(c3ccccc3Cl)CC2)cc1C(=O)N1CCN(c2ccccc2)CC1 ; Cn1cc(CN2CCN(c3ccccc3Cl)CC2)cc1C(=O)N1CCN(c2ccccc2)CC1 ; COCCCn1cc(CN2CCN(c3ccccc3Cl)CC2)cc1C(=O)N1CCN(c2ccccc2)CC1 ; CCCCn1cc(CN2CCN(c3ccccc3Cl)CC2)cc1C(=O)N1CCN(c2ccccc2)CC1] 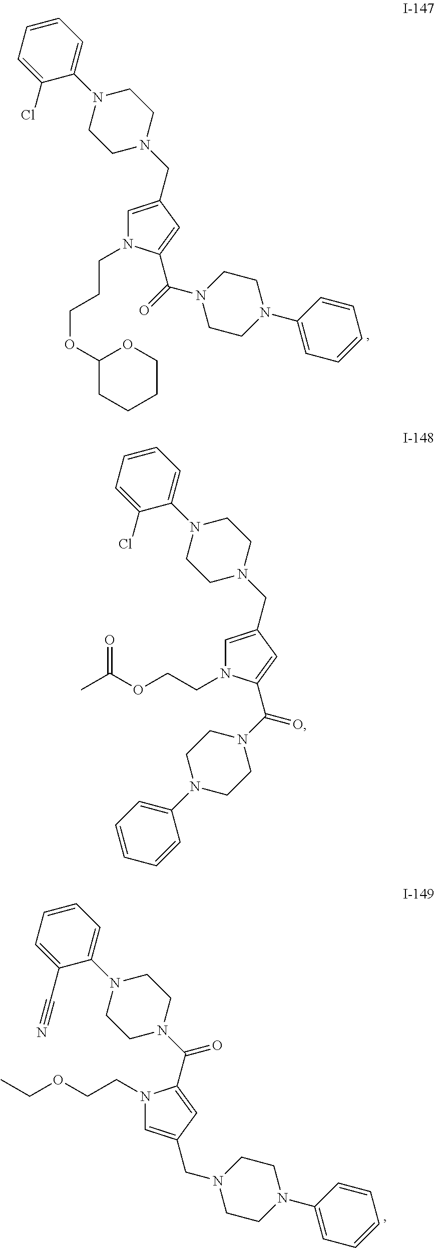 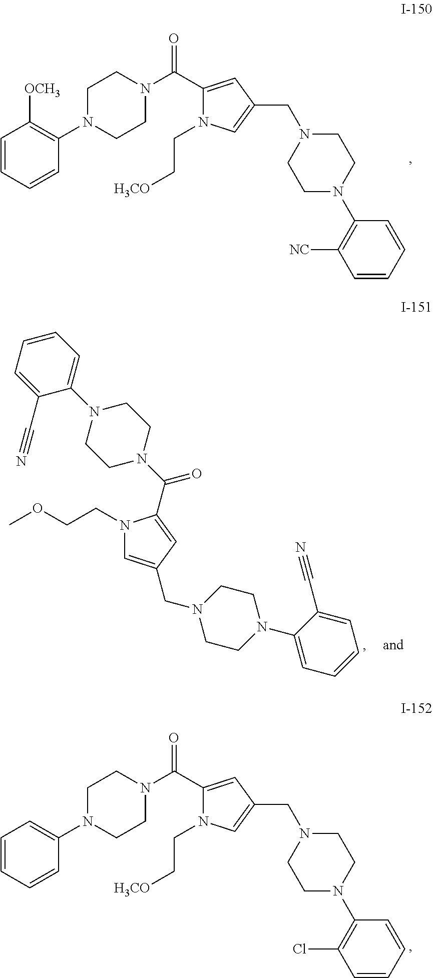 [SMILES: COCCn1cc(CN2CCN(c3ccccc3C#N)CC2)cc1C(=O)N1CCN(c2ccccc2OC)CC1 ; COCCn1cc(CN2CCN(c3ccccc3C#N)CC2)cc1C(=O)N1CCN(c2ccccc2C#N)CC1 ; COCCn1cc(CN2CCN(c3ccccc3Cl)CC2)cc1C(=O)N1CCN(c2ccccc2)CC1]
or a pharmaceutically acceptable salt there of.

25. A pharmaceutically acceptable composition comprising a compound of claim 1, and a pharmaceutically acceptable carrier, adjuvant, or vehicle.

26. A method for treating a mTORC-mediated disorder in a patient in need thereof, comprising administering to said patient the compound of claim 1, or a pharmaceutical composition thereof.

27. A method of treating cystic fibrosis in a patient in need thereof, comprising administering to said patient the compound of claim 1, wherein said compound is an inhibitor of glucose transporters 1, 2, 3, 4 and 5, or a pharmaceutical composition thereof.
